(19) United States
(12) Patent Application Publication
(10) Pub. No.: US 2026/0308519 A1
(43) Pub. Date: Oct. 8, 2026

(54) MODULATION OF THE BTLA-HVEM AXIS TO ENHANCE ADOPTIVE CELL TRANSFER IMMUNOTHERAPY

(71) Applicant: THE TRUSTEES OF THE UNIVERSITY OF PENNSYLVANIA, Philadelphia, PA (US)

(72) Inventors: Marco Ruella, Ardmore, PA (US); Puneeth Guruprasad, Philadelphia, PA (US)

(21) Appl. No.: 19/485,188

(22) PCT Filed: May 17, 2024
(86) PCT No.: PCT/US2024/029978

§ 371 Date: Nov. 17, 2025

Related U.S. Application Data
(60) Provisional application No. 63/503,054, filed on May 18, 2023.

(51) Int. Cl. C12N 5/0783; A61K 35/17; A61K 40/11; A61K 40/31; A61K 40/42; A61P 35/00; C07K 14/705

(52) U.S. Cl. CPC C12N 5/0636; A61K 35/17; A61K 40/11; A61K 40/31; A61K 40/4211; A61P 35/00; C07K 14/70578; C07K 14/70596; C12N 2510/00

(57) ABSTRACT

The present disclosure provides an immune cell genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM). Also provided are methods and uses of the immune cell for treating cancer. Related nucleic acids, vectors, and pharmaceutical compositions are also provided.

### CROSS-REFERENCE TO RELATED APPLICATION

The present application claims priority under 35 U.S.C. § 119(e) to U.S. Provisional Patent Application No. 63/503,054, filed May 18, 2023, which application is hereby incorporated by reference in its entirety herein.

### REFERENCE TO AN ELECTRONIC SEQUENCE LISTING

The present application contains a Sequence Listing which has been submitted in XML format via Patent Center and is hereby incorporated by reference in its entirety. Said XML file, created on May 14, 2024, is named 046483-7425WO1 Sequence Listing.xml and is 77,824 bytes in size.

### BACKGROUND OF THE INVENTION

Adoptive cell transfer immunotherapy and, in particular, chimeric antigen receptor (CAR) T-cell immunotherapy, has shown unprecedented responses and significant improvement in the clinical outcomes of patient with relapsed/refractory (r/r) CD19+ B cell malignancies (Lee, et al., The Lancet (2015) 385(9967):517-28; Maude et al., New England Journal of Medicine (2018) 378(5):439-48; Park et al., New England Journal of Medicine (2018) 378(5)449-59; Schuster, et al., New England Journal of Medicine (2019) 380(1):45-56; Turtle, et al., Science Translational Medicine (2016) 8(355):355ra116-355ra116). Despite the remarkable clinical results of anti-CD19 CART (CART19), greater than 60% of lymphoma patients treated with CART19 still do not respond or eventually relapse (Schuster, et al., New England Journal of Medicine (2019) 380(1):45-56). Additionally, the vast majority of patients treated with the currently approved CART products have failed these treatments, and CAR T cells lack efficacy in the fight against solid tumors due to a number of challenges. A major barrier to the efficacy of cellular immunotherapies in cancer is the suppressive tumor microenvironment (TME). There is a need in the art for enhancing the anti-tumor efficacy of adoptive cell transfer immunotherapies in order to improve the clinical outcome of patients. The present invention addresses this need.

### SUMMARY OF THE INVENTION

In one aspect, the present disclosure provides an immune cell genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM).

In some embodiments, the immune cell further comprises a partial knockdown or knockout of expression of an endogenous BTLA gene.

In some embodiments, the immune cell further comprises a partial knockdown or knockout of expression of an endogenous HVEM gene.

In some embodiments, the knockout is mediated by a gene editing system.

In some embodiments, the knockout is mediated by a CRISPR system.

In some embodiments, the BTLA comprises reduced ability to bind SHP-1 and SHP-2, and the HVEM comprises reduced ability to bind to BTLA.

In some embodiments, the immune cell comprises an expression vector comprising a first nucleotide sequence encoding the CAR or the TCR, a second nucleotide sequence encoding the BTLA or the HVEM, and a third nucleotide sequence comprising a ribosomal skipping sequence, wherein the third nucleotide sequence is located between the first and second nucleotide sequences.

In some embodiments, the immune cell comprises an expression vector comprising a first nucleotide sequence encoding the CAR or the TCR, and the immune cell further comprises a second nucleotide sequence encoding the BTLA or the HVEM, wherein the second nucleotide sequence is integrated into genomic DNA of the immune cell.

In some embodiments, the expression vector is a lentiviral vector.

In some embodiments, the second nucleotide sequence encoding the BTLA or the HVEM is integrated into genomic DNA of the immune cell via adenovirus-mediated gene transfer or CRISPR-mediated gene knock-in.

In some embodiments, the BTLA is a human BTLA variant comprising Y257F and Y282F amino acid substitutions.

In some embodiments, the HVEM is a human HVEM variant comprising E52K and/or G89F amino acid substitution(s).

In some embodiments, the immune cell is a T cell, an autologous cell, a human cell, or any combination thereof.

In some embodiments, the CAR comprises an extracellular antigen binding domain, a transmembrane domain, and an intracellular domain.

In some embodiments, the extracellular antigen binding domain comprises a single-chain variable fragment (scFv).

In some embodiments, the CAR or the TCR targets a tumor antigen.

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), gp100, HER2, HLA-A2, HPV E6, ICAM1, IL3Ra, IL13Ra2, LAGE-1, Lewis Y, LMP1 (EBV), MAGE-A1, MAGE-A3, MAGE-A4, MAGE-A6, MAGE-A10, MART-1, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PRAME, PSCA, PSMA, ROR1, ROR2, TIM-3, TM4SF1, TnMuc1, VEGFR2, and any combination thereof.

In some embodiments, the intracellular domain of the CAR comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain of the CAR comprises an intracellular signaling domain of a protein selected from the group consisting of a CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular domain of the CAR comprises a costimulatory domain of CD28, a costimulatory domain of 4-1BB, an intracellular signaling domain of CD3 zeta, or any combination thereof.

In some embodiments, the CAR targets CD19, CD30, or HER-2.

In some embodiments, the TCR targets gp100.

In one aspect, the present disclosure provides a pharmaceutical composition comprising a population of the immune cell described herein and a pharmaceutically acceptable carrier.

In one aspect, the present disclosure provides the pharmaceutical composition described herein, for use in a method of treating cancer in a subject.

In one aspect, the present disclosure provides a method of treating cancer in a subject in need thereof, the method comprising administering to the subject a therapeutically effective amount of immune cells genetically modified to express a chimeric antigen receptor (CAR) or a T cell receptor (TCR), wherein the immune cells comprise altered signaling of B and T lymphocyte attenuator (BTLA) and/or herpes virus entry mediator (HVEM).

In some embodiments, the immune cells further comprise a partial knockdown or knockout of expression of an endogenous BTLA gene.

In some embodiments, the immune cells further comprise a partial knockdown or knockout of expression of an endogenous HVEM gene.

In some embodiments, the knockout is mediated by a gene editing system.

In some embodiments, the knockout is mediated by a CRISPR system.

In some embodiments, the BTLA comprises reduced ability to bind SHP-1 and SHP-2, and wherein the HVEM comprises reduced ability to bind to BTLA.

In some embodiments, the immune cells comprise an expression vector comprising a first nucleotide sequence encoding the CAR or the TCR, a second nucleotide sequence encoding the BTLA or the HVEM, and a third nucleotide sequence comprising a ribosomal skipping sequence, wherein the third nucleotide sequence is located between the first and second nucleotide sequences.

In some embodiments, the immune cells comprise an expression vector comprising a first nucleotide sequence encoding the CAR or the TCR, and the immune cells further comprise a second nucleotide sequence encoding the BTLA or the HVEM, wherein the second nucleotide sequence is integrated into genomic DNA of the immune cell.

In some embodiments, the expression vector is a lentiviral vector.

In some embodiments, the second nucleotide sequence encoding the BTLA or the HVEM is integrated into genomic DNA of the immune cells via adenovirus-mediated gene transfer or CRISPR-mediated gene knock-in.

In some embodiments, the BTLA is a human BTLA variant comprising Y257F and Y282F amino acid substitutions.

In some embodiments, the HVEM is a human HVEM variant comprising E52K and/or G89F amino acid substitution(s).

In some embodiments, the immune cells are T cells, autologous cells, human cells, or any combination thereof.

In some embodiments, the CAR comprises an extracellular antigen binding domain, a transmembrane domain, and an intracellular domain.

In some embodiments, the extracellular antigen binding domain comprises a single-chain variable fragment (scFv).

In some embodiments, the CAR or the TCR targets a tumor antigen.

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), gp100, HER2, HLA-A2, HPV E6, ICAM1, IL3Ra, IL13Ra2, LAGE-1, Lewis Y, LMP1 (EBV), MAGE-A1, MAGE-A3, MAGE-A4, MAGE-A6, MAGE-A10, MART-1, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PRAME, PSCA, PSMA, ROR1, ROR2, TIM-3, TM4SF1, TnMuc1, VEGFR2, and any combination thereof.

In some embodiments, the intracellular domain of the CAR comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KR).

In some embodiments, the intracellular domain of the CAR comprises an intracellular signaling domain of a protein selected from the group consisting of a CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular domain of the CAR comprises a costimulatory domain of CD28, a costimulatory domain of 4-1BB, an intracellular signaling domain of CD3 zeta, or any combination thereof.

In some embodiments, the CAR targets CD19, CD30, or HER-2.

In some embodiments, the TCR targets gp100.

In some embodiments, the cancer is an HVEM-positive cancer.

In some embodiments, the subject is a human.

### BRIEF DESCRIPTION OF THE DRAWINGS

The foregoing and other features and advantages of the present invention will be more fully understood from the following detailed description of illustrative embodiments taken in conjunction with the accompanying drawings.

FIGS. 1A-1E show that the BTLA-HVEM axis is enriched in Hodgkin Lymphoma. FIG. 1A: UMAP projection of scRNA-seq data of 4 cHL patients (Penn) and 5 reactive lymph nodes from Aoki et al. (Aoki, et al. Cancer Discovery 10, 406-421 (2020)). scRNA-seq was performed using the 10× Genomics platform. Data was processed using Seurat (Hao, et al., Cell 184, 3573-3587.e3529 (2021); Stuart, et al., Cell 177, 1888-1902.e1821 (2019)). FIG. 1B: CellPhoneDB (Efremova, et al., Nat Protoc 15, 1484-1506 (2020)) prediction of interacting ligand-receptor pairs between effector CD4+ T cells and Treg or monocytes, plotting enrichment in HL over healthy RLN. “Enrichment” for interactions between two cellular subsets is defined as the [sum of sig means (HL patients)—sum of sig means (RLN healthy subjects)], where the “sig_means” value is that which is calculated by CellPhoneDB for the given interaction. Interactions with sig means>0 and that are shared between HL patients or RLN samples are included in this analysis. FIG. 1C: BTLA or HVEM expression across the TME, using Seurat's FeaturePlot function. FIG. 1D: Analysis of overall and disease-free survival of all TCGA cohorts based on BTLA expression normalized to CD3E. Cutoffs are defined by quartiles. This is performed using GEPIA2 (gepia2.cancer-pku.cn) (Tang, et al., Nucleic Acids Res 47, W556-w560 (2019)). FIG. 1E: Schematic of the BTLA-HVEM axis, highlighting both trans and cis interaction.

FIGS. 2A-2F show analyses of single-cell clusters in HL Patient cohort and validation cohort. FIG. 2A: UMAP of all clusters, and sub-clusters, amongst merged HL and RLN populations from exploratory (Penn) and validation (Aoki, et al. Cancer Discovery 10, 406-421 (2020)) scRNA-seq cohorts. FIG. 2B: UMAP of n=22 HL patients from Aoki et al. (Aoki, et a. Cancer Discovery 10, 406-421 (2020)). FIG. 2C: Ranked interactions across entire TME of n=22 HL patients, regardless of subset. To sort overall interactions, a RankSum=rank₁+rank₂ was calculated. Here, rank1 is based on the number of times an interaction was found in the cohort (e.g., CellPhoneDB detected significant interaction in 14/22 patients); and rank2 is based on the average “rank” value returned by CellPhoneDB. FIGS. 2D-2F: CellPhoneDB rankings, calculated by adding “sig_means” values from individual subsets from each of 22 patients. FIG. 2D: sum of sig_means from individual T cell subsets vs Treg, respectively, for all patients. FIG. 2E: total sum of sig_means from interactions of each active CD4 T cell subset (i.e., CD4 proliferating, CD4 TCM, CD4 TEM) vs Treg, respectively, for all patients. Importantly, other interactions involving TNFRSF14 (i.e., CD160-TNFRSF14 (inhibitory) and TNFSF14-TNFRSF14 (stimulatory)) appear enriched in Treg interactions. FIG. 2F: total sum of sig_means from interactions of each active CD4 T cell subset (i.e., CD4 proliferating, CD4 TCM, CD4 TEM) vs monocyte populations (CD14 and CD16 monocytes).

FIGS. 3A-3H show screening of CAR30 scFv binders. FIG. 3A: 11 anti-CD30 CAR constructs were developed and screened for transduction efficiency in primary human T cells from three healthy human donors. CAR30 surface expression was detected using an anti-G4S-linker antibody. HRS-3 is a control gold-standard clone. Low transduction efficiency candidates (T1-8, T1-9, T2-2) were not assessed further. FIG. 3B: Short-term tumor killing of CAR30 candidates from two donors against CD30-HEK293T. FIG. 3C: Short-term tumor killing of CAR30 candidates from two donors against SU-DHL-1. FIG. 3D: IFNγ concentration (ELISA) of supernatant following 24 hr coculture with SU-DHL-1 (effector: target=0.3). FIG. 3E: Expansion of UTD, HRS-3, T1-36, and T1-159, revealing fratricide of T1-36 during manufacturing. FIG. 3F: Assessment of off-target toxicity of lead candidates using CD30− cell lines (OCI-Ly18, Nalm6, and Raji-B). Tumor killing is shown after 48 hr against OCI-Ly18 (left) and Nalm6 (middle, effector: target=2), and IFNγ concentration (ELISA) is shown after 24 hr against Raji-B (right, effector: target=1). FIG. 3G: Tumor volumes of in vivo study to comparing T1-159 and HRS-3 CAR30 T cells. Briefly, 15e6 HDLM-2 cells were inoculated into the right flank of NSG mice, and infused at week 8 with 3e5 CAR30+ (T1-159 or HRS-3) T cells (n=4 mice per CAR30 construct). Caliper measurements were taken once per week following infusion. Statistical significance was determined by multiple unpaired t-test. FIG. 3H: Schema depicting overall selection process which led to a lead CAR30 construct, T1-159.

FIGS. 4A-4G show manufacturing and additional assays of BTLA KO CART30 cells. FIG. 4A: Schematic depicting timeline for CRISPR-edited human CAR T cell manufacturing. FIG. 4B: Representative analysis of indels in exon 2 of BTLA following CRISPR-Cas9 KO, using the TIDE (Tracking of Indels by DEcomposition) software (https://tide.nki.nl/) (Brinkman, et al., Nucleic Acids Res 42, e168 (2014)). FIG. 4C: Prediction of off-targeting knockouts from optimized BTLA sgRNA, showing no additional targets (Bae, et al., Bioinformatics 30, 1473-1475 (2014)). FIG. 4D: Expression of T1-159 CAR30 in primary human T cells at day 6 (de-bead) of expansion, measured via flow cytometry and an anti-G4S linker antibody. FIG. 4E: Expansion metrics of T1-159 CAR30 for a single donor (ND518), including population doublings (upper left), total cells (upper right), cell size (lower left), and viability (lower right). FIG. 4F: IL-2 and IFNγ concentration (ELISA) in supernatant following 24 hr coculture (effector: target=1; 50,000 HDLM-2 cells, n=3 per condition). Statistical significance was determined by one-way ANOVA with post hoc Tukey tests. FIG. 4G: left: cytotoxicity of WT UTD, BTLA KO UTD, WT CART30, and BTLA KO CART30 against HDML-2 at 48 hr (n=2 per condition); right: Concentration of TNF in supernatant after 24 hr of coculture with HDLM-2 (effector: target=1, n=3 per condition). Statistical significance was determined by one-way ANOVA with post hoc Tukey tests. ns, not significant. EP: electroporation.

FIGS. 5A-5L show that BTLA knockout in CD30-directed CAR T cells augments anti-tumor function against Hodgkin lymphoma. FIG. 5A: Flow cytometry of BTLA surface expression on CAR30 (clone T1-159) T cells after mock electroporation (left) or CRISPR-Cas9 mediated deletion of BTLA (right). Also shows are (i) flow cytometry of BTLA surface expression on CAR19 T cells after mock electroporation (CAR19 WT) or CRISPR-Cas9 mediated deletion of BTLA (BTLA KO), (ii) flow cytometry of HVEM surface expression on CAR19 T cells after mock electroporation (CAR19 WT) or CRISPR-Cas9 mediated deletion of HVEM (HVEM KO), (iii) a chart showing the knockout (KO) score for BTLA and HVEM KO in CAR19 T cells and (iv) a table showing the guide RNA (gRNA) sequences used to generate the knockouts (SEQ ID NO: 1 and SEQ ID NO: 2). FIG. 5B: T cell subset phenotyping of WT CAR30 T cells and BTLA KO CAR30 T cells at the end of ex vivo expansion, during a resting phase, in a representative healthy donor (TCM—T central memory; TEM—T effector memory, TEMRA—T effector memory re-expressing CD45RA). FIG. 5C: Comparison % TCM T cells in three independent healthy donors. Statistical significance was determined by paired t-test. FIG. 5D: Long-term coculture of UTD, WT, or BTLA KO CAR30 T cells with CD30+ cHL cell line, HDLM-2; seeding effector: target ratio of 0.5 (n=2 per condition). left: fold change of HDLM-2 cells in coculture; right: fold change of total T cells in coculture. Also shown are (i) a graph showing that BTLA KO CAR30 T cells and HVEM KO CAR30 T cells each have significantly improved tumor control in vitro and (ii) a graph showing that HVEM KO CAR30 T cells and, in particular, BTLA KO CAR30 T cells, each have significantly improved expansion in vitro. FIG. 5E: UTD, WT, or BTLA KO CART30 were labelled with CellTraceViolet (CTV) and seeded in an effector: target ratio of 0.25 with irradiated HDLM-2 cells (90 Gy) (n=2 per condition). Representative flow histogram of proliferation at t=120 h. FIG. 5F: Fold change of total CD3+ T cells in coculture from CTV proliferation experiment shown in FIG. 5E. FIG. 5G: Data showing that BTLA KO in CAR19 T cells attenuates recruitment of SHP-1, along with MFI of phospho-(p) SHP-1/MFI of pSHP-1 at t=0 min (i.e., fold-change of MFI pSHP-1 over MFI pSHP-1 at 0 min) following coculture of CART30 with HDLM-2 from 0 to 30 min (n=2 technical replicates). FIG. 5H: Design of in vivo study using HDLM-2 and NSG mice. 15×10⁶ HDLM-2 cells were injected subcutaneously into the right flank of NSG mice. At day 62 post-implantation, 2.5×10⁵ UTD (n=6), WT (n=7), or BTLA KO (n=9) CAR30+ T cells were adoptively transferred via tail vein injection. FIG. 5I: Average (+/−SD) tumor growth curve for each condition. FIG. 5J: Average (+/−SD) total human CD45+ CD3+ T cells per 100 μL peripheral blood during the course of in vivo expansion. Statistics are shown for the final time-point (week 9) data only. FIG. 5K: At day 7 post-CART30 infusion, peripheral blood of each mouse was collected via retro-orbital bleeding, and serum was isolated. Concentration of key cytokines in individual mouse serum was analyzed via Luminex®. FIG. 5L: Overall survival of mice. Statistical significance was determined by one-way analysis of variance (ANOVA) with post hoc Tukey tests (FIG. 5F, FIG. 5K) between CART30 and BTLA KO conditions, two-way ANOVA (FIGS. 5D, 5G, 5I, 5J), or long-rank Mantel Cox test (FIG. 5L). **, P<0.01; **** P<0.0001.

FIGS. 6A-6J show that BTLA knockout enhances 4-1BBζ CART19 anti-tumor function in non-Hodgkin lymphoma. FIG. 6A: Analysis of TCGA DLBCL cohort based on BTLA expression normalized to CD3E, performed via GEPIA2 (gepia2.cancer-pku.cn). FIG. 6B: Bulk RNA sequencing of FACS-isolated OCI-Ly18 following 48 hr coculture with CART19 (effector:target=0.125, n=3 technical replicates). FIG. 6C: Tumor killing after 48 hr coculture of UTD, WT, or BTLA KO CART19 against click-beetle-green (CBG)+ OCI-Ly18, at the shown effector: target ratios (50,000 OCI-Ly18 per well; n=2 technical replicates). Percent killing was calculated using luminescence values. FIG. 6D: Cell-to-cell avidity between UTD (n=4 runs), WT (n=3), and BTLA KO (n=3) CAR19 T cells and OCI-Ly18 using the LUMICKS Z-Movi platform. Statistics are calculated at 500 pN. FIG. 6E: Design of in vivo study using OCI-Ly18 and NSG mice. 5×10⁶ OCI-Ly18 cells were inoculated subcutaneously into the right flank of NSG mice. At day 14 post-implantation, 3×10⁶ UTD (n=5), WT (n=6), or BTLA KO (n=7) CAR19+ T cells infused via tail vein. FIG. 6F: A graph showing significantly reduced tumor size in mice injected with BTLA KO CAR19 T cells or BTLA/HVEM dual KO CAR19 T cells and a chart showing bioluminescent intensity at day 10 post-CART19 infusion of mice imaged laterally (right flank facing up). FIG. 6G: Average OCI-Ly18 tumor volumes, also showing individual mice tumor growth curves, following CART19 infusion. Statistics compare mean of WT CART19 and BTLA KO CART19 at day 14. FIG. 6H: Total human CD45+ CD3+ T cells per 100 μL peripheral blood at days 7 and 14 post-CAR19 infusion, per mouse and per condition. FIG. 6I: Charts showing significantly enhanced cytokine production in BTLA KO CAR30 T cells, and a chart of cytokine concentrations of individual mouse serum collected at day 7 post-CART19 infusion analyzed via Luminex®. FIG. 6J: Overall survival of mice. Statistical significance was determined by DESeq2 (Wald test) (FIG. 6B), one-way ANOVA with post hoc Tukey tests (FIGS. 6C, 6D, 6F, 6G, 6I), two-way ANOVA (FIG. 6H), or long-rank Mantel Cox test (FIGS. 6A, 6J). ns, not significant; *, P<0.05; **, P<0.01; ***, P<0.001; ****, P<0.0001.

FIGS. 7A-7H show data related to BTLA knockout in CART19 against DLBCL. FIG. 7A: Bulk RNA sequencing of FACS-isolated SU-DHL-4 following 48 hr coculture with CART19 (effector: target=0.125, n=3 technical replicates). FIG. 7B: Luminescence-based in vitro cytotoxicity (72 hr) against SU-DHL-4. FIGS. 7C-7G relate to a chronic-antigen-exposure experiment of WT or BTLA KO CART19 with OCILy18. FIG. 7C: (i) bulk RNA sequencing results of purified WT or BTLA KO CAR T cells from chronic antigen-stimulation experiment (n=2 donors) showing reduced RNA expression of FOXP3 and exhaustion markers (such as CTLA-4 and HAVCR2) after two weeks in BTLA KO CAR T cells (DeSEQ2-calculated DEGs between WT and BTLA KO CAR T cells), (ii) data showing that BTLA KO restricts induction of FOXP3 in CAR30 T cells upon extended (5 days) co-culture with HDLM-2 (HVEM-high) tumor cells (E:T=0.5), and (iii) Design of the CAE experiment. 1e6 CAR T cells were seeded with OCILy18 in on day 0 (effector: target=0.5, n=3 technical replicates). On days 3,7, and 11, flow cytometry was performed to determine the absolute counts of CAR T cells per well. OCI-Ly18 was then added to adjust the effector: target ratio to 0.5 in each well. On day 14, cocultures were collected, centrifuged, and washed 2× in PBS prior to fixing and staining for CyTOF (Standard Biotools MaxPar Kits). FIG. 7D: UMAP projections of CD4+ and CD8+ cells, gated previously on CD3+. Of note, CD4+ cluster 6 and CD8+ cluster 9 are notably, visually distinct between WT and BTLA KO cells. FIG. 7E: Distribution (or percent participation) of CD4+ T cells in WT and BTLA KO amongst ten independent clusters. FIG. 7F: Distribution (or percent participation) of CD8+ T cells in WT and BTLA KO amongst ten independent clusters. For FIGS. 7E-7F, a total of 10 clusters were found for both CD4 and CD8 subsets, and the differences between CD4+ cluster 6 and CD8+ cluster 9 are statistical. FIG. 7G: Radar plots of MFI for each surface marker within CD4+ cluster 6 and CD8+ cluster 9. FIG. 7H: Raw Luminex® values used to generate heatmap in FIG. 6J. Statistical significance was determined by multiple unpaired t test (FIGS. 7E-7G), or one-way ANOVA with post hoc Tukey tests (FIG. 7H). ns, not significant; *, P<0.05; **, P<0.01; ***, P<0.001; ****, P<0.0001.

FIGS. 8A-8F show that the BTLA-HVEM axis does not restrain CAR T cells in B cell leukemia. FIG. 8A: Luminescence-based in vitro tumor killing of Nalm6 at 48 hr of coculture with UTD, CART19, or BTLA KO CART19 (n=2 technical replicates). FIG. 8B: In vitro tumor killing at 48 hr in Nalm6 transduced to constitutively express HVEM (Nalm6-HVEM) (n=2 technical replicates). FIG. 8C: Design of in vivo Nalm6 study. FIG. 8D: Bioluminescent intensity (IVIS Lumina S5) of mice treated with UTD, WT CART19, or BTLA KO CART19 following infusion. FIG. 8E: Overall survival of Nalm6 bearing mice. FIG. 8F: ELISA of mouse serum collected on day 7 post-infusion, measuring IFNγ (left) and IL-2 (right). Statistical significance was determined by individual one-way ANOVA with post hoc Tukey tests (FIGS. 8A, 8B, 8F), multiple unpaired t-test (FIG. 8D), or long-rank Mantel Cox test (FIG. 8E). ns, not significant; *, P<0.01 **, P<0.01.

FIGS. 9A-9P show that BTLA restricts CAR T cells in the functional TME. FIG. 9A: A20 lymphoma tumor growth of Balb/c mice engrafted on day 0 and injected intraperitoneally 3 times per week with anti-BTLA agonistic antibody (10 mg/kg, clone 6A6) (n=5 mice) or isotype control (n=5 mice). FIG. 9B: Comparison of the genomic locations of the BTLA gene in humans and mice. FIG. 9C: Schematic of A20 murine lymphoma model. Briefly, 2×10⁶ were subcutaneously engrafted into Balb/c mice on day −15. On day −6, spleens were harvested from a separate group of Balb/c mice for murine CAR T cell production. 24 hr (day −1) prior to CAR T cell infusion, mice were administered intraperitoneal cyclophosphamide (100 mg/kg) for lymphodepletion. On day 0, 1×10⁶ UTD (n=5), WT murine CAR19+ T (n=7), or BTLA KO (n=7) murine CAR19+ T cells were infused via tail vein. FIG. 9D: Flow cytometry of murine T cell subsets (CD44, CD62L) following 6-day expansion protocol. FIG. 9E: Average A20 tumor growth curves with replicates of each individual mouse following murine CART19 infusion. FIG. 9F: Overall survival of A20-bearing Balb/c mice. FIG. 9G: Percent weight changes of muCART19 and BTLA KO treated mice from initial weight on the day of infusion. FIGS. 9H-9M relate to analysis of WT or BTLA knockout CD45.1+ TILs on day 7 post-infusion of 1×10⁶ muCART19, with mock-electroporated (WT) muCART19 (n=8) or BTLA KO muCART19 (n=9). The common gating strategy for representative plots is Lymphocytes>Singlets>Live>CD45.1+. FIG. 9H: Exhaustion phenotyping of CD45.1+ TILs showing representative plots for PD-1 and LAG-3 positivity. FIG. 9I: Exhaustion phenotyping of CD45.1+ TILs showing quantification of PD-1, LAG-3 double positive quadrant, split by condition. FIG. 9J: Quantification of CD45.1+ infiltrates per gram of tumor weight, split by condition. FIG. 9K: Functional phenotyping of CD45.1+ TILs via intracellular staining, following 5 hr ex vivo stimulation with PMA/ionomycin (eBioscience™ 500× Cell Stimulation Cocktail)+protein transport inhibitor (BD GolgiStop) showing representative plot of TNF positivity, split by condition. FIG. 9L: Functional phenotyping of CD45.1+ TILs via intracellular staining, following 5 hr ex vivo stimulation with PMA/ionomycin (eBioscience™ 500× Cell Stimulation Cocktail)+protein transport inhibitor (BD GolgiStop) showing quantification of TNF positivity, split by condition. FIG. 9M: Functional phenotyping of CD45.1+ TILs via intracellular staining, following 5 hr ex vivo stimulation with PMA/ionomycin (eBioscience™ 500× Cell Stimulation Cocktail)+protein transport inhibitor (BD GolgiStop) showing quantification of percent GZMB+ CD45.1+ TILs. FIG. 9N: Schema of Treg suppression assay design, based on the sorting of GFP+/− T cells derived from Foxp3^{EGFP} spleens. FIG. 9O: Representative histograms of T cell proliferation following 72 hr of culture with anti-CD3/CD28 stimulation beads (1:1 bead:Tconv) in the presence or absence of GFP+ Treg. Tconv are gated on Lymphocytes>Singlets>Live/Dead>GFP-negative>CellTraceViolet. FIG. 9P: Quantification of Treg suppression on WT or BTLA KO T cells for various Tconv to Treg ratios (1:1, 2:1, 4:1, 8,1). Statistical significance was determined by one-way ANOVA with post hoc Tukey tests (FIG. 9G, including UTD), unpaired t-test (FIGS. 9I, 9J, 9L, 9M, 9P), two-way ANOVA (FIGS. 9A, 9E), or long-rank Mantel Cox test (FIG. 9F). ns, not significant; ****, P<0.001. Tconv: conventional (GFP−) T cells.

FIGS. 10A-10F relate to CRISPR-Cas9 KO of primary murine CAR T cells and A20 TME composition. FIG. 10A: Manufacturing protocol for CRISPR-edited murine CAR T cells FIG. 10B: Knockout efficiency of BTLA in primary murine CAR T cells following expansion prior to CART infusion. FIG. 10C: Cell viability (% Live Cells or % propidium-iodide-negative) during murine CAR T cell expansion. FIG. 10D: A20 subcutaneous tumors (~200 mm³) were established in Foxp3^{EGFP} Balb/c mice, and infused with WT (n=4 mice) or BTLA KO (n=4 mice) CD45.1+ Balb/c-derived muCART19. 7 days post-infusion, tumors were resected and analyzed via flow cytometry. The gating strategy used to identify and quantify host (CD45.2+) Treg is shown. FIG. 10E: A20 subcutaneous tumors (~200 mm³) were established in Foxp3^{EGFP} Balb/c mice, and infused with WT (n=4 mice) or BTLA KO (n=4 mice) CD45.1+ Balb/c-derived muCART19. 7 days post-infusion, tumors were resected and analyzed via flow cytometry. Quantification of various subsets in A20 TME: Treg fraction of host CD4 (upper left), total host CD11b+ monocytes (upper right), fraction of M1 (F4/80+, CD206−) and M2 (F4/80+, CD206+) macrophages (lower left), and fraction of neutrophils (Ly6C+, Ly6G+, lower right). FIG. 10F: M2 macrophage suppression assay to monitor CART30 tumor killing in the presence of M2-differentiated macrophages. In brief, healthy donor monocytes were cultured in media containing 10 ng/mL GM-CSF for 7 days, and then cultured in media containing IL-4 (20 ng/mL) for 24 hr, as previously reported (Ruella, et al., Cancer Discovery 7, 1154-1167 (2017)). M2-polarized macrophages were then seeded with HDLM-2 cells and CART30 in the given effector:target:macrophage ratios. Statistical significance was determined by unpaired t-tests (FIG. 10E), or two-way ANOVA (FIG. 10F).

FIGS. 11A-11J show that BTLA knockout drives CART19 effector programming in vivo. FIG. 11A: Design of an in vivo-based scRNA-seq experiment to characterize circulating CART19. On day −10, 5×10⁶ luciferase+ OCILy18 cells were implanted subcutaneously into NSG mice. On day 0, 4×10⁶ cells WT (n=8 mice) or BTLA KO (n=6 mice) 4-1BBζ CART19 cells were infused into tumor bearing mice. FIG. 11B: Tumor burden was monitored via bioluminescent imaging. On day 16, mice were bled via retro-orbital sinus; human T cells from individual mice were barcoded, pooled based on condition (WT or BTLA KO) and sorted via FACS. FIG. 11C: UMAP of circulating CART19 subsets following data merging, filtering, and clustering using the Seurat pipeline (Hao, et al., Cell 184, 3573-3587.e3529 (2021); Stuart, et al., Cell 177, 1888-1902.e1821 (2019)). FIG. 11D: Distribution of clusters between WT and BTLA KO, showing near absence of CD4+ T_{naive/mem}, CD8+_{eff-mem}, and ROR-high cells in BTLA KO cells. FIG. 11E: Individual feature plots used to define T cell subsets. T cell subsets were broadly defined by CD4 or CD8 expression; individual expression of key effector or naïve/memory genes is also shown. FIG. 11F: UMAP, split by either WT or BTLA KO cells. The number of cells being analyzed in each group is very similar (WT: n=2409; BTLA KO: n=2487), reflective of the effort to load the 10× Chromium Controller evenly for each condition. FIG. 11G: DEGs between BTLA KO and WT CART19 in effector CD4+. FIG. 11H: DEGs between BTLA KO and WT CART19 in effector CD8+. FIG. 11I: Pathway analysis of DEGs between BTLA KO and WT CART19 cells. FIG. 11J: Total human CD45+ CD3+ T cells per 100 L peripheral blood at days 31 and 45 post-CAR19 infusion, per mouse and per condition. 2/6 mice in the BTLA KO arm were euthanized after showing mild signs of GvHD (fur loss), hence n=4 data points at day 45. Statistical significance was calculated using Seurat's FindMarkers function (FIGS. 11G, 11H), the gene ontology (GO) enrichment analysis via clusterProfiler (Wu, et al., The Innovation 2, 100141 (2021)) (FIG. 11I), unpaired t-test (FIG. 11B), or two-way ANOVA (FIG. 11J). T_{eff}: T effector; Tₘₑₘ: memory T cell; T_{eff-mem}: effector memory T cell.

FIGS. 12A-12H show single cell RNA sequencing of sorted CART19 tumor infiltrates in OCI-Ly18 tumors. FIG. 12A: Detailed methods and FACS (BD Melody) gating strategies used to process and sort circulating T cells from peripheral blood (Proliferation model in FIGS. 11A-11J). FIG. 12B: Detailed methods and FACS (BD Melody) gating strategies used to process and sort tumor infiltrating T cells (TILs, Exhaustion model, shown in FIG. 12C). FIG. 12C: Schema of Exhaustion model study design. Briefly, 5×10⁶ OCI-Ly18 were implanted into NSG mice on day −14. On day 0, 4×10⁶ WT (n=2 mice) or BTLA KO (n=2 mice) CART19 were infused via tail vein. On day 11 post-infusion, tumors were resected, minced, and filtered (70 μm & 40 μm). Mice tumor suspensions were pooled per condition (2 per arm); TILs were FACS-purified, washed, resuspended in 0.04% BSA in 1×PBS, and loaded onto 10× Genomics chips. After capture, library preparation, and NGS, data were filtered as described in the Methods section. FIG. 12D: UMAP of scRNA-seq data from CAR TILs, split by condition (WT, n=1730 cells; BTLA KO, n=1007 cells). FIG. 12E: Defining features of UMAP sections used to label clusters in FIG. 12F. FIG. 12F: UMAP clusters. FIG. 12G: Left: DEGs between WT and BTLA KO TILs in IFNG-high cluster; Right: enriched pathways in BTLA KO CAR TILs within IFNG-high cluster. FIG. 12H: (i) DEGs between WT and BTLA KO TILs within the IL13-high cluster (DEGs: differentially expressed genes), and (ii) a graph showing that BTLA KO augments phosphorylation of effector signaling in CAR T cells. Statistical significance was determined using Seurat's FindMarkers function (FIGS. 12G, 12H).

FIGS. 13A-13L show that BTLA inhibits CAR T cells via ITIMs Y257 and Y282. FIG. 13A: Schematic of proposed point mutations in BTLA intracellular tyrosine motifs to abolish recruitment of SHP-1 and SHP-2. These two mutations are Y257F and Y282F, respectively. FIG. 13B: Expression of BOX and FOXY constructs in primary human T cells that received baseline knockout of BTLA. left: Transgene constructs to co-express 4-1BBζ-CAR19 and iRFP, WT BTLA (“BOX”), or mutated BTLA (“FOXY”) via 2A self-cleaving peptide; right: Expression of BTLA in resting P2A iRFP, BOX, and FOXY CART19 cells. FIG. 13C: 24 h ELISA for IL-2 on supernatant of coculture with CART19 cells and OCI-Ly18 (seeding effector: target ratio=0.25, based on 50,000 cancer cells; n=4 technical replicates). FIG. 13D: Representative confocal images for lipid bilayer colocalization studies, showing overlap of BTLA and SHP-2. Briefly, CD19 protein with or without HVEM proteins were anchored onto glass planar supported lipid bilayers. BOX and FOXY CART19 cells were settled onto platforms for 15 min prior to fixation and staining with either BTLA and SHP-1 or SHP-2. FIG. 13E: Quantification of fluorescent overlap from BTLA and SHP-1 for all configurations of the lipid bilayer platform. Here, the Manders Correlation Coefficient (MCC) values are presented. FIG. 13F: Quantification of fluorescent overlap from BTLA and SHP-2 for all configurations of the lipid bilayer platform. Here, the Manders Correlation Coefficient (MCC) values are presented. FIG. 13G: Design of in vivo experiment to study long-term persistence of BTLA-modified CAR T cells using OCI-Ly18 and NSG mice. 5×10⁶ OCI-Ly18 cells were implanted subcutaneously in the right flank of NSG mice. On day 4 post-implantation, mice were imaged laterally, and randomized on the basis of bioluminescent intensity. On day 5 post-implantation, mice were infused with 4×10⁶ CAR+ T cells that were UTD (n=6), CAR19 P2A iRFP+ (n=6), BTLA KO CAR19 P2A iRFP (n=6), CAR19 P2A BTLA (“BOX”, n=6), or CAR19 P2A mutBTLA (“FOXY”, n=6). FIG. 13H: Average right-flank tumor growth curves with individual replicates in the first 40 days after initial tumor implantation. Statistics between CAR19 P2A iRFP and BOX are shown at day 25 post initial tumor challenge. FIG. 13I: Average overall survival (i) in the first 40 days after initial tumor implantation. Statistics between CAR19 P2A iRFP and BOX are shown at day 25 post initial tumor challenge. For FIGS. 13J-13L, at day 40, surviving mice (i.e., CAR19 P2A iRFP (n=6), BTLA KO CAR19 P2A iRFP (n=6), or CAR19 P2A mutBTLA (“FOXY”, n=6)) that cleared the initial tumor challenge were subject to re-challenge with 4×10⁶ OCI-Ly18, now as a subcutaneous injection in the left flank. FIG. 13J: Mice were serially bled on days 0 (immediately before tumor inoculation), 4, and 8 after re-challenge; T cells were quantified after gating on human CD45+ CD3+ cells. FIG. 13K: k,l. Average left-flank tumor growth curves with individual replicates following re-challenge. Statistics between CAR19 P2A iRFP, BTLA KO P2A iRFP, and FOXY are shown at day 34 post re-challenge. FIG. 13L: overall survival following re-challenge. Statistics between CAR19 P2A iRFP, BTLA KO P2A iRFP, and FOXY are shown at day 34 post re-challenge. Statistical significance was determined via one-way ANOVA with post hoc Tukey tests (FIGS. 13C, 13E, 13F, 13H, 13K), two-way ANOVA (FIG. 13J), or long-rank Mantel Cox test (FIGS. 13I, 13L). ns, not significant; *, P<0.05; ****, P<0.0001.

FIGS. 14A-14H show additional confocal images and Grb2 mechanistic data. FIG. 14A: Graphical abstract of lipid bilayer platform and confocal imaging to colocalize BTLA and SHP1/SHP2 from BOX and FOXY CART19. FIG. 14B: Representative confocal images of SHP-1 and BTLA. c. Representative raw images from confocal studies. For FIGS. 14D-14E, briefly, human CD19-transduced HVEM+ DM-6 melanoma cells were seeded into culture dishes and allowed to form a stable monolayer for 48 hr. 5e5 BOX or FOXY CART19 cells were settled onto CD19+ HVEM+ DM-6 monolayers for 0 m, 15 m, and 30 m, and subsequently rinsed off using cold 1×PBS. Cells were then centrifuged, washed 2× with cold 1×PBS, and then lysed in buffer (RIPA+protease inhibitor+phosphatase inhibitor). Standard Western Blot was conducted, first to measure p-Akt. Membranes were then stripped, and re-stained for total Akt. Blots were read and quantification was performed using the LI-COR imaging system (Image Studio™). FIG. 14D: Quantification of phosphorylated-Akt (p-Akt) following CD19 and HVEM stimulation. FIG. 14E: Quantification of pAkt/Akt for two independent experiments, using two distinct donors. For FIGS. 14F-14H, Jurkat-NFAT-GFP reporter cells were transduced with either the BOX CAR19 or FOXY CAR19 constructs. FIG. 14F: BOX-Jurkat or FOXY-Jurkat CART19 were cocultured with Nalm6-HVEM in a 1:1 effector: target ratio and monitored each in real-time for the first 15 minutes of coculture via flow cytometry (gated on CD3+>GFP). This is a single, representative example. FIG. 14G: BOX-Jurkat or FOXY-Jurkat CART19 were cocultured with either Nalm6 or Nalm6-HVEM (effector: target=0.25:1), and GFP+ MFI was measured at 6 hr using flow cytometry. FIG. 14H: Same experiment and data as in FIG. 14G, but now including the 20 hr timepoint. Statistical significance was determined by two-way ANOVA (FIGS. 14E, 14G, 14H). ns, not significant; *, P<0.01.

FIGS. 15A-15L relate to dual inhibition of BTLA and PD-1 in CAR T cells. FIG. 15A: (i) a chart showing significantly decreased tumor burden in mice treated with BTLA KO CAR19 T cells over those treated with PD1 KO CAR T cells or controls, and (ii) data from FIG. 5I now including PD-1 KO condition (n=8 mice). FIG. 15B: Data from FIG. 5L now including PD-1 KO condition (n=8 mice). FIG. 15C: Data from FIG. 6J, now including PD-1 KO condition (n=7 mice). FIGS. 15D-15K show that dual checkpoint inhibition of BTLA and PD-1 in CAR30 T cells promotes potent tumor control. FIG. 15D: DepMap Expression Score of HVEM(TNFRSF14) and PD-L1 in select CD30+ cell lines. FIG. 15E: Tumor killing at 72 hr (short-term coculture of UTD or CAR30 T cells with Pembrolizumab against HDLM-2; seeding effector: target ratio=0.25). FIG. 15F: Proposed schema for dual BTLA/PD-1 inhibition of CAR30 T cells using CRISPR-Cas9-mediated deletion of BTLA and inhibition of PD-1 using Pembro FIG. 15G: Design of in vivo study using HDLM-2 and NSG mice. Briefly, 5×10⁶ CBG+ HDLM-2 cells were intravenously infused on day −22. On day 0, mice displayed moderate to high bioluminescent signal (~10⁷). First, mice were split into groups that would receive saline (PBS) or Pembrolizumab (10 mg/kg) as weekly intraperitoneal injections for three weeks, starting on day 0. Mice were then treated with 5×10⁵ UTD or CAR30+ T cells, and the subsequent conditions were UTD (n=4), UTD+Pembro (n=4), WT CART30+Saline (n=4), WT CART30+Pembro (n=6), BTLA KO CART30+Saline (n=5), or BTLA KO CART30+Pembro (n=6). FIG. 15H: Median bioluminescent intensity (tumor burden) of each condition, with individual mouse curves in shown in the background. FIG. 15I: Overall survival of mice in each condition. FIG. 15J: Peripheral blood human CD45+CD3+ counts on day 16 post-CART30 infusion. FIG. 15K: Tumor killing (short-term coculture at 48 hr of WT or BTLA KO CAR30 T cells against KM-H2 (HL) and SU-DH-L1 (ALCL)+Vehicle (saline) or Pembro (100 μg/mL) (seeding effector: target ratio=0.125). FIG. 15L: Expression of BTLA and PD-1 in CD4+ pan-cancer TILs from the online portal of Zheng et al., Science, 2021 (http://cancer-pku.cn:3838/PanC_T/) (Zheng, et al., Science 374, abe6474 (2021)). Statistical significance was determined by one-way ANOVA with post hoc Tukey tests (FIG. 15A (week 9 only), FIGS. 15E, 15J, 15K), or long-rank Mantel Cox test (FIGS. 15C, 15I). ns, not significant; ****, P<0.0001.

FIGS. 16A-16O show that BTLA-deficient CAR T cells (and exogenous TCR T cells) treat solid malignancies. FIG. 16A: TCGA correlates of TNFRSF14/HVEM tumor expression. Transcript expression in log 2(TPM+1) of HVEM (top) and PD-L1 (bottom) across all TCGA cohorts. FIG. 16B: TCGA correlates of TNFRSF14/HVEM tumor expression. Disease-free survival of all TCGA cancers, split either by median HVEM expression (left), or median PD-L1 expression (right). FIG. 16C: Disease-free survival of TCGA OV cohort, based on BTLA (left) or PDCD1 (right) RNA expression normalized to CD3E (upper and lower quartile cutoffs). For FIGS. 16D-16F, BTLA KO anti-HER2 (scFv 4D5) CAR T cells were manufactured and cocultured with HER2+ SK-OV-3 (ovarian) or PC-3 (prostate) cancer cell lines. FIG. 16D: 4000 SK-OV-3 cells were seeded into 96-well plates 24 hr prior to the addition of 1000 4D-5 CAR+ T cells (effector: target=0.25, n=5 technical replicates). GFP+ intensity was monitored every 3 hr using the Incucyte Live-Cell Analysis System. FIG. 16E: (i) Disease-free survival of TCGA PRAD cohort, based on BTLA expression normalized to CD3E (median cutoff), (ii) a graph of Incucyte Live Cell Analysis assay data showing significantly enhanced in vitro cytotoxicity of BTLA KO anti-HER-2 CAR T cells against HER2+ prostate adenocarcinoma cells, and (iii) a chart of the 48 hour time point Incucyte assay data showing significantly enhanced in vitro cytotoxicity of BTLA KO anti-HER-2 CAR T cells against HER2+ prostate adenocarcinoma cells. FIG. 16F: 4000 PC-3 cells were seeded into 96-well flat bottom plate (E-Plate 96 PET, ACEA Biosciences). 24 hr later, 1000 4D-5 CAR+ T cells were seeded into respective wells (effector: target=0.25, n=3 technical replicates). Results were obtained using the xCELLigence System. FIG. 16G: Knockout of HVEM from PC-3 eliminates benefit of BTLA KO anti-HER2 CAR T. 4000 WT or HVEM-deficient CBG+PC-3 cells were seeded into a 96-well plate. Anti-HER2 CAR T cells were added 24 hr later at the listed effector: target ratios, and luminescence was measured at 72 hr of coculture. HVEM-deficient PC-3 cells were generated through electroporation of Cas9 and a HVEM sgRNA (Lonza 4D-Nucleofector™, Pulse Code: DS137) FIG. 16H: OS of TCGA PAAD cohort, based on BTLA expression normalized to CD3E (median cutoff). FIG. 16I: Basal HVEM expression on AsPC-1 cells. FIG. 16J: Luminescence-based cytotoxicity of anti-mesothelin CAR (CARM5, see NCT03323944) T cells against pancreatic cancer. WT or BTLA KO anti-mesothelin CAR T (i.e., CARM5) were manufactured and cocultured with mesothelin+ pancreatic cancer (CBG+ AsPC-1) at various effector to target ratios (4000 AsPC-1 cells, seeded 24 hr prior). Luminescence was measured at 48 hr and 72 hr of coculture. FIG. 16K: (i) a graph of Incucyte Live Cell Analysis assay data showing significantly enhanced in vitro cytotoxicity of BTLA KO anti-GP100 TCR T cells (the T cell receptor alpha locus (TRAC) is also knocked out in the cells), and (ii) in vitro tumor growth of B16-muCD19 cells cocultured with UTD, WT muCART19, or BTLA KO muCART19 at the specified effector: target ratios. Briefly 4,000 B16 cells transduced to overexpress murine CD19 were plated in 96-well flat bottom plate (E-Plate 96 PET, ACEA Biosciences). 24 hr later, murine CAR T cells (produced from C57BL/6 T cells) were de-beaded from expansion, and seeded into respective wells (n=3 per condition). Results were obtained using the xCELLigence System. FIGS. 16L-16O show data from in vivo B16-muCD19 melanoma study. Briefly, 2×10⁵ B16-muCD19+ cells were implanted into C57BL/6 mice on day −5. On the same day, production of murine WT/mock-electroporated or BTLA KO CART19 began. On day −1, 100 mg/kg cyclophosphamide was administered via intraperitoneal injection. On day 0, 4×10⁶ muCAR19+ T cells were retro-orbitally injected into mice. FIG. 16L: Tumor volumes following infusion of murine WT (n=8) muCART19 cells. FIG. 16M: Tumor volumes following infusion of murine BTLA KO (n=9) muCART19 cells. FIG. 16N: Tumor sizes plotted for day 14 following murine CAR T cell infusion. FIG. 16O: Overall survival of mice in B16-muCD19 study. Statistical significance was determined by one-way ANOVA with post hoc Tukey tests (FIGS. 16D, 16F, 16K at endpoints), two-way ANOVA (FIG. 16J), unpaired or multiple unpaired t-test (FIGS. 16G, 16N), or long-rank Mantel Cox test (FIGS. 16B, 16C, 16E, 16H, 16O). ns, not significant; *, P<0.05; **, P<0.01; ***, P<0.001; ****, P<0.0001.

FIGS. 17A-17H show that Expression of BTLA in patient CART19 cells correlates with clinical response. FIGS. 17A-17D relate to bulk RNA-sequencing of infusion products of 35 patients treated with 4-1BBζ CART19 (CTL019) in NCT02030834. FIG. 17A: Normalized BTLA expression in complete responders (CR) versus patients with partial response/progressive disease (PR/PD). Here, response is the patient's best overall response. FIG. 17B: Differential expression of BTLA in patients who experience early relapse (defined as either non-responders or patients who relapse within 3 months of infusion), versus the remainder of the cohort. FIG. 17C: Progression-free survival (PFS), split by top quartile and bottom three quartiles of normalized BTLA expression. FIG. 17D: Overall survival (OS), split by top quartile and bottom three quartiles of normalized BTLA expression. FIG. 17E: Serum concentration of soluble-HVEM in n=12 patients from the same clinical trial, measured by the SomaScan® Assay. FIGS. 17F-17H show reanalysis of CyTOF data of sorted CD28ζ CAR19+ T cells (Axi-cel) from DLBCL patients (Good et al. Nat Med, 2022). FIG. 17F: t-sne clustering of CD4+ CAR+ T cells at day 7 post-infusion, highlighting the Helios+ BTLA+ TIGIT+ cluster 5. CyTOF data were analyzed using the OMIQ software (Dotmatics). FIG. 17G: Proportion (percentage) of cluster 5 amongst CD4+CAR+ T cells, for CR and PR/PD/SD at day 7. FIG. 17H: Proportion of BTLA+ cells among CD4+CAR+ T cells, for CR and PR/PD/SD at day 7. Statistical significance was determined by unpaired t-test (FIGS. 17A, 17E, 17G, 17H), or log-rank Mantel-Cox test (FIGS. 17C, 17D). CR: complete response; PR: partial response, PD: progressive disease; SD: stable disease.

FIGS. 18A-18C show that HVEM switch receptors in CAR T cells can selectively bind LIGHT. FIG. 18A shows expression of CD69 in WT or HVEM KO CAR19 T cells after 2 hr of 1:1 anti-CD3/CD28 bead:T cell ratio with or without 60 ng/mL soluble LIGHT. FIG. 18B is a schematic of the human HVEM variants (i.e., HVEM “switch receptors” comprising point mutation G89F or E52K to abrogate binding with BTLA and enhance affinity to LIGHT (top) and a schematic of transgene constructs of CAR19 co-expressing WT HVEM or HVEM variants (i.e., G89F HVEM or E52K HVEM) via a 2A self-cleaving peptide. FIG. 18C shows in vitro tumor killing at 48 hr against OCI-Ly18 of UTD, CAR19, CAR19 HVEM KO, CAR19 HVEM-overexpression, CAR19-HVEM-G89F, and CAR19-HVEM-E52K T cells.

### DETAILED DESCRIPTION

B and T lymphocyte attenuator (BTLA) is an immunomodulatory molecule widely expressed on the surface of immune cells that shares structural homology to both PD1 and CTLA-4 (Xu, et al., J Cell Biol 219 (2020); Watanabe, et al., Nature Immunology 4, 670-679 (2003)). Upon trans engagement with its ligand herpes virus entry mediator (HVEM) on target cells, BTLA recruits two potent tyrosine phosphatases, SHP-1 and SHP-2, to disable early T cell activation (Cheung, et al., J Immunol 183, 7286-7296 (2009)). As demonstrated herein, BTLA expression on T cells contributes to inhibition and chronic exhaustion of T cells in cancer (Jiang, Li, & Zhu, Cell Death & Disease 6, e1792-e1792 (2015)), thereby acting as a significant immune checkpoint that blunts the anti-tumor response of adoptive cell transfer immunotherapies. Particularly in HL, BTLA is enriched in the CD4+ cells and contributes to regulatory T cell phenotype with dampened activation (Wein, et al., Cancer Immunology Research 5, 1122-1132 (2017)). In parallel, HVEM expression has been deemed a key checkpoint in several solid tumors including skin, lung, breast, esophageal, gastric, and colorectal cancers, even substituting as the dominant immune checkpoint in PD-L1 low tumor settings (Ren, et al., Lung Cancer 125, 115-120 (2018); Malissen, et al., Oncoimmunology 8, e1665976 (2019); Lan, et al., Onco Targets Ther 10, 919-926 (2017); Migita, et al., Cancer 120, 808-817 (2014); Tsang, et al., Ann Surg Oncol 24, 4042-4050 (2017); Zhang, et al., Oncol Res 24, 189-196 (2016); Inoue, et al., Anticancer Res 35, 1361-1367 (2015)). The present invention provides compositions and methods to overcome the immunomodulatory activity of BTLA-HVEM signaling in immune cells in order to enhance the anti-cancer efficacy of adoptive cell transfer immunotherapies.

In one aspect, the invention provides an immune cell genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM).

In another aspect, the invention provides a method of treating cancer in a subject in need thereof, the method comprising administering to the subject a therapeutically effective amount of immune cells, wherein the immune cells are genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM).

In other aspects, provided herein are related compositions (e.g., pharmaceutical compositions) and kits.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Methods and techniques using T Cells with chimeric antigen receptors (CAR T cells) are described in e.g., Ruella, et al., J. Clin. Invest., 126(10):3814-3826 (2016) and Kalos, et al., 3 (95), 95ra73:1-11 (2011), the contents of which are hereby incorporated by reference in their entireties.

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used herein, may be a human or non-human mammal. Non-human mammals include, for example, livestock and pets, such as ovine, bovine, porcine, canine, feline and murine mammals, as well as simian and non-human primate mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, a regulatory T cell, a natural killer T cell, and a gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Altered BTLA-HVEM Signaling in Immune Cells

The efficacy of T cell-based immunotherapies is limited by immunosuppressive pressures in the tumor microenvironment (TME). As disclosed herein, predominant roles were identified for the interaction between BTLA on effector T cells and HVEM (TNFRSF14) on immunosuppressive TME cells, such as regulatory T cells and tumor-associated monocytes. Correspondingly, high BTLA expression in chimeric antigen receptor (CAR) T cells correlated with poor clinical response to treatment as disclosed herein. Deletion of BTLA in CAR T cells resulted in improved tumor control and persistence in models of lymphoma and solid malignancies as disclosed herein. Without wishing to be bound by theory, it is postulated that, mechanistically, BTLA inhibits CAR T cells via recruitment of tyrosine phosphatases SHP-1 and SHP-2, upon trans engagement with HVEM, as disclosed herein. BTLA knockout thus promotes CAR signaling and subsequently enhances effector function. Overall, the findings disclosed herein indicate that the BTLA-HVEM axis is a crucial immune checkpoint in CAR T cell immunotherapy.

In one aspect, the present invention provides an immune cell (e.g. a T cell), or population thereof, genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM). The invention also provides a population of cells comprising the immune cell or population thereof.

In some embodiments, the immune cell or population thereof is characterized by one or more of the following characteristics, as compared to a control immune cell which is not engineered (e.g., genetically modified) to comprise altered signaling of BTLA and/or HVEM:
(i) increased proliferation in vitro and/or in vivo;
(ii) increased cytotoxicity against tumor cells in vitro and/or in vivo;
(iii) enhanced production of pro-inflammatory cytokines (e.g., TNF-α, IFN-γ, and/or IL-2) and/or inflammatory markers (e.g., IL-5 and/or GM-CSF);
(iv) enhanced activation of PLC-gamma, STAT3, and/or STAT6;
(v) diminished synapse-mediated recruitment of SHP-1;
(vi reduced expression of FOXP3 and/or exhaustion markers (e.g., CTLA-4, HAVCR2, and/or PD1);
(vii) increased avidity to tumor cells and increased tumor infiltration;
(viii) enhanced ability to decrease tumor burden in a subjected treated with a therapeutically effective amount of the immune cell or population thereof, wherein the subject has cancer; and/or
(vix) enhanced ability to increase overall survival of a subjected treated with a therapeutically effective amount of the immune cell or population thereof, wherein the subject has cancer.

It is contemplated herein that altered signaling of BTLA and/or HVEM can be achieved in the immune cell by a variety of approaches, such as by gene knockdown or gene knockout of BTLA and/or HVEM, and/or by overexpression of a BTLA variant and/or an HVEM variant.

In certain embodiments, gene expression of endogenous BTLA in the immune cell or population thereof is at least partially knocked down or knocked out. In certain embodiments, gene expression of endogenous HVEM in the immune cell or population thereof is at least partially knocked down or knocked out. In some embodiments, the BTLA and/or HVEM gene expression is knocked down or knocked out in the immune cell or population thereof by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or by 100%.

Various techniques for gene knockdown (e.g., siRNA, shRNA) or gene knockout are well known to those of skill in the art. In some embodiments, gene expression of endogenous BTLA or endogenous HVEM is at least partially knocked out via a gene editing system. Exemplary gene editing systems include those comprising a zinc finger nuclease, a transcription activator-like effector nuclease (TALEN), or a clustered regulatory interspaced short palindromic repeats (CRISPR) system (e.g., a CRISPR/Cas9 system). In some embodiments, gene expression of endogenous BTLA or endogenous HVEM is at least partially knocked out via CRISPR-mediated gene knockout. Exemplary guide RNA sequences suitable for knocking out BTLA and HVEM in human T cells via CRISPR-mediated gene knockout (e.g., via a CRISPR-Cas system) include:

(BTLA KO)
(SEQ ID NO: 1)
5′-UGUUAGCACAGUAUUUCACA-3′
(HVEM KO)
(SEQ ID NO: 2)
5′-CAAGGAGGACGAGUACCCAG-3′.

In some embodiments, the immune cell is a T cell genetically modified to express a chimeric antigen receptor (CAR) or a T cell receptor (TCR); further wherein gene expression of endogenous BTLA is at least partially knocked out. In some embodiments, the endogenous BTLA is at least partially knocked out via CRISPR-mediated gene knockout. In some embodiments, the CRISPR-mediated gene knockout utilizes a gRNA comprising SEQ ID NO: 1.

In some embodiments, the immune cell is a T cell genetically modified to express a chimeric antigen receptor (CAR) or a T cell receptor (TCR); further wherein gene expression of endogenous HVEM is at least partially knocked out. In some embodiments, the endogenous HVEM is at least partially knocked out via CRISPR-mediated gene knockout. In some embodiments, the CRISPR-mediated gene knockout utilizes a gRNA comprising SEQ ID NO: 2.

In certain embodiments, the immune cell is genetically modified to express a BTLA variant and/or an HVEM variant that alters BTLA and/or HVEM signaling in the immune cell. The BTLA variant suitable for use in the invention has reduced ability to bind SIP-1 and SHP-2. The BTLA variant further retains the ability to bind to Grb2. The HVEM variant suitable for use in the invention has reduced ability to bind to BTLA. The HVEM variant further retains ability to bind to LIGHT. In some embodiments, the BTLA variant is a human BTLA variant comprising Y257F and/or Y282F amino acid substitution(s) as previously described (Gavrieli, et al., Biochem Biophys Res Commun., 312(4):1236-43 (2003)). In some embodiments, the HVEM variant is a human HVEM variant comprising E52K and/or G89F amino acid substitution(s), as previously described (Shrestha, et al., Structure, 28(11):1197-1205.e2 (2020)).

In some embodiments, the immune cell genetically modified to express the BTLA variant is further genetically modified such that gene expression of endogenous BTLA in the immune cell is at least partially knocked down or knocked out.

In some embodiments, the immune cell genetically modified to express the HVEM variant is further genetically modified such that gene expression of endogenous HVEM in the immune cell is at least partially knocked down or knocked out.

In some embodiments, the immune cell comprises an expression vector (such as a lentiviral vector) comprising a first nucleotide sequence encoding the CAR or the TCR, a second nucleotide sequence encoding the BTLA variant or the HVEM variant, and a third nucleotide sequence comprising a ribosomal skipping sequence, wherein the third nucleotide sequence is located between the first and second nucleotide sequences.

In some embodiments, the immune cell comprises an expression vector comprising a first nucleotide sequence encoding the CAR or the TCR, and the immune cell further comprises a second nucleotide sequence encoding the BTLA variant or the HVEM variant, wherein the second nucleotide sequence is integrated into genomic DNA of the immune cell. Various techniques for integrating DNA into genomic DNA are known in the art. Any suitable vector for gene knock-in can be used to integrate the BTLA or HVEM variant into genomic DNA of the immune cell. In some embodiments, the nucleotide sequence encoding the BTLA variant or the HVEM variant is integrated into genomic DNA of the immune cell via adenovirus-mediated gene transfer. In some embodiments, the nucleotide sequence encoding the BTLA variant or the HVEM variant is integrated into genomic DNA of the immune cell via CRISPR-mediated gene knock-in.

Human BTLA comprises the following amino acid sequence:

(SEQ ID NO: 3)
MKTLPAMLGTGKLFWVFFLIPYLDIWNIHGKESCDVQLYIKRQSEHSILA
GDPFELECPVKYCANRPHVTWCKLNGTTCVKLEDRQTSWKEEKNISFFIL
HFEPVLPNDNGSYRCSANFQSNLIESHSTTLYVTDVKSASERPSKDEMAS
RPWLLYRLLPLGGLPLLITTCFCLFCCLRRHQGKQNELSDTAGREINLVD
AHLKSEQTEASTRQNSQVLLSETGIYDNDPDLCFRMQEGSEVYSNPCLEE
NKPGIVYASLNHSVIGPNSRLARNVKEAPTEYASICVRS.

In some embodiments, human BTLA is encoded by the following nucleotide sequence:

(SEQ ID NO: 4)
ATGAAAACACTCCCGGCTATGTTAGGAACCGGTAAGCTGTTCTGGGTCTT
CTTTCTCATTCCGTACTTGGATATTTGGAATATTCACGGCAAGGAGTCTT
GCGACGTGCAATTGTACATTAAACGGCAGAGCGAGCATAGCATACTGGCC
GGCGACCCTTTCGAGCTGGAGTGTCCCGTCAAGTATTGCGCCAATAGACC
ACACGTTACCTGGTGTAAATTAAACGGCACTACTTGCGTTAAGCTGGAAG
ACAGGCAGACCAGCTGGAAAGAGGAAAAGAATATCAGCTTCTTTATCCTG
CACTTCGAGCCTGTCTTGCCAAACGATAACGGTAGTTATAGATGCTCAGC
CAACTTCCAATCCAACCTGATAGAGTCACATTCTACCACCCTGTACGTAA
CGGACGTCAAGTCAGCTAGTGAGCGCCCTAGTAAAGATGAGATGGCCTCT
CGCCCTTGGCTTCTCTACCGATTGCTCCCACTCGGCGGGTTACCACTGTT
AATAACCACTTGCTTTTGTTTATTTTGTTGTCTTCGCAGACATCAGGGGA
AACAGAACGAGCTGAGCGATACCGCTGGCAGAGAGATCAATTTGGTCGAT
GCCCATTTGAAAAGCGAACAGACCGAGGCCTCAACACGGCAGAACTCACA
GGTCTTGCTGTCTGAGACCGGCATATACGACAACGATCCGGATCTCTGCT
TTCGGATGCAAGAGGGATCCGAGGTATACTCCAACCCCTGTCTTGAGGAG
AATAAGCCTGGTATAGTGTACGCCAGTCTTAATCACAGTGTGATCGGTCC
TAATTCCCGCCTCGCGCGGAACGTCAAAGAGGCCCCTACCGAGTACGCTA
GTATTTGCGTCAGATCT.

In some embodiments, the BTLA variant comprising a Y257F amino acid substitution comprises the following amino acid sequence:

(SEQ ID NO: 5)
MKTLPAMLGTGKLFWVFFLIPYLDIWNIHGKESCDVQLYIKRQSEHSILA
GDPFELECPVKYCANRPHVTWCKLNGTTCVKLEDRQTSWKEEKNISFFIL
HFEPVLPNDNGSYRCSANFQSNLIESHSTTLYVTDVKSASERPSKDEMAS
RPWLLYRLLPLGGLPLLITTCFCLFCCLRRHQGKQNELSDTAGREINLVD
AHLKSEQTEASTRQNSQVLLSETGIYDNDPDLCFRMQEGSEVYSNPCLEE
NKPGIVFASLNHSVIGPNSRLARNVKEAPTEYASICVRS.

In some embodiments, the BTLA variant comprising a Y282F amino acid substitution comprises the following amino acid sequence:

(SEQ ID NO: 6)
MKTLPAMLGTGKLFWVFFLIPYLDIWNIHGKESCDVQLYIKRQSEHSILA
GDPFELECPVKYCANRPHVTWCKLNGTTCVKLEDRQTSWKEEKNISFFIL
HFEPVLPNDNGSYRCSANFQSNLIESHSTTLYVTDVKSASERPSKDEMAS
RPWLLYRLLPLGGLPLLITTCFCLFCCLRRHQGKQNELSDTAGREINLVD
AHLKSEQTEASTRQNSQVLLSETGIYDNDPDLCFRMQEGSEVYSNPCLEE
NKPGIVYASLNHSVIGPNSRLARNVKEAPTEFASICVRS.

In some embodiments, the BTLA variant comprising Y257F and Y282F amino acid substitutions comprises the following amino acid sequence:

(SEQ ID NO: 7)
MKTLPAMLGTGKLFWVFFLIPYLDIWNIHGKESCDVQLYIKRQSEHSILA
GDPFELECPVKYCANRPHVTWCKLNGTTCVKLEDRQTSWKEEKNISFFIL
HFEPVLPNDNGSYRCSANFQSNLIESHSTTLYVTDVKSASERPSKDEMAS
RPWLLYRLLPLGGLPLLITTCFCLFCCLRRHQGKQNELSDTAGREINLVD
AHLKSEQTEASTRQNSQVLLSETGIYDNDPDLCFRMQEGSEVYSNPCLEE
NKPGIVFASLNHSVIGPNSRLARNVKEAPTEFASICVRS.

In some embodiments, the BTLA variant comprising Y257F and Y282F amino acid substitutions is encoded by the following nucleotide sequence:

(SEQ ID NO: 8)
ATGAAAACGCTTCCCGCCATGCTGGGGACAGGAAAGTTATTTTGGGTCTT
CTTTCTCATACCTTACTTGGATATTTGGAATATTCACGGCAAGGAGAGCT
GCGACGTCCAATTGTACATTAAACGGCAGAGTGAGCATAGTATACTGGCT
GGCGACCCTTTCGAGTTGGAGTGTCCAGTAAAGTATTGCGCCAATAGACC
ACACGTTACCTGGTGTAAATTAAACGGCACTACCTGCGTCAAGCTCGAGG
ACCGGCAGACCAGCTGGAAAGAGGAAAAGAATATCAGTTTCTTTATCCTG
CACTTCGAGCCCGTCTTGCCGAACGATAACGGCAGCTATAGATGCTCCGC
TAACTTCCAATCCAACCTGATCGAGTCTCATAGCACTACACTGTACGTTA
CCGACGTGAAGTCCGCTAGTGAGCGGCCTTCTAAAGATGAGATGGCCTCT
CGCCCATGGTTGTTGTACAGGCTGCTCCCCCTCGGCGGGCTGCCCTTACT
GATAACGACATGCTTTTGTTTATTTTGTTGTCTTCGAAGACATCAGGGGA
AACAGAACGAGCTGAGCGATACCGCTGGGCGAGAGATCAATCTCGTGGAC
GCCCATCTCAAAAGCGAACAGACTGAGGCTTCTACTCGGCAGAACTCTCA
GGTTCTCCTGTCCGAGACCGGTATATACGACAACGATCCCGATCTCTGCT
TTCGCATGCAAGAGGGATCCGAGGTGTACAGTAACCCTTGTCTCGAGGAG
AATAAGCCTGGGATCGTCTTTGCCTCATTAAATCACAGCGTGATCGGGCC
AAATAGCCGGCTCGCGCGAAACGTCAAGGAAGCTCCTACGGAGTTTGCTA
GCATCTGCGTACGATCC.

Human HVEM comprises the following amino acid sequence:

(SEQ ID NO: 9)
MEPPGDWGPPPWRSTPKTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH.

In some embodiments, human HVEM is encoded by the following nucleotide sequence:

(SEQ ID NO: 10)
ATGGAACCACCGGGAGATTGGGGCCCACCACCATGGCGCAGCACACCAAAGACAG
ATGTTCTCCGCCTCGTTCTTTACCTGACATTTCTCGGTGCTCCTTGTTATGCACCCGC
ATTACCTAGTTGTAAAGAAGATGAATATCCCGTAGGGAGTGAATGTTGTCCTAAATG
TTCTCCCGGATACCGGGTCAAAGAAGCGTGTGGTGAACTCACTGGTACCGTATGCGA
GCCTTGTCCACCTGGAACTTATATCGCTCATTTGAACGGGCTGAGTAAATGCCTTCA
ATGTCAGATGTGCGATCCGGCTATGGGATTGAGGGCCTCTAGGAATTGTTCACGGAC
GGAAAATGCAGTCTGCGGGTGTTCTCCTGGGCATTTCTGTATTGTGCAAGATGGCGA
TCATTGTGCTGCATGTAGGGCATATGCTACGAGCAGTCCTGGGCAAAGAGTTCAAAA
GGGCGGAACTGAAAGCCAAGATACGCTTTGCCAAAATTGTCCCCCAGGAACGTTTA
GTCCGAACGGCACACTTGAAGAGTGCCAACATCAAACAAAATGTTCCTGGTTGGTC
ACTAAAGCAGGGGCGGGAACTTCTTCTTCTCATTGGGTTTGGTGGTTCCTTAGCGGC
AGTCTGGTGATTGTTATAGTGTGTTCTACCGTCGGGCTGATTATTTGCGTAAAGCGG
CGGAAACCTCGGGGAGACGTGGTGAAAGTTATTGTTAGCGTACAACGCAAGCGTCA
AGAAGCTGAGGGAGAAGCTACCGTGATCGAAGCATTGCAAGCTCCCCCTGATGTAA
CAACCGTTGCAGTAGAAGAAACTATTCCGAGCTTTACTGGCCGATCACCCAATCAT

In some embodiments, the HVEM variant comprising an E52K amino acid substitution comprises the following amino acid sequence:

(SEQ ID NO: 11)
MEPPGDWGPPPWRSTPKTDVLRLVLYLTFLGAPCYAPALPSCKEDEYPVG
SKCCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH.

In some embodiments, the HVEM variant comprising an E52K amino acid substitution is encoded by the following nucleotide sequence:

(SEQ ID NO: 12)
ATGGAACCACCTGGGGATTGGGGCCCGCCACCATGGAGATCAACGCCAAAGACAGA
TGTGCTGAGATTGGTACTCTACCTGACTTTTCTCGGGGCACCATGTTATGCACCTGCG
CTCCCTTCTTGTAAAGAAGATGAATATCCTGTCGGGAGTAAATGTTGTCCGAAATGT
TCACCCGGGTACCGGGTTAAAGAAGCATGTGGCGAACTTACCGGGACCGTCTGCGA
GCCATGTCCGCCGGGTACATATATCGCACATCTGAACGGGCTCTCCAAATGCCTTCA
ATGTCAGATGTGCGATCCCGCTATGGGTCTTAGAGCCTCTCGCAATTGTTCACGTAC
CGAAAATGCTGTCTGCGGATGTTCACCCGGACACTTTTGTATAGTGCAAGATGGTGA
TCATTGTGCTGCTTGTCGGGCCTATGCTACAAGCAGTCCCGGTCAACGCGTTCAGAA
AGGCGGAACAGAATCTCAAGATACGCTCTGCCAAAATTGTCCGCCCGGCACTTTTAG
CCCTAACGGCACGTTAGAAGAGTGCCAACATCAAACAAAATGTTCCTGGCTCGTCA
CTAAAGCTGGGGCCGGCACTTCAAGTTCTCATTGGGTGTGGTGGTTCTTGAGCGGCA
GTCTGGTAATTGTGATCGTGTGTTCAACTGTCGGGCTGATAATTTGCGTTAAGCGGC
GAAAACCCCGCGGGGACGTCGTGAAAGTCATAGTTAGTGTGCAAAGAAAGAGGCAA
GAAGCCGAGGGAGAAGCTACTGTAATAGAAGCTCTTCAAGCTCCCCCAGATGTTAC
TACTGTTGCTGTAGAAGAAACCATCCCTTCTTTTACCGGCCGTTCTCCTAATCAT.

In some embodiments, the HVEM variant comprising a G89F amino acid substitution comprises the following amino acid sequence:

(SEQ ID NO: 13)
MEPPGDWGPPPWRSTPKTDVLRLVLYLTFLGAPCYAPALPSCKEDEYPVG
SECCPKCSPGYRVKEACGELTGTVCEPCPPGTYIAHLNFLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH.

In some embodiments, the HVEM variant comprising a G89F amino acid substitution is encoded by the following nucleotide sequence:

(SEQ ID NO: 14)
ATGGAACCACCTGGTGATTGGGGCCCTCCTCCTTGGCGTAGCACTCCCAAGACGGAT
GTACTGAGATTAGTTCTCTACTTGACCTTTCTGGGTGCGCCATGTTATGCTCCGGCAC
TCCCCTCTTGTAAAGAAGATGAATATCCCGTTGGAAGCGAATGTTGTCCAAAATGTT
CTCCTGGGTACCGAGTCAAAGAAGCTTGTGGAGAACTCACCGGGACTGTTTGCGAG
CCTTGTCCACCCGGAACTTATATCGCTCATCTGAACTTTCTGAGTAAATGCCTTCAAT
GTCAGATGTGCGATCCCGCAATGGGACTTAGGGCCTCAAGAAATTGTTCTCGGACCG
AAAATGCTGTCTGCGGGTGTAGTCCTGGGCACTTTTGTATTGTTCAAGATGGTGATC
ATTGTGCGGCCTGTAGAGCCTATGCAACAAGCAGTCCAGGGCAACGGGTCCAGAAA
GGCGGTACTGAATCCCAAGATACATTGTGCCAAAATTGTCCCCCAGGAACATTTTCC
CCTAACGGCACTCTCGAAGAGTGCCAACATCAAACAAAATGTAGTTGGCTTGTAACT
AAAGCAGGCGCCGGCACTTCATCAAGCCATTGGGTCTGGTGGTTCCTGAGTGGATCT
CTGGTGATAGTGATCGTGTGTAGCACCGTCGGACTCATTATCTGCGTCAAGCGCAGG
AAACCTCGAGGCGACGTGGTGAAAGTCATTGTGAGTGTGCAACGAAAGCGGCAAGA
AGCCGAGGGAGAAGCAACCGTGATCGAAGCGTTGCAAGCTCCACCTGATGTTACTA
CAGTCGCAGTAGAAGAAACGATTCCAAGTTTTACAGGCCGAAGTCCTAATCAT.

In some embodiments, the HVEM variant comprising E52K and G89F amino acid substitutions comprises the following amino acid sequence:

(SEQ ID NO: 15)
MEPPGDWGPPPWRSTPKTDVLRLVLYLTFLGAPCYAPALPSCKEDEYPVG
SKCCPKCSPGYRVKEACGELTGTVCEPCPPGTYIAHLNFLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH.

Suitable immune cells for use in the invention include, but are not limited to, a T cell, a natural killer (NK) cell, and a macrophage. In some embodiments, the T cell is a CD8+ T cell, a natural killer T (NKT) cells, or a gamma-delta (γδ) T cell.

In certain embodiments, the immune cell is a human cell. In certain embodiments, the immune cell is an autologous cell. In certain embodiments, the immune cell is an autologous cell obtained from a human. In certain embodiments, the immune cell is a T cell. In certain embodiments, the immune cell is a human T cell. In certain embodiments, the immune cell is an autologous human T cell.

In certain embodiments, the CAR or the TCR targets a tumor antigen. In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), gp100, HER2, HLA-A2, HPV E6, ICAM1, IL3Ra, IL13Ra2, LAGE-1, Lewis Y, LMP1 (EBV), MAGE-A1, MAGE-A3, MAGE-A4, MAGE-A6, MAGE-A10, MART-1, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PRAME, PSCA, PSMA, ROR1, ROR2, TTM-3, TM4SF1, TnMuc1, VEGFR2, and any combination thereof.

In some embodiments, the CAR targets CD19, CD30, or HER-2.

In some embodiments, the TCR targets gp100.

### Chimeric Antigen Receptors

In some embodiments, the CAR is a recombinant CAR. In other embodiments, the CAR is encoded by a heterologous sequence (e.g., transgene) present in the immune cell.

In certain embodiments, the CAR comprises an extracellular antigen binding domain, a transmembrane domain, and an intracellular domain. The antigen binding domain of the CAR is operably linked to another domain of the CAR, such as a hinge, a transmembrane domain or an intracellular domain, each described elsewhere herein, for expression in the cell. In one embodiment, a first nucleotide sequence encoding the antigen binding domain is operably linked to a second nucleotide sequence encoding a hinge and/or transmembrane domain, and further operably linked to a third nucleotide sequence encoding an intracellular domain.

The antigen binding domain described herein can be combined with any of the transmembrane domains described herein, any of the intracellular domains or cytoplasmic domains described herein, or any of the other domains described herein that may be included in a CAR of the present invention, such as a hinge domain or a spacer sequence.

The CAR of the present invention may also include a leader sequence as described herein. The CAR of the present invention may also include a hinge domain as described herein. The CAR of the present invention may also include one or more spacer domains or linkers as described herein which may serve to link one domain of the CAR to the next domain.

### Antigen Binding Domain

The antigen binding domain of a CAR is an extracellular region of the CAR for binding to a specific target antigen including proteins, carbohydrates, and glycolipids. The CAR of the invention comprises an antigen binding domain that is capable of binding a tumor antigen. Suitable tumor antigens are known in the art and include, but are not limited to, alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof. In some embodiments, the tumor antigen is CD19. In some embodiments, the antigen-binding domain is an anti-CD19 antigen binding domain which is capable of binding CD19, such as the FMC63 scFv known in the art.

The antigen binding domain can include any domain that binds to the antigen (e.g., tumor antigen) and may include, but is not limited to, a monoclonal antibody (mAb), a polyclonal antibody, a synthetic antibody, a human antibody, a humanized antibody, a non-human antibody, a single-domain antibody, a full-length antibody or any antigen-binding fragment thereof, a Fab, and a single-chain variable fragment (scFv). In some embodiments, the antigen binding domain comprises an aglycosylated antibody or a fragment thereof or scFv thereof.

As used herein, the term “single-chain variable fragment” or “scFv” is a fusion protein of the variable regions of the heavy (VH) and light (VL) chains of an immunoglobulin (e.g., mouse or human) covalently linked to form a VH::VL heterodimer. The variable heavy (VH) and light (VL) chains are either joined directly or joined by a peptide linker, which connects the N-terminus of the VH with the C-terminus of the VL, or the C-terminus of the VH with the N-terminus of the VL. In some embodiments, the antigen binding domain (e.g., tumor antigen binding domain) comprises an scFv having the configuration from N-terminus to C-terminus, VH—linker—VL. In some embodiments, the antigen binding domain comprises an scFv having the configuration from N-terminus to C-terminus, VL—linker—VH or VH—linker—VL. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers. Those of skill in the art would be able to select the appropriate linker sequence for use in the present invention. In one embodiment, an antigen binding domain of the present invention comprises a heavy chain variable region (VH) and a light chain variable region (VL), wherein the VH and VL are separated by a linker sequence.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In other embodiments, the antigen binding domain comprises an antibody mimetic protein such as, for example, designed ankyrin repeat protein (DARPin), affibody, monobody, (i.e., adnectin), affilin, affimer, affitin, alphabody, avimer, Kunitz domain peptide, or anticalin. Constructs with specific binding affinities can be generated using DARPin libraries e.g., as described in Seeger, et al., Protein Sci., 22:1239-1257 (2013).

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof, or a humanized murine antibody or a fragment thereof.

In certain embodiments, the antigen binding domain comprises a heavy chain variable region that comprises three heavy chain complementarity determining regions (HCDRs) and a light chain variable region that comprises three light chain complementarity determining regions (LCDRs). In certain embodiments, the antigen binding domain comprises a linker.

### Transmembrane Domain

CARs of the present invention may comprise a transmembrane domain that connects the antigen binding domain of the CAR to the intracellular domain of the CAR. The transmembrane domain of the CAR is a region that is capable of spanning the plasma membrane of a cell (e.g., an immune cell or precursor thereof). In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), ICOS, CD278, Toll-like receptor 1 (TLR1), TLR2, TLR3, TLR4, TLR5, TLR6, TLR7, TLR8, TLR9 or a transmembrane domain derived from a killer immunoglobulin-like receptor (KIR).

In certain embodiments, the transmembrane domain comprises a transmembrane domain of CD8. In certain embodiments, the transmembrane domain of CD8 is a transmembrane domain of CD8α.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in the CAR.

In some embodiments, the transmembrane domain further comprises a hinge region. The CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, the CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). The hinge region can comprise an amino acid sequence of a human IgG1, IgG2, IgG3, or IgG4, hinge region (see, e.g., Yan et al., J. Biol. Chem. (2012) 287: 5891-5897). In one embodiment, the hinge region can comprise an amino acid sequence derived from human CD8, or a variant thereof.

### Intracellular Signaling Domain

The CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may comprise an intracellular signaling domain of a protein selected from human CD3 zeta chain, FcyRIII, FcsRI, DAP10, DAP12, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, such as any synthetic sequence thereof, that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in the CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of the CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fe epsilon receptor I gamma chain; Fc receptor gamma-chain; fc-epsilon RI-gamma; fcRgamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, A1504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire molecule. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular domains described herein can be combined with any of the antigen binding domains described herein, any of the transmembrane domains described herein, or any of the other domains described herein that may be included in the CAR.

In certain embodiments, the intracellular domain comprises a costimulatory domain of 4-1BB. In certain embodiments, the intracellular domain comprises an intracellular domain of CD3ζ or a variant thereof. In certain embodiments, the intracellular domain comprises a costimulatory domain of 4-1BB and an intracellular domain of CD3ζ.

Tolerable variations of the individual CAR domain sequences (leader, antigen binding domain, hinge, transmembrane, and/or intracellular domains) will be known to those of skill in the art. For example, in certain embodiments the CAR domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known sequence.

In one aspect, the invention provides a chimeric antigen receptor (CAR) comprising a tumor antigen binding domain, a transmembrane domain, and an intracellular domain. In certain embodiments, the CAR comprises an anti-CD19 antigen binding domain, a transmembrane domain, a 4-1BB costimulatory domain, and a CD3z intracellular domain.

In some embodiments, the CAR is the CTL019 CAR comprising the following amino acid sequence:

(SEQ ID NO: 16)
MALPVTALLLPLALLLHAARPDIQMTQTTSSLSASLGDRVTISCRASQDISKYLNWYQQ
KPDGTVKLLIYHTSRLHSGVPSRFSGSGSGTDYSLTISNLEQEDIATYFCQQGNTLPYTFG
GGTKLEITGGGGSGGGGSGGGGSEVKLQESGPGLVAPSQSLSVTCTVSGVSLPDYGVSW
IRQPPRKGLEWLGVIWGSETTYYNSALKSRLTIIKDNSKSQVFLKMNSLQTDDTAIYYCA
KHYYYGGSYAMDYWGQGTSVTVSSTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEED
GCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKD
TYDALHMQALPPR.

In some embodiments, the CAR is the CTL019 CAR encoded by the following nucleotide sequence:

| (SEQ ID NO: 17) | |
|---|---|
| atggccttac cagtgaccgc cttgctcctg ccgctggcct tgctgctcca cgccgccagg | 60 |
| ccggacatcc agatgacaca gactacatcc tccctgtctg cctctctggg agacagagtc | 120 |
| accatcagtt gcagggcaag tcaggacatt agtaaatatt taaattggta tcagcagaaa | 180 |
| ccagatggaa ctgttaaact cctgatctac catacatcaa gattacactc aggagtccca | 240 |
| tcaaggttca gtggcagtgg gtctggaaca gattattctc tcaccattag caacctggag | 300 |
| caagaagata ttgccactta cttttgccaa cagggtaata cgcttccgta cacgttcgga | 360 |
| ggggggacca agctggagat cacaggtggc ggtggctcgg gcggtggtgg gtcgggtggc | 420 |
| ggcggatctg aggtgaaact gcaggagtca ggacctggcc tggtggcgcc ctcacagagc | 480 |
| ctgtccgtca catgcactgt ctcaggggtc tcattacccg actatggtgt aagctggatt | 540 |
| cgccagcctc cacgaaaggg tctggagtgg ctgggagtaa tatggggtag tgaaaccaca | 600 |
| tactataatt cagctctcaa atccagactg accatcatca aggacaactc caagagccaa | 660 |
| gttttcttaa aaatgaacag tctgcaaact gatgacacag ccatttacta ctgtgccaaa | 720 |
| cattattact acggtggtag ctatgctatg gactactggg gccaaggaac ctcagtcacc | 780 |
| gtctcctcaa ccacgacgcc agcgccgcga ccaccaacac cggcgcccac catcgcgtcg | 840 |
| cagcccctgt ccctgcgccc agaggcgtgc cggccagcgg cggggggcgc agtgcacacg | 900 |
| agggggctgg acttcgcctg tgatatctac atctgggcgc ccttggccgg gacttgtggg | 960 |
| gtccttctcc tgtcactggt tatcaccctt tactgcaaac ggggcagaaa gaaactcctg | 1020 |
| tatatattca aacaaccatt tatgagacca gtacaaacta ctcaagagga agatggctgt | 1080 |
| agctgccgat ttccagaaga agaagaagga ggatgtgaac tgagagtgaa gttcagcagg | 1140 |
| agcgcagacg cccccgcgta caagcagggc cagaaccagc tctataacga gctcaatcta | 1200 |
| ggacgaagag aggagtacga tgttttggac aagagacgtg gccgggaccc tgagatgggg | 1260 |
| ggaaagccga gaaggaagaa ccctcaggaa ggcctgtaca atgaactgca gaaagataag | 1320 |
| atggcggagg cctacagtga gattgggatg aaaggcgage gccggagggg caaggggcac | 1380 |
| gatggccttt accagggtct cagtacagcc accaaggaca cctacgacgc ccttcacatg | 1440 |
| caggccctgc cccctcgc. | 1458 |

In certain embodiments, the CAR is an anti-CD30 CAR comprising an anti-CD30 antigen binding domain. In some embodiments, the anti-CD30 antigen binding domain comprises an anti-CD30 scFv. In some embodiments, the anti-CD30 scFv comprises an amino acid sequence selected from SEQ ID NO: 18, SEQ ID NO: 19, and SEQ ID NO: 20. In some embodiments, the anti-CD30 scFv comprises SEQ ID NO: 18. In some embodiments, the anti-CD30 scFv comprises SEQ ID NO: 19. In some embodiments, the anti-CD30 scFv comprises SEQ ID NO: 20.

(SEQ ID NO: 18)
EVQLLESGGGLVQPGGSLRLSCAASGFTFSSYGMSWVRQAPGKGLEWVSSIYYYSGGTYYADSV
KGRFTISRDNSKNTLYLQMNSLRAEDTAVYYCARNDYRNNSDFDYWGQGTLVTVSSGGGGSGGG
GSGGGGSQSVLTQPPSASGTPGQRVTISCSGSSSNIGSNYVYWYQQLPGTAPKLLIYRNNQRPS
GVPDRFSGSKSGTSASLAISGLRSEDEADYYCAAYYPYYYVFGGGTKLTVLGGQAG.
(SEQ ID NO: 19)
EVQLLESGGGLVQPGGSLRLSCAASGFTFSYYYMSWVRQAPGKGLEWVSYIGGGGSGTYYADSV
KGRFTISRDNSKNTLYLQMNSLRAEDTAVYYCARGPYYGYFDYWGQGTLVTVSSGGGGSGGGGS
GGGGSQSVLTQPPSASGTPGQRVTISCSGSSSNIGSNYVYWYQQLPGTAPKLLIYRNNQRPSGV
PDRFSGSKSGTSASLAISGLRSEDEADYYCAAYPSYDSYVFGGGTKLTVLGGQAG.
(SEQ ID NO: 20)
EVKLQQSGAELARPGASVKMSCKASGYTFTTYTIHWVRQRPGHDLEWIGYINPSSGYSDYNQNF
KGKTTLTADKSSNTAYMQLNSLTSEDSAVYYCARRADYGNYEYTWFAYWGQGTTVTVSSGGGGS
GGGGSGGGGSDIELTQSPKFMSTSVGDRVNVTCKASQNVGTNVAWFQQKPGQSPKVLIYSASYR
YSGVPDRFTGSGSGTDFTLTISNVQSEDLAEYFCQQYHTYPLTFGGGTKLEIKGQAG.

In some embodiments, the anti-CD30 scFv is encoded by a nucleotide sequence selected from SEQ ID NO: 21, SEQ ID NO: 22, and SEQ ID NO: 23. In some embodiments, the anti-CD30 scFv is encoded by SEQ ID NO: 21. In some embodiments, the anti-CD30 scFv is encoded by SEQ ID NO: 22. In some embodiments, the anti-CD30 scFv is encoded by SEQ ID NO: 23.

(SEQ ID NO: 21)
GAGGTGCAGCTGTTGGAGTCTGGGGGAGGCTTGGTACAGCCTGGGGGGTCCCTGAGACTCTCCT
GTGCAGCCTCTGGATTCACCTTTAGCTCTTATGGTATGAGCTGGGTCCGCCAGGCTCCAGGGAA
GGGGCTGGAGTGGGTCTCATCTATCTACTACTACTCTGGTGGTACGTATTACGCTGATTCTGTA
AAAGGTCGGTTCACCATCTCCAGAGACAATTCCAAGAACACGCTGTATCTGCAAATGAACAGCC
TGAGAGCCGAGGACACGGCCGTGTATTACTGTGCGCGTAACGACTACCGTAACAACTCTGACTT
CGACTACTGGGGCCAGGGTACACTGGTCACCGTGAGCTCAGGTGGAGGCGGTTCAGGCGGAGGT
GGATCCGGCGGTGGCGGATCGCAGTCTGTGCTGACTCAGCCACCCTCAGCGTCTGGGACCCCCG
GGCAGAGGGTCACCATCTCTTGTAGTGGCTCTTCATCTAATATTGGCTCTAATTATGTCTACTG
GTACCAGCAGCTCCCAGGAACGGCCCCCAAACTCCTCATCTATAGAAATAACCAGCGGCCAAGC
GGGGTCCCTGACCGATTCTCTGGCTCCAAGTCTGGCACCTCAGCCTCCCTGGCCATCAGTGGGC
TCCGGTCCGAGGATGAGGCCGATTATTACTGTGCTGCTTACTACCCGTACTACTATGTCTTCGG
CGGAGGCACCAAGCTGACGGTCCTAGGTGGCCAGGCCGGCC.
(SEQ ID NO: 22)
GAGGTGCAGCTGTTGGAGTCTGGGGGAGGCTTGGTACAGCCTGGGGGGTCCCTGAGACTCTCCT
GTGCAGCCTCTGGATTCACCTTTAGCTACTATTACATGAGCTGGGTCCGCCAGGCTCCAGGGAA
GGGGCTGGAGTGGGTCTCATACATCGGTGGTGGTGGTTCTGGTACGTATTACGCTGATTCTGTA
AAAGGTCGGTTCACCATCTCCAGAGACAATTCCAAGAACACGCTGTATCTGCAAATGAACAGCC
TGAGAGCCGAGGACACGGCCGTGTATTACTGTGCGCGTGGTCCGTACTACGGTTACTTCGACTA
CTGGGGCCAGGGTACACTGGTCACCGTGAGCTCAGGTGGAGGCGGTTCAGGCGGAGGTGGATCC
GGCGGTGGCGGATCGCAGTCTGTGCTGACTCAGCCACCCTCAGCGTCTGGGACCCCCGGGCAGA
GGGTCACCATCTCTTGTAGTGGCTCTTCATCTAATATTGGCTCTAATTATGTCTACTGGTACCA
GCAGCTCCCAGGAACGGCCCCCAAACTCCTCATCTATAGAAATAACCAGCGGCCAAGCGGGGTC
CCTGACCGATTCTCTGGCTCCAAGTCTGGCACCTCAGCCTCCCTGGCCATCAGTGGGCTCCGGT
CCGAGGATGAGGCCGATTATTACTGTGCTGCTTACCCGTCTTACGACTCTTATGTCTTCGGCGG
AGGCACCAAGCTGACGGTCCTAGGTGGCCAGGCCGGCC.
(SEQ ID NO: 23)
GAGGTGAAGCTTCAACAGTCCGGAGCAGAACTCGCCCGCCCGGGCGCTAGCGTGAAAATGAGCT
GCAAGGCCTCTGGATATACCTTCACCACCTATACCATTCACTGGGTGAGACAGAGACCTGGCCA
TGACCTGGAGTGGATCGGCTATATAAACCCAAGCAGTGGCTACTCCGACTACAATCAGAATTTC
AAAGGCAAAACAACACTGACCGCGGACAAGTCTTCAAATACTGCCTACATGCAGCTGAACTCTC
TGACAAGCGAGGATTCAGCAGTGTATTACTGCGCTAGGCGGGCTGACTACGGCAACTACGAGTA
TACCTGGTTTGCATACTGGGGCCAGGGAACAACGGTTACAGTGTCCTCAGGCGGCGGAGGGAGC
GGGGGTGGTGGGAGTGGAGGCGGGGGGAGTGATATCGAACTGACGCAGTCACCTAAGTTTATGA
GCACATCCGTGGGGGATCGAGTCAATGTCACGTGTAAGGCCAGCCAGAATGTAGGCACTAACGT
GGCCTGGTTTCAACAGAAGCCCGGTCAAAGCCCCAAAGTTCTGATCTACAGCGCTTCCTATAGG
TACAGTGGCGTGCCAGATAGATTCACCGGATCCGGAAGCGGAACCGATTTCACTCTCACTATTT
CAAACGTTCAATCGGAAGATTTGGCCGAGTATTTTTGTCAGCAGTATCACACATACCCCTTAAC
TTTCGGTGGCGGTACTAAGCTGGAAATTAAAGGCCAGGCCGGCC.

In some embodiments, the CAR is an anti-CD30 CAR. In some embodiments, the anti-CD30 CAR comprises a CD8 leader, an anti-CD30 scFv, a CD8 hinge and transmembrane domain, and an intracellular domain comprising a 4-1BB intracellular costimulatory domain and a CD3 zeta intracellular signaling domain. In some embodiments, the anti-CD30 CAR comprises an amino acid sequence selected from SEQ ID NO: 24, SEQ ID NO: 25, and SEQ ID NO: 26. In some embodiments, the CAR is an anti-CD30 CAR comprising SEQ ID NO: 24. In some embodiments, the CAR is an anti-CD30 CAR comprising SEQ ID NO: 25. In some embodiments, the CAR is an anti-CD30 CAR comprising SEQ ID NO: 26.

(SEQ ID NO: 24)
MALPVTALLLPLALLLHAARPEVQLLESGGGLVQPGGSLRLSCAASGFTFSSYGMSWVRQAPGK
GLEWVSSIYYYSGGTYYADSVKGRFTISRDNSKNTLYLQMNSLRAEDTAVYYCARNDYRNNSDF
DYWGQGTLVTVSSGGGGSGGGGSGGGGSQSVLTQPPSASGTPGQRVTISCSGSSSNIGSNYVYW
YQQLPGTAPKLLIYRNNQRPSGVPDRESGSKSGTSASLAISGLRSEDEADYYCAAYYPYYYVFG
GGTKLTVLGGQAGQTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR.
(SEQ ID NO: 25)
MALPVTALLLPLALLLHAARPEVQLLESGGGLVQPGGSLRLSCAASGFTFSYYYMSWVRQAPGK
GLEWVSYIGGGGSGTYYADSVKGRFTISRDNSKNTLYLQMNSLRAEDTAVYYCARGPYYGYFDY
WGQGTLVTVSSGGGGSGGGGSGGGGSQSVLTQPPSASGTPGQRVTISCSGSSSNIGSNYVYWYQ
QLPGTAPKLLIYRNNQRPSGVPDRESGSKSGTSASLAISGLRSEDEADYYCAAYPSYDSYVEGG
GTKLTVLGGQAGQTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR.
(SEQ ID NO: 26)
MALPVTALLLPLALLLHAARPEVKLQQSGAELARPGASVKMSCKASGYTFTTYTIHWVRQRPGH
DLEWIGYINPSSGYSDYNQNFKGKTTLTADKSSNTAYMQLNSLTSEDSAVYYCARRADYGNYEY
TWFAYWGQGTTVTVSSGGGGSGGGGSGGGGSDIELTQSPKFMSTSVGDRVNVTCKASQNVGTNV
AWFQQKPGQSPKVLIYSASYRYSGVPDRFTGSGSGTDFTLTISNVQSEDLAEYFCQQYHTYPLT
FGGGTKLEIKGQAGQTTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR.

In some embodiments, the CAR is an anti-CD30 CAR encoded by a nucleotide sequence selected from SEQ ID NO: 27, SEQ ID NO: 28, and SEQ ID NO: 29. In some embodiments, the CAR is an anti-CD30 CAR encoded by SEQ ID NO: 27. In some embodiments, the CAR is an anti-CD30 CAR encoded by SEQ ID NO: 28. In some embodiments, the CAR is an anti-CD30 CAR encoded by SEQ ID NO: 29.

(SEQ ID NO: 27)
ATGGCCTTACCAGTGACCGCCTTGCTCCTGCCGCTGGCCTTGCTGCTCCACGCCGCCAGGCCGG
AGGTGCAGCTGTTGGAGTCTGGGGGAGGCTTGGTACAGCCTGGGGGGTCCCTGAGACTCTCCTG
TGCAGCCTCTGGATTCACCTTTAGCTCTTATGGTATGAGCTGGGTCCGCCAGGCTCCAGGGAAG
GGGCTGGAGTGGGTCTCATCTATCTACTACTACTCTGGTGGTACGTATTACGCTGATTCTGTAA
AAGGTCGGTTCACCATCTCCAGAGACAATTCCAAGAACACGCTGTATCTGCAAATGAACAGCCT
GAGAGCCGAGGACACGGCCGTGTATTACTGTGCGCGTAACGACTACCGTAACAACTCTGACTTC
GACTACTGGGGCCAGGGTACACTGGTCACCGTGAGCTCAGGTGGAGGCGGTTCAGGCGGAGGTG
GATCCGGCGGTGGCGGATCGCAGTCTGTGCTGACTCAGCCACCCTCAGCGTCTGGGACCCCCGG
GCAGAGGGTCACCATCTCTTGTAGTGGCTCTTCATCTAATATTGGCTCTAATTATGTCTACTGG
TACCAGCAGCTCCCAGGAACGGCCCCCAAACTCCTCATCTATAGAAATAACCAGCGGCCAAGCG
GGGTCCCTGACCGATTCTCTGGCTCCAAGTCTGGCACCTCAGCCTCCCTGGCCATCAGTGGGCT
CCGGTCCGAGGATGAGGCCGATTATTACTGTGCTGCTTACTACCCGTACTACTATGTCTTCGGC
GGAGGCACCAAGCTGACGGTCCTAGGTGGCCAGGCCGGCCAGACAACGACACCTGCTCCCAGAC
CGCCTACTCCCGCCCCAACCATTGCATCTCAGCCACTCTCTCTGAGACCCGAAGCGTGTAGACC
TGCGGCCGGGGGCGCTGTCCACACAAGAGGCTTAGACTTCGCCTGCGATATCTATATCTGGGCC
CCACTCGCAGGCACTTGTGGAGTGCTGCTGCTTTCACTCGTGATAACCCTGTACTGCAAAAGGG
GGAGAAAGAAGCTGCTGTATATTTTTAAACAACCATTTATGAGACCTGTTCAGACTACCCAGGA
AGAAGACGGTTGTAGTTGCAGATTCCCCGAGGAGGAAGAAGGAGGTTGCGAGTTGAGAGTAAAG
TTCAGCAGATCCGCAGATGCCCCTGCTTACCAGCAGGGTCAAAACCAGCTTTACAACGAGCTGA
ATTTAGGTAGAAGAGAGGAATATGACGTGTTGGATAAAAGAAGAGGAAGAGACCCGGAAATGGG
CGGCAAGCCTCGAAGAAAAAATCCCCAAGAGGGACTCTACAATGAGCTGCAGAAGGACAAAATG
GCTGAAGCCTACAGCGAGATCGGCATGAAGGGAGAAAGACGCAGAGGGAAAGGGCATGATGGGC
TTTATCAGGGCTTGTCCACCGCTACAAAGGATACTTATGACGCACTACACATGCAGGCCCTGCC
ACCCCGT.
(SEQ ID NO: 28)
ATGGCCTTACCAGTGACCGCCTTGCTCCTGCCGCTGGCCTTGCTGCTCCACGCCGCCAGGCCGG
AGGTGCAGCTGTTGGAGTCTGGGGGAGGCTTGGTACAGCCTGGGGGGTCCCTGAGACTCTCCTG
TGCAGCCTCTGGATTCACCTTTAGCTACTATTACATGAGCTGGGTCCGCCAGGCTCCAGGGAAG
GGGCTGGAGTGGGTCTCATACATCGGTGGTGGTGGTTCTGGTACGTATTACGCTGATTCTGTAA
AAGGTCGGTTCACCATCTCCAGAGACAATTCCAAGAACACGCTGTATCTGCAAATGAACAGCCT
GAGAGCCGAGGACACGGCCGTGTATTACTGTGCGCGTGGTCCGTACTACGGTTACTTCGACTAC
TGGGGCCAGGGTACACTGGTCACCGTGAGCTCAGGTGGAGGCGGTTCAGGCGGAGGTGGATCCG
GCGGTGGCGGATCGCAGTCTGTGCTGACTCAGCCACCCTCAGCGTCTGGGACCCCCGGGCAGAG
GGTCACCATCTCTTGTAGTGGCTCTTCATCTAATATTGGCTCTAATTATGTCTACTGGTACCAG
CAGCTCCCAGGAACGGCCCCCAAACTCCTCATCTATAGAAATAACCAGCGGCCAAGCGGGGTCC
CTGACCGATTCTCTGGCTCCAAGTCTGGCACCTCAGCCTCCCTGGCCATCAGTGGGCTCCGGTC
CGAGGATGAGGCCGATTATTACTGTGCTGCTTACCCGTCTTACGACTCTTATGTCTTCGGCGGA
GGCACCAAGCTGACGGTCCTAGGTGGCCAGGCCGGCCAGACAACGACACCTGCTCCCAGACCGC
CTACTCCCGCCCCAACCATTGCATCTCAGCCACTCTCTCTGAGACCCGAAGCGTGTAGACCTGC
GGCCGGGGGCGCTGTCCACACAAGAGGCTTAGACTTCGCCTGCGATATCTATATCTGGGCCCCA
CTCGCAGGCACTTGTGGAGTGCTGCTGCTTTCACTCGTGATAACCCTGTACTGCAAAAGGGGGA
GAAAGAAGCTGCTGTATATTTTTAAACAACCATTTATGAGACCTGTTCAGACTACCCAGGAAGA
AGACGGTTGTAGTTGCAGATTCCCCGAGGAGGAAGAAGGAGGTTGCGAGTTGAGAGTAAAGTTC
AGCAGATCCGCAGATGCCCCTGCTTACCAGCAGGGTCAAAACCAGCTTTACAACGAGCTGAATT
TAGGTAGAAGAGAGGAATATGACGTGTTGGATAAAAGAAGAGGAAGAGACCCGGAAATGGGCGG
CAAGCCTCGAAGAAAAAATCCCCAAGAGGGACTCTACAATGAGCTGCAGAAGGACAAAATGGCT
GAAGCCTACAGCGAGATCGGCATGAAGGGAGAAAGACGCAGAGGGAAAGGGCATGATGGGCTTT
ATCAGGGCTTGTCCACCGCTACAAAGGATACTTATGACGCACTACACATGCAGGCCCTGCCACC
CCGT.
(SEQ ID NO: 29)
ATGGCCTTACCAGTGACCGCCTTGCTCCTGCCGCTGGCCTTGCTGCTCCACGCCGCCAGGCCGG
AGGTGAAGCTTCAACAGTCCGGAGCAGAACTCGCCCGCCCGGGCGCTAGCGTGAAAATGAGCTG
CAAGGCCTCTGGATATACCTTCACCACCTATACCATTCACTGGGTGAGACAGAGACCTGGCCAT
GACCTGGAGTGGATCGGCTATATAAACCCAAGCAGTGGCTACTCCGACTACAATCAGAATTTCA
AAGGCAAAACAACACTGACCGCGGACAAGTCTTCAAATACTGCCTACATGCAGCTGAACTCTCT
GACAAGCGAGGATTCAGCAGTGTATTACTGCGCTAGGCGGGCTGACTACGGCAACTACGAGTAT
ACCTGGTTTGCATACTGGGGCCAGGGAACAACGGTTACAGTGTCCTCAGGCGGCGGAGGGAGCG
GGGGTGGTGGGAGTGGAGGCGGGGGGAGTGATATCGAACTGACGCAGTCACCTAAGTTTATGAG
CACATCCGTGGGGGATCGAGTCAATGTCACGTGTAAGGCCAGCCAGAATGTAGGCACTAACGTG
GCCTGGTTTCAACAGAAGCCCGGTCAAAGCCCCAAAGTTCTGATCTACAGCGCTTCCTATAGGT
ACAGTGGCGTGCCAGATAGATTCACCGGATCCGGAAGCGGAACCGATTTCACTCTCACTATTTC
AAACGTTCAATCGGAAGATTTGGCCGAGTATTTTTGTCAGCAGTATCACACATACCCCTTAACT
TTCGGTGGCGGTACTAAGCTGGAAATTAAAGGCCAGGCCGGCCAGACAACGACACCTGCTCCCA
GACCGCCTACTCCCGCCCCAACCATTGCATCTCAGCCACTCTCTCTGAGACCCGAAGCGTGTAG
ACCTGCGGCCGGGGGCGCTGTCCACACAAGAGGCTTAGACTTCGCCTGCGATATCTATATCTGG
GCCCCACTCGCAGGCACTTGTGGAGTGCTGCTGCTTTCACTCGTGATAACCCTGTACTGCAAAA
GGGGGAGAAAGAAGCTGCTGTATATTTTTAAACAACCATTTATGAGACCTGTTCAGACTACCCA
GGAAGAAGACGGTTGTAGTTGCAGATTCCCCGAGGAGGAAGAAGGAGGTTGCGAGTTGAGAGTA
AAGTTCAGCAGATCCGCAGATGCCCCTGCTTACCAGCAGGGTCAAAACCAGCTTTACAACGAGC
TGAATTTAGGTAGAAGAGAGGAATATGACGTGTTGGATAAAAGAAGAGGAAGAGACCCGGAAAT
GGGCGGCAAGCCTCGAAGAAAAAATCCCCAAGAGGGACTCTACAATGAGCTGCAGAAGGACAAA
ATGGCTGAAGCCTACAGCGAGATCGGCATGAAGGGAGAAAGACGCAGAGGGAAAGGGCATGATG
GGCTTTATCAGGGCTTGTCCACCGCTACAAAGGATACTTATGACGCACTACACATGCAGGCCCT
GCCACCCCGT.

In some embodiments, the anti-CD30 CAR is expressed from a pLV3.1_T1-159 vector comprising the following sequence:

(SEQ ID NO: 30)
AGCTTAATGTAGTCTTATGCAATACTCTTGTAGTCTTGCAACATGGTAACGATGAGTTAGCAAC
ATGCCTTACAAGGAGAGAAAAAGCACCGTGCATGCCGATTGGTGGAAGTAAGGTGGTACGATCG
TGCCTTATTAGGAAGGCAACAGACGGGTCTGACATGGATTGGACGAACCACTGAATTGCCGCAT
TGCAGAGATATTGTATTTAAGTGCCTAGCTCGATACATAAACGGGTCTCTCTGGTTAGACCAGA
TCTGAGCCTGGGAGCTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACTAGAGATCCCTCAGA
CCCTTTTAGTCAGTGTGGAAAATCTCTAGCAGTGGCGCCCGAACAGGGACTTGAAAGCGAAAGG
GAAACCAGAGGAGCTCTCTCGACGCAGGACTCGGCTTGCTGAAGCGCGCACGGCAAGAGGCGAG
GGGCGGCGACTGGTGAGTACGCCAAAAATTTTGACTAGCGGAGGCTAGAAGGAGAGAGATGGGT
GCGAGAGCGTCAGTATTAAGCGGGGGAGAATTAGATCGCGATGGGAAAAAATTCGGTTAAGGCC
AGGGGGAAAGAAAAAATATAAATTAAAACATATAGTATGGGCAAGCAGGGAGCTAGAACGATTC
GCAGTTAATCCTGGCCTGTTAGAAACATCAGAAGGCTGTAGACAAATACTGGGACAGCTACAAC
CATCCCTTCAGACAGGATCAGAAGAACTTAGATCATTATATAATACAGTAGCAACCCTCTATTG
TGTGCATCAAAGGATAGAGATAAAAGACACCAAGGAAGCTTTAGACAAGATAGAGGAAGAGCAA
AACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAG
CACCCACCAAGGCAAAGAGAAGAGTGGTGCAGAGAGAAAAAAGAGCAGTGGGAATAGGAGCTTT
GTTCCTTGGGTTCTTGGGAGCAGCAGGAAGCACTATGGGCGCAGCGTCAATGACGCTGACGGTA
CAGGCCAGACAATTATTGTCTGGTATAGTGCAGCAGCAGAACAATTTGCTGAGGGCTATTGAGG
CGCAACAGCATCTGTTGCAACTCACAGTCTGGGGCATCAAGCAGCTCCAGGCAAGAATCCTGGC
TGTGGAAAGATACCTAAAGGATCAACAGCTCCTGGGGATTTGGGGTTGCTCTGGAAAACTCATT
TGCACCACTGCTGTGCCTTGGAATGCTAGTTGGAGTAATAAATCTCTGGAACAGATTGGAATCA
CACGACCTGGATGGAGTGGGACAGAGAAATTAACAATTACACAAGCTTAATACACTCCTTAATT
GAAGAATCGCAAAACCAGCAAGAAAAGAATGAACAAGAATTATTGGAATTAGATAAATGGGCAA
GTTTGTGGAATTGGTTTAACATAACAAATTGGCTGTGGTATATAAAATTATTCATAATGATAGT
AGGAGGCTTGGTAGGTTTAAGAATAGTTTTTGCTGTACTTTCTATAGTGAATAGAGTTAGGCAG
GGATATTCACCATTATCGTTTCAGACCCACCTCCCAACCCCGAGGGGACCCGACAGGCCCGAAG
GAATAGAAGAAGAAGGTGGAGAGAGAGACAGAGACAGATCCATTCGATTAGTGAACGGATCTCG
ACGGTATCGGTTAACTTTTAAAAGAAAAGGGGGGATTGGGGGGTACAGTGCAGGGGAAAGAATA
GTAGACATAATAGCAACAGACATACAAACTAAAGAATTACAAAAACAAATTACAAAAATTCAAA
ATTTTATCGATCACGAGACTAGCCTCGAGAAGCTTGATATCGAATTCCACGTCGACTTATGTGA
GGCTCCGGTGCCCGTCAGTGGGCAGAGCGCACATCGCCCACAGTCCCCGAGAAGTTGGGGGGAG
GGGTCGGCAATTGAACCGGTGCCTAGAGAAGGTGGCGCGGGGTAAACTGGGAAAGTGATGTCGT
GTACTGGCTCCGCCTTTTTCCCGAGGGTGGGGGAGAACCGTATATAAGTGCAGTAGTCGCCGTG
AACGTTCTTTTTCGCAACGGGTTTGCCGCCAGAACACAGGTAAGTGCCGTGTGTGGTTCCCGCG
GGCCTGGCCTCTTTACGGGTTATGGCCCTTGCGTGCCTTGAATTACTTCCACCTGGCTGCAGTA
CGTGATTCTTGATCCCGAGCTTCGGGTTGGAAGTGGGTGGGAGAGTTCGAGGCCTTGCGCTTAA
GGAGCCCCTTCGCCTCGTGCTTGAGTTGAGGCCTGGCCTGGGCGCTGGGGCCGCCGCGTGCGAA
TCTGGTGGCACCTTCGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGGGCCAAGATCTGCAC
ACTGGTATTTCGGTTTTTGGGGCCGCGGGCGGCGACGGGGCCCGTGCGTCCCAGCGCACATGTT
CGGCGAGGCGGGGCCTGCGAGCGCGGCCACCGAGAATCGGACGGGGGTAGTCTCAAGCTGGCCG
GCCTGCTCTGGTGCCTGGCCTCGCGCCGCCGTGTATCGCCCCGCCCTGGGCGGCAAGGCTGGCC
CGGTCGGCACCAGTTGCGTGAGCGGAAAGATGGCCGCTTCCCGGCCCTGCTGCAGGGAGCTCAA
AATGGAGGACGCGGCGCTCGGGAGAGCGGGCGGGTGAGTCACCCACACAAAGGAAAAGGGCCTT
TCCGTCCTCAGCCGTCGCTTCATGTGACTCCACGGAGTACCGGGCGCCGTCCAGGCACCTCGAT
TAGTTCTCGAGCTTTTGGAGTACGTCGTCTTTAGGTTGGGGGGAGGGGTTTTATGCGATGGAGT
TTCCCCACACTGAGTGGGTGGAGACTGAAGTTAGGCCAGCTTGGCACTTGATGTAATTCTCCTT
GGAATTTGCCCTTTTTGAGTTTGGATCTTGGTTCATTCTCAAGCCTCAGACAGTGGTTCAAAGT
TTTTTTCTTCCATTTCAGGTGTCGTGAGGATCCACTAGTGCCACCATGGCCTTACCAGTGACCG
CCTTGCTCCTGCCGCTGGCCTTGCTGCTCCACGCCGCCAGGCCGGAGGTGCAGCTGTTGGAGTC
TGGGGGAGGCTTGGTACAGCCTGGGGGGTCCCTGAGACTCTCCTGTGCAGCCTCTGGATTCACC
TTTAGCTCTTATGGTATGAGCTGGGTCCGCCAGGCTCCAGGGAAGGGGCTGGAGTGGGTCTCAT
CTATCTACTACTACTCTGGTGGTACGTATTACGCTGATTCTGTAAAAGGTCGGTTCACCATCTC
CAGAGACAATTCCAAGAACACGCTGTATCTGCAAATGAACAGCCTGAGAGCCGAGGACACGGCC
GTGTATTACTGTGCGCGTAACGACTACCGTAACAACTCTGACTTCGACTACTGGGGCCAGGGTA
CACTGGTCACCGTGAGCTCAGGTGGAGGCGGTTCAGGCGGAGGTGGATCCGGCGGTGGCGGATC
GCAGTCTGTGCTGACTCAGCCACCCTCAGCGTCTGGGACCCCCGGGCAGAGGGTCACCATCTCT
TGTAGTGGCTCTTCATCTAATATTGGCTCTAATTATGTCTACTGGTACCAGCAGCTCCCAGGAA
CGGCCCCCAAACTCCTCATCTATAGAAATAACCAGCGGCCAAGCGGGGTCCCTGACCGATTCTC
TGGCTCCAAGTCTGGCACCTCAGCCTCCCTGGCCATCAGTGGGCTCCGGTCCGAGGATGAGGCC
GATTATTACTGTGCTGCTTACTACCCGTACTACTATGTCTTCGGCGGAGGCACCAAGCTGACGG
TCCTAGGTGGCCAGGCCGGCCAGACAACGACACCTGCTCCCAGACCGCCTACTCCCGCCCCAAC
CATTGCATCTCAGCCACTCTCTCTGAGACCCGAAGCGTGTAGACCTGCGGCCGGGGGCGCTGTC
CACACAAGAGGCTTAGACTTCGCCTGCGATATCTATATCTGGGCCCCACTCGCAGGCACTTGTG
GAGTGCTGCTGCTTTCACTCGTGATAACCCTGTACTGCAAAAGGGGGAGAAAGAAGCTGCTGTA
TATTTTTAAACAACCATTTATGAGACCTGTTCAGACTACCCAGGAAGAAGACGGTTGTAGTTGC
AGATTCCCCGAGGAGGAAGAAGGAGGTTGCGAGTTGAGAGTAAAGTTCAGCAGATCCGCAGATG
CCCCTGCTTACCAGCAGGGTCAAAACCAGCTTTACAACGAGCTGAATTTAGGTAGAAGAGAGGA
ATATGACGTGTTGGATAAAAGAAGAGGAAGAGACCCGGAAATGGGCGGCAAGCCTCGAAGAAAA
AATCCCCAAGAGGGACTCTACAATGAGCTGCAGAAGGACAAAATGGCTGAAGCCTACAGCGAGA
TCGGCATGAAGGGAGAAAGACGCAGAGGGAAAGGGCATGATGGGCTTTATCAGGGCTTGTCCAC
CGCTACAAAGGATACTTATGACGCACTACACATGCAGGCCCTGCCACCCCGTTGATTAATTAAC
TCGAGCTCGAGTCTAGAGGGCCCGCGGTTCGAAGGTAAGCCTATCCCTAACCCTCTCCTCGGTC
TCGATTCTACGCGTACCGGTTAGTAATGATCGACAATCAACCTCTGGATTACAAAATTTGTGAA
AGATTGACTGGTATTCTTAACTATGTTGCTCCTTTTACGCTATGTGGATACGCTGCTTTAATGC
CTTTGTATCATGCTATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGGTGTGCACTGTGTTT
GCTGACGCAACCCCCACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGACGTCCTTTCCATGG
CTGCTCGCCTGTGTTGCCACCTGGATTCTGCGCGGGACGTCCTTCTGCTACGTCCCTTCGGCCC
TCAATCCAGCGGACCTTCCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGCCTGGAATTCGAGCTCGG
TACCTTTAAGACCAATGACTTACAAGGCAGCTGTAGATCTTAGCCACTTTTTAAAAGAAAAGGG
GGGACTGGAAGGGCTAATTCACTCCCAACGAAGACAAGATCTGCTTTTTGCTTGTACTGGGTCT
CTCTGGTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTGGTAA
CTAGAGATCCCTCAGACCCTTTTAGTCAGTGTGGAAAATCTCTAGCAGTAGTAGTTCATGTCAT
CTTATTATTCAGTATTTATAACTTGCAAAGAAATGAATATCAGAGAGTGAGAGGAACTTGTTTA
TTGCAGCTTATAATGGTTACAAATAAAGCAATAGCATCACAAATTTCACAAATAAAGCATTTTT
TTCACTGCATTCTAGTTGTGGTTTGTCCAAACTCATCAATGTATCTTATCATGTCTGGCTCTAG
CTATCCCGCCCCTAACTCCGCCCATCCCGCCCCTAACTCCGCCCAGTTCCGCCCATTCTCCGCC
CCATGGCTGACTAATTTTTTTTATTTATGCAGAGGCCGAGGCCGCCTCGGCCTCTGAGCTATTC
CAGAAGTAGTGAGGAGGCTTTTTTGGAGGCCTAGGGACGTACCCAATTCGCCCTATAGTGAGTC
GTATTACGCGCGCTCACTGGCCGTCGTTTTACAACGTCGTGACTGGGAAAACCCTGGCGTTACC
CAACTTAATCGCCTTGCAGCACATCCCCCTTTCGCCAGCTGGCGTAATAGCGAAGAGGCCCGCA
CCGATCGCCCTTCCCAACAGTTGCGCAGCCTGAATGGCGAATGGGACGCGCCCTGTAGCGGCGC
ATTAAGCGCGGCGGGTGTGGTGGTTACGCGCAGCGTGACCGCTACACTTGCCAGCGCCCTAGCG
CCCGCTCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCAAAAAACT
TGATTAGGGTGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATGAGCT
GATTTAACAAAAATTTAACGCGAATTTTAACAAAATATTAACGCTTACAATTTAGGTGGCACTT
TTCGGGGAAATGTGCGCGGAACCCCTATTTGTTTATTTTTCTAAATACATTCAAATATGTATCC
GCTCATGAattaattcttagaaaaactcatcgagcatcaaatgaaactgcaatttattcatatc
aggattatcaataccatatttttgaaaaagccgtttctgtaatgaaggagaaaactcaccgagg
cagttccataggatggcaagatcctggtatcggtctgcgattccgactcgtccaacatcaatac
aacctattaatttcccctcgtcaaaaataaggttatcaagtgagaaatcaccatgagtgacgac
tgaatccggtgagaatggcaaaagtttatgcatttctttccagacttgttcaacaggccagcca
ttacgctcgtcatcaaaatcactcgcatcaaccaaaccgttattcattcgtgattgcgcctgag
cgagacgaaatacgcgatcgctgttaaaaggacaattacaaacaggaatcgaatgcaaccggcg
caggaacactgccagcgcatcaacaatattttcacctgaatcaggatattcttctaatacctgg
aatgctgttttcccggggatcgcagtggtgagtaaccatgcatcatcaggagtacggataaaat
gcttgatggtcggaagaggcataaattccgtcagccagtttagtctgaccatctcatctgtaac
atcattggcaacgctacctttgccatgtttcagaaacaactctggcgcatcgggcttcccatac
aatcgatagattgtcgcacctgattgcccgacattatcgcgagcccatttatacccatataaat
cagcatccatgttggaatttaatcgcggcctagagcaagacgtttcccgttgaatatggctcat
aacaccccttgtattactgtttatgtaagcagacagttttattgtTCATGACCAAAATCCCTTA
ACGTGAGTTTTCGTTCCACTGAGCGTCAGACCCCGTAGAAAAGATCAAAGGATCTTCTTGAGAT
CCTTTTTTTCTGCGCGTAATCTGCTGCTTGCAAACAAAAAAACCACCGCTACCAGCGGTGGTTT
GTTTGCCGGATCAAGAGCTACCAACTCTTTTTCCGAAGGTAACTGGCTTCAGCAGAGCGCAGAT
ACCAAATACTGTTCTTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCGGGCTGAACGGGGGG
TTCGTGCACACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTTTTGCTCACA
TGTTCTTTCCTGCGTTATCCCCTGATTCTGTGGATAACCGTATTACCGCCTTTGAGTGAGCTGA
TACCGCTCGCCGCAGCCGAACGACCGAGCGCAGCGAGTCAGTGAGCGAGGAAGCGGAAGAGCGC
CCAATACGCAAACCGCCTCTCCCCGCGCGTTGGCCGATTCATTAATGCAGCTGGCACGACAGGT
TTCCCGACTGGAAAGCGGGCAGTGAGCGCAACGCAATTAATGTGAGTTAGCTCACTCATTAGGC
ACCCCAGGCTTTACACTTTATGCTTCCGGCTCGTATGTTGTGTGGAATTGTGAGCGGATAACAA
TTTCACACAGGAAACAGCTATGACCATGATTACGCCAAGCGCGCAATTAACCCTCACTAAAGGG
AACAAAAGCTGGAGCTGCA.

In some embodiments, the anti-CD30 CAR is expressed from a pLV3.1_T1-36 vector comprising the following sequence:

(SEQ ID NO: 31)
AGCTTAATGTAGTCTTATGCAATACTCTTGTAGTCTTGCAACATGGTAACGATGAGTTAGCAAC
ATGCCTTACAAGGAGAGAAAAAGCACCGTGCATGCCGATTGGTGGAAGTAAGGTGGTACGATCG
TGCCTTATTAGGAAGGCAACAGACGGGTCTGACATGGATTGGACGAACCACTGAATTGCCGCAT
TGCAGAGATATTGTATTTAAGTGCCTAGCTCGATACATAAACGGGTCTCTCTGGTTAGACCAGA
TCTGAGCCTGGGAGCTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACTAGAGATCCCTCAGA
CCCTTTTAGTCAGTGTGGAAAATCTCTAGCAGTGGCGCCCGAACAGGGACTTGAAAGCGAAAGG
GAAACCAGAGGAGCTCTCTCGACGCAGGACTCGGCTTGCTGAAGCGCGCACGGCAAGAGGCGAG
GGGCGGCGACTGGTGAGTACGCCAAAAATTTTGACTAGCGGAGGCTAGAAGGAGAGAGATGGGT
GCGAGAGCGTCAGTATTAAGCGGGGGAGAATTAGATCGCGATGGGAAAAAATTCGGTTAAGGCC
AGGGGGAAAGAAAAAATATAAATTAAAACATATAGTATGGGCAAGCAGGGAGCTAGAACGATTC
GCAGTTAATCCTGGCCTGTTAGAAACATCAGAAGGCTGTAGACAAATACTGGGACAGCTACAAC
CATCCCTTCAGACAGGATCAGAAGAACTTAGATCATTATATAATACAGTAGCAACCCTCTATTG
TGTGCATCAAAGGATAGAGATAAAAGACACCAAGGAAGCTTTAGACAAGATAGAGGAAGAGCAA
AACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAG
CACCCACCAAGGCAAAGAGAAGAGTGGTGCAGAGAGAAAAAAGAGCAGTGGGAATAGGAGCTTT
GTTCCTTGGGTTCTTGGGAGCAGCAGGAAGCACTATGGGCGCAGCGTCAATGACGCTGACGGTA
CAGGCCAGACAATTATTGTCTGGTATAGTGCAGCAGCAGAACAATTTGCTGAGGGCTATTGAGG
CGCAACAGCATCTGTTGCAACTCACAGTCTGGGGCATCAAGCAGCTCCAGGCAAGAATCCTGGC
TGTGGAAAGATACCTAAAGGATCAACAGCTCCTGGGGATTTGGGGTTGCTCTGGAAAACTCATT
TGCACCACTGCTGTGCCTTGGAATGCTAGTTGGAGTAATAAATCTCTGGAACAGATTGGAATCA
CACGACCTGGATGGAGTGGGACAGAGAAATTAACAATTACACAAGCTTAATACACTCCTTAATT
GAAGAATCGCAAAACCAGCAAGAAAAGAATGAACAAGAATTATTGGAATTAGATAAATGGGCAA
GTTTGTGGAATTGGTTTAACATAACAAATTGGCTGTGGTATATAAAATTATTCATAATGATAGT
AGGAGGCTTGGTAGGTTTAAGAATAGTTTTTGCTGTACTTTCTATAGTGAATAGAGTTAGGCAG
GGATATTCACCATTATCGTTTCAGACCCACCTCCCAACCCCGAGGGGACCCGACAGGCCCGAAG
GAATAGAAGAAGAAGGTGGAGAGAGAGACAGAGACAGATCCATTCGATTAGTGAACGGATCTCG
ACGGTATCGGTTAACTTTTAAAAGAAAAGGGGGGATTGGGGGGTACAGTGCAGGGGAAAGAATA
GTAGACATAATAGCAACAGACATACAAACTAAAGAATTACAAAAACAAATTACAAAAATTCAAA
ATTTTATCGATCACGAGACTAGCCTCGAGAAGCTTGATATCGAATTCCACGTCGACTTATGTGA
GGCTCCGGTGCCCGTCAGTGGGCAGAGCGCACATCGCCCACAGTCCCCGAGAAGTTGGGGGGAG
GGGTCGGCAATTGAACCGGTGCCTAGAGAAGGTGGCGCGGGGTAAACTGGGAAAGTGATGTCGT
GTACTGGCTCCGCCTTTTTCCCGAGGGTGGGGGAGAACCGTATATAAGTGCAGTAGTCGCCGTG
AACGTTCTTTTTCGCAACGGGTTTGCCGCCAGAACACAGGTAAGTGCCGTGTGTGGTTCCCGCG
GGCCTGGCCTCTTTACGGGTTATGGCCCTTGCGTGCCTTGAATTACTTCCACCTGGCTGCAGTA
CGTGATTCTTGATCCCGAGCTTCGGGTTGGAAGTGGGTGGGAGAGTTCGAGGCCTTGCGCTTAA
GGAGCCCCTTCGCCTCGTGCTTGAGTTGAGGCCTGGCCTGGGCGCTGGGGCCGCCGCGTGCGAA
TCTGGTGGCACCTTCGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGGGCCAAGATCTGCAC
ACTGGTATTTCGGTTTTTGGGGCCGCGGGCGGCGACGGGGCCCGTGCGTCCCAGCGCACATGTT
CGGCGAGGCGGGGCCTGCGAGCGCGGCCACCGAGAATCGGACGGGGGTAGTCTCAAGCTGGCCG
GCCTGCTCTGGTGCCTGGCCTCGCGCCGCCGTGTATCGCCCCGCCCTGGGCGGCAAGGCTGGCC
CGGTCGGCACCAGTTGCGTGAGCGGAAAGATGGCCGCTTCCCGGCCCTGCTGCAGGGAGCTCAA
AATGGAGGACGCGGCGCTCGGGAGAGCGGGCGGGTGAGTCACCCACACAAAGGAAAAGGGCCTT
TCCGTCCTCAGCCGTCGCTTCATGTGACTCCACGGAGTACCGGGCGCCGTCCAGGCACCTCGAT
TAGTTCTCGAGCTTTTGGAGTACGTCGTCTTTAGGTTGGGGGGAGGGGTTTTATGCGATGGAGT
TTCCCCACACTGAGTGGGTGGAGACTGAAGTTAGGCCAGCTTGGCACTTGATGTAATTCTCCTT
GGAATTTGCCCTTTTTGAGTTTGGATCTTGGTTCATTCTCAAGCCTCAGACAGTGGTTCAAAGT
TTTTTTCTTCCATTTCAGGTGTCGTGAGGATCCACTAGTGCCACCATGGCCTTACCAGTGACCG
CCTTGCTCCTGCCGCTGGCCTTGCTGCTCCACGCCGCCAGGCCGGAGGTGCAGCTGTTGGAGTC
TGGGGGAGGCTTGGTACAGCCTGGGGGGTCCCTGAGACTCTCCTGTGCAGCCTCTGGATTCACC
TTTAGCTACTATTACATGAGCTGGGTCCGCCAGGCTCCAGGGAAGGGGCTGGAGTGGGTCTCAT
ACATCGGTGGTGGTGGTTCTGGTACGTATTACGCTGATTCTGTAAAAGGTCGGTTCACCATCTC
CAGAGACAATTCCAAGAACACGCTGTATCTGCAAATGAACAGCCTGAGAGCCGAGGACACGGCC
GTGTATTACTGTGCGCGTGGTCCGTACTACGGTTACTTCGACTACTGGGGCCAGGGTACACTGG
TCACCGTGAGCTCAGGTGGAGGCGGTTCAGGCGGAGGTGGATCCGGCGGTGGCGGATCGCAGTC
TGTGCTGACTCAGCCACCCTCAGCGTCTGGGACCCCCGGGCAGAGGGTCACCATCTCTTGTAGT
GGCTCTTCATCTAATATTGGCTCTAATTATGTCTACTGGTACCAGCAGCTCCCAGGAACGGCCC
CCAAACTCCTCATCTATAGAAATAACCAGCGGCCAAGCGGGGTCCCTGACCGATTCTCTGGCTC
CAAGTCTGGCACCTCAGCCTCCCTGGCCATCAGTGGGCTCCGGTCCGAGGATGAGGCCGATTAT
TACTGTGCTGCTTACCCGTCTTACGACTCTTATGTCTTCGGCGGAGGCACCAAGCTGACGGTCC
TAGGTGGCCAGGCCGGCCAGACAACGACACCTGCTCCCAGACCGCCTACTCCCGCCCCAACCAT
TGCATCTCAGCCACTCTCTCTGAGACCCGAAGCGTGTAGACCTGCGGCCGGGGGCGCTGTCCAC
ACAAGAGGCTTAGACTTCGCCTGCGATATCTATATCTGGGCCCCACTCGCAGGCACTTGTGGAG
TGCTGCTGCTTTCACTCGTGATAACCCTGTACTGCAAAAGGGGGAGAAAGAAGCTGCTGTATAT
TTTTAAACAACCATTTATGAGACCTGTTCAGACTACCCAGGAAGAAGACGGTTGTAGTTGCAGA
TTCCCCGAGGAGGAAGAAGGAGGTTGCGAGTTGAGAGTAAAGTTCAGCAGATCCGCAGATGCCC
CTGCTTACCAGCAGGGTCAAAACCAGCTTTACAACGAGCTGAATTTAGGTAGAAGAGAGGAATA
TGACGTGTTGGATAAAAGAAGAGGAAGAGACCCGGAAATGGGCGGCAAGCCTCGAAGAAAAAAT
CCCCAAGAGGGACTCTACAATGAGCTGCAGAAGGACAAAATGGCTGAAGCCTACAGCGAGATCG
GCATGAAGGGAGAAAGACGCAGAGGGAAAGGGCATGATGGGCTTTATCAGGGCTTGTCCACCGC
TACAAAGGATACTTATGACGCACTACACATGCAGGCCCTGCCACCCCGTTGATTAATTAACTCG
AGCTCGAGTCTAGAGGGCCCGCGGTTCGAAGGTAAGCCTATCCCTAACCCTCTCCTCGGTCTCG
ATTCTACGCGTACCGGTTAGTAATGATCGACAATCAACCTCTGGATTACAAAATTTGTGAAAGA
TTGACTGGTATTCTTAACTATGTTGCTCCTTTTACGCTATGTGGATACGCTGCTTTAATGCCTT
TGTATCATGCTATTGCTTCCCGTATGGCTTTCATTTTCTCCTCCTTGTATAAATCCTGGTTGCT
GTCTCTTTATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGACAGGGGC
TCGGCTGTTGGGCACTGACAATTCCGTGGTGTTGTCGGGGAAGCTGACGTCCTTTCCATGGCTG
CTCGCCTGTGTTGCCACCTGGATTCTGCGCGGGACGTCCTTCTGCTACGTCCCTTCGGCCCTCA
ATCCAGCGGACCTTCCTTCCCGCGGCCTGCTGCCGGCTCTGCGGCCTCTTCCGCGTCTTCGCCT
TCGCCCTCAGACGAGTCGGATCTCCCTTTGGGCCGCCTCCCCGCCTGGAATTCGAGCTCGGTAC
CTTTAAGACCAATGACTTACAAGGCAGCTGTAGATCTTAGCCACTTTTTAAAAGAAAAGGGGGG
ACTGGAAGGGCTAATTCACTCCCAACGAAGACAAGATCTGCTTTTTGCTTGTACTGGGTCTCTC
TGGTTAGACCAGATCTGAGCCTGGGAGCTCTCTGGCTAACTAGGGAACCCACTGCTTAAGCCTC
AATAAAGCTTGCCTTGAGTGCTTCAAGTAGTGTGTGCCCGTCTGTTGTGTGACTCTGGTAACTA
GAGATCCCTCAGACCCTTTTAGTCAGTGTGGAAAATCTCTAGCAGTAGTAGTTCATGTCATCTT
ATTATTCAGTATTTATAACTTGCAAAGAAATGAATATCAGAGAGTGAGAGGAACTTGTTTATTG
CAGCTTATAATGGTTACAAATAAAGCAATAGCATCACAAATTTCACAAATAAAGCATTTTTTTC
ACTGCATTCTAGTTGTGGTTTGTCCAAACTCATCAATGTATCTTATCATGTCTGGCTCTAGCTA
TCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCTCTGAGCTATTCCAG
AAGTAGTGAGGAGGCTTTTTTGGAGGCCTAGGGACGTACCCAATTCGCCCTATAGTGAGTCGTA
TTACGCGCGCTCACTGGCCGTCGTTTTACAACGTCGTGACTGGGAAAACCCTGGCGTTACCCAA
CTTAATCGCCTTGCAGCACATCCCCCTTTCGCCAGCTGGCGTAATAGCGAAGAGGCCCGCACCG
ATCGCCCTTCCCAACAGTTGCGCAGCCTGAATGGCGAATGGGACGCGCCCTGTAGCGGCGCATT
AAGCGCGGCGGGTGTGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGTTG
GAGTCCACGTTCTTTAATAGTGGACTCTTGTTCCAAACTGGAACAACACTCAACCCTATCTCGG
TCTATTCTTTTGATTTATAAGGGATTTTGCCGATTTCGGCCTATTGGTTAAAAAATGAGCTGAT
TTAACAAAAATTTAACGCGAATTTTAACAAAATATTAACGCTTACAATTTAGGTGGCACTTTTC
GGGGAAATGTGCGCGGAACCCCTATTTGTTTATTTTTCTAAATACATTCAAATATGTATCCGCT
CATGAattaattcttagaaaaactcatcgagcatcaaatgaaactgcaatttattcatatcagg
attatcaataccatatttttgaaaaagccgtttctgtaatgaaggagaaaactcaccgaggcag
ttccataggatggcaagatcctggtatcggtctgcgattccgactcgtccaacatcaatacaac
ctattaatttcccctcgtcaaaaataaggttatcaagtgagaaatcaccatgagtgacgactga
atccggtgagaatggcaaaagtttatgcatttctttccagacttgttcaacaggccagccatta
cgctcgtcatcaaaatcactcgcatcaaccaaaccgttattcattcgtgattgcgcctgagcga
gacgaaatacgcgatcgctgttaaaaggacaattacaaacaggaatcgaatgcaaccggcgcag
gaacactgccagcgcatcaacaatattttcacctgaatcaggatattcttctaatacctggaat
gctgttttcccggggatcgcagtggtgagtaaccatgcatcatcaggagtacggataaaatgct
tgatggtcggaagaggcataaattccgtcagccagtttagtctgaccatctcatctgtaacatc
attggcaacgctacctttgccatgtttcagaaacaactctggcgcatcgggcttcccatacaat
cgatagattgtcgcacctgattgcccgacattatcgcgagcccatttatacccatataaatcag
catccatgttggaatttaatcgcggcctagagcaagacgtttcccgttgaatatggctcataac
accccttgtattactgtttatgtaagcagacagttttattgtTCATGACCAAAATCCCTTAACG
TGAGTTTTCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATACC
AAATACTGTTCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGTTC
GTGCACACAGCCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGT
TCTTTCCTGCGTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCGGAAGAGCGCCCA
ATACGCAAACCGCCTCTCCCCGCGCGTTGGCCGATTCATTAATGCAGCTGGCACGACAGGTTTC
CCGACTGGAAAGCGGGCAGTGAGCGCAACGCAATTAATGTGAGTTAGCTCACTCATTAGGCACC
CCAGGCTTTACACTTTATGCTTCCGGCTCGTATGTTGTGTGGAATTGTGAGCGGATAACAATTT
CACACAGGAAACAGCTATGACCATGATTACGCCAAGCGCGCAATTAACCCTCACTAAAGGGAAC
AAAAGCTGGAGCTGCA.

In some embodiments, the anti-CD30 CAR is expressed from a pLV3.1_HRS CAR vector comprising the following sequence:

(SEQ ID NO: 32)
AGCTTAATGTAGTCTTATGCAATACTCTTGTAGTCTTGCAACATGGTAACGATGAGTTAGCAAC
ATGCCTTACAAGGAGAGAAAAAGCACCGTGCATGCCGATTGGTGGAAGTAAGGTGGTACGATCG
TGCCTTATTAGGAAGGCAACAGACGGGTCTGACATGGATTGGACGAACCACTGAATTGCCGCAT
TGCAGAGATATTGTATTTAAGTGCCTAGCTCGATACATAAACGGGTCTCTCTGGTTAGACCAGA
TCTGAGCCTGGGAGCTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACTAGAGATCCCTCAGA
CCCTTTTAGTCAGTGTGGAAAATCTCTAGCAGTGGCGCCCGAACAGGGACTTGAAAGCGAAAGG
GAAACCAGAGGAGCTCTCTCGACGCAGGACTCGGCTTGCTGAAGCGCGCACGGCAAGAGGCGAG
GGGCGGCGACTGGTGAGTACGCCAAAAATTTTGACTAGCGGAGGCTAGAAGGAGAGAGATGGGT
GCGAGAGCGTCAGTATTAAGCGGGGGAGAATTAGATCGCGATGGGAAAAAATTCGGTTAAGGCC
AGGGGGAAAGAAAAAATATAAATTAAAACATATAGTATGGGCAAGCAGGGAGCTAGAACGATTC
GCAGTTAATCCTGGCCTGTTAGAAACATCAGAAGGCTGTAGACAAATACTGGGACAGCTACAAC
CATCCCTTCAGACAGGATCAGAAGAACTTAGATCATTATATAATACAGTAGCAACCCTCTATTG
TGTGCATCAAAGGATAGAGATAAAAGACACCAAGGAAGCTTTAGACAAGATAGAGGAAGAGCAA
AACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAG
CACCCACCAAGGCAAAGAGAAGAGTGGTGCAGAGAGAAAAAAGAGCAGTGGGAATAGGAGCTTT
GTTCCTTGGGTTCTTGGGAGCAGCAGGAAGCACTATGGGCGCAGCGTCAATGACGCTGACGGTA
CAGGCCAGACAATTATTGTCTGGTATAGTGCAGCAGCAGAACAATTTGCTGAGGGCTATTGAGG
CGCAACAGCATCTGTTGCAACTCACAGTCTGGGGCATCAAGCAGCTCCAGGCAAGAATCCTGGC
TGTGGAAAGATACCTAAAGGATCAACAGCTCCTGGGGATTTGGGGTTGCTCTGGAAAACTCATT
TGCACCACTGCTGTGCCTTGGAATGCTAGTTGGAGTAATAAATCTCTGGAACAGATTGGAATCA
CACGACCTGGATGGAGTGGGACAGAGAAATTAACAATTACACAAGCTTAATACACTCCTTAATT
GAAGAATCGCAAAACCAGCAAGAAAAGAATGAACAAGAATTATTGGAATTAGATAAATGGGCAA
GTTTGTGGAATTGGTTTAACATAACAAATTGGCTGTGGTATATAAAATTATTCATAATGATAGT
AGGAGGCTTGGTAGGTTTAAGAATAGTTTTTGCTGTACTTTCTATAGTGAATAGAGTTAGGCAG
GGATATTCACCATTATCGTTTCAGACCCACCTCCCAACCCCGAGGGGACCCGACAGGCCCGAAG
GAATAGAAGAAGAAGGTGGAGAGAGAGACAGAGACAGATCCATTCGATTAGTGAACGGATCTCG
ACGGTATCGGTTAACTTTTAAAAGAAAAGGGGGGATTGGGGGGTACAGTGCAGGGGAAAGAATA
GTAGACATAATAGCAACAGACATACAAACTAAAGAATTACAAAAACAAATTACAAAAATTCAAA
ATTTTATCGATCACGAGACTAGCCTCGAGAAGCTTGATATCGAATTCCACGTCGACTTATGTGA
GGCTCCGGTGCCCGTCAGTGGGCAGAGCGCACATCGCCCACAGTCCCCGAGAAGTTGGGGGGAG
GGGTCGGCAATTGAACCGGTGCCTAGAGAAGGTGGCGCGGGGTAAACTGGGAAAGTGATGTCGT
GTACTGGCTCCGCCTTTTTCCCGAGGGTGGGGGAGAACCGTATATAAGTGCAGTAGTCGCCGTG
AACGTTCTTTTTCGCAACGGGTTTGCCGCCAGAACACAGGTAAGTGCCGTGTGTGGTTCCCGCG
GGCCTGGCCTCTTTACGGGTTATGGCCCTTGCGTGCCTTGAATTACTTCCACCTGGCTGCAGTA
CGTGATTCTTGATCCCGAGCTTCGGGTTGGAAGTGGGTGGGAGAGTTCGAGGCCTTGCGCTTAA
GGAGCCCCTTCGCCTCGTGCTTGAGTTGAGGCCTGGCCTGGGCGCTGGGGCCGCCGCGTGCGAA
TCTGGTGGCACCTTCGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGGGCCAAGATCTGCAC
ACTGGTATTTCGGTTTTTGGGGCCGCGGGCGGCGACGGGGCCCGTGCGTCCCAGCGCACATGTT
CGGCGAGGCGGGGCCTGCGAGCGCGGCCACCGAGAATCGGACGGGGGTAGTCTCAAGCTGGCCG
GCCTGCTCTGGTGCCTGGCCTCGCGCCGCCGTGTATCGCCCCGCCCTGGGCGGCAAGGCTGGCC
CGGTCGGCACCAGTTGCGTGAGCGGAAAGATGGCCGCTTCCCGGCCCTGCTGCAGGGAGCTCAA
AATGGAGGACGCGGCGCTCGGGAGAGCGGGCGGGTGAGTCACCCACACAAAGGAAAAGGGCCTT
TCCGTCCTCAGCCGTCGCTTCATGTGACTCCACGGAGTACCGGGCGCCGTCCAGGCACCTCGAT
TAGTTCTCGAGCTTTTGGAGTACGTCGTCTTTAGGTTGGGGGGAGGGGTTTTATGCGATGGAGT
TTCCCCACACTGAGTGGGTGGAGACTGAAGTTAGGCCAGCTTGGCACTTGATGTAATTCTCCTT
GGAATTTGCCCTTTTTGAGTTTGGATCTTGGTTCATTCTCAAGCCTCAGACAGTGGTTCAAAGT
TTTTTTCTTCCATTTCAGGTGTCGTGAGGATCCACTAGTGCCACCATGGCCTTACCAGTGACCG
CCTTGCTCCTGCCGCTGGCCTTGCTGCTCCACGCCGCCAGGCCGGAGGTGAAGCTTCAACAGTC
CGGAGCAGAACTCGCCCGCCCGGGCGCTAGCGTGAAAATGAGCTGCAAGGCCTCTGGATATACC
TTCACCACCTATACCATTCACTGGGTGAGACAGAGACCTGGCCATGACCTGGAGTGGATCGGCT
ATATAAACCCAAGCAGTGGCTACTCCGACTACAATCAGAATTTCAAAGGCAAAACAACACTGAC
CGCGGACAAGTCTTCAAATACTGCCTACATGCAGCTGAACTCTCTGACAAGCGAGGATTCAGCA
GTGTATTACTGCGCTAGGCGGGCTGACTACGGCAACTACGAGTATACCTGGTTTGCATACTGGG
GCCAGGGAACAACGGTTACAGTGTCCTCAGGCGGCGGAGGGAGCGGGGGTGGTGGGAGTGGAGG
CGGGGGGAGTGATATCGAACTGACGCAGTCACCTAAGTTTATGAGCACATCCGTGGGGGATCGA
GTCAATGTCACGTGTAAGGCCAGCCAGAATGTAGGCACTAACGTGGCCTGGTTTCAACAGAAGC
CCGGTCAAAGCCCCAAAGTTCTGATCTACAGCGCTTCCTATAGGTACAGTGGCGTGCCAGATAG
ATTCACCGGATCCGGAAGCGGAACCGATTTCACTCTCACTATTTCAAACGTTCAATCGGAAGAT
TTGGCCGAGTATTTTTGTCAGCAGTATCACACATACCCCTTAACTTTCGGTGGCGGTACTAAGC
TGGAAATTAAAGGCCAGGCCGGCCAGACAACGACACCTGCTCCCAGACCGCCTACTCCCGCCCC
AACCATTGCATCTCAGCCACTCTCTCTGAGACCCGAAGCGTGTAGACCTGCGGCCGGGGGCGCT
GTCCACACAAGAGGCTTAGACTTCGCCTGCGATATCTATATCTGGGCCCCACTCGCAGGCACTT
GTGGAGTGCTGCTGCTTTCACTCGTGATAACCCTGTACTGCAAAAGGGGGAGAAAGAAGCTGCT
GTATATTTTTAAACAACCATTTATGAGACCTGTTCAGACTACCCAGGAAGAAGACGGTTGTAGT
TGCAGATTCCCCGAGGAGGAAGAAGGAGGTTGCGAGTTGAGAGTAAAGTTCAGCAGATCCGCAG
ATGCCCCTGCTTACCAGCAGGGTCAAAACCAGCTTTACAACGAGCTGAATTTAGGTAGAAGAGA
GGAATATGACGTGTTGGATAAAAGAAGAGGAAGAGACCCGGAAATGGGCGGCAAGCCTCGAAGA
AAAAATCCCCAAGAGGGACTCTACAATGAGCTGCAGAAGGACAAAATGGCTGAAGCCTACAGCG
AGATCGGCATGAAGGGAGAAAGACGCAGAGGGAAAGGGCATGATGGGCTTTATCAGGGCTTGTC
CACCGCTACAAAGGATACTTATGACGCACTACACATGCAGGCCCTGCCACCCCGTTGATTAATT
AACTCGAGCTCGAGTCTAGAGGGCCCGCGGTTCGAAGGTAAGCCTATCCCTAACCCTCTCCTCG
GTCTCGATTCTACGCGTACCGGTTAGTAATGATCGACAATCAACCTCTGGATTACAAAATTTGT
GAAAGATTGACTGGTATTCTTAACTATGTTGCTCCTTTTACGCTATGTGGATACGCTGCTTTAA
TGCCTTTGTATCATGCTATTGCTTCCCGTATGGCTTTCATTTTCTCCTCCTTGTATAAATCCTG
GTTGCTGTCTCTTTATGAGGAGTTGTGGCCCGTTGTCAGGCAACGTGGCGTGGTGTGCACTGTG
TTTGCTGACGCAACCCCCACTGGTTGGGGCATTGCCACCACCTGTCAGCTCCTTTCCGGGACTT
TCGCTTTCCCCCTCCCTATTGCCACGGCGGAACTCATCGCCGCCTGCCTTGCCCGCTGCTGGAC
AGGGGCTCGGCTGTTGGGCACTGACAATTCCGTGGTGTTGTCGGGGAAGCTGACGTCCTTTCCA
TGGCTGCTCGCCTGTGTTGCCACCTGGATTCTGCGCGGGACGTCCTTCTGCTACGTCCCTTCGG
CCCTCAATCCAGCGGACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCGCCTGGAATTCGAGCT
CGGTACCTTTAAGACCAATGACTTACAAGGCAGCTGTAGATCTTAGCCACTTTTTAAAAGAAAA
GGGGGGACTGGAAGGGCTAATTCACTCCCAACGAAGACAAGATCTGCTTTTTGCTTGTACTGGG
TCTCTCTGGTTAGACCAGATCTGAGCCTGGGAGCTCTCTGGCTAACTAGGGAACCCACTGCTTA
AGCCTCAATAAAGCTTGCCTTGAGTGCTTCAAGTAGTGTGTGCCCGTCTGTTGTGTGACTCTGG
TAACTAGAGATCCCTCAGACCCTTTTAGTCAGTGTGGAAAATCTCTAGCAGTAGTAGTTCATGT
CATCTTATTATTCAGTATTTATAACTTGCAAAGAAATGAATATCAGAGAGTGAGAGGAACTTGT
TTATTGCAGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTATCATGTCTGGCTC
TAGCTATCCCGCCCCTAACTCCGCCCATCCCGCCCCTAACTCCGCCCAGTTCCGCCCATTCTCC
GCCCCATGGCTGACTAATTTTTTTTATTTATGCAGAGGCCGAGGCCGCCTCGGCCTCTGAGCTA
TTCCAGAAGTAGTGAGGAGGCTTTTTTGGAGGCCTAGGGACGTACCCAATTCGCCCTATAGTGA
GTCGTATTACGCGCGCTCACTGGCCGTCGTTTTACAACGTCGTGACTGGGAAAACCCTGGCGTT
ACCCAACTTAATCGCCTTGCAGCACATCCCCCTTTCGCCAGCTGGCGTAATAGCGAAGAGGCCC
GCACCGATCGCCCTTCCCAACAGTTGCGCAGCCTGAATGGCGAATGGGACGCGCCCTGTAGCGG
CGCATTAAGCGCGGCGGGTGTGGTGGTTACGCGCAGCGTGACCGCTACACTTGCCAGCGCCCTA
GCGCCCGCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACCCCAAAAA
ACTTGATTAGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCCTA
TCTCGGTCTATTCTTTTGATTTATAAGGGATTTTGCCGATTTCGGCCTATTGGTTAAAAAATGA
GCTGATTTAACAAAAATTTAACGCGAATTTTAACAAAATATTAACGCTTACAATTTAGGTGGCA
CTTTTCGGGGAAATGTGCGCGGAACCCCTATTTGTTTATTTTTCTAAATACATTCAAATATGTA
TCCGCTCATGAattaattcttagaaaaactcatcgagcatcaaatgaaactgcaatttattcat
atcaggattatcaataccatatttttgaaaaagccgtttctgtaatgaaggagaaaactcaccg
aggcagttccataggatggcaagatcctggtatcggtctgcgattccgactcgtccaacatcaa
tacaacctattaatttcccctcgtcaaaaataaggttatcaagtgagaaatcaccatgagtgac
gactgaatccggtgagaatggcaaaagtttatgcatttctttccagacttgttcaacaggccag
ccattacgctcgtcatcaaaatcactcgcatcaaccaaaccgttattcattcgtgattgcgcct
gagcgagacgaaatacgcgatcgctgttaaaaggacaattacaaacaggaatcgaatgcaaccg
gcgcaggaacactgccagcgcatcaacaatattttcacctgaatcaggatattcttctaatacc
tggaatgctgttttcccggggatcgcagtggtgagtaaccatgcatcatcaggagtacggataa
aatgcttgatggtcggaagaggcataaattccgtcagccagtttagtctgaccatctcatctgt
aacatcattggcaacgctacctttgccatgtttcagaaacaactctggcgcatcgggcttccca
tacaatcgatagattgtcgcacctgattgcccgacattatcgcgagcccatttatacccatata
aatcagcatccatgttggaatttaatcgcggcctagagcaagacgtttcccgttgaatatggct
cataacaccccttgtattactgtttatgtaagcagacagttttattgtTCATGACCAAAATCCC
TTAACGTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGCTTCAGCAGAGCGCA
GATACCAAATACTGTTCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATACCTACAGCGT
GAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGGGGGCGGAGC
CTATGGAAAAACGCCAGCAACGCGGCCTTTTTACGGTTCCTGGCCTTTTGCTGGCCTTTTGCTC
ACATGTTCTTTCCTGCGTTATCCCCTGATTCTGTGGATAACCGTATTACCGCCTTTGAGTGAGC
TGATACCGCTCGCCGCAGCCGAACGACCGAGCGCAGCGAGTCAGTGAGCGAGGAAGCGGAAGAG
CGCCCAATACGCAAACCGCCTCTCCCCGCGCGTTGGCCGATTCATTAATGCAGCTGGCACGACA
GGTTTCCCGACTGGAAAGCGGGCAGTGAGCGCAACGCAATTAATGTGAGTTAGCTCACTCATTA
GGCACCCCAGGCTTTACACTTTATGCTTCCGGCTCGTATGTTGTGTGGAATTGTGAGCGGATAA
CAATTTCACACAGGAAACAGCTATGACCATGATTACGCCAAGCGCGCAATTAACCCTCACTAAA
GGGAACAAAAGCTGGAGCTGCA.

### T Cell Receptors

In some embodiments, the TCR is a recombinant TCR. In other embodiments, the TCR is encoded by a heterologous sequence (e.g., transgene) present in the immune cell.

In some embodiments, the TCR targets (i.e., has antigenic specificity for) an antigen, for example, a tumor antigen. As used herein, the phrase “having antigenic specificity,” or like phrase, means that the TCR can specifically bind to and recognize the antigen, or an epitope thereof.

Natural TCRs are generally heterodimers. In humans, in 95% of T cells, the TCR comprises an alpha (α) chain and a beta (β) chain (encoded by TRA and TRB, respectively), whereas in 5% of T cells, the TCR comprises gamma and delta (γ/δ) chains (encoded by TRG and TRD, respectively). Natural TCR complexes are an octameric assembly of type-I single-spanning membrane proteins arranged into four dimeric modules: the variable ligand-binding TCRαβ module (in most T cells) (or the TCR γ/δ module) and the three invariant signaling modules CD3δε, CD3γε, and CD3ζ dimer. The TCRαβ module binds to pMHC ligands on APC or target cell surfaces, but these proteins lack intrinsic signaling capability and, as such, rely on the signaling modules to transmit information through their cytoplasmic immunoreceptor tyrosine-based activation motifs (ITAMs). See, e.g., Chandler et al. Int J Mol Sci (2020) 21:7424, which is incorporated by reference herein. Natural TCRs can be cloned and modified using standard molecular biology and genetic engineering techniques known in the art.

Various engineered TCR forms are also known in the art, including TCR mimic antibodies (Chang et al., Expert Opin Biol Ther. (2016) 16(8):979-87), TCR-like CARs and TCR-CARs (Walseng et al., Sci Rep. (2017) 7:1-10; Akatsuka et al., Front Immunol. (2020) 11:257; Poorebrahim et al., Cancer Gene Ther. (2021) 28(6):581-589. Unlike CARs, TCRs are not restricted to the cell surface antigens, but can detect and bind to the peptides presented by MHC molecules (pMHCs). This feature provides a wide range of potential targets for TCRs such as tumor-specific neoepitopes. Of note, redirection of TCR-based CARs on the highly tumor-specific neoepitopes can prevent “off-tumor” toxicities that are commonly associated with CAR therapies.

In some embodiments, the TCR is a natural TCR. In some embodiments, the TCR is a modified TCR. In some embodiments, the TCR is an engineered TCR, such as a TCR mimic or antibody-like structure, a CAR-like TCR, or a CAR-TCR. In some embodiments, the TCR is a murine TCR. In some embodiments, the TCR is a human TCR. In some embodiments, the TCR is a hybrid TCR having one or more portions of a human TCR (e.g., a constant portion or a variable portion) and one or more portions of a murine TCR (e.g., a constant portion or a variable portion). Alternatively, the portion can be a few amino acids of a human TCR, such that the TCR, which is mostly murine, is “humanized.” Methods of making such hybrid TCRs are known in the art (see, for example, Cohen et al., Cancer Res., (2006) 66:8878-8886).

In some embodiments, the TCR targets a tumor antigen. In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), gp100, HER2, HLA-A2, HPV E6, ICAM1, IL3Ra, IL13Ra2, LAGE-1, Lewis Y, LMP1 (EBV), MAGE-A1, MAGE-A3, MAGE-A4, MAGE-A6, MAGE-A10, MART-1, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PRAME, PSCA, PSMA, ROR1, ROR2, TIM-3, TM4SF1, TnMuc1, VEGFR2, and any combination thereof.

In some embodiments, the TCR targets (i.e., has antigenic specificity for) a gp100 melanoma antigen, e.g., human gp100. In some embodiments, the tumor antigen is gp100. gp100, also known in the art as SILV, SI, SIL, ME20, PMEL17, or D12S53E. gp100, is a protein known to play an important role in regulating mammalian pigmentation (Hoashi et al., J. Biol. Chem. (2005) 280:14006-14016) and is known as a cancer antigen expressed by human tumors, including melanoma and colorectal tumors (Tartaglia et al., Vaccine (2001) 19(17-19):2571-5). The amino acid and nucleotide sequences of human gp100 are published in the GenBank database of the National Center for Biotechnology Information (NCBI) as GenBank Accession No. NP_008859 (amino acid sequence) and GenBank Accession No. NM_006928.3 (nucleotide sequence).

TCRs having antigenic specificity for gp100 (i.e., anti-gp100 TCRs) are known in the art, such as the TCRs described in, e.g., US20140219978A1. In some embodiments, the TCR is a pmel-1 TCR. The pmel-1 mouse model was developed as a system to model treatment of malignant melanoma using adoptive cell therapy (ACT) (Overwijk, et al., J Exp Med. (2003), 198(4):569-80). The target antigen, pmel-17, is an ortholog of the melanocyte differentiation antigen gp100, which is often overexpressed in human melanomas.

In some embodiments, the TCR targets (i.e., has antigenic specificity for) an NY-ESO-1 antigen. NY-ESO-1 is a cancer-testis antigen overexpressed in synovial sarcoma, myxoid liposarcoma, melanoma and other tumors. In some embodiments, the TCR is an NYESO1-TCR clone 1G4 (Robbins, et al., J Immunol, 2008, 180:6116-6131).

In some embodiments, the TCR targets an antigen selected from MAGE-A3/A6, MAGE-A10, AFP, PRAME, MART-1, and HPV E6.

### Nucleic Acids and Expression Vectors

The present disclosure provides certain nucleic acids for encoding proteins of the present invention, e.g., a CAR, a TCR, BTLA or variant thereof, and HVEM or a variant thereof.

In certain embodiments, a nucleic acid of the present disclosure comprises a first nucleotide sequence and a second nucleotide sequence. The first and second nucleotide sequences may be separated a linker (i.e., a third nucleotide sequence). A linker for use in the present disclosure allows for multiple proteins to be encoded by the same nucleic acid sequence (e.g., a multicistronic or bicistronic sequence), which are translated as a polyprotein that is dissociated into separate protein components. In certain embodiments, the nucleic acid comprises from 5′ to 3′ the first nucleotide sequence, the linker, and the second nucleotide sequence. In certain embodiments, the nucleic acid comprises from 5′ to 3′ the second nucleotide sequence, the linker, and the first nucleotide sequence.

In some embodiments, the linker comprises a nucleic acid sequence that encodes an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention.

In some embodiments, the linker comprises a nucleic acid sequence that encodes a self-cleaving peptide, also referred to as a ribosomal skipping sequence. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use in the present invention.

In some embodiments, the construct includes a linker that optionally, further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence. Various furin consensus recognition sequences (or “furin cleavage sites”) are known to those of skill in the art. Those of skill in the art would be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding a Furin cleavage site and F2A, a linker comprising a nucleic acid sequence encoding a Furin cleavage site and E2A, a linker comprising a nucleic acid sequence encoding a Furin cleavage site and P2A, a linker comprising a nucleic acid sequence encoding a Furin cleavage site and T2A. Those of skill in the art would be able to select the appropriate combination for use in the present invention. In such embodiments, the linker may further comprise a spacer sequence between the Furin cleavage site and the 2A peptide. In some embodiments, the linker comprises a Furin cleavage site 5′ to a 2A peptide. In some embodiments, the linker comprises a 2A peptide 5′ to a Furin cleavage site. Various spacer sequences are known in the art, including, without limitation, glycine serine (GS) spacers (also known as GS linkers). Those of skill in the art would be able to select the appropriate spacer sequence for use in the present invention.

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid encoding a CAR is operably linked to a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is used for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pST-Ki, pST-KiT, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present invention may comprise a nucleic acid encoding for a CAR. In some embodiments, the expression vector (e.g., lentiviral vector) will comprise additional elements that will aid in the functional expression of the CAR encoded therein. In some embodiments, an expression vector comprising a nucleic acid encoding for a CAR further comprises a mammalian promoter. In one embodiment, the vector further comprises an elongation-factor-1-alpha promoter (EF-la promoter). Use of an EF-la promoter may increase the efficiency in expression of downstream transgenes (e.g., a CAR encoding nucleic acid sequence). Physiologic promoters (e.g., an EF-la promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15: 9053. Introducing RNA comprising a nucleotide sequence encoding a CAR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of a CAR as described herein, e.g., in a mammalian cell. In some embodiments, a nucleic acid of the present disclosure provides for amplification of the CAR-encoding nucleic acid.

### Methods of Treatment

The immune cell (e.g., T cells) described herein may be included in a composition for immunotherapy. The composition may include a pharmaceutical composition and further include a pharmaceutically acceptable carrier. A therapeutically effective amount of the pharmaceutical composition comprising the immune cells provided herein may be administered to a subject in need thereof.

In one aspect, the invention includes a method for adoptive cell transfer therapy comprising administering to a subject in need thereof a population of immune cells of the present invention. In one aspect, the invention provides a method of treating cancer in a subject in need thereof, the method comprising administering to the subject a therapeutically effective amount of the immune cells (i.e., a population of the immune cells) of the invention.

Methods for administration of immune cells for adoptive cell therapy are known and may be used in connection with the provided methods and compositions. For example, adoptive immune cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338; Lee et al., Int J. Mol Sci. (2021) 22(9):4590; Banerjee et al., JCO Clin Cancer Inform. (2021) 5:668-678; Robbins et al., Stem Cell Res Ther. (2021) 12(1):350; Wrona et al., Int J Mol Sci. (2021) 22(11):5899; Atrash and Moyo, Onco Targets Ther. (2021) 14:2185-2201; Martinez Bedoya et al., Front Immunol. (2021) 12:640082; Morgan et al., Front Immunol. (2020) 11:1965; Chicaybam et al., Cancers (Basel) (2020) 12(9):2360; and Rafiq et al., Nat Rev Clin Oncol. (2020) 17(3):147-167. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The immune cell of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the cells or pharmaceutical compositions of the invention include certain leukemia or lymphoid malignancies, benign and malignant tumors, and malignancies e.g., sarcomas, carcinomas, and melanomas. Exemplary cancers include but are not limited to B-cell malignancies such as B-cell lymphomas and leukemias and the like, as well as colorectal cancer, breast cancer, ovarian cancer, renal cancer, non-small cell lung cancer, melanoma, lymphoma, and hepatocellular cancers. The cancers may be non-solid tumors (such as hematological tumors) or solid tumors. Adult tumors/cancers and pediatric tumors/cancers are also included. In one embodiment, the cancer is a solid tumor or a hematological tumor. In certain embodiments, the cancer is a leukemia and/or a lymphoma. In certain embodiments, the cancer cells express CD19.

The cells to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10′ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8+ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁻ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of cells is administered to a subject in need thereof, in a single dose or multiple doses. In some embodiments, a dose of cells is administered in multiple doses, e.g., once a week or every 7 days, once every 2 weeks or every 14 days, once every 3 weeks or every 21 days, once every 4 weeks or every 28 days. In an exemplary embodiment, a single dose of cells is administered to a subject in need thereof. In an exemplary embodiment, a single dose of cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the cells of the invention (e.g., the immune cell genetically modified to express a CAR) may be administered to a subject in combination with an immune checkpoint antibody (e.g., an anti-PD1, anti-CTLA-4, or anti-PDL1 antibody). For example, the cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the cell comprising the CAR. In certain embodiments, combination treatment comprising an immune checkpoint modulator may increase the therapeutic efficacy of a therapy comprising a cell of the present invention.

Following administration of the cells, the biological activity of the genetically modified cell populations in some embodiments is measured, e.g., by any of a number of known methods. Parameters to assess include specific binding of an genetically modified or natural T cell or other immune cell to antigen, in vivo, e.g., by imaging, or ex vivo, e.g., by ELISA or flow cytometry. In certain embodiments, the ability of the genetically modified cells to destroy target cells can be measured using any suitable method known in the art, such as cytotoxicity assays described in, for example, Kochenderfer et al., J. Immunotherapy, 32(7): 689-702 (2009); Herman et al. J. Immunological Methods, 285(1): 25-40 (2004); Kiesgen et al., Nat Protoc. (2021) 16(3):1331-1342; and Maldini et al., J Immunol Methods (2020) 484-485:112830. In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the immune cells of the invention. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the genetically modified cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In one aspect, the invention includes a method of treating cancer in a subject in need thereof, comprising administering to the subject any one of the immune or precursor cells disclosed herein. Yet another aspect of the invention includes a method of treating cancer in a subject in need thereof, comprising administering to the subject an immune or precursor cell prepared by any one of the methods disclosed herein.

### Sources of Immune Cells

In certain embodiments, a source of immune cells (e.g. T cells) is obtained from a subject for ex vivo manipulation and/or in vivo transduction. Sources of target cells for ex vivo manipulation may also include, e.g., autologous or heterologous donor blood, cord blood, or bone marrow. For example, the source of immune cells may be from the subject to be treated with the immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human. Methods for in vivo transduction of immune cells for CAR expression are described, e.g., in Pfeiffer et al., EMBO Mol Med. (2018) 10(11):e9158; Weidner et al., Nat Protoc. (2021) 16(7):3210-3240; Frank et al., Blood Advances (2020) 4(22):5702-5715; Nawaz et al., Blood Cancer J. (2021) 11(6):119.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a macrophage, or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell, or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the genetically modified cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or genetically modified. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. n yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### Expansion of Immune Cells

Whether prior to or after modification of cells to express a CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### Pharmaceutical Compositions and Formulations

Also provided are populations of immune cells of the invention, compositions containing such cells and/or enriched for such cells, such as in which cells expressing CAR make up at least 10%, 20%, 30%, 40%, 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: The BTLA-HVEM Axis Restricts CAR T Cell Immunotherapy In Cancer

### Materials and Methods

### CARs and Exogenous TCR

The experiments employed herein utilized T cells genetically modified to express (via a lentiviral expression vector) an anti-CD19 CAR (CAR19 T cells), an anti-CD30 CAR (CAR30 T cells), or an anti-HER-2 CAR. The anti-CD30 CAR utilized in these experiments comprises a CD8 leader, an anti-CD30 scFv, a CD8 hinge and transmembrane domain, and an intracellular domain comprising a 4-1BB intracellular costimulatory domain and a CD3 zeta intracellular signaling domain. The anti-CD30 CAR comprises an anti-CD30 scFv comprising SEQ ID NO: 18, which is encoded by SEQ ID NO: 21. The anti-CD30 CAR comprises SEQ ID NO: 24, which is encoded by SEQ ID NO: 27. Additionally, T cells expressing an anti-GP100 T cell receptor (TCR) (with simultaneous knockout of the T cell receptor alpha locus (TRAC)) were utilized.

### Suspension Cell Lines

Three human CD19+ B-cell malignant cell lines used include (B-ALL Nalm6; DLBCL: OCI-Ly18, SU-DHL-4). Two human CD30+ Hodgkin Lymphoma Reed-Stemberg Cell Lines were used (classical HL: HDLM-2; mixed cellularity: KM-H2). One human CD30+ anaplastic large T cell lymphoma was used (SU-DHL-1). One murine CD19+ B-cell malignant cell line was used (murine large B cell lymphoma: A20). One acute T cell leukemia was used (Jurkat NFAT-GFP reporter), and had been originally obtained from System Biosciences Inc. (TR850A-1) (Ruella, et al., J Clin Invest 126, 3814-3826 (2016)). These cells were transduced with CAR19-P2A-BTLA or CAR19-P2A-BTLA^{Y257F,Y282F} lentiviruses. All human suspension cell lines were grown and cultured in standard culture media “R10” (RPMI 1640+10% FBS, 1% Penicillin/Streptomycin, 1% HEPES, 1% GlutaMAX) at 37° C. in 5% ambient C02 and maintained at a concentration of 0.3-1×10⁶ cells/mL. All human suspension cell lines were transduced with a lentiviral vector containing a 2A-self cleaving peptide-linked transgene encoding both click-beetle-green (CBG) luciferase and GFP. The A20 model was cultured in R10 media supplemented with 0.05 mM 2-mercaptoethanol under standard conditions.

### Adherent Cell Lines

One GP-100+ human melanoma cell lines was used (DM-6). One human HER-2+ ovarian cancer cell line was used (ovarian adenocarcinoma: SK-OV-3). One human HER-2+ prostate cancer cell line was used (prostate adenocarcinoma: PC-3). One human Mesothelin+ pancreatic cancer cell line was used (AsPC-1). One murine melanoma transduced with murine CD19 was used (B16-muCD19). The DM-6, PC-3, and AsPC-1 models were grown and cultured in standard culture media R10 at 37° C. in 5% ambient C02 and maintained at a confluency of ~60-70%. The SK-OV-3 and B16-muCD19 models were grown and cultured in “DMEM10” (Dulbecco's Modified Eagle Medium (DMEM)+10% FBS, 1% Penicillin/Streptomycin, 1% HEPES, 1% GlutaMAX), and maintained at a confluency of ~60-70% under standard conditions.

All suspension and adherent cell lines were originally procured from ATCC or DSMZ, authenticated by the University of Arizona Genetics Core, and ensured to be free of Mycoplasma contamination (Lonza).

### Primary Human and Murine T Cells

Primary human T cells were obtained through the University of Pennsylvania's Human Immunology Core. Primary murine T cells were isolated from freshly resected spleens of 6 to 8-week-old Balb/c or C57BL/6 mice. Spleens were then harvested, grinded, and passed through a 70 μm mesh filter. Splenocytes were pelleted, resuspended in 2 mL of 1× RBC Lysis Buffer (BioLegend) per spleen, incubated for 2-3 minutes before washing with 10 mL R10+0.05 mM 2-mercpatoethanol per spleen. Bulk murine T cells were then purified from splenocytes using the Miltenyi Biotec Pan-T cell Isolation Kit II (negative selection) according to the manufacturer's instructions.

### Lentiviral Vector Production

Replication-defective, third-generation lentiviral vectors were produced using HEK293T cells (ATCC ACS-4500). Briefly, 8×10⁶ cells were seeded per T175 culture flask in standard R10 media and incubated overnight at 37° C. After 24 hr, the HEK193T cells (~70-80% confluency) were transfected using a mixture of Lipofectamine 2000 (116 μL, Invitrogen), pMDG.1 (7 μg), pRSV.rev (18 μg), pMDLg/p.RRE (18 μg) packaging plasmids and 15 μg of transfer/expression plasmid (e.g., CAR), per flask. Lipofectamine and plasmid DNA were diluted in 4.6 mL Opti-MEM Reduced Serum media (Thermo-Fisher) prior to transfer into lentiviral production flasks. At 24 hr and 48 hr following transfection, the culture media from each flask was collected, filtered (0.45 m), and concentrated using high-speed ultracentrifugation (8,000×g for 16 hr or 25,000×g for 2.5 hr). Lentiviral concentrates were divided into aliquots, snap-frozen, and stored in −80° C.

### Retrovirus Production

A MSGV (Murine stem cell virus-based splice-gag vector) encoding murine CD19-directed 4-1BB co-stimulated CD3ζCAR construct (muCAR19-MSGV) was used to generate murine gamma retrovirus from Platinum-E (Plat-E) cells. Plat-E cells were cultured in DMEM10+1 g/mL puromycin+10 g/mL blasticidin. 24 hr prior to transfection, 8×10⁶ cells Plat-E cells were seeded per T175 culture flask, and the media was exchanged with DMEM10 (without puromycin or blasticidin). On the day of Plat-E transfection (~70-80% confluency), a mixture of Lipofectamine 2000 (60 μL, Invitrogen) and muCAR19-MSGV retroviral construct (15 g) was diluted in 3 mL Opti-MEM Reduced Serum media (Thermo-Fisher) per retroviral production flask and transferred to said flasks. At both 48- and 72-hr post-transfection, media containing virus was collected, and filtered (0.45 μm). The viral supernatant from 48- and 72-hr were combined, flash-frozen, and stored in −80° C.

Electroporation of Cas9—sgRNA RNP of Human and Murine T Cells

Primary human and murine T cells were electroporated using the Lonza 4D-Nucleofector System. In order to form a Cas9/sgRNA Ribonucleoprotein (RNP) complex, 10 μg of TrueCut v2 Cas9 (Thermo-Fisher) and 5 μg single-guide RNA (IDT) were combined and incubated for 10 min at room temperature. Primary human or murine T cells were collected, pelleted at 300×g for 5 min, and resuspended at 1-5×10⁶ per 100 μL of Lonza Buffer P3 (P3 Primary Cell Solution: Supplement 1=4.55:1 vol/vol). 100 μL of T cells in Buffer P3 were gently pipette-mixed with pre-incubated RNP (or Cas9 alone for “mock” electroporation conditions). The T cell/RNP mixture was transferred to a Lonza 100 μL Nucleocuvette and electroporated. For primary human T cell editing, pulse code EO-115 was used; for murine T cell editing, pulse code DN-100 was used. Following electroporation, T cells were transferred from cuvettes and recovered in warm culture media at ~5×10⁶ per mL. R10 recovery media for human T cells was supplemented with 20 ng/mL human IL-7 and 20 ng/mL of human IL-15. R10 recovery media for murine T cells was supplemented with 10 ng/mL murine IL-2, 10 ng/mL of murine IL-7, and 0.05 mM 2-mercaptoethanol.

### Manufacturing of CRISPR-Cas9-Edited Human CAR T Cells

CD4+ and CD8+ cells were combined at a 1:1 ratio. Electroporation of human T cells was performed as aforementioned (day 0) and maintained overnight at 37° C. 24 hr after electroporation (day 1), T cells were transferred into a larger vessel at 1×10⁶ T cells per mL, and stimulated using anti-human CD3/CD28 Dynabeads (Invitrogen) at a 2:1 Dynabead: T cell ratio. 48 hr after electroporation (day 2), lentiviral vector containing the given CAR transgene was added using a multiplicity of infection (MOI) of ~1.5-2.5 viral particles/T cell. On day 6 post-electroporation, Dynabeads were magnetically separated from T cells. Upon removal of Dynabeads, flow cytometry was performed to measure the transduction efficiency of CAR (% CAR+) and of the efficiency of a given CRISPR-Cas9 knockout relative to the “mock” electroporation (WT) condition (i.e., % KO). During T cell expansion, T cells were counted every two days and fed culture media to maintain a concentration of ~7×10⁵ T cells per mL. Upon demonstrating resting state growth kinetics and cell size (~250-300 fL), T cells were cryopreserved.

In all experiments using CAR30 (except in screening CAR30 constructs), the CAR30 construct was composed of the T1-159 scFv, 4-1BB costimulatory domain, and CD3ζ stimulatory domains (AbClon). All experiments using CAR19 involved the CTL019 CAR, composed of the FMC63 scFv, 4-1BB costimulatory domain, and CD3ζ domains, unless otherwise noted. The anti-HER2 CAR construct was composed of the 4D5 scFv, 4-1BB costimulatory domain, and CD3ζ domains. The anti-mesothelin CAR construct was composed of the M5 scFv, 4-1BB costimulatory domain, and CD3ζ domains.

### Manufacturing of CRISPR-Edited Murine CAR T Cells

Murine CAR T cell production followed a 5-6 day manufacturing protocol. Briefly, after isolating murine T cells from a Balb/c or C57BL/6 mouse, electroporation of murine T cells was performed as aforementioned, using the Lonza pulse code DN-100, and incubated at 37° C. for 1 hour with R10+0.05 mM 2-mercaptoethanol and 10 ng/mL of murine IL-7 and IL-2. Following incubation, anti-Mouse CD3/CD28 Dynabeads (Invitrogen) were added in a 2:1 Dynabead: T cell ratio. The same day, untreated 24-well plates were coated with 500 μL of RetroNectin (Takara Bio, 1:50 dilution in PBS), sealed, and placed in 4° C. overnight.

One day after murine T cell isolation, RetroNectin-coated wells were aspirated. 500 μL 2% BSA in PBS was added to each well and allowed to incubate for 30 min at room temperature. Then, the BSA solution was aspirated, and 500 μL muCAR19-MSGV retrovirus was added to each well. Plates were sealed and centrifuged with muCAR19-MSGV retrovirus for 1 hour at 2000×g at 4° C. After centrifugation, plates were incubated at 37° C. for 30 min. Then, activated murine T cells and mouse Dynabeads from Day 1 were resuspended and distributed amongst retrovirus-coated wells (0.5-1×10⁶ T cells per well in 24-well plate).

On days 1 through 6 of murine CAR T manufacturing, murine T cells were fed with R10+0.05 mM 2-mercaptoethanol and 10 ng/mL of murine IL-7 and IL-2. On day 5, mouse Dynabeads were magnetically separated from murine T cells. Upon removal of Dynabeads, flow cytometry was performed to measure the transduction efficiency of CAR (% CAR+, anti-G4S scFv linker antibody) and of the efficiency of the given CRISPR-Cas9 knockout relative to the “mock” electroporation (WT) condition (i.e., % KO). Murine CAR T cells were then prepared and resuspended in PBS for infusion into either Balb/c or C57BL/6 mice.

The muCAR19-MSGV construct was composed of an anti-murine CAR19 scFv, 4-1BB costimulatory domain, and CD3ζ domains.

Single-Guide RNA Oligonucleotides used for Gene-Editing

Al single-guide RNA oligos were either produced via in vitro transcription using the Precision gRNA Synthesis Kit (Invitrogen) or purchased from IDT.
For human BTLA (CD272): 5′ TGTTAGCACAGTATTTCACA 3′(SEQ ID NO: 1)
For human PD-1 (CD270): 5′ GGCGCCCTGGCCAGTCGTCTGGG 3′ (SEQ ID NO: 33)
For murine BTLA: 5′ (compatible gRNA for both C57BL/6 and Balb/c backgrounds): 5′ AGCTGAGGGCTAACCTCAAG 3′ (SEQ ID NO: 34)
For human HVEM/TNFRSF14 (CD270): 5′ CAAGGAGGACGAGTACCCAG 3′ (SEQ ID NO: 2)

### Bioluminescence-Based Cytotoxicity Assay

Human cancer cell lines (HDLM-2, KM-H2, SU-DHL-1, OCI-Ly18, SU-DHL-4, Nalm6, DM-6, AsPC-1, PC-3, SK-OV-3) were engineered to express click-beetle-green (CBG) and green fluorescent protein (GFP) using a CBG-2A-GFP lentiviral vector construct. Cancer cell survival was monitored using bioluminescent quantification. Briefly, D-luciferin potassium salt (Perkin-Elmer) was added to cultures (final concentration 15 μg/mL) and incubated at 37° C. for 10 minutes. Bioluminescent intensity was quantified using a BioTek Synergy H4 Imager and the corresponding BioTek Gen5 software. Percent of specific lysis (% Tumor killing) was calculated relative to a control of cancer/target cells alone without effector T cells.

### Flow-Cytometry-Based (Extended Coculture) Cytotoxicity Assay

CAR T cells were combined with target cancer cells at a given effector: target ratio, and cocultures were evaluated for absolute count of T cells and cancer cells by flow cytometry using Flow-Count Fluorospheres (Beckman-Coulter) at two- or three-day intervals. Cocultures were maintained at an absolute concentration of 1×10⁶ cells/mL.

### IncuCyte®-Based Cytotoxicity Assay

24 hr prior to coculture with CAR T cells, GFP+ adherent cell lines (SK-OV-3) were seeded at 4,000 cells/well in a 96-well plate (100 μL R10 media). After 24 hr, cancer cells were treated with untransduced T cells, or CAR T cells with or without a genetic deletion. Each condition was plated in at least triplicate. Cells were imaged at 3-4-hour intervals using the IncuCyte SX5 Live-Cell Analysis System and a 20× objective for <160 hr with data collection by the Incucyte ZOOM software. GFP+ count, intensity, and total confluence were recorded at each time point. One-way ANOVA with post hoc Tukey tests was used to determine statistical significance for each experimental effector T cell condition relative to untransduced T cells at the experimental endpoint.

xCELLigence-Based Cytotoxicity Assay

24 hr prior to coculture with CAR T cells, adherent cells (PC-3, B16-muCD19) were seeded at 4,000 cells/well in an E-Plate 96 PET, ACEA Biosciences. Cancer cells were then treated with CAR T cells at given effector:target ratios. The plate was then loaded onto the xCELLigence platform A single sweep to establish sensor connections was followed by sweeps at 15 min intervals for up to 120 hr. Cell Index values are proportional to electrical impedance per well.

### CyTOF Experiments

CAR T cell cocultures were centrifuged and washed twice with 1×PBS+2% FBS. Cells were then fixed and stained for surface markers (listed in Reporting Summary) according to the manufacturer's instructions from the MaxPar Human T Cell Phenotyping and Immuno-Oncology EX Kits protocols (Standard BioTools). Data acquisition was conducted on the CyTOF Helios Platform (Standard BioTools), and data were output as standard .fcs files.

### CyTOF Data Analysis

Data were analyzed using OMIQ cytometry analysis platform (OMIQ by Dotmatics). Gated populations of CD3+ T cells expressing CD8+ or CD4+ markers were analyzed separately. Boolean gates on CD4+ or CD8+ T cells co-expressing exhaustion or costimulatory markers were defined using the Boolean gate filter in OMIQ. Data was then exported and plotted. UMAP analysis was performed using 30K CD4+ or CD8+ T cells each, by feeding the markers used in phenotyping the cells as features into the UMAP clustering algorithm. The resulting UMAP was then fed into the FlowSOM clustering algorithm in addition to the markers used in defining the UMAP for each cell subset analyzed. For each cell subset (CD4+ or CD8+ T cells), a new self-organizing map was generated using Elbow meta-clustering on the UMAP axis. Ten clusters were observed for each CD4+ and CD8+ T cell subset. Clustered heatmap and median fluorescence intensity of each marker expression per cluster as well as percent participation/representation (%) of each cluster were calculated.

### CellTraceViolet (CTV) Proliferation Assays

T cells were resuspended in PBS with CTV stain (1:1000 dilution), at a concentration of 1e6 per mL, and incubated for 15 min at 37 C. T cells were washed with 10 mL RI0 media, resuspended, and plated in coculture with cancer cells or Treg (see Treg suppression assay).

### Cell-to-Cell Avidity Assays

Z-Movi-compatible acoustofluidic chips were coated with poly-L-lysine for 3 h prior to attaching a monolayer of tumor cells. CellTrace far-red labelled CAR T cells were allowed to incubate on the monolayer of tumor cells for 5 min, and then a ramping acoustic force was applying. Cell-detachment was analyzed using ImageJ and R. Avidity experiments were conducted according to the manufacturers (LUMICKS™) instructions and recommendations.

### Glass-Supported Planar Lipid Bilayer Confocal Imaging

Supported lipid bilayers were prepared by fusing small liposome droplets on clean glass coverslips as previously described [Braendstrup, et al., Cytotherapy 22, 57-69 (2020)]. Briefly, the liposome was trapped in a μ-Slide VI 0.4 chamber (Ibidi, Germany). Lipid bilayers were first blocked with 5% casein for 20 min and then incubated with 6.3 nM of streptavidin (Thermo Fisher) for 15 min. After washing with imaging buffer (HEPES-buffered saline), lipids were incubated with biotinylated CD19 protein (R&D Systems) and HVEM protein (Acro Biosystems) conjugated with AlexaFluors 488 and 647, respectively, at room temperature for 30 min. After washing with the imaging buffer, lipid bilayers were blocked with 2.5 μM of D-biotin to saturate the streptavidin-binding sites. CAR T cells were then stimulated on lipid bilayers for 15 min at 37° C. Cells were then fixed in 4% paraformaldehyde for 10 min and then permeabilized in 0.5% Triton-X100 and 10% normal donkey serum in PBS for 30 min at room temperature. Then, the cells were stained with primary antibodies against BTLA (ab254287, Abcam) and SHP-1 (sc-7289, Santa Cruz) or SHP-2 (sc-7384, Santa Cruz). An Olympus FV3000 confocal microscope with 60× (NA 1.35) oil objective was used to obtain confocal image data.

### Treg Suppression Assays

Murine T cells were isolated from the spleens of C.Cg-Foxp3′²¹ h/J (i.e., Foxp3^{EGFP}) mice (purchased Jackson Laboratory, 6-8 weeks old). T cells were then FACS-sorted into GFP+ (Treg) or GFP− (Tconventional or Tconv). Tconv were tagged with CellTraceViolet stain in PBS for 15 min at 37 C, washed, and electroporated with either Cas9 alone (Mock EP), or Cas9+BTLA-sgRNA (BTLA KO). Mock EP and BTLA KO Tconv were seeded into a 96-well plate with anti-CD3/CD28 stimulation beads (1:1 bead:Tconv) for 72 hr, with or without GFP+ Treg. Quantification of Treg suppression was calculated as follows: % Suppression=[100−% CTVlow (coculture of Tconv and Treg)/(% CTVlow (Tconv alone)].

### General Flow Cytometry

Cells were resuspended in FACS staining buffer (PBS+2% FBS) using the following antibodies: human CD3 (clone OKT3, Biolegend), anti-BTLA (clone MIH26, Biolegend), human CD45 (clone Biolegend), or mouse CD45 (clone 30-F11, Biolegend). CAR expression was detected using a PE-conjugated anti-G4S linker antibody. To monitor cell proliferation, cells were stained with CellTraceViolet according to manufacturer's instructions (Invitrogen). For quantification of absolute cell numbers (tumor or T cells) acquired during flow cytometry, FlowCount FluoroSpheres were used (BeckmanCoulter). Cell viability was established using Propidium iodide (PI), Live/Dead Violet Fixable stain (Thermo-Fisher), or ViaKrome 808 Fixable Viability Dye (Beckman Coulter). Unless otherwise specified, all gating strategies began in the following order: SSC-A vs. FSC-A (Lymphocytes), FSC-A vs. FSC-H (Singlets), FSC-A vs. Live/Dead (Viability). All data were acquired on Beckman Coulter CytoFLEX S Flow Cytometer. All data analysis was performed using FlowJo version 10.8.1 (FlowJo, LLC).

### Immunodeficient Mice and Xenograft Models

Six- to 8-week-old NSG (NOD SCID gamma) mice were obtained from the Stem Cell and Xenograft Core at the University of Pennsylvania. NSG mice were housed in pathogen-free conditions. To engraft and establish the HDLM-2 subcutaneous tumor model, 15×10⁶ HDLM-2 cells were prepared in a 150 L solution (50% PBS, 50% Matrigel, Corning) and implanted under the skin of the right flank. Tumor size was monitored by digital caliper according to the following equation: Tumor volume (mm³)=½(L×W²), where L (mm) is the major axis of the tumor and W (mm) is the minor axis perpendicular to L. 62 days after implantation, subcutaneous tumors had reached ~100-200 mm³ in size. On day 62, mice were randomized and infused with 2.5×10⁵ CAR30+ T cells were infused via tail vein. Tumor size was monitored each week. In addition, the HDLM-2 model was engrafted as systemic model via intravenous injection as our lab has described previously (Ruella, et al., Cancer Discovery 7, 1154-1167 (2017)). Briefly, 5×10⁶ HDLM-2 cells were infused into mice via tail-vein injection. Total tumor burden (BLI) was monitored weekly using the IVIS Lumina S5 (PerkinElmer). At day 22, mice were randomized into groups, and 5×10⁵ CAR30+ T cells were infused via tail vein. Pembrolizumab (10 mg/kg, ~200 μg/mouse) or saline was delivered via intraperitoneal injection once per week for three weeks.

The OCI-Ly18 subcutaneous model was established as our lab has described previously (Lee, et al., Cancer Discov 12, 2372-2391 (2022)). Briefly, in the stress-test model, 5×10⁶ OCI-Ly18 cells were implanted under the skin of the right flank. Tumor size was monitored as aforementioned. 14 days after implantation, subcutaneous tumors had reached a size ~20-40 mm³. Mice were randomized and infused with 3×10⁶ CAR19+ T cells via tail vein. In the early-treatment/long-term persistence model, 5×10⁶ OCI-Ly18 cells were subcutaneously implanted, and 4 days after tumor implantation mice were imaged after placing mice on their left side (left lateral recumbent position) using the IVIS Lumina S5. Mice were randomized into groups and infused with 4×10⁶ CAR+ cells. Mice were monitored for tumor growth, and surviving mice at 40 days after the initial tumor challenge were re-challenged with 4×10⁶ OCI-Ly18 on the opposite (left) flank. Mice were then monitored for tumor growth post-re-challenge until the experimental endpoint.

Animals were continuously monitored for signs of disease progression and overt toxicity, such as xeno-Graft-Versus-Host-Disease (GVHD), as evidenced by >20% loss in body weight, excessive fur loss, diarrhea, conjunctivitis, disease-induced hind limb paralysis. Animals were also continuously monitored for signs of distress affecting gait or normal posture. NIH Guidelines were followed for animal care and use. All experimental protocols and endpoints, including tumor volume thresholds of up to 4.2 cm³ for the OCI-Ly18 model, were approved by the University of Pennsylvania Animal Care and Use Committee (IACUC).

### Syngeneic Mouse Models

Six- to 8-week-old Balb/c mice were purchased from The Jackson Laboratory and contained in conventional housing (BSL-1). To establish the A20 murine lymphoma subcutaneous model, 2×10⁶ A20 cells were prepared in a 100 L solution (50% PBS, 50% Matrigel, Corning) and implanted under the skin of the right flank in Balb/c mice. Fourteen (14) days post-implantation, one day prior to adoptive cell transfer, mice were administered 100 mg/kg cyclophosphamide via intraperitoneal injection. On day 15, mice were randomized (subcutaneous tumors had reached ~100-200 mm³) and infused with 1×10⁶ CAR+ T cells.

Six- to 8-week-old C57BL/6 mice were also purchased from The Jackson Laboratory and likewise, housed in BSL-1. To establish B16-muCD19+ tumors, 2×10⁵ tumor cells were prepared in a 100 μL PBS solution and implanted under the skin of the right flank of C57BL/6 mice. On day 4 post-implantation, 100 mg/kg cyclophosphamide was administered via intraperitoneal injection. On day 5 post-implantation, mice were randomized and retro-orbitally infused with 4×10⁶ CAR+ T cells.

In both models, tumors were monitored twice per week and euthanized according to IACUC standards as described above.

### Single-Cell RNA Sequencing and Analysis

Fresh tumor tissue was collected from lymph nodes of four adult patients with classical Hodgkin lymphoma. Informed patient consent for this protocol was obtained. Tumor biopsies were minced, fragmented into single-cell suspensions using a 0.45-μm filter, and washed 3× with 0.04% BSA in PBS. Single-cell suspensions were loaded onto the 10× Genomics Chromium Controller. Gene expression libraries were prepared using the Chromium Single Cell 5′ Reagent Kit with v1.1 Chemistry (10× Genomics) according to the manufacturer's instructions. Libraries were sequenced using the Illumina NovaSeq 6000. Raw scRNA-seq data were pre-processed using the Cell Ranger software (version 5.0.1; 10× Genomics) transcript reads were mapped to the GRCh38 human reference genome. Filtered gene expression was processed using the Seurat package (version 4.0.1) (Hao, et al., Cell 184, 3573-3587.e3529 (2021); Stuart, et al., Cell 177, 1888-1902.e1821 (2019)). To eliminate putative low-quality cells from the data set, the median absolute deviation (MAD)-based definition of outliers was used: cells with fewer than 200 expressed genes, or with an abnormally high number of UMI counts (3 MADs), or with high mitochondrial RNA expression (3 MADs) were discarded from downstream analysis. Each HL patient dataset was first merged and batch-corrected using the IntegrateData function in Seurat. Data were then were normalized and scaled using the NormalizeData and ScaleData functions. Variable features were identified using the FindVariableGenes function, and the principal components (PC) were calculated using the RunPCA function. The top 15 PCs were used to drive unsupervised clustering analysis via UMAP using the RunUMAP function (resolution=0.4). Raw sequencing data of n=22 cHL patients and n=5 reactive LN from Aoki et al. (Aoki, et al. Cancer Discovery 10, 406-421 (2020)) was obtained via Material Transfer Agreement and approval from the University of Pennsylvania's Institutional Review Board. The CellPhoneDB platform was run as terminal commands using standard parameters (iterations=10, threads=2) according to the developer's notes (Efremova, et al., Nat Protoc 15, 1484-1506 (2020)).

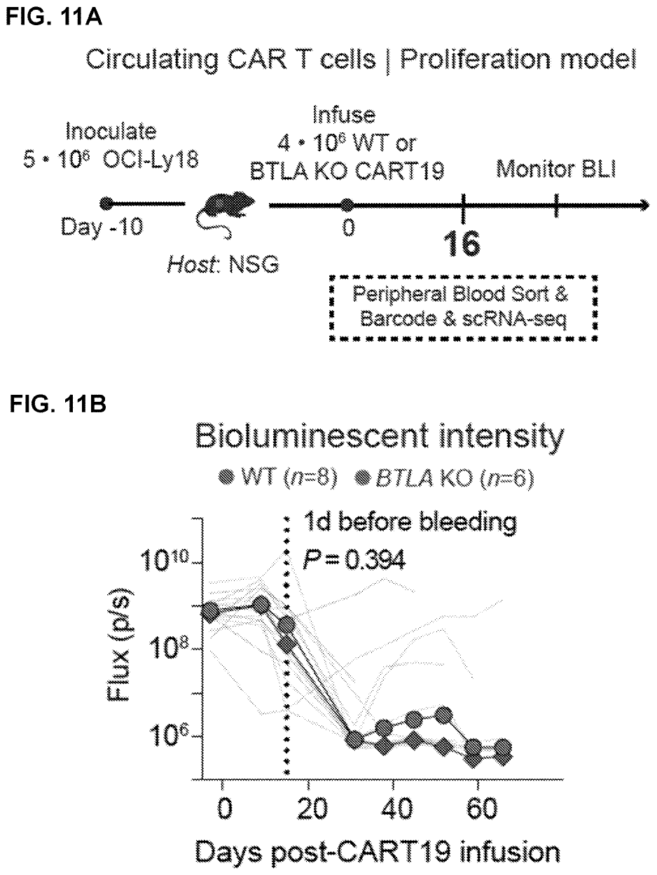

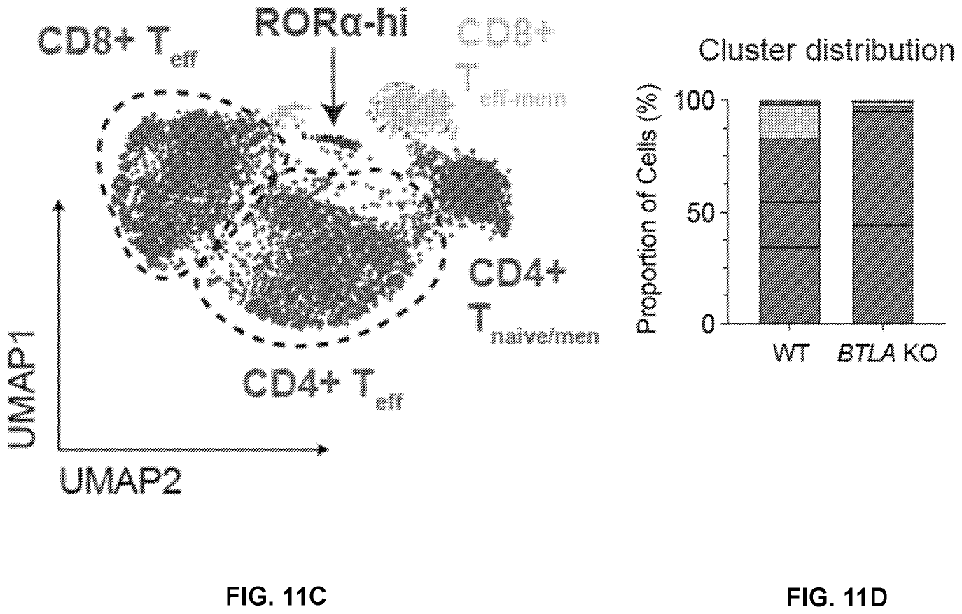

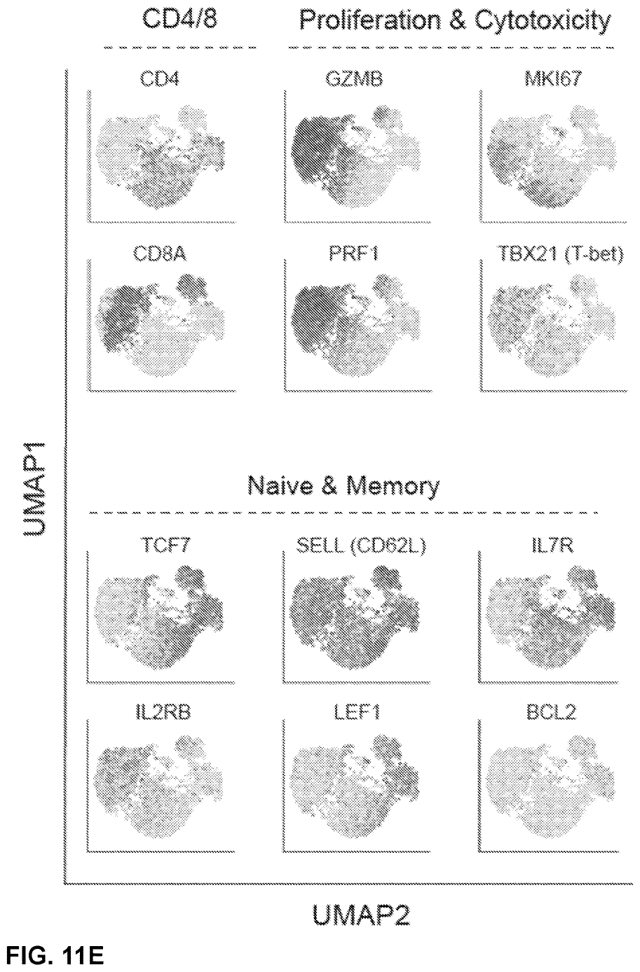

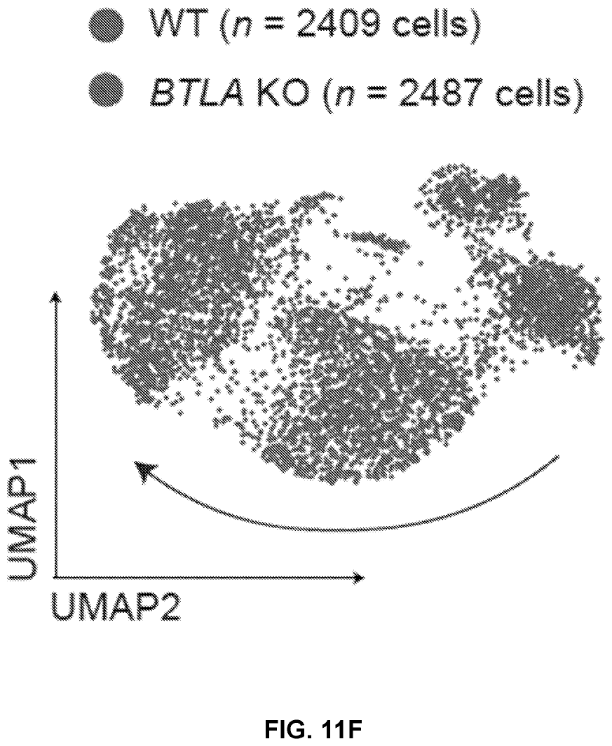

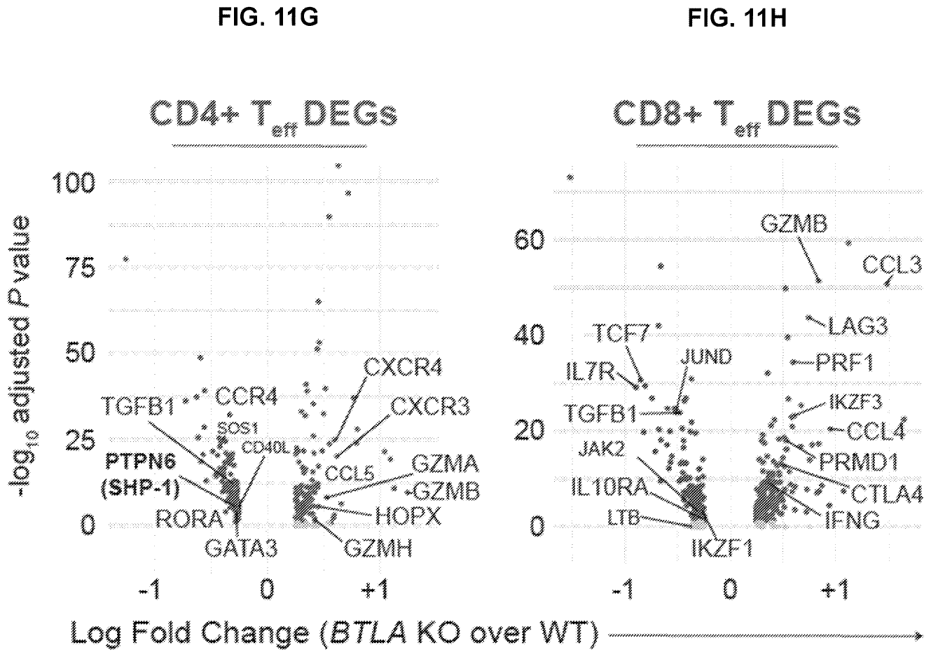

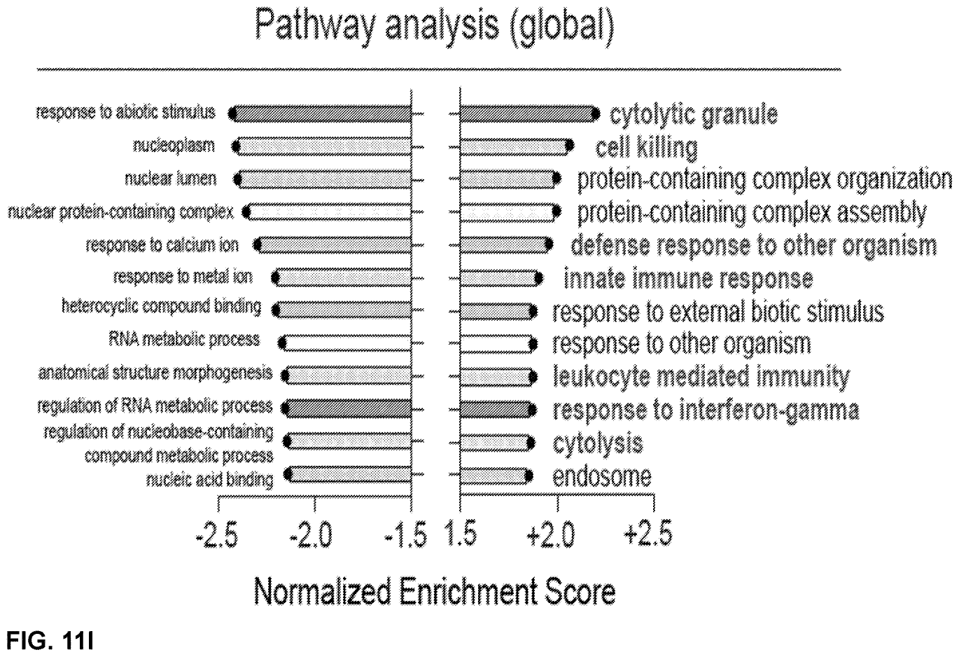

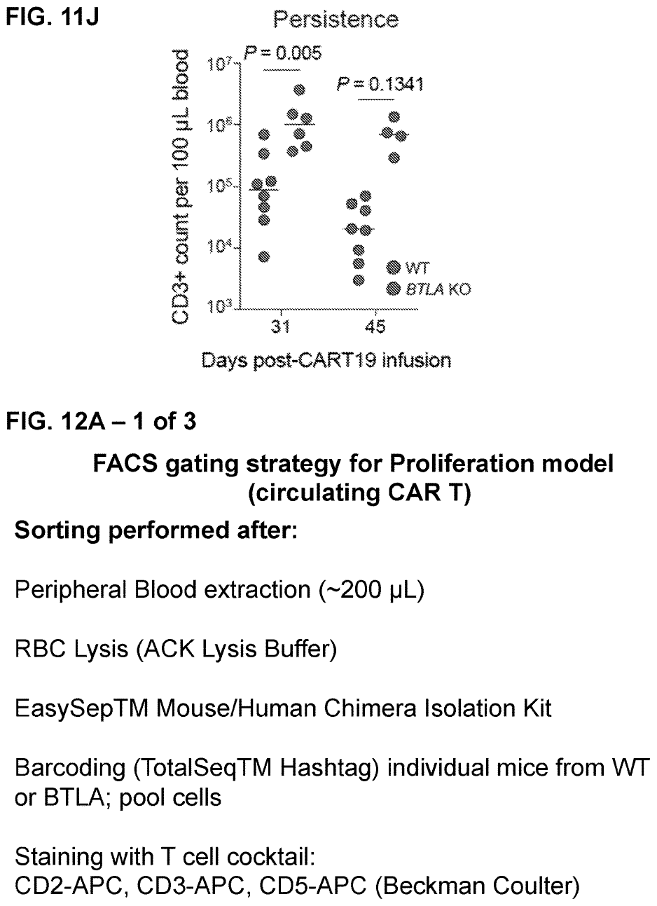

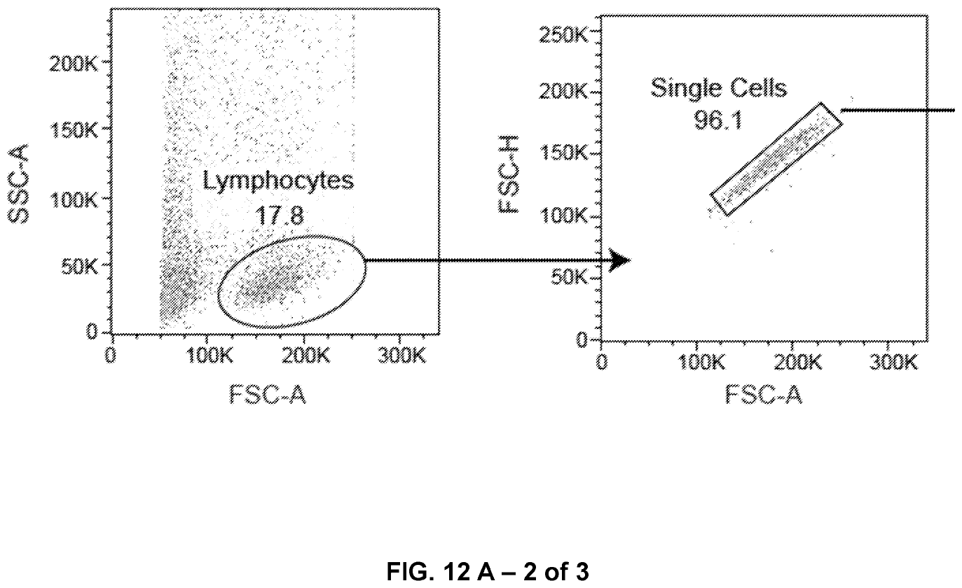

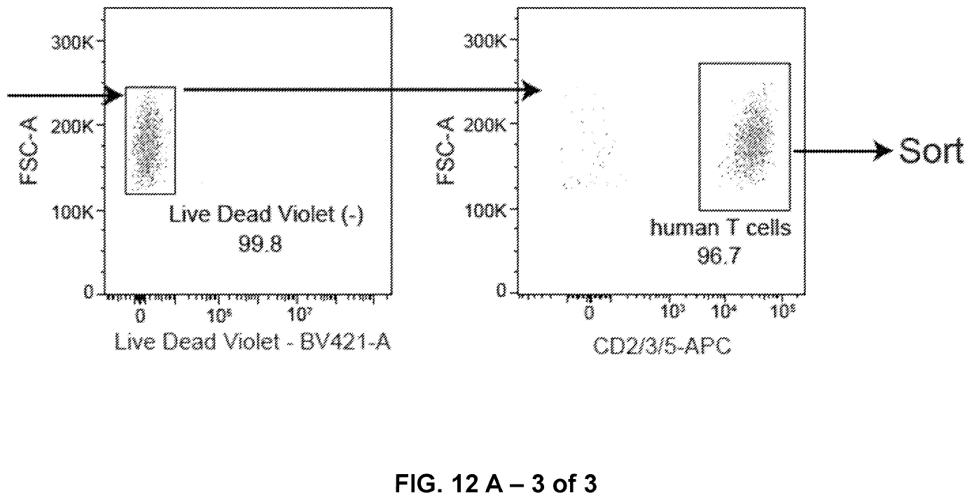

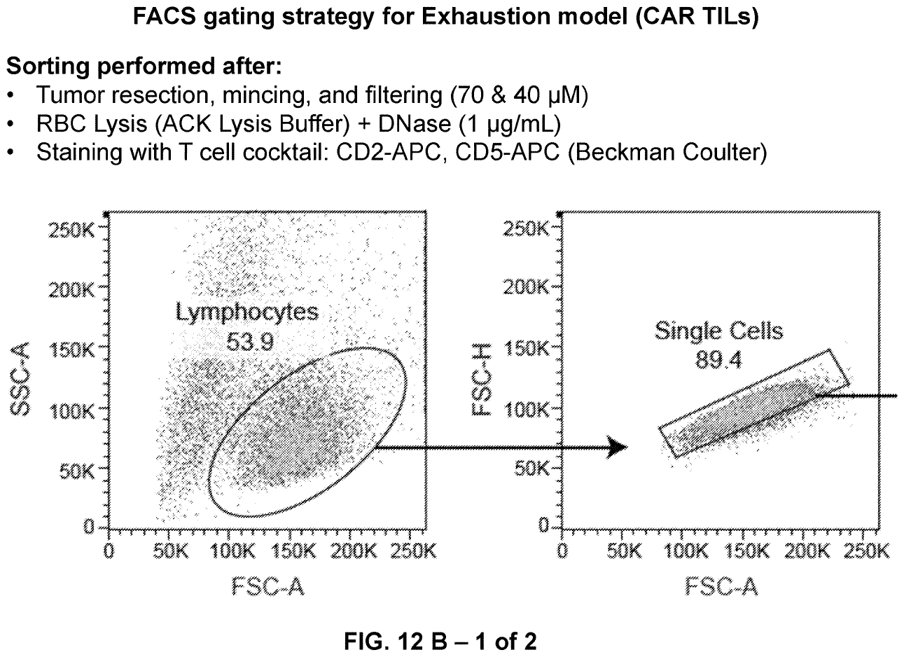

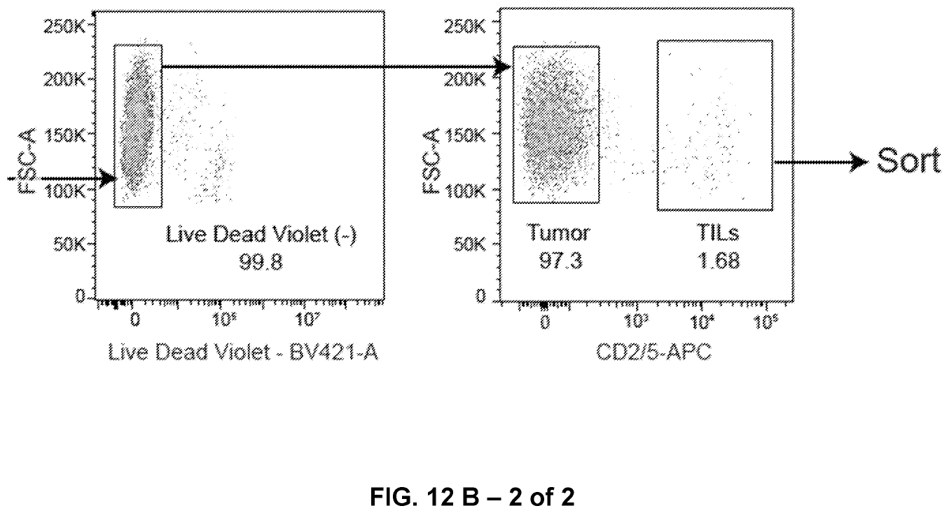

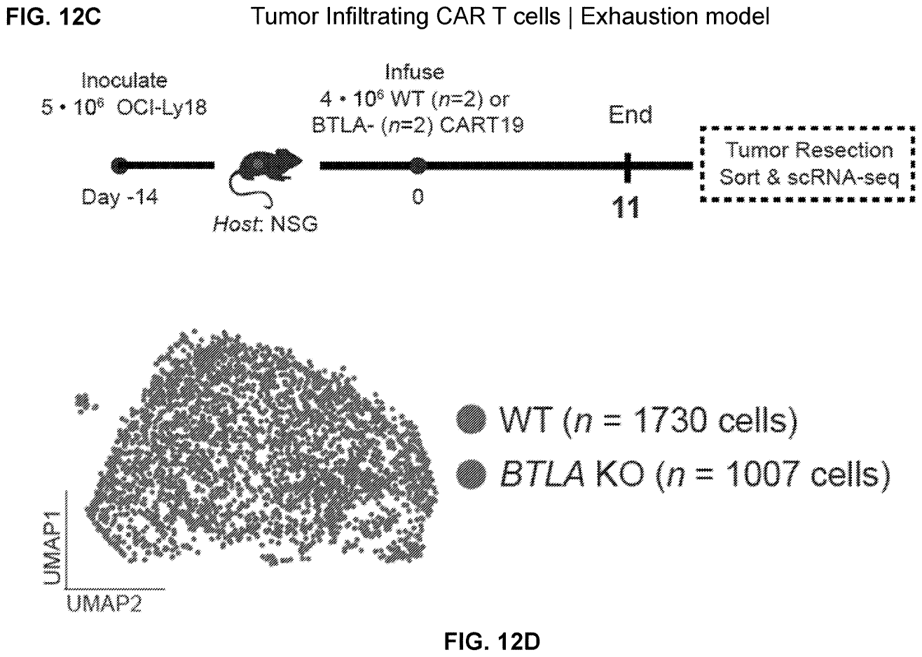

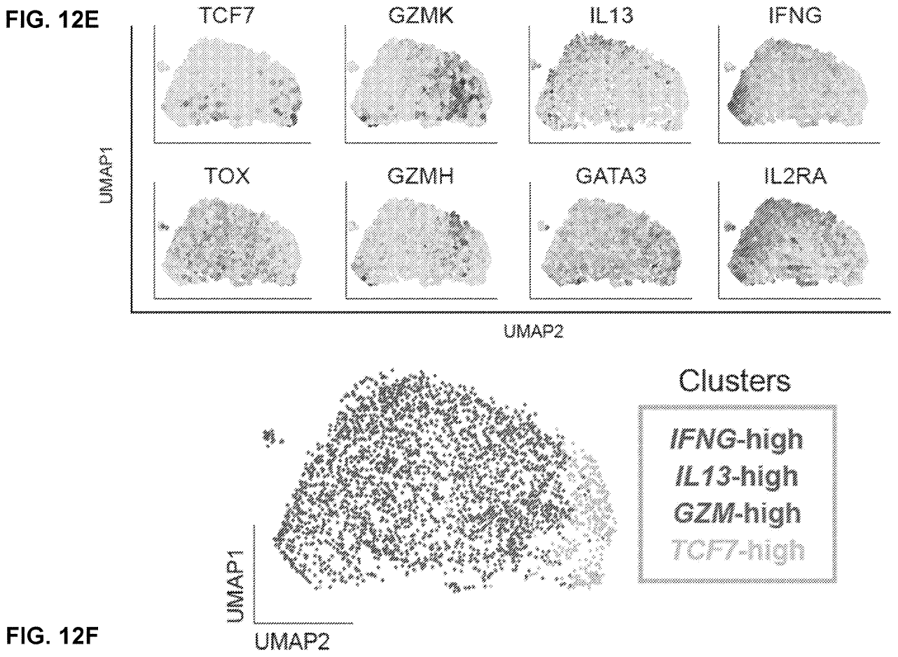

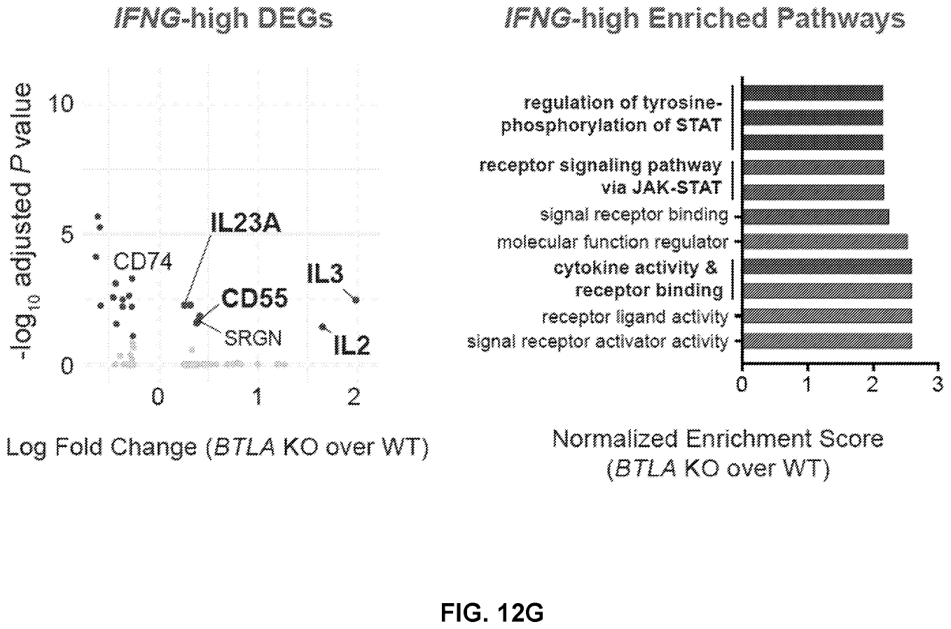

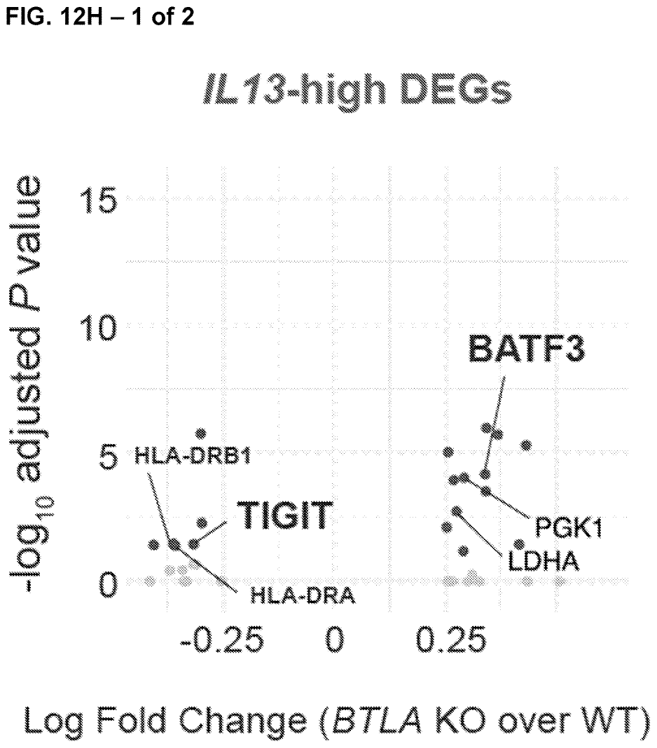

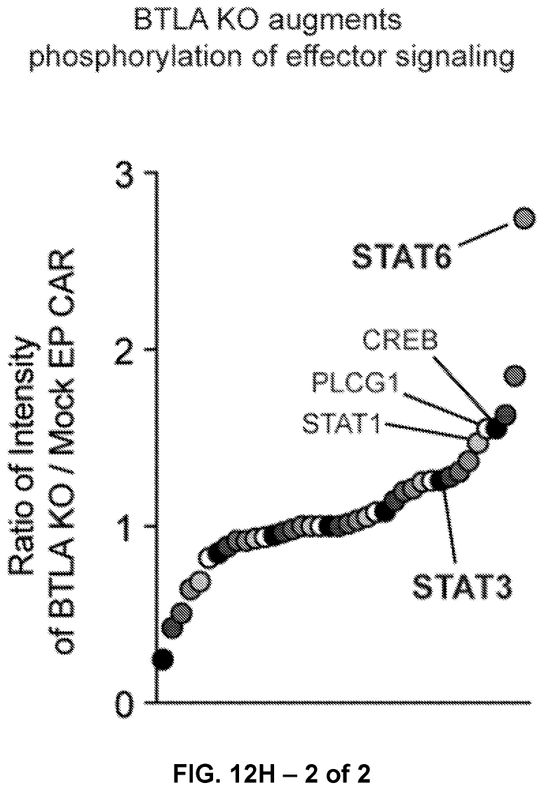

Additionally, for sequencing data in FIGS. 11A-11J, peripheral blood was collected from all mice and red blood cell lysis was performed. Samples were then processed as per manufacturer protocols using EasySep™ Mouse/Human Chimera Isolation Kit (Stem Cell Technologies; Cat #19849) to isolate human cells. After enrichment, as an additional measure of purification, anti-hCD3, anti-hCD5, and anti-hCD2 antibodies were applied to stain for human T cells and subsequently sorted using a BD FACSMelody™ Cell Sorter. Cells were then washed 2× with 0.04% BSA in PBS and loaded onto the 10× Chromium controller. Library preparation was performed using the v2 Chemistry kit (10× Genomics). After NGS and mapping, data were filtered in Seurat with the following parameters prior to clustering: 200<number of RNA features<5500; percent mitochondrial genes<10. For sequencing data in FIGS. 12B-12H, only anti-hCD2 and anti-hCD5 antibodies were used to stain bulk tumor single-cell suspensions for human T cell infiltrates. After sorting human T cells using the BD FACSMelody™, cells were washed 2× with 0.04% BSA in PBS and captured on the 10× Chromium controller; the v2 Chemistry kit was used for library preparation. scRNA-seq data were filtered as such: 200<number of RNA features<7500; percent mitochondrial genes<7. In addition, the filters MS4A1<1 and CD2>1 were applied to remove any residual contaminating tumor cells. Clustering in both experiments was driven by a predefined list of canonical/classical T cell genes as described (Szabo et al., Nature Communications 10, 4706 (2019)). Additional details on cell processing and handling are provided in the description of the drawings and in FIGS. 12A-12B.

### General Statistical Analysis

Sample size was not predetermined using any statistical methods. Additionally, no data were artificially removed from statistical analysis. All in vitro data presented are representative of at least two independent experiments except for ex vivo-based scRNA-seq experiments. Sequencing results were processed in R version 4.0 or higher. All comparisons between two groups were performed using either unpaired Student's t-test unless otherwise specified in the description of the drawings. All comparisons between three or more groups were performed using one-way ANOVA with post hoc Tukey tests, unless otherwise specified. All results are represented as mean±SD unless otherwise noted. Survival data were analyzed using the log-rank (Mantel-Cox) test. Data analysis was performed using Prism v9.2.0 or higher (GraphPad software).

### Results

### The BTLA-HVEM Axis Between Effector T Cells and Immunosuppressive Cells is Enriched in Hodgkin Lymphoma

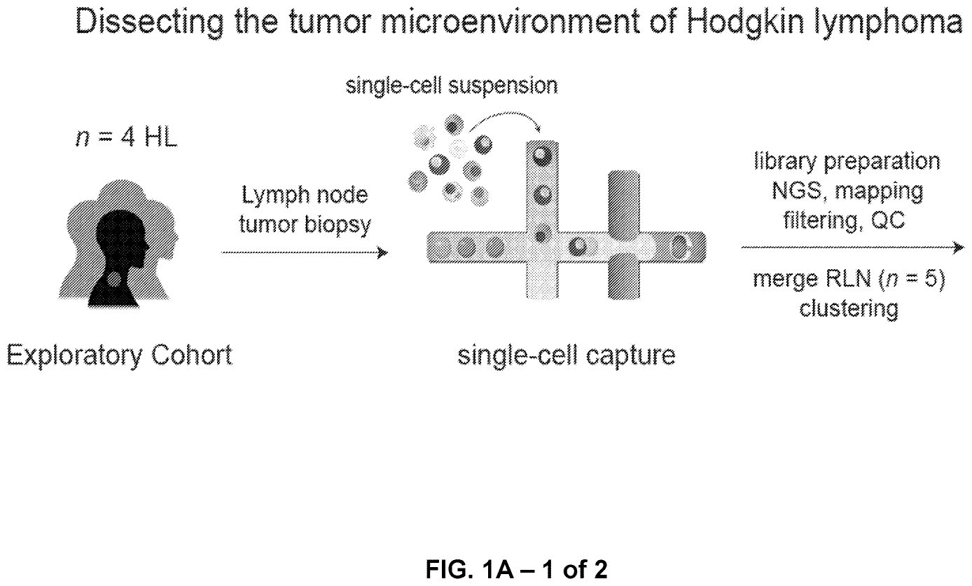

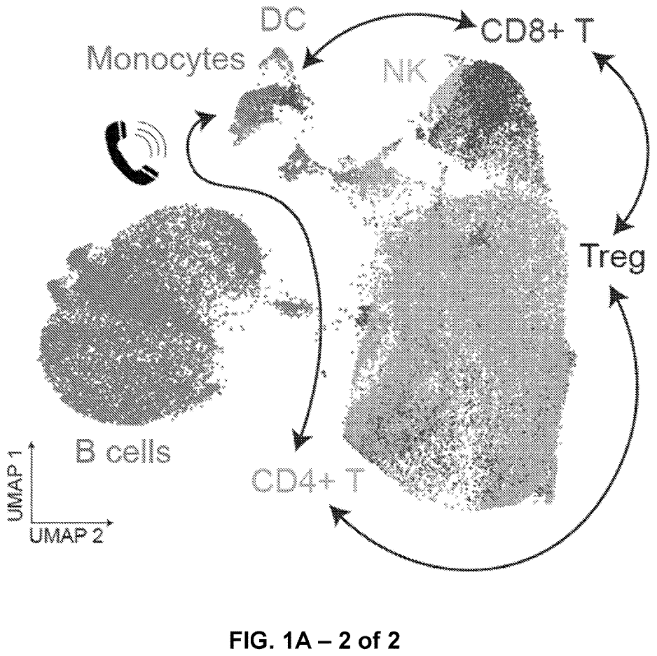

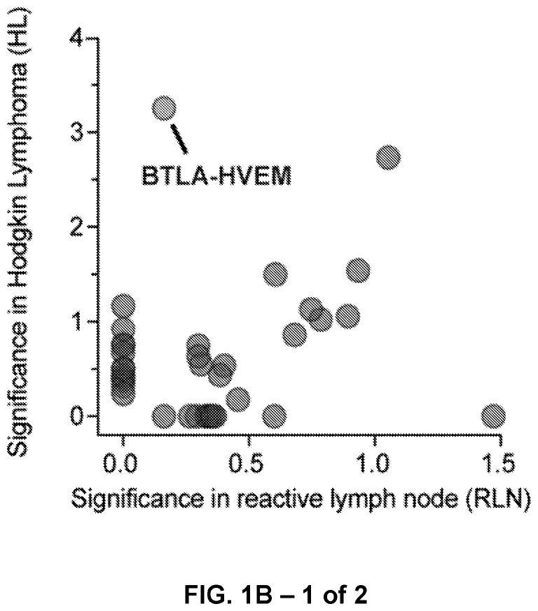

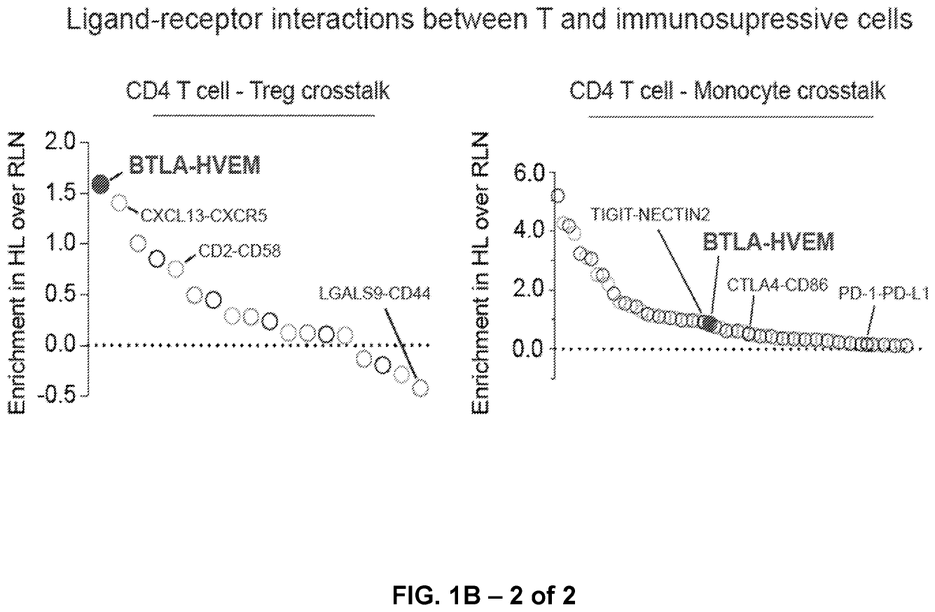

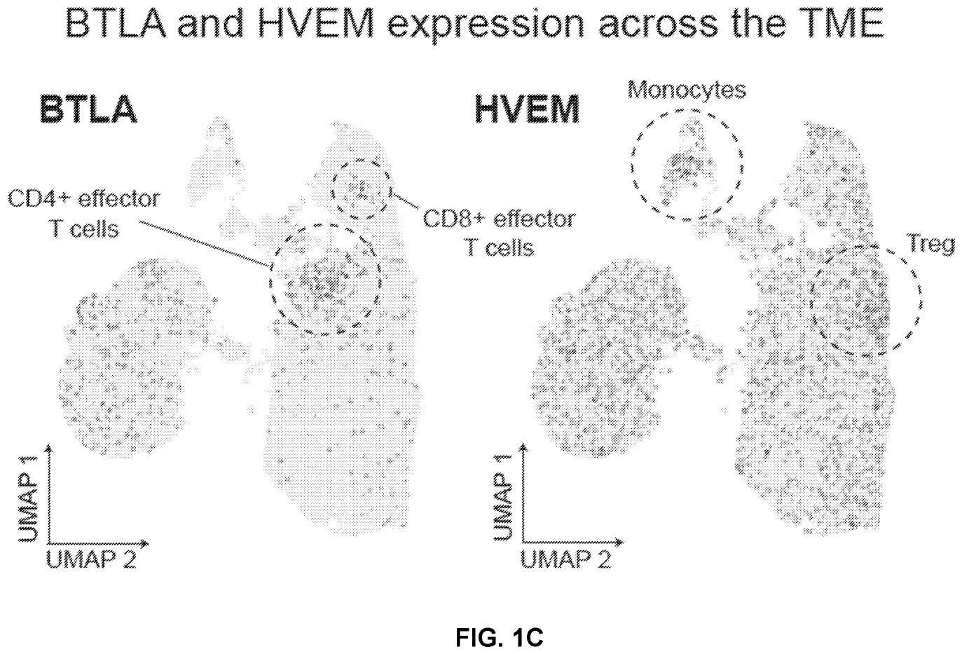

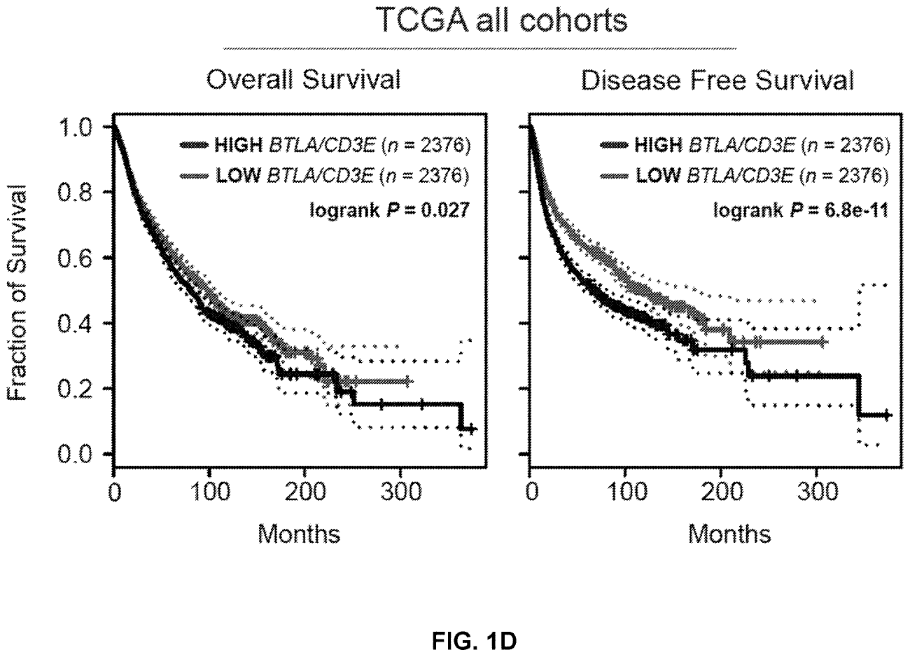

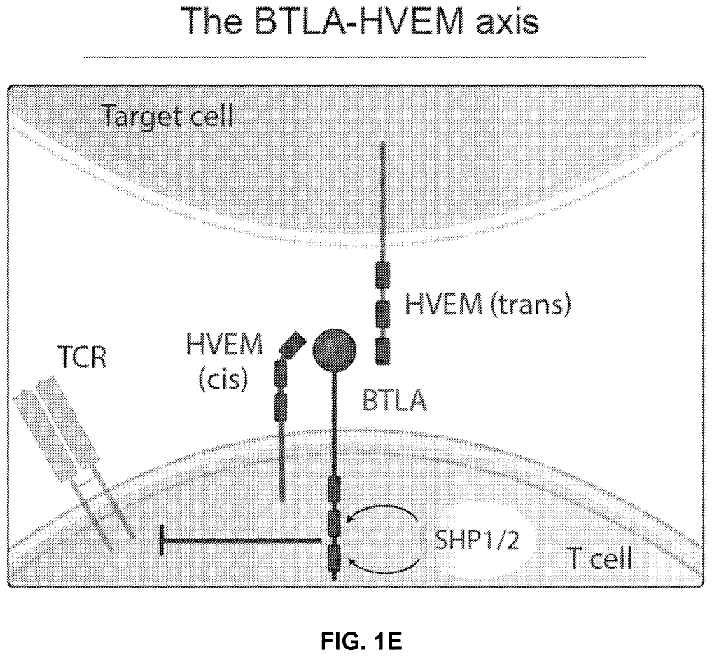

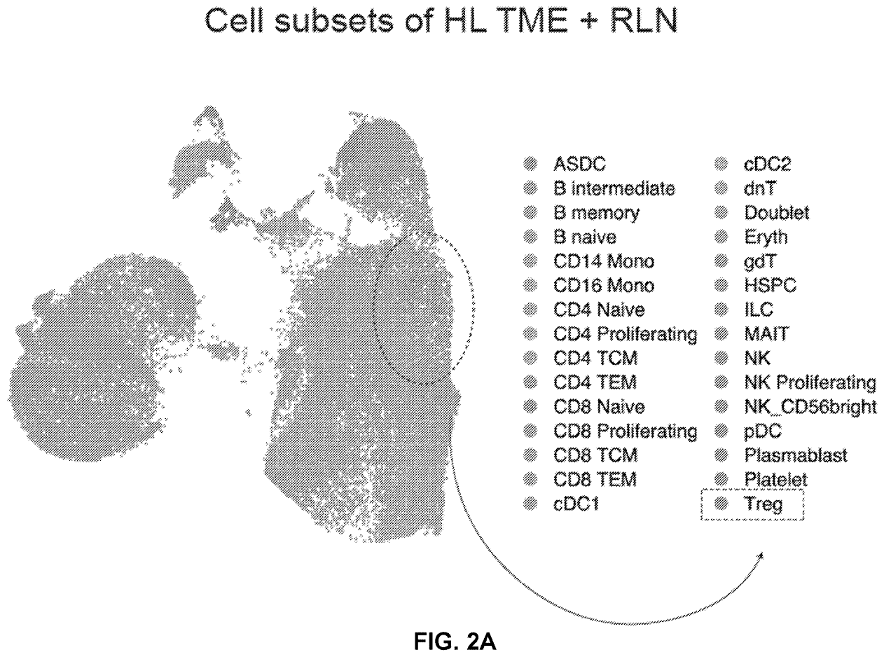

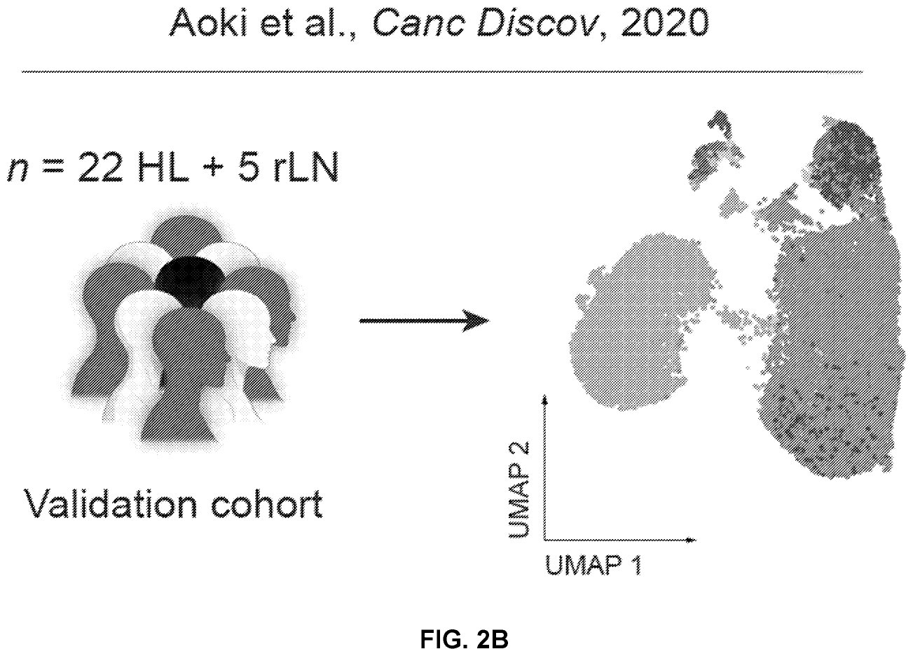

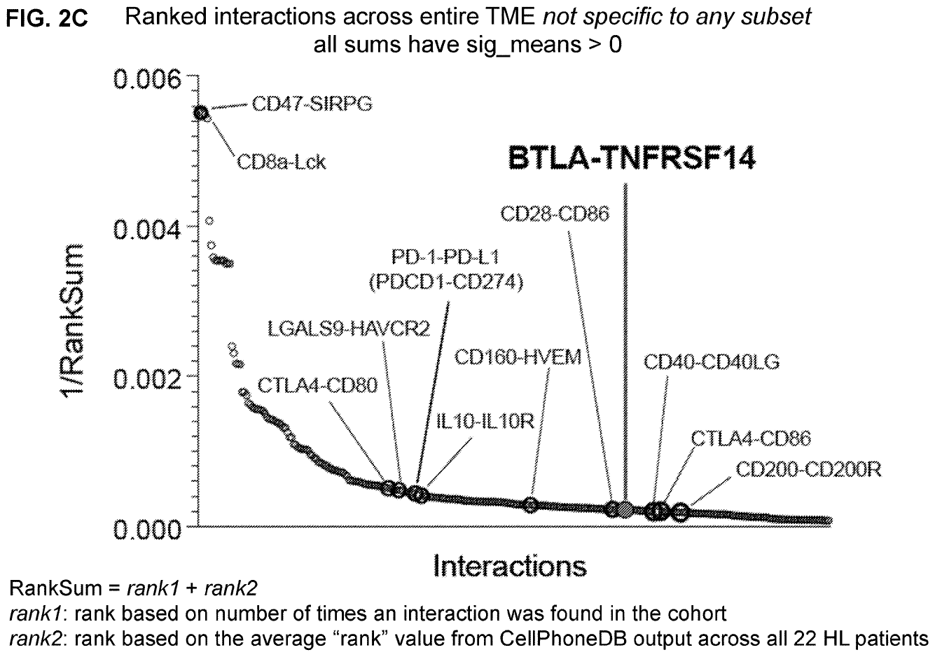

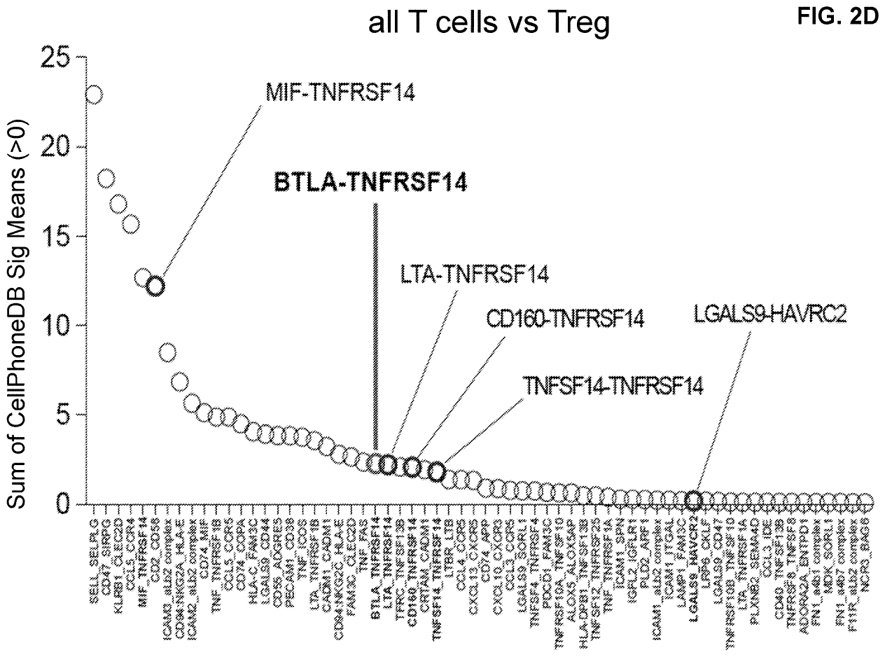

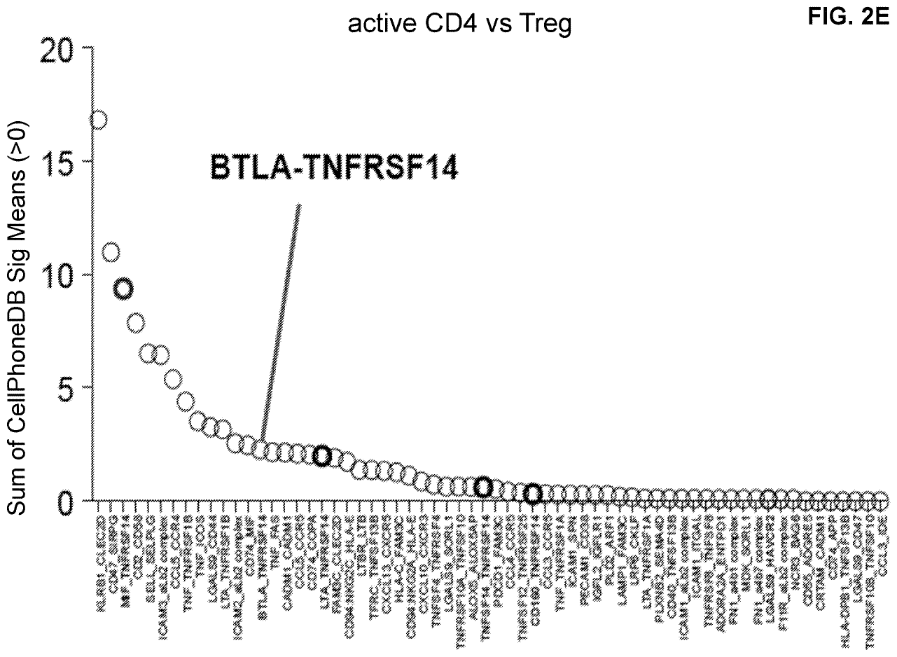

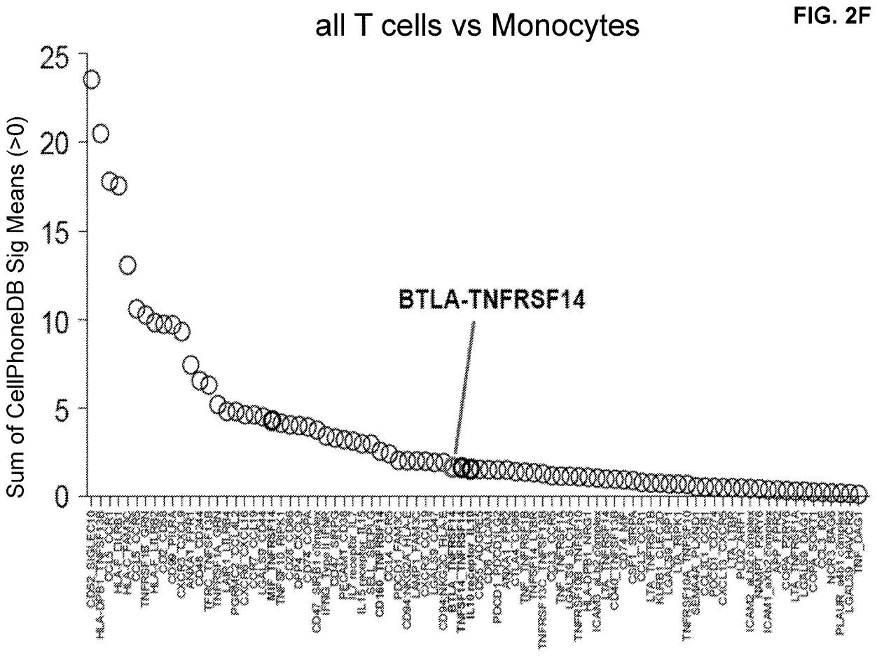

One of the major barriers to an effective response to T cell-based immunotherapies in cancer is the suppressive tumor microenvironment (TME). In the past decade, single-cell transcriptomics has been used extensively to characterize the TME. These studies have demonstrated the inherent heterogeneity of tumors and how the nature of immune cell infiltrates correlates with responses to T cell-based immunotherapies (Guruprasad, et al., Journal of Experimental Medicine 218 (2020)). Hodgkin's lymphoma (HL) represents an ideal tumor model to study the TME as it is characterized by profound infiltration of immunosuppressive immune cells that play a critical role in the disease's progression and resistance to therapy (Aldinucci, et al, J Pathol 221, 248-263 (2010); Connors, et al., Nat Rev Dis Primers 6, 61 (2020)). In order to characterize the HL TME with the intention of pinpointing the dominant inhibitory interactions that may affect the function of effector T cells, fresh tumor tissue samples were obtained from a discovery cohort of four adult HL patients and 5′ single-cell RNA sequencing was performed. These data were merged with that of five healthy donor reactive lymph node (RLN) biopsies (Aoki, et al., Cancer Discovery 10, 406-421 (2020)). Next, quality control, data normalization, and unsupervised classification of cell clusters were performed, in which cells with similar gene expression profiles were grouped (detailed in Methods). Here, several immune cell types shared between the HL TME and RLN were identified, including helper (CD4+) and cytotoxic (CD8+) T cells, regulatory T (Treg) cells, B cells, monocytes, and NK cells (FIG. 1A, FIG. 2A). Next, using the CellPhoneDB algorithm (Efremova, et al., Nat Protoc 15, 1484-1506 (2020)), the potential ligand-receptor interactions between effector T cells and regulatory T cells or monocytes were measured. Profound enrichment of BTLA-HVEM (TNFRSF14) between effector CD4+ T cells and Treg or monocytes was observed among the interactions inferred in HL relative to those in RLN (FIG. 1B) (Wein, et al., Cancer Immunology Research 5, 1122-1132 (2017)). Among interactions with monocytes, the BTLA-HVEM interaction was represented comparably to other well-defined checkpoints including TIGIT-NECTIN2, CTLA-4-CD86, and PD-1-PD-L1. Additionally, BTLA expression on T cells was localized to effector CD4+ or CD8+, while HVEM expression was diffusely expressed across the TME but mildly upregulated in Treg and monocytes (FIG. 1C). To validate these results, a larger validation cohort of HL patients (n=22) (Aoki, et al. Cancer Discovery 10, 406-421 (2020)) was analyzed and similar enrichment of the BTLA-HVEM axis between effector T cells and Treg or monocytes was observed (FIGS. 2B-2F). Additionally, to gauge the clinical relevance of this axis, a retrospective analysis of The Cancer Genome Atlas (TCGA) data was performed, combining all solid and liquid cancer cohorts (Tang, et al., Nucleic Acids Res 47, W556-w560 (2019)). It was found that low BTLA RNA expression normalized to CD3E (to control for the different levels of T-cell infiltration) correlates with improved overall and disease-free survival across most TCGA cohorts (FIG. 1D). Overall, these results indicated that BTLA expression on effector T cells and HVEM expression on immunosuppressive milieu may be enriched in the HL TME (FIG. 1E). Thus, whether modulating the expression of BTLA in T cells could augment their potency in HL was also investigated.

### BTLA-Deficient CAR T Cells are Highly Active and Potent Against Hodgkin Lymphoma

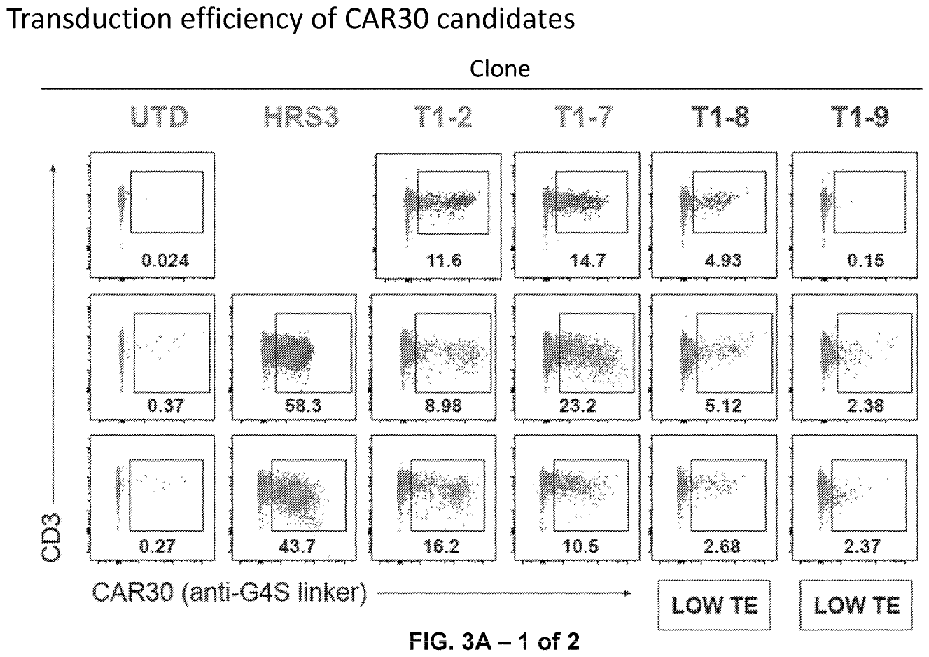

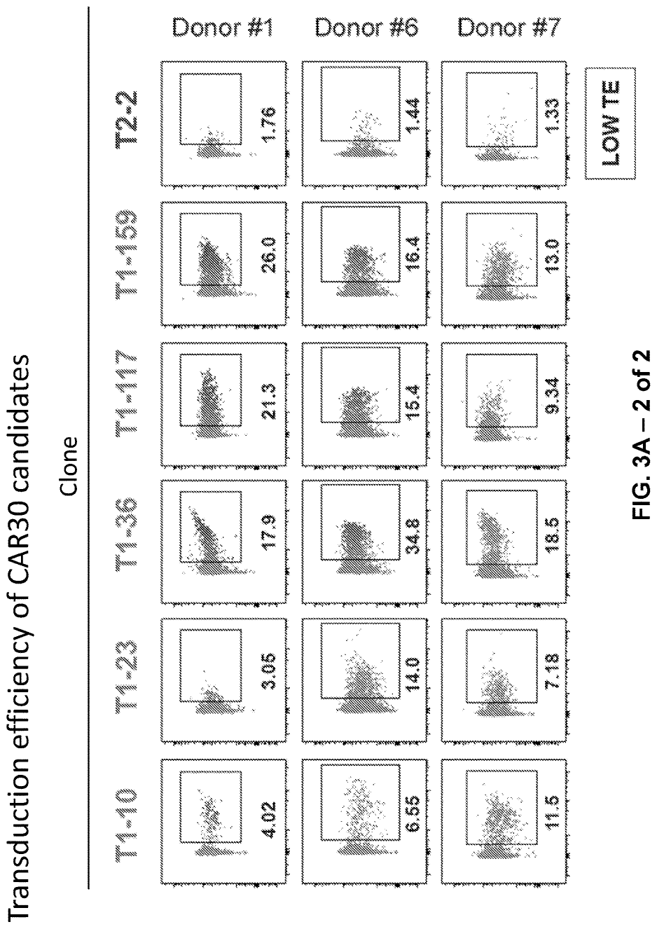

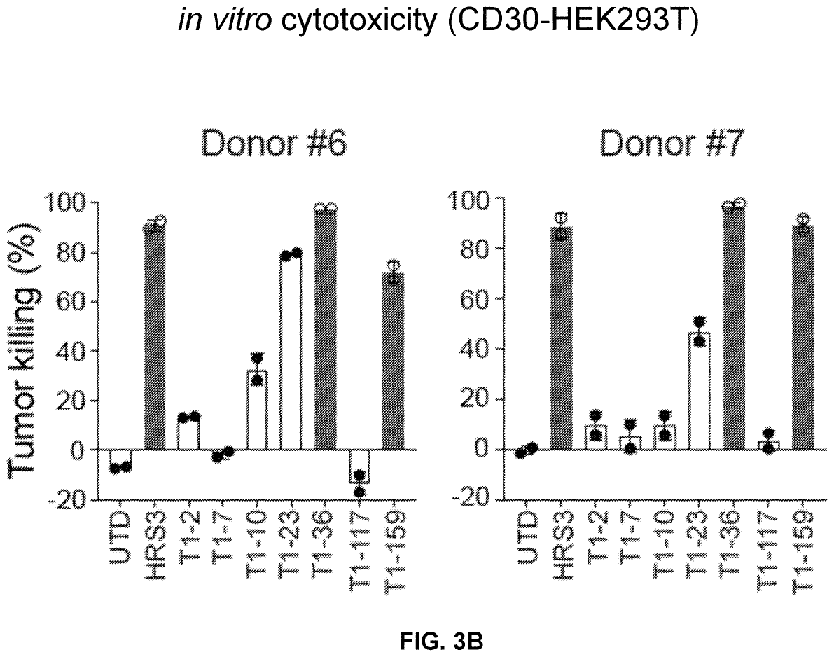

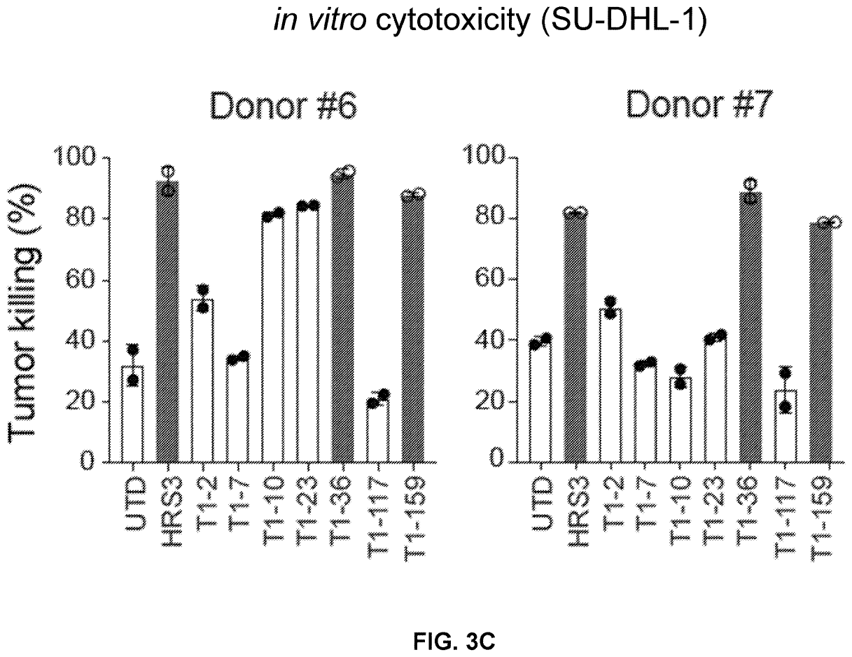

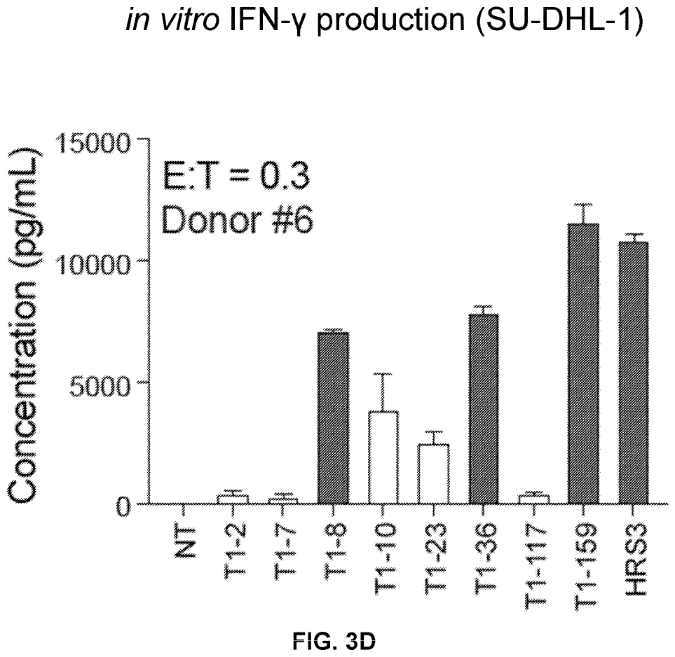

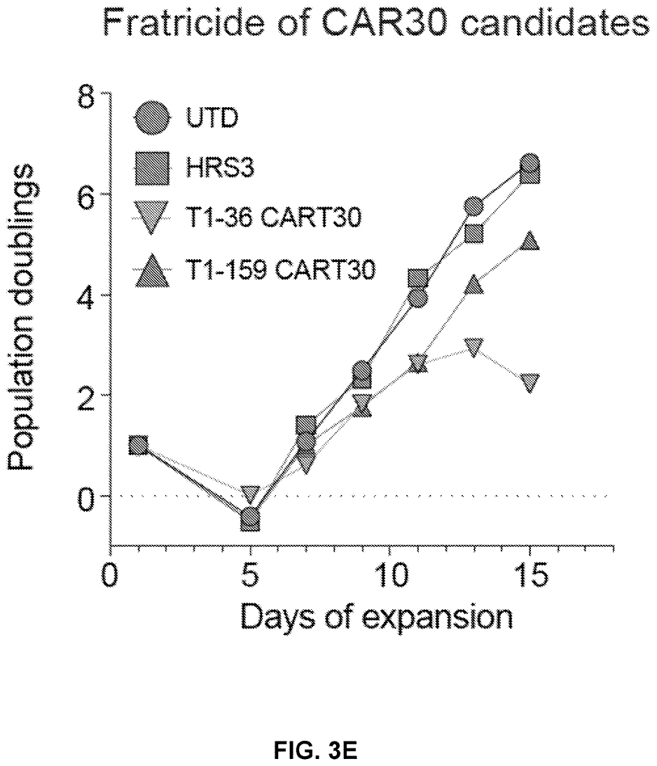

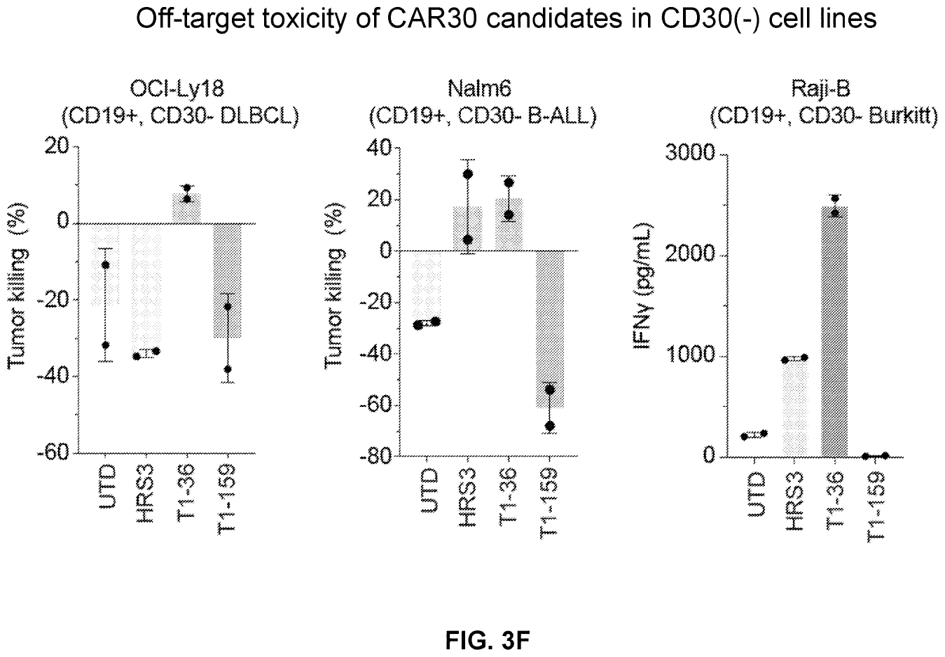

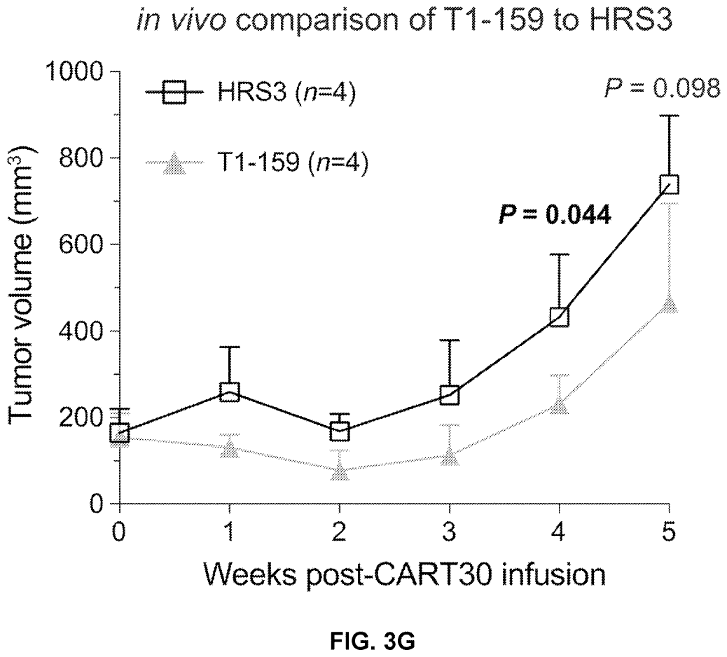

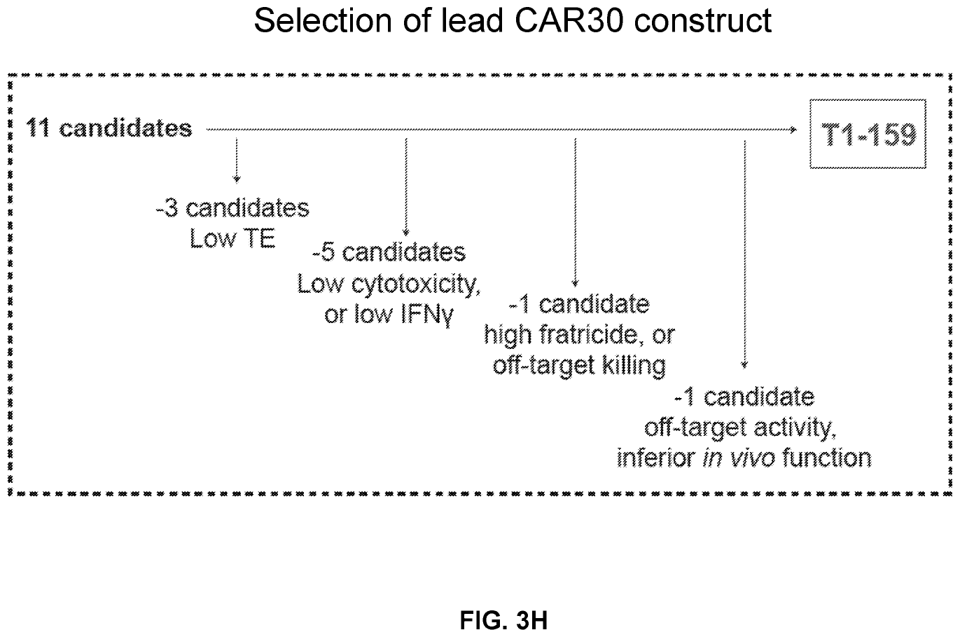

Resolutive treatments for relapsed/refractory Hodgkin lymphoma represent a dire unmet clinical need. To address this, CAR T cells directed against CD30 on malignant Hodgkin and Reed Sternberg cells in HL (Di Stasi, et al., Blood 113, 6392-6402 (2009)) have recently entered clinical trials, but responses are extremely short-lived and beget poor progression-free survival (Ramos, et al., J Clin Oncol 38, 3794-3804 (2020); Brudno, et al., Blood Advances 8, 802-814 (2024)). The immunosuppressive TME in HL plays a major role in T cell functions through a plethora of mechanisms, including the increased abundance of soluble factors (e.g., 11-10, TGF-β), physical barriers (e.g., fibronectin), and inhibitory ligand-receptor interactions. Engineering CAR T cells to circumvent these suppressive TME interactions may provide substantial benefit in HL patients. Having described an enrichment of the BTLA-HVEM interaction in the HL TME, it is contemplated herein that genetic deletion of BTLA in CD30-directed CAR (CAR30) T cells would enhance their function in HL by abolishing trans interactions with HVEM on immunosuppressive cells (and possibly interactions in cis interactions as well). In order to develop a best-in-class gene-edited CART30, 11 anti-CD30 CAR scFv binders were screened. The top CAR30 construct candidates were selected based on several factors: transduction efficiency (>10%), higher cytolytic capacity and cytokine secretion against CD30+ cell lines, minimal susceptibility to fratricide, low off-target toxicity, and improved in vivo functionality over the clinically-employed HRS-3 scFv (Ramos, et al., J Clin Oncol 38, 3794-3804 (2020)) clone (FIGS. 3A-3H). This extensive screening led to the choice of the lead CAR30 scFv candidate, T1-159. Therefore, T1-159, from now on simply called CART30 herein, was used for all the subsequent in vitro and in vivo studies.

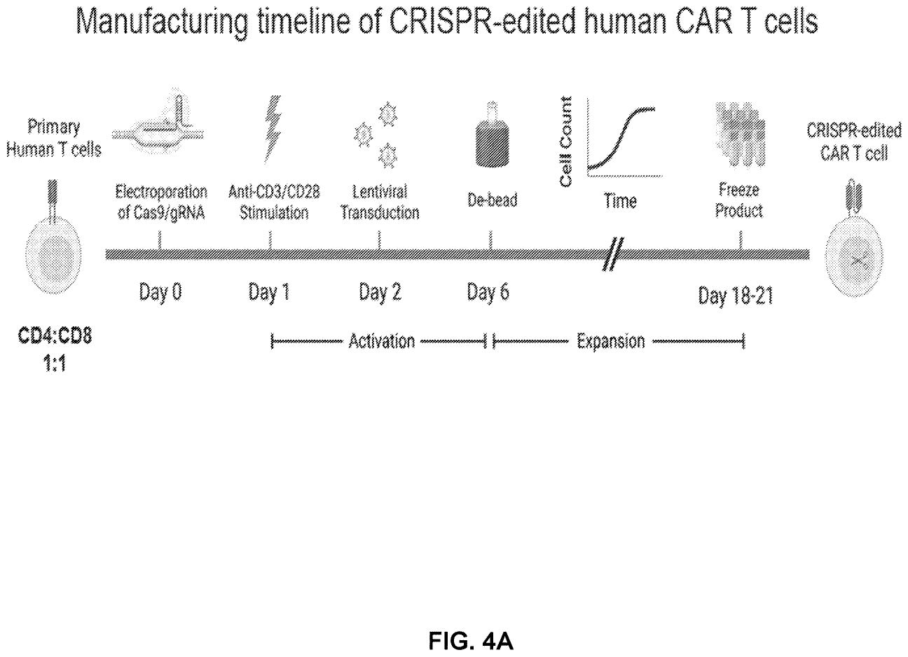

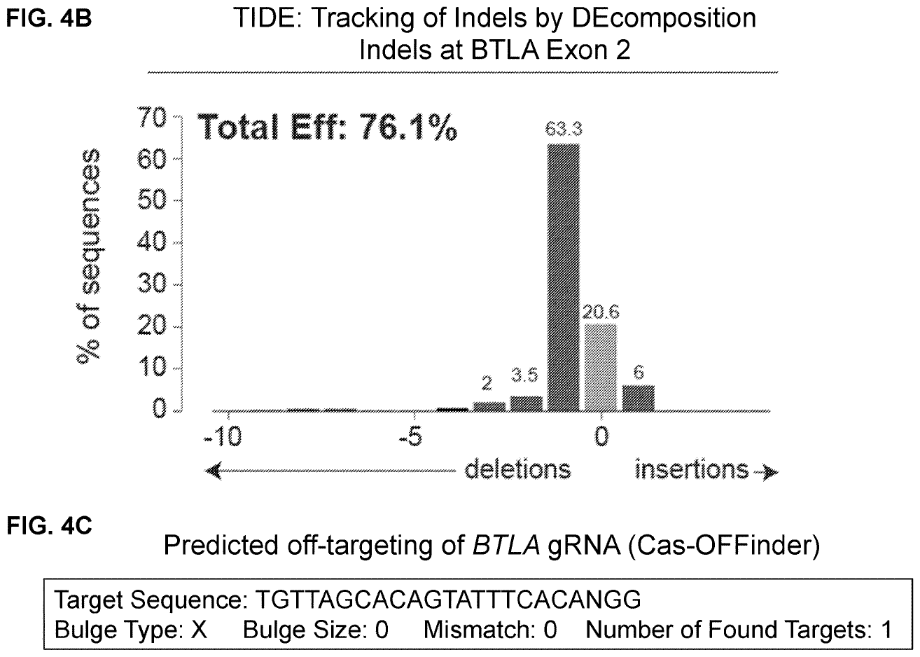

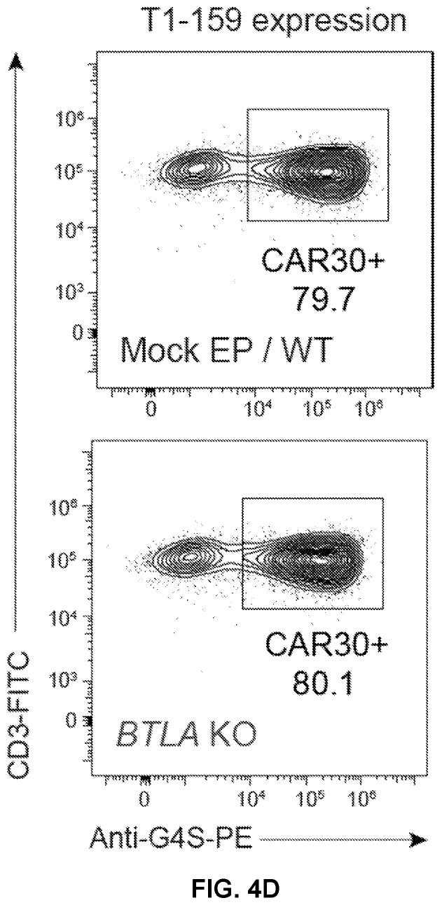

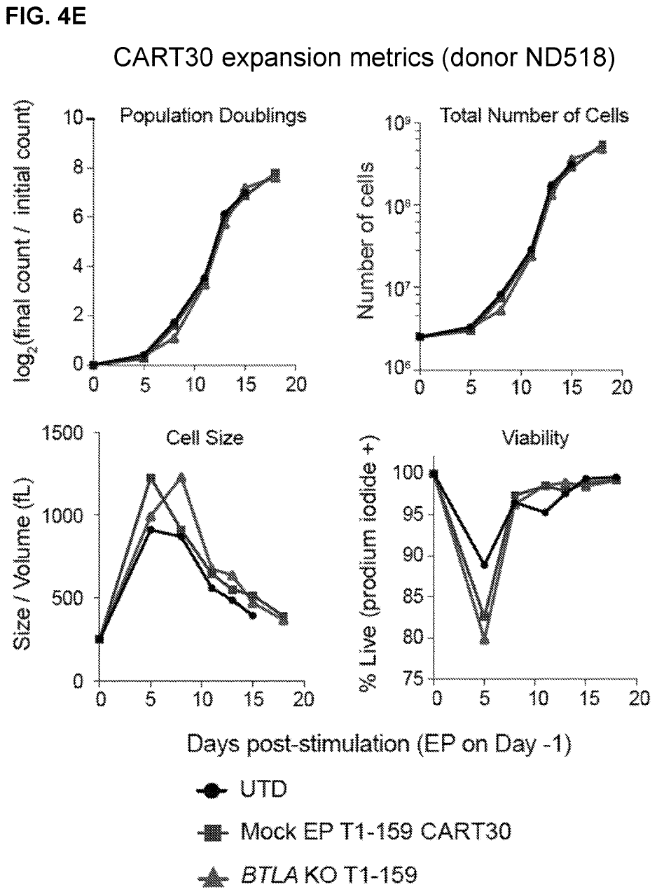

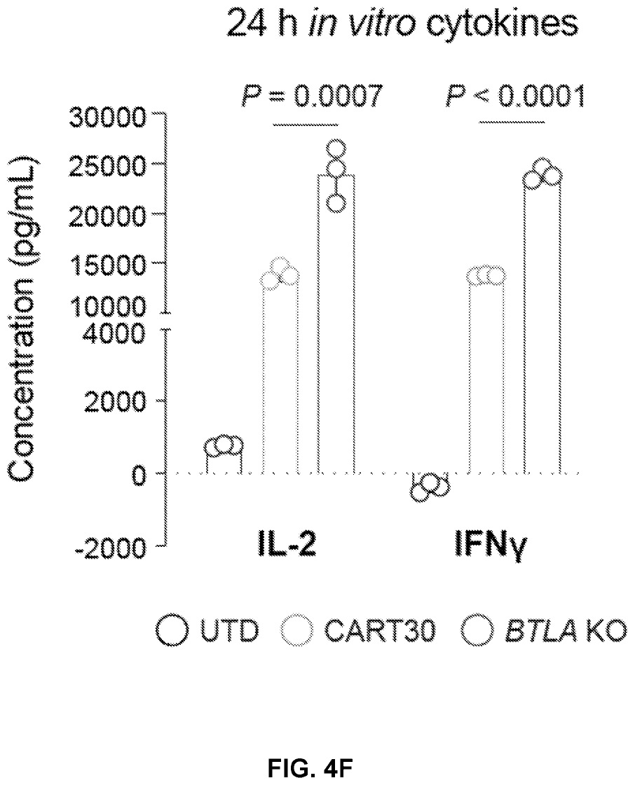

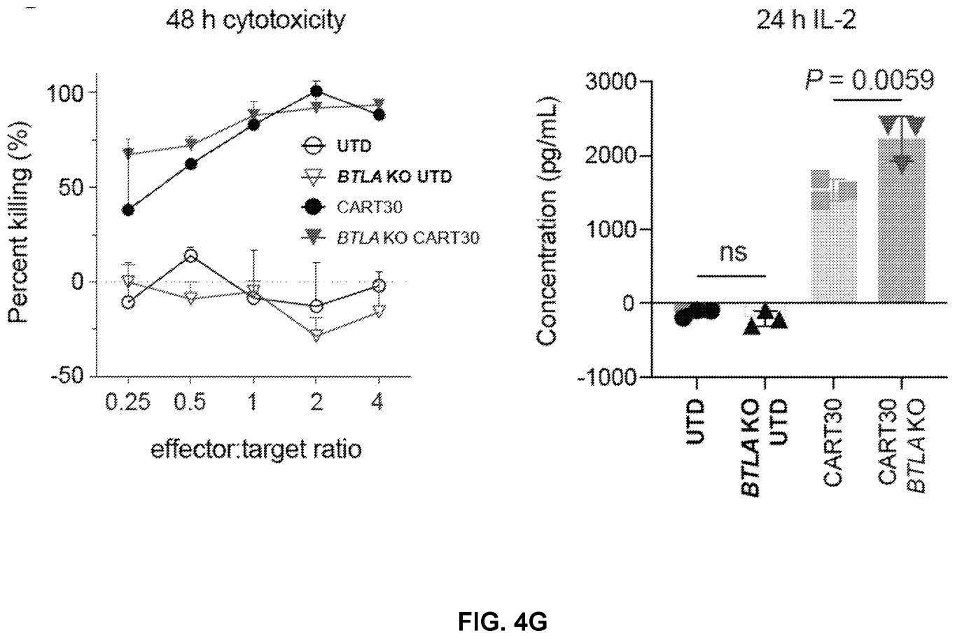

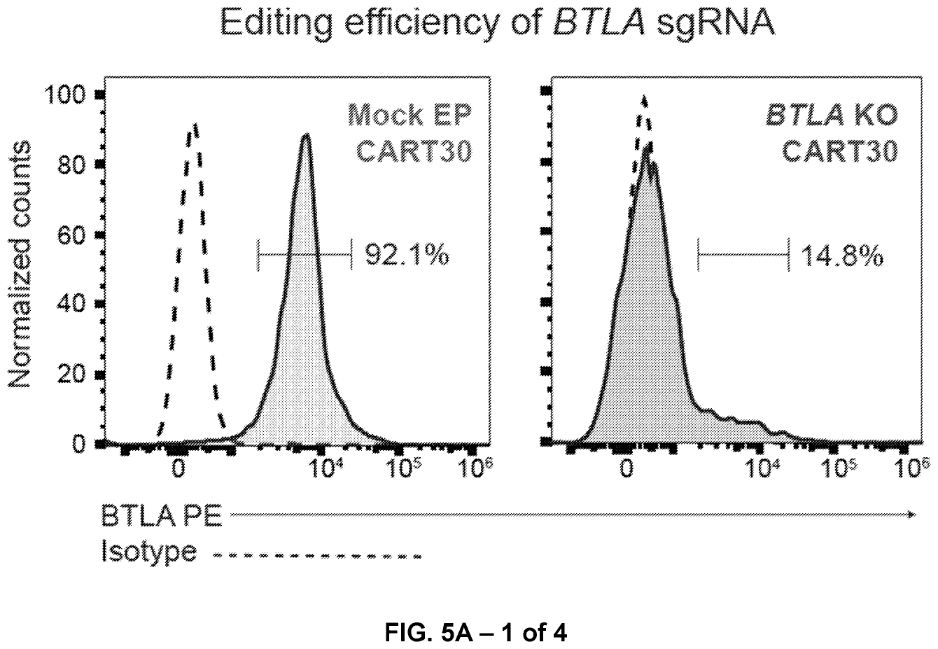

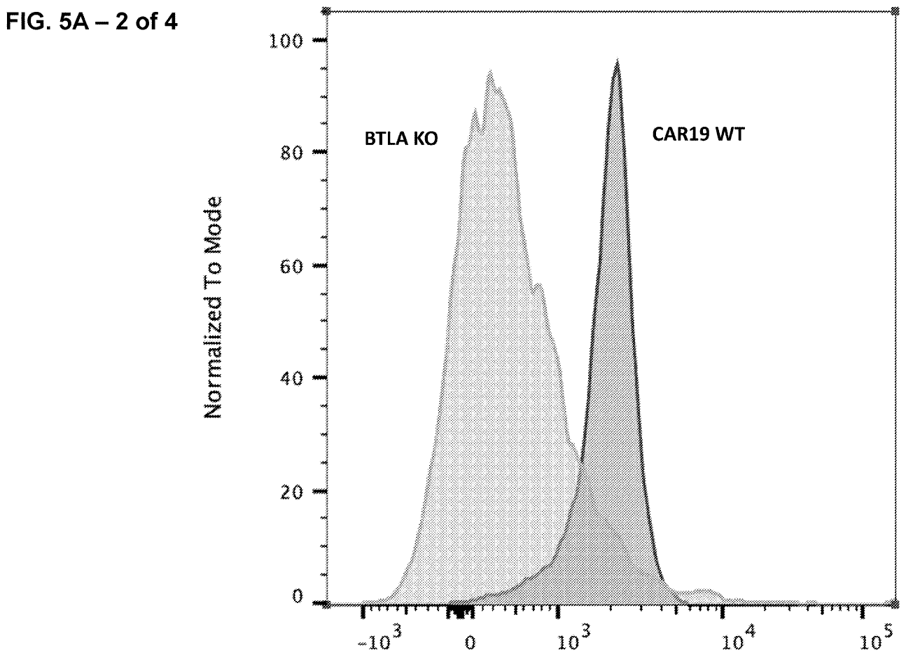

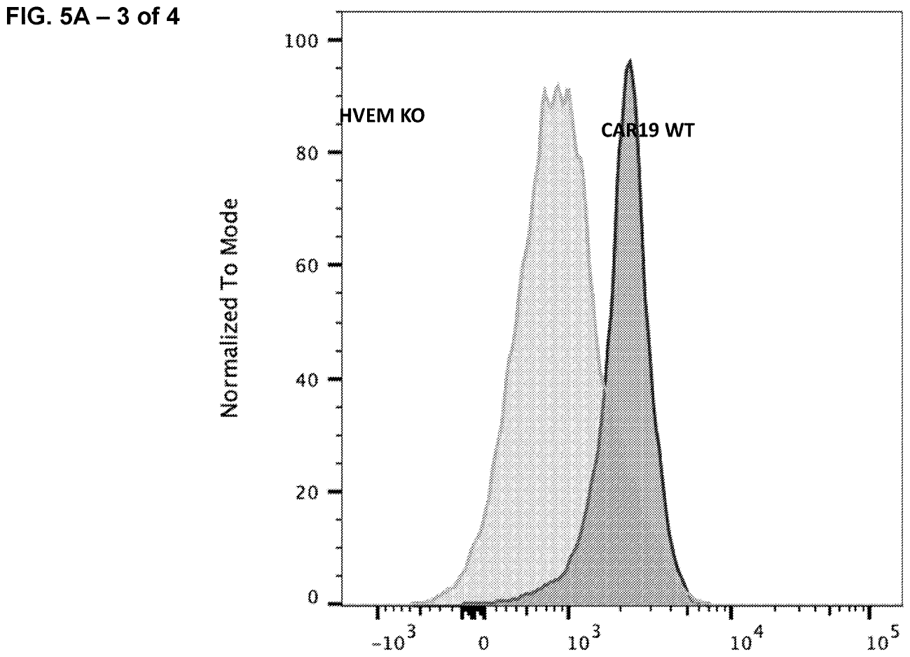

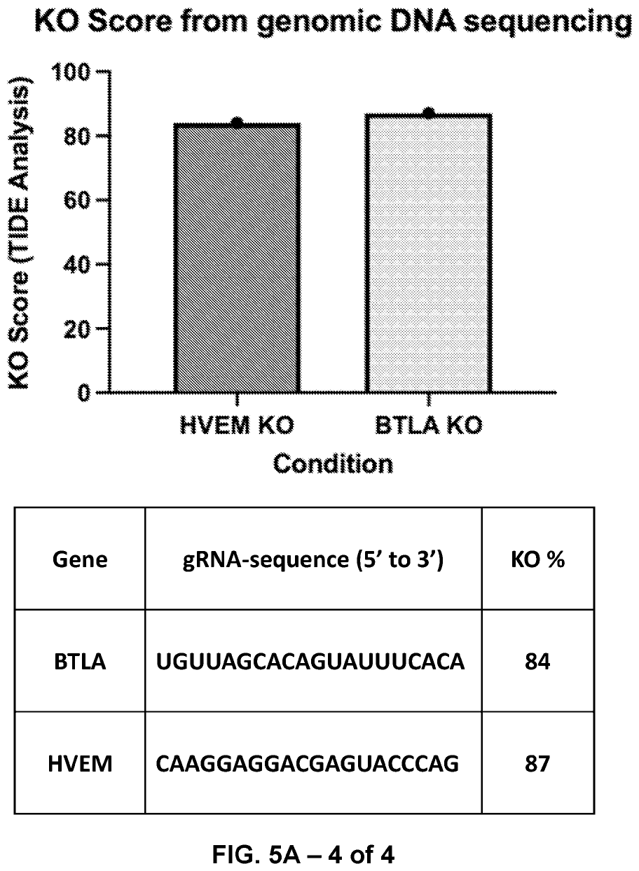

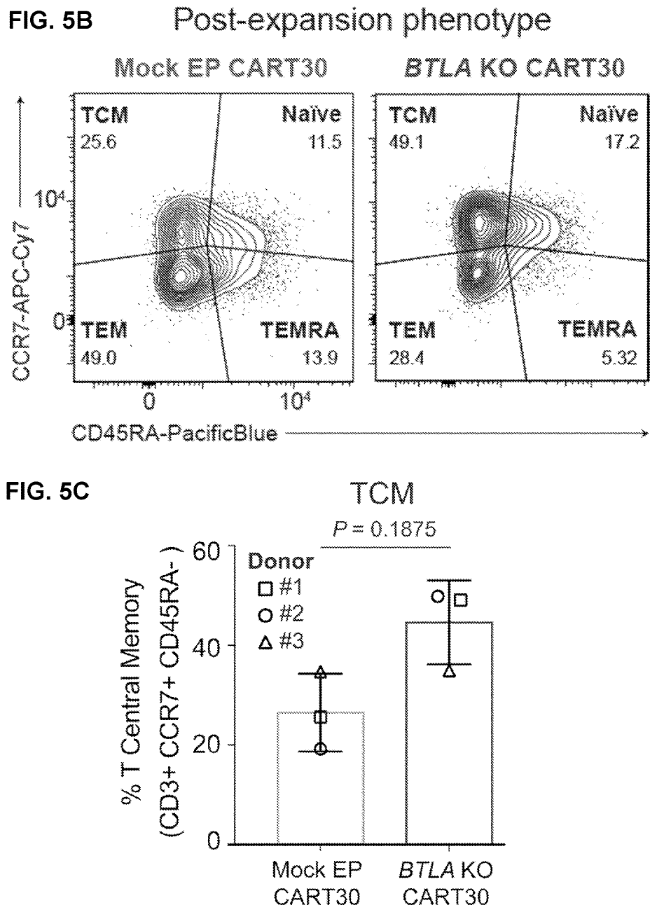

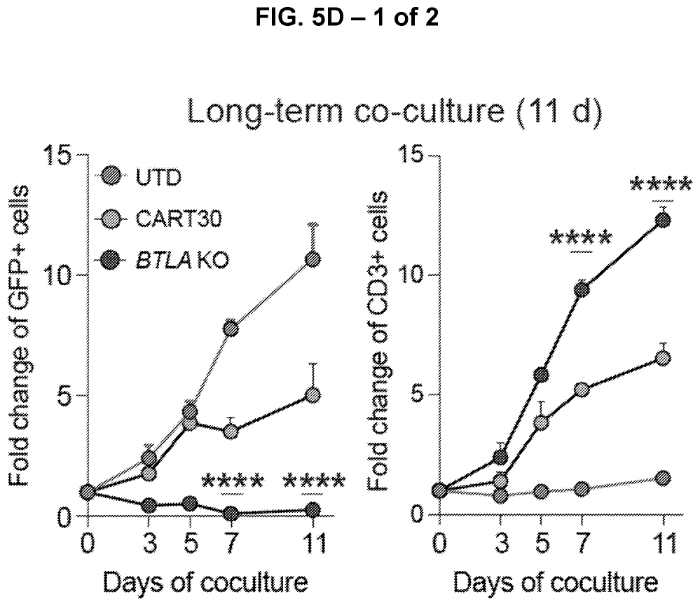

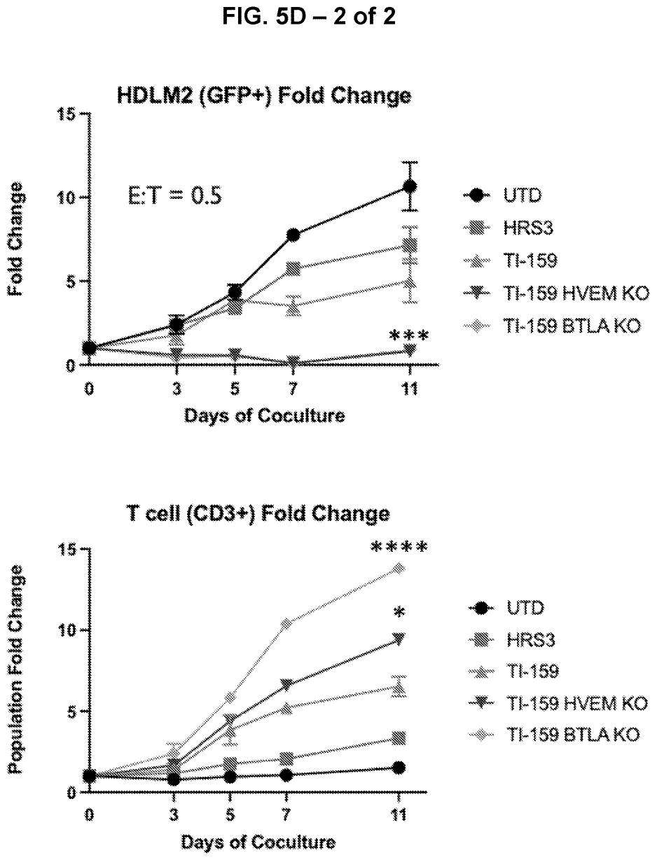

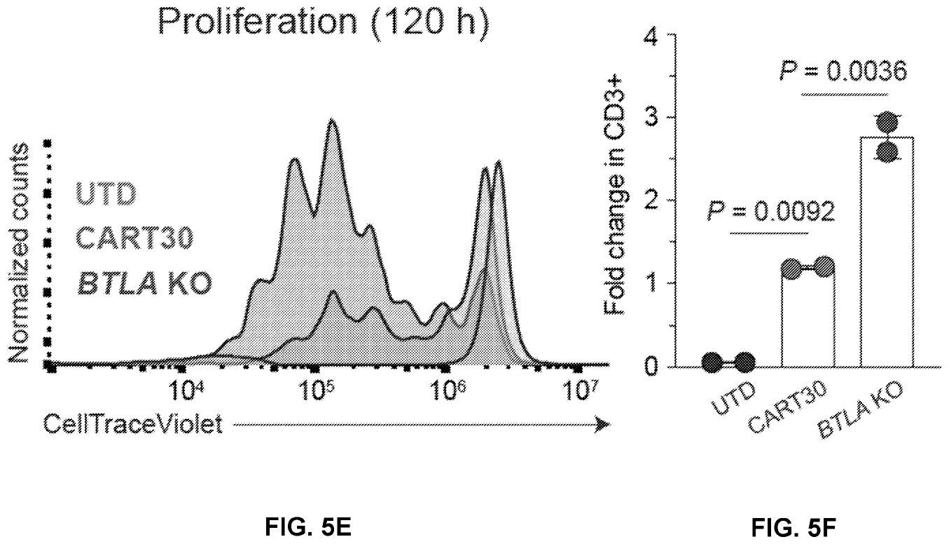

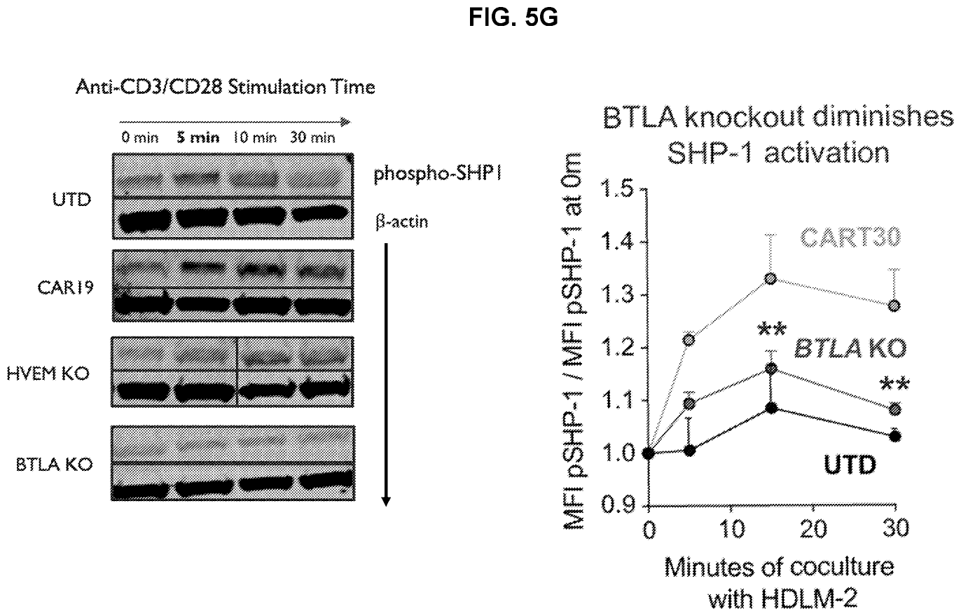

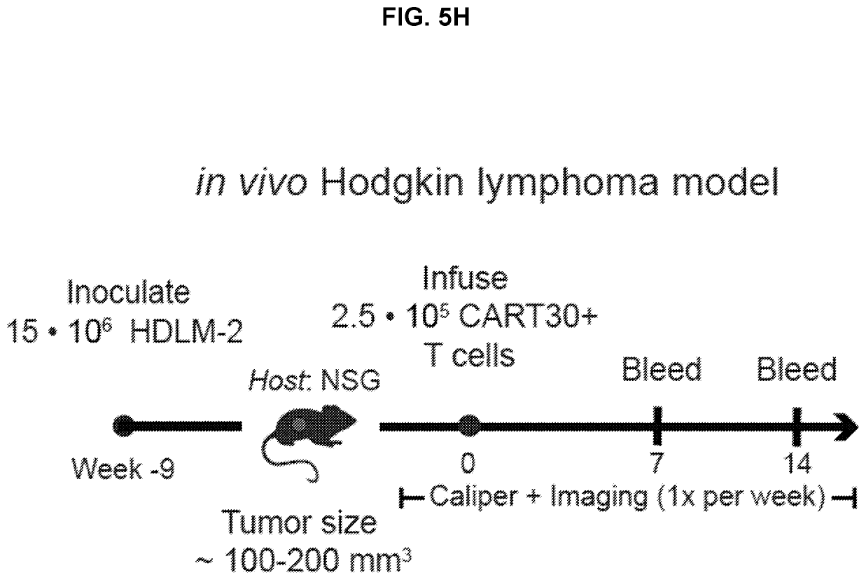

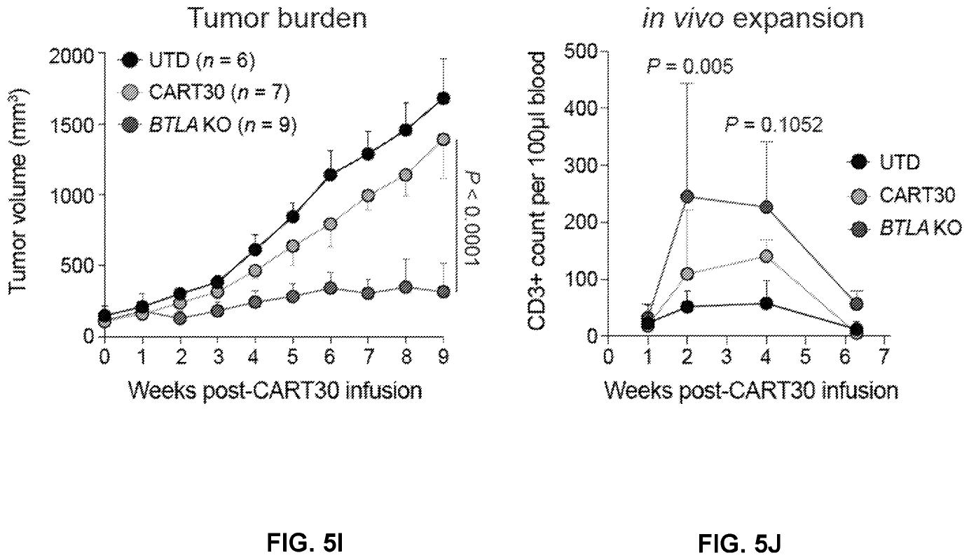

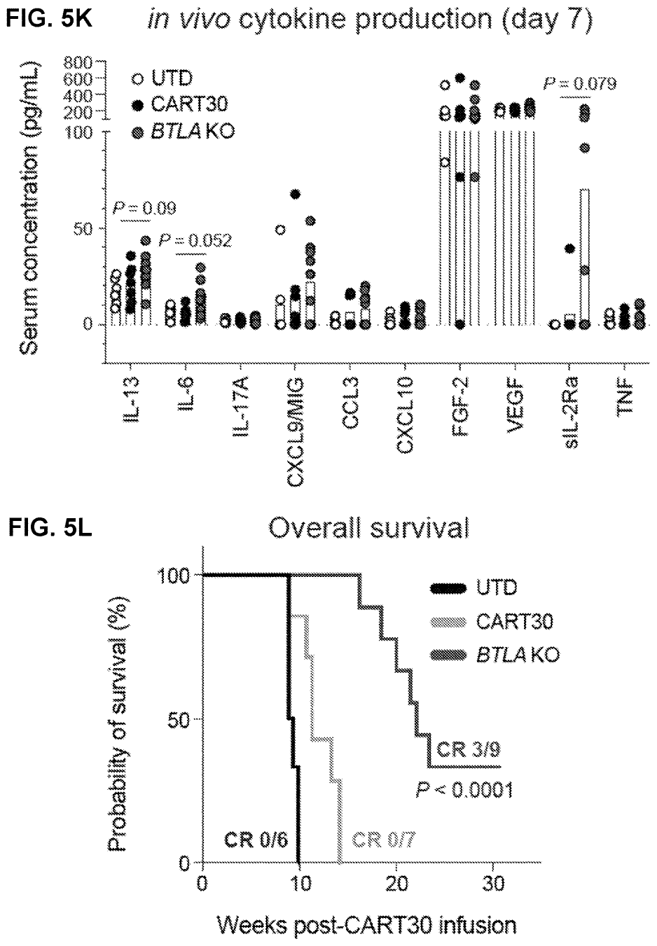

Next, a platform for CRISPR-Cas9 knockout of BTLA or HVEM in CART30 was developed. This protocol involved electroporation of a BTLA-specific guide-RNA (or an HVEM-specific guide-RNA) and Cas9 complex into primary human T cells, followed by lentiviral transduction of the T1-159 CAR30 construct. (FIG. 4A). This manufacturing protocol was capable of generating highly transduced (>80%) CAR30+ T cells with high BTLA or HVEM knockout (>75% KO), as confirmed by flow cytometry and analysis of genomic DNA using TIDE analysis (FIGS. 4B-4E and FIG. 5A). At the end of CAR T cell manufacturing, BTLA KO CART30 cells included a higher proportion of T central memory cells relative to mock-electroporated (WT) CART30 cells (FIGS. 5B-5C). This finding may be indicative of less differentiation during CAR T cell manufacturing.

Next, the effects of BTLA KO or HVEM KO in CAR30 T cells on in vitro cytotoxicity and proliferation of the T cells were determined. The results show that BTLA KO CAR30 T cells and HVEM KO CAR30 T cells each have significantly improved tumor control in vitro (FIG. 5D). Additionally, HVEM KO CAR30 T cells and, in particular, BTLA KO CAR30 T cells, each have significantly improved expansion in vitro (FIG. 5D). To further test the cytotoxic capacity of BTLA knockout in CART30 cells in vitro, control untransduced (UTD) T cells, WT CART30, and BTLA KO CART30 were cocultured with HDLM-2, a CD30+HL line that bears constitutive expression of HVEM (Wein, et al., Cancer Immunology Research 5, 1122-1132 (2017)). At day 11 of coculture, BTLA KO CART30 cells maintained significantly increased control of HDLM-2 growth compared to controls. Moreover, BTLA KO CART30 proliferated approximately two-fold more than WT CART30 (FIG. 5D). Consistent with these findings, BTLA KO CART30 cells experienced a higher number of proliferation cycles when challenged with radiation-sensitized HDLM-2 for 120 hr (FIG. 5E and FIG. 5F). Additionally, BTLA knockout enhanced effector cytokine secretion of CART30 in vitro (FIG. 4F). To begin to understand the mechanism behind these observations, it was found that BTLA KO CART30 demonstrated dampened phosphorylation of SHP-1 to the immune synapse and increased avidity to tumor cells upon coculture with HDLM-2 (FIG. 5G). This result helps to substantiate the known, potent role of SHIP-1 in propagating the inhibition by BTLA in T cells (Murphy, et al., Nature Reviews Immunology 6, 671-681 (2006); Xu, et al., J Cell Biol 219 (2020)). Of importance, BTLA KO in UTD did not confer any non-specific or non-CAR mediated cytotoxic capacity relative to mock-electroporated UTD T cells, suggesting that the enhanced activity of BTLA KO in CART30 is mediated by the formation of the CAR-antigen synapse (FIG. 4G).

Next, to validate these observations in vivo, NSG (NOD scid gamma) mice were subcutaneously inoculated with HDLM-2 and then randomized to receive either UTD, WT CART30, or BTLA KO CART30 upon tumor engraftment (FIG. 5H). At 9 weeks post-infusion of CART30, mice treated with BTLA KO CART30 had dramatically improved tumor control relative to those treated with WT CART30 cells measured by tumor volume (FIG. 5I). Moreover, in line with the in vitro results, BTLA KO CART30 caused an increase in CAR T expansion in the peripheral blood over WT CART30 (FIG. 5J). In order to better study the function of BTLA KO CAR T cells, cytokine profiling of mouse serum was performed at 7 days post-CART30 infusion. Elevated levels of pro-inflammatory markers were observed in the serum of BTLA KO CART30 treated mice (e.g. IL-13, IL-6, sIL2Rα), suggesting higher inflammatory CAR T cell activity shortly after infusion (FIG. 5K). This enhanced tumor control, peripheral expansion, and cytokine production in BTLA KO CART30 treated mice was reflected by a significantly improved overall survival (FIG. 5L). In summary, these data demonstrate that deletion of BTLA in anti-CD30 CAR T cells significantly improve their anti-tumor function in HL.

### BTLA Ablation Enhances CART19 Function Against Non-Hodgkin Lymphoma

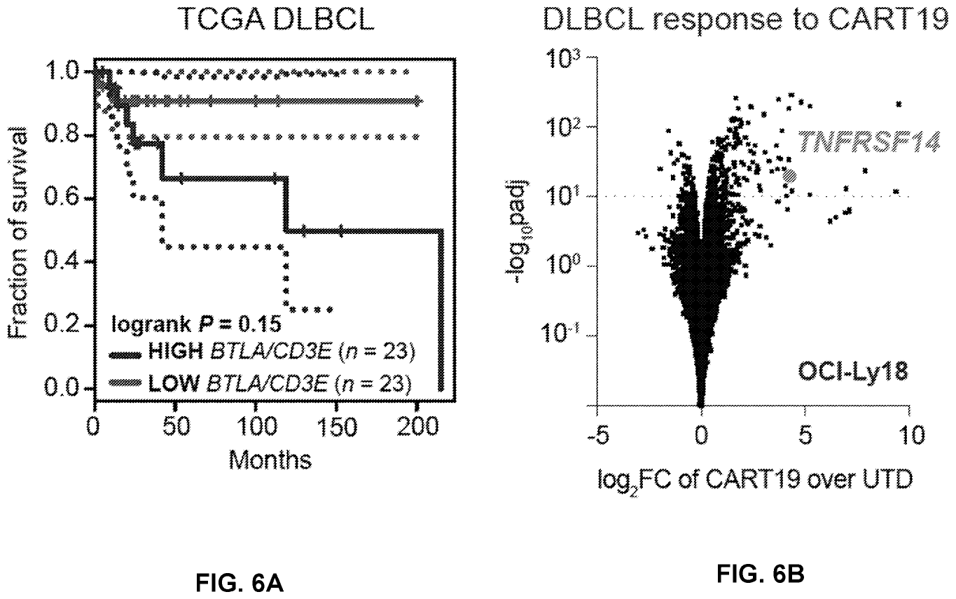

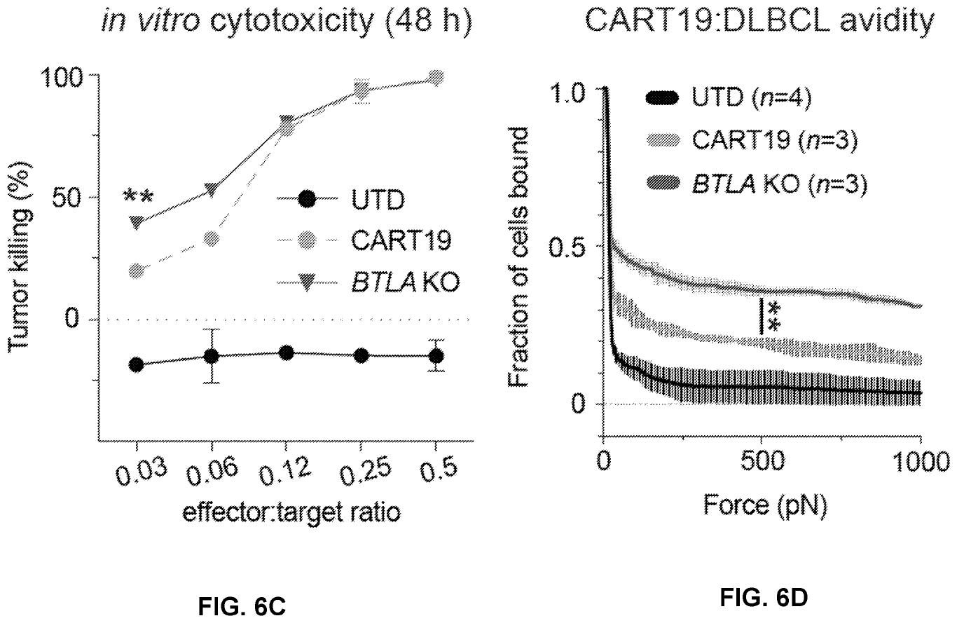

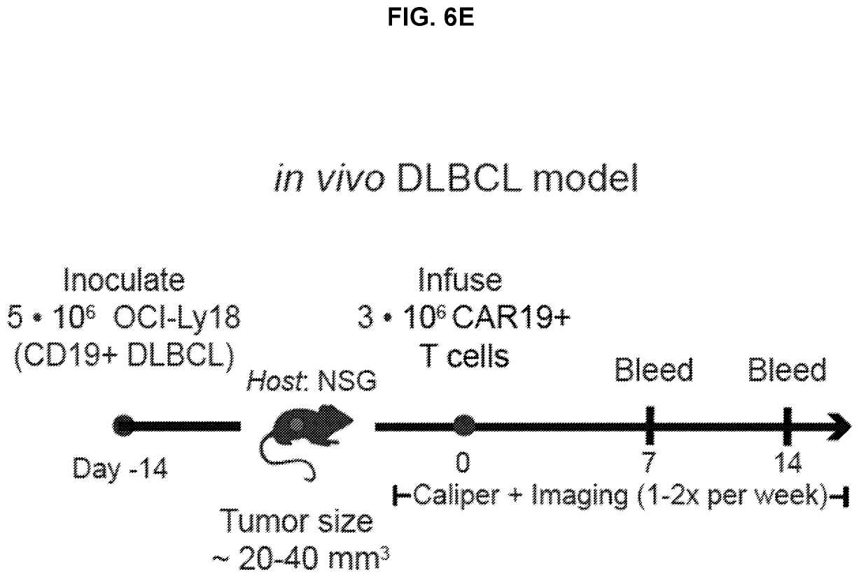

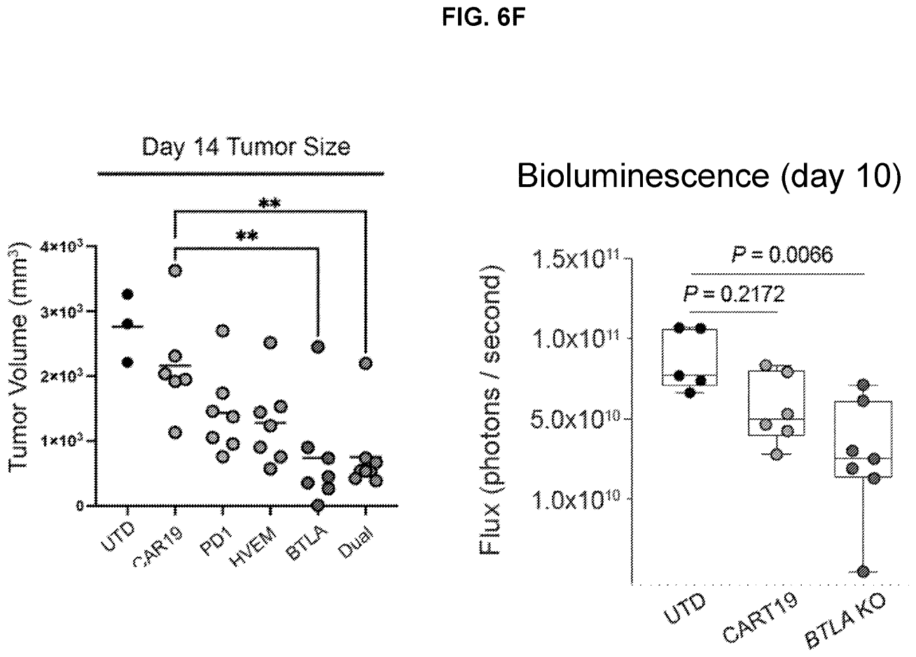

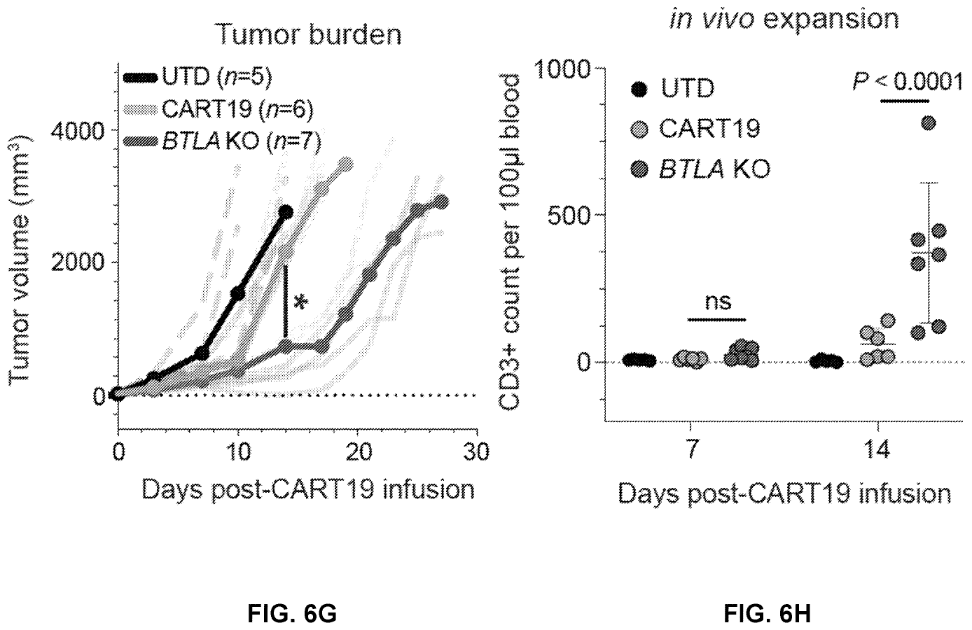

Because of the promising results in HL, whether the BTLA-HVEM pathway had a role in non-Hodgkin lymphomas (NHL) was evaluated. Four anti-CD19 CAR T cell products (CART19) are FDA-approved for NHL (Cappell, et al., Nature Reviews Clinical Oncology 20, 359-371 (2023)). Recent literature suggests that the dysregulation of TNFRSF14/HVEM in NHL contributes to tumorigenesis and progression (Pasqualucci, et al., Blood 131, 2307-2319 (2018); Chapuy, et al., Nat Med 24, 679-690 (2018); Mintz, et al., Immunity 51, 310-323.e317 (2019)). Moreover, upregulation of BTLA in circulating T cells has been suggested to be associated with reduced T cell cytotoxicity and disease progression in hematological malignancies like diffuse large B cell lymphoma (DLBCL) and chronic lymphocytic leukemia (CLL) (Quan, et al., Exp Hematol 60, 47-56.e41 (2018); Sordo-Bahamonde, et al., Cancer Immunology, Immunotherapy 72, 2529-2539 (2023)). Indeed, the retrospective analysis of TCGA disclosed herein demonstrated that high BTLA expression normalized to T cell infiltration (ratio of BTLA/CD3E) correlates with poor outcome in DLBCL (logrank P=0.15) (FIG. 6A).

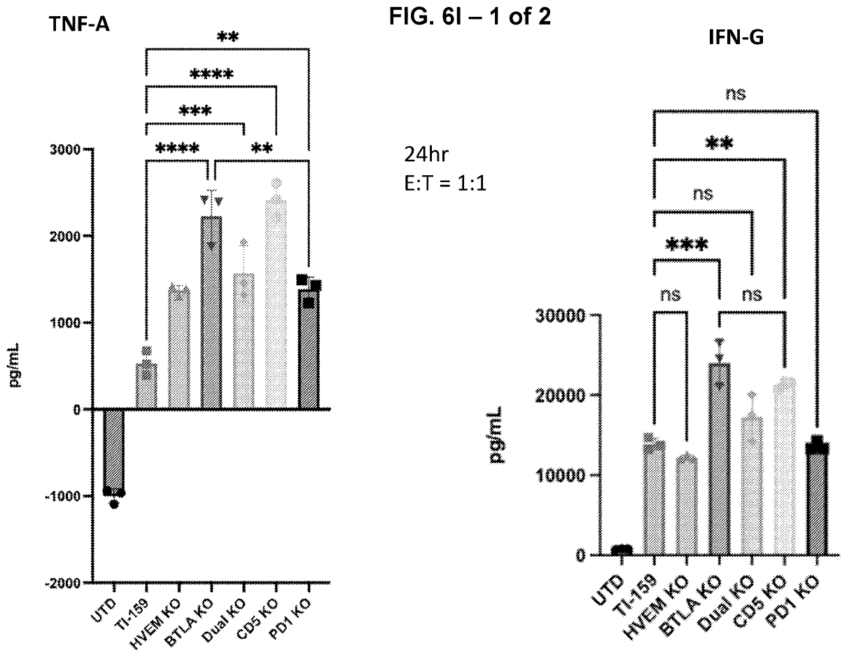

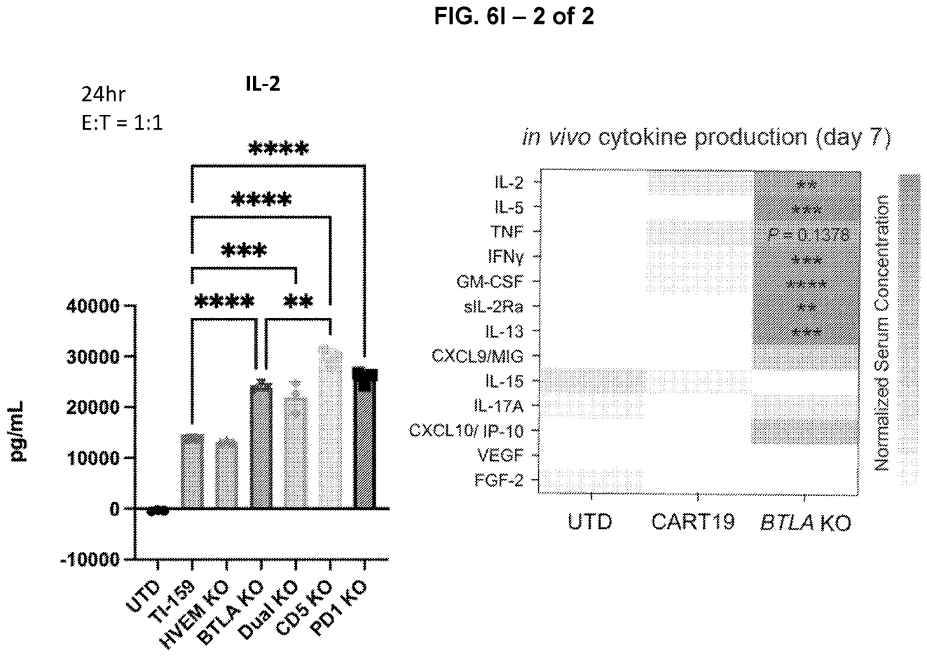

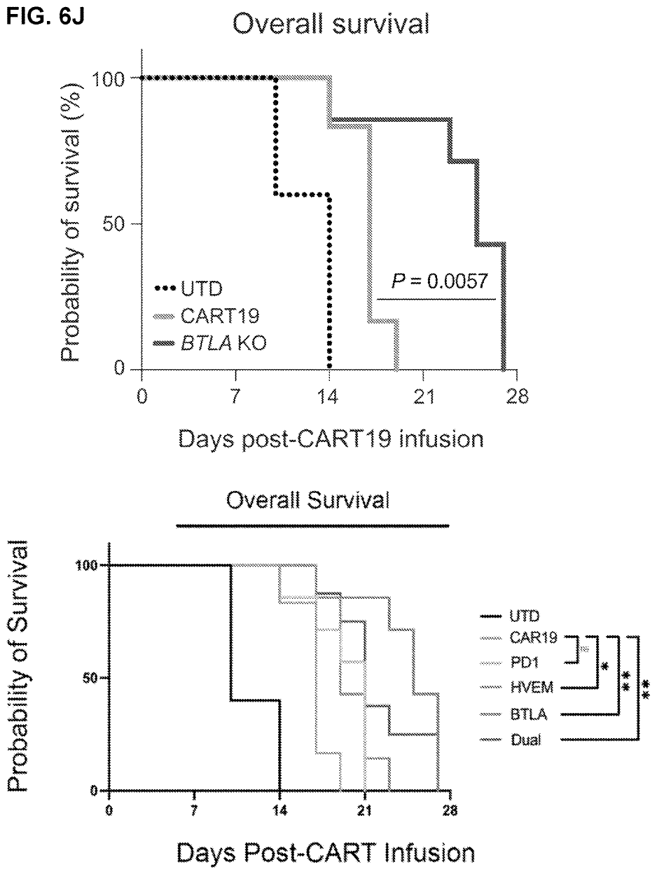

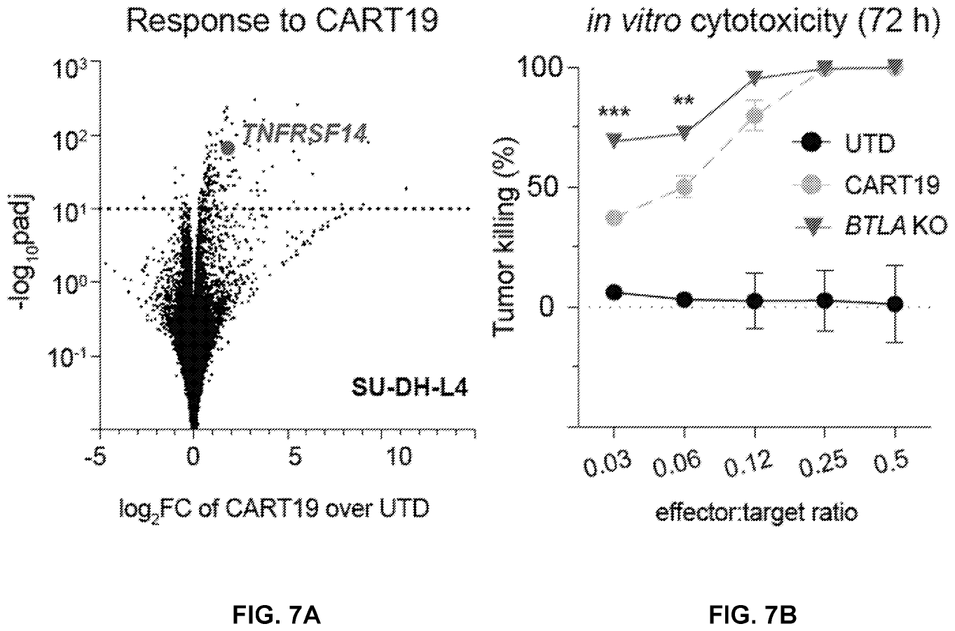

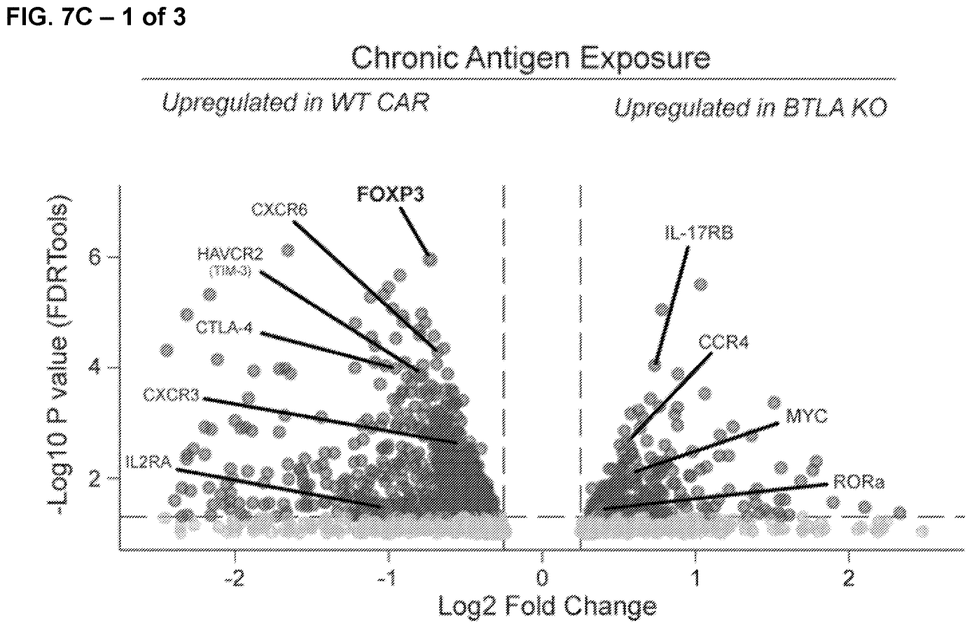

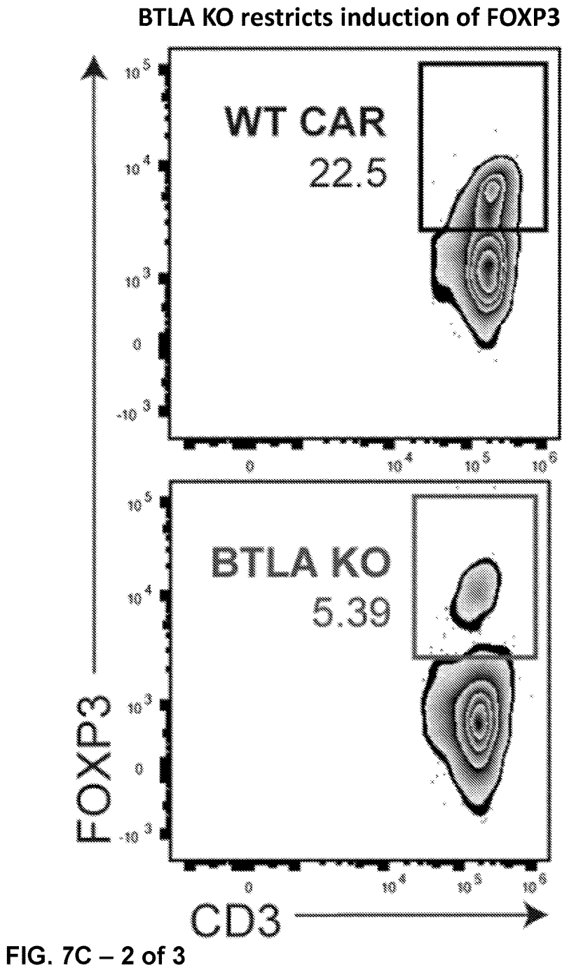

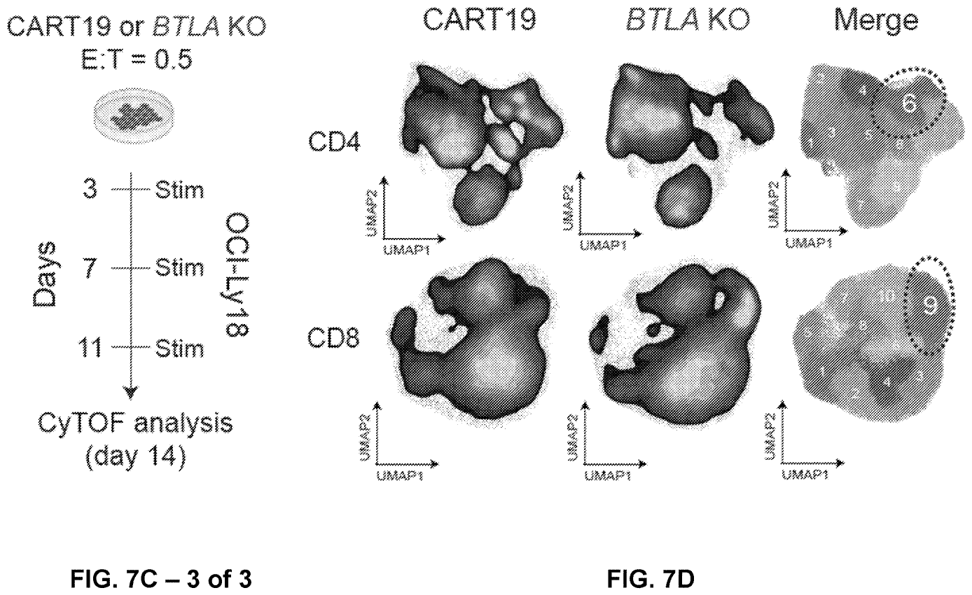

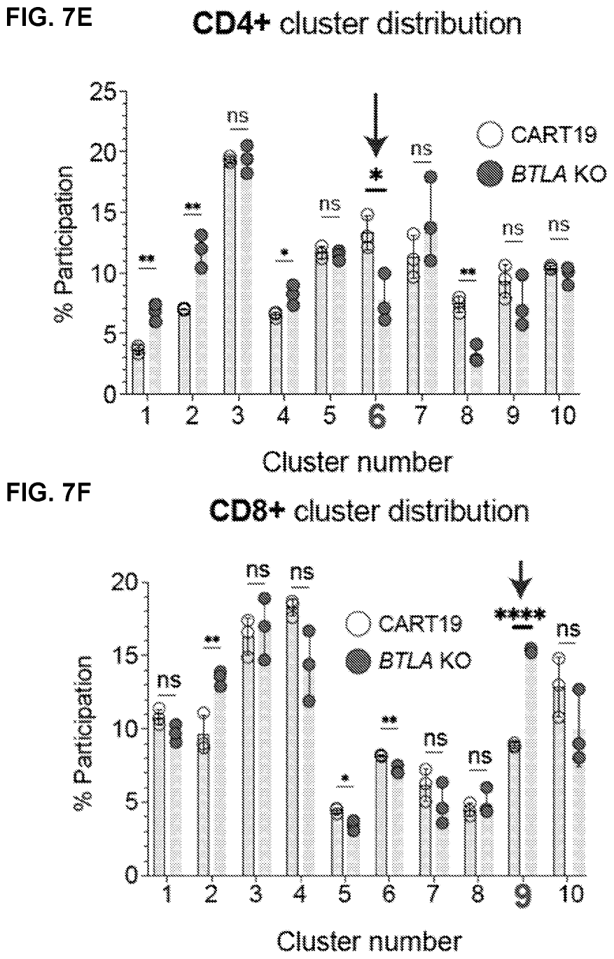

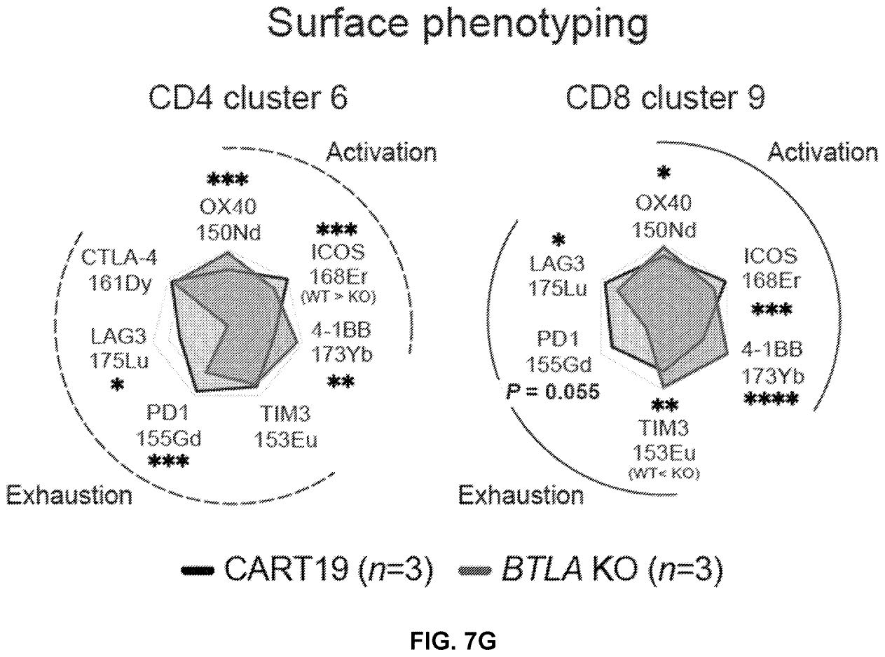

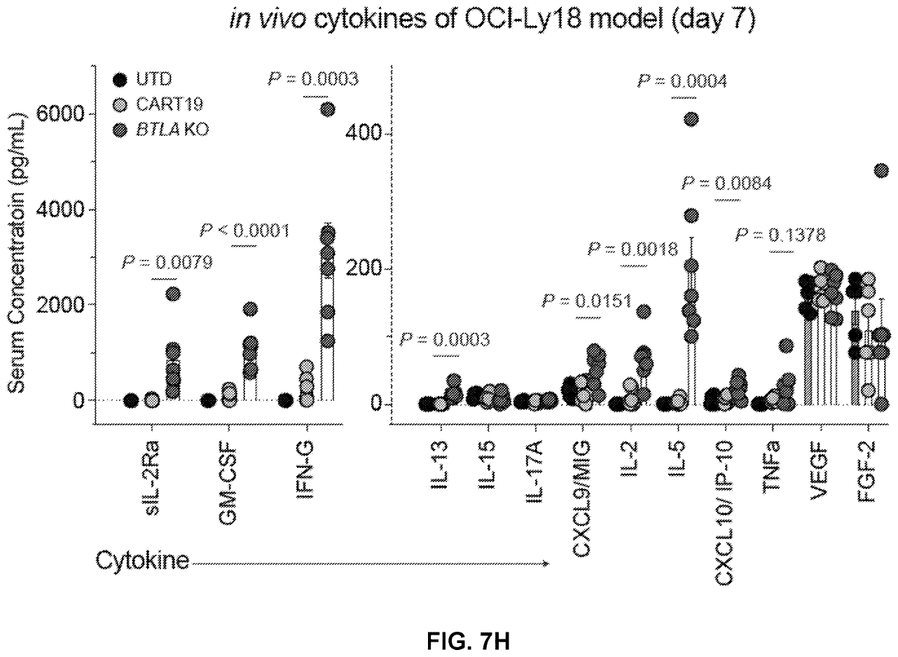

As models of NHL, the OCI-Ly18 and SU-DH-L4 DLBCL cell lines were utilized. Interestingly, it was first observed that coculture with CART19 cells induces significant expression of HVEM in both of these models (FIG. 6B, FIG. 7A), and so it was hypothesized that HVEM upregulation may be an escape mechanism in some DLBCLs by inhibiting CART19 through BTLA. Thus, BTLA-deficient CART19 were generated using the aforementioned manufacturing schema and lentiviral transduction of the 4-1BB-ζ CAR19 transgene (Milone, et al., Mol Ther 17, 1453-1464 (2009); Kalos, et al., Science Translational Medicine 3, 95ra73-95ra73 (2011)). In short-term 48-hour coculture with OCI-Ly18 and SU-DH-L4, BTLA KO CART19 led to improved tumor cell killing over WT CART19 at low effector: target ratios (FIG. 6C, FIG. 7B). BTLA-deficient CAR T cells also showed higher avidity for OCI-Ly18 over WT CAR T cells, suggesting that BTLA may be a negative regulator of CAR-antigen synapse strength (FIG. 6D).

Recent reports have suggested that the HVEM-BTLA axis may induce conversion of naïve T cells into immune-suppressive FOXP3+ Treg (Jones, A. et al., Immunity 45, 1066-1077 (2016); Bourque, J. & Hawiger, D., Front Immunol 10, 1163 (2019)). To study the relationship of BTLA and FOXP3, a chronic stimulation experiment was performed in which WT CAR T and BTLA KO CAR T cells were co-cultured with HVEM-high OCI-Ly18 tumor cells (E;T=0.25) for two weeks. On days 3, 7, 10, and 12, CAR T cells were reseeded with tumor cells (E:T=0.25). On day 15, T cells from each condition were purified, and bulk RNA sequencing was performed. At the end of two weeks of chronic antigen stimulation, BTLA KO CAR T cells had reduced RNA expression of FOXP3 and exhaustion markers (e.g., CTLA-4 and HAVCR2) (FIG. 7C). That BTLA KO prevented protein expression of FOXP3 in CAR T cells upon extended co-culture with HVEM-high tumor cells was confirmed by assessing FOXP3 expression in WT CAR30 and BTLA KO CAR30 T cells following 5 days of co-culture with HDLM-2 (E:T=0.5) (FIG. 7C). These data support the role of BTLA KO in preventing CAR T cell conversion into FOXP3+ CAR T cells. To further assess resistance to T cell exhaustion, the WT and BTLA KO CART19 were subjected to multiple rounds of exposure to OCI-Ly18 in vitro for 14 days and the surface phenotype of the CAR T cells was then measured via time-of-flight mass cytometry (CyTOF) (FIG. 7C). The results show that BTLA-deficient CART19 exhibited stronger expression of activation markers (OX40, 4-1BB), whereas WT CART19 cells projected more towards those of exhaustion (PD-1, LAG-3) (FIGS. 7D-7G) indicating that BTLA ablation improves the endurance of CAR T cells.

To extend these findings in vivo, subcutaneous tumors of OCI-Ly18 were established into NSG mice, and the mice were randomized to be infused with UTD, WT CART19, or BTLA KO CART19 (FIG. 6E). Mice injected with BTLA KO CAR19 T cells or BTLA/HVEM dual KO CAR19 T cells had significantly reduced tumor size. (FIG. 6F). Bioluminescent imaging of mice taken on day 10 post-CART19 infusion showed decreased tumor burden in mice that received BTLA KO CART19 (FIG. 6F). Notably, a delay of tumor growth was observed in BTLA KO CART19 treated mice (FIG. 6G) (P=0.015 between BTLA KO and WT CART19 on day 14). Correspondingly, BTLA KO augmented in vivo expansion and effector cytokine secretion (e.g., IL-2, TNF, IFN-γ) (FIG. 6H, FIG. 6I, FIG. 7H); ultimately these mice experienced improved overall survival over WT CART19 (FIG. 6J).

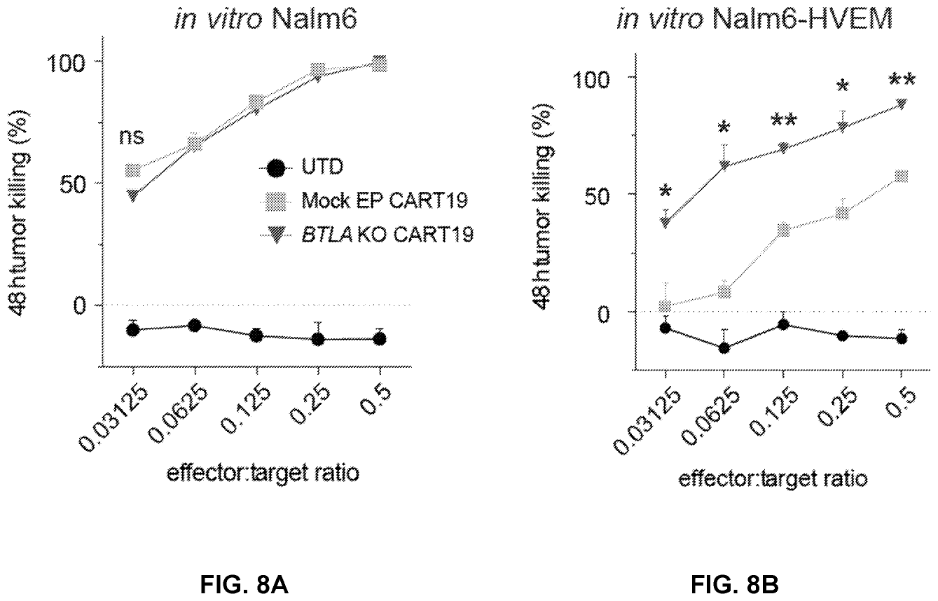

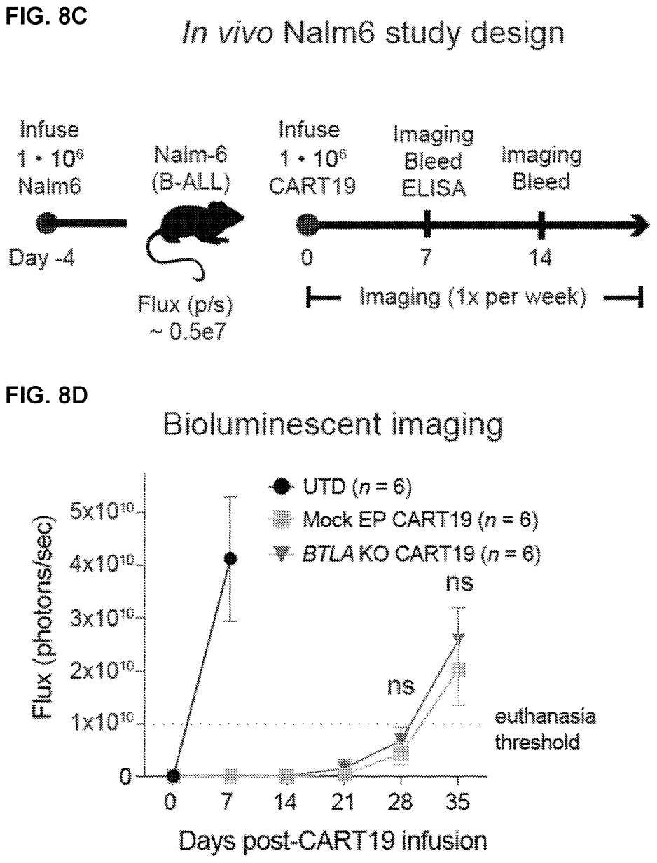

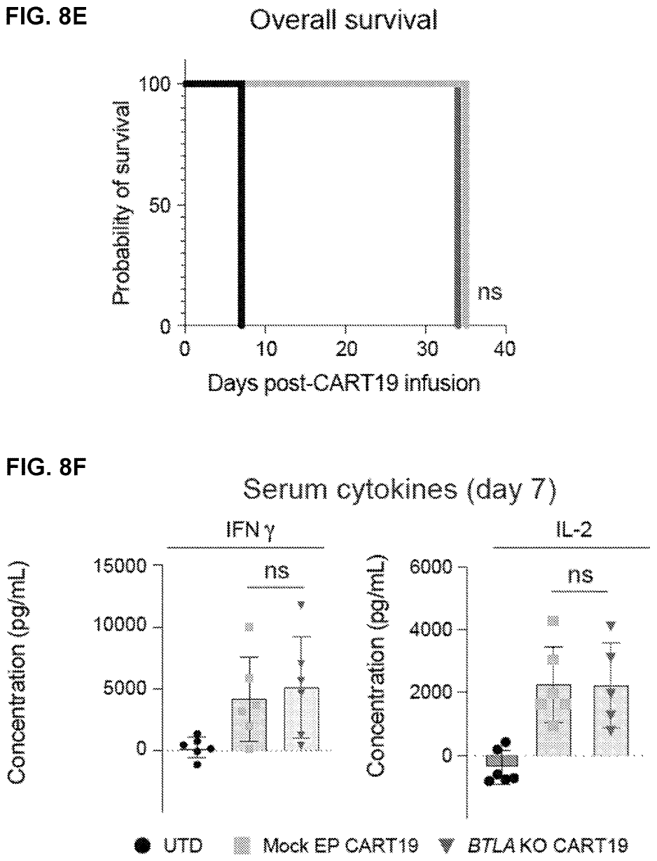

Of note, supporting the role of HVEM on tumor cells as a key mediator of CAR T inhibition via interaction with BTLA, knockout of BTLA had no impact on CART19 efficacy against Nalm6, a HVEM-low B-cell acute lymphoblastic leukemia cell line (FIG. 8A, FIGS. 8C-8F). Conversely, overexpression of HVEM in Nalm6 led to suppression of WT CART19 but not BTLA KO CAR19 T cells (FIG. 8B), further highlighting the role of the trans BTLA-HVEM interaction in CAR T inhibition. Overall, these findings suggest that BTLA expression on CART19 cells can restrict their anti-tumor function against NHL models.

BTLA Knockout Augments CAR T Function within the Tumor Microenvironment

Once the increased efficacy of BTLA KO CART in multiple xenograft models was demonstrated, the function of these cells in the immunosuppressive microenvironment was next tested. Unlike PD-L1 whose expression is predominately restricted to tumor and myeloid cells (e.g., myeloid-derived suppressor cells and M2 macrophages), HVEM has a broader expression that includes not only tumor and the myeloid compartments, but also B cells, regulatory T cells, and dendritic cells (Tao, et al., J Immunol 180, 6649-6655 (2008); Han, et al., EBioMedicine 43, 159-170 (2019); Yu, et al., Front Immunol 10, 617 (2019)). These cell types all represent potential interacting partners with BTLA-expressing effector T cells. Thus, BTLA on tumor infiltrating T cells may interact with this myriad of partners in the TME and drive T cells towards exhaustion.

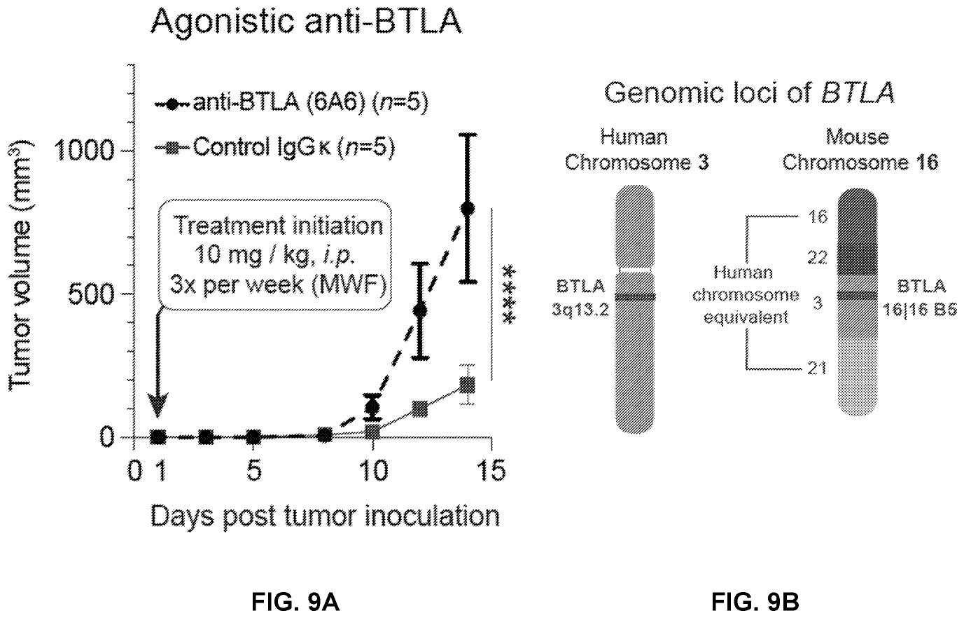

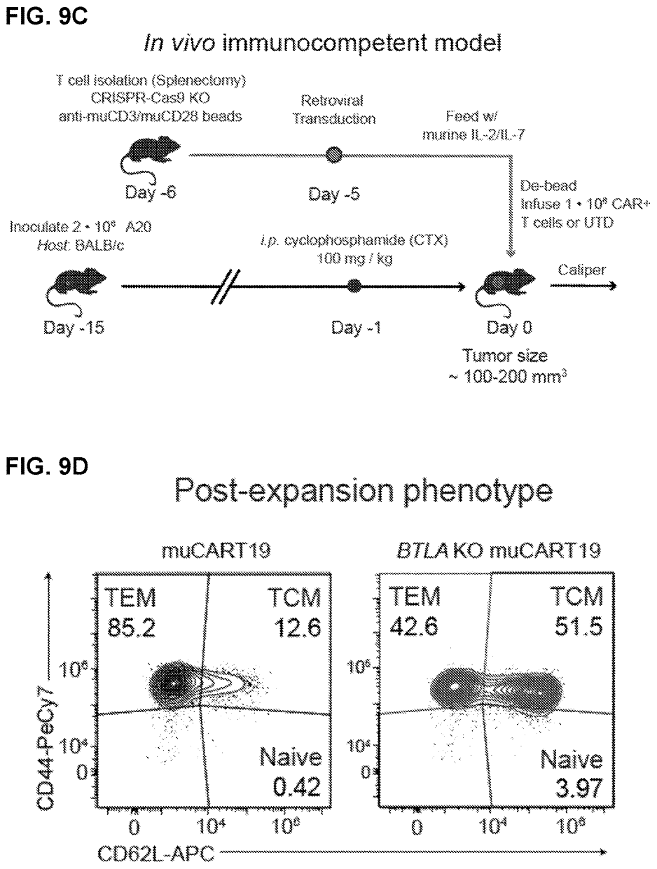

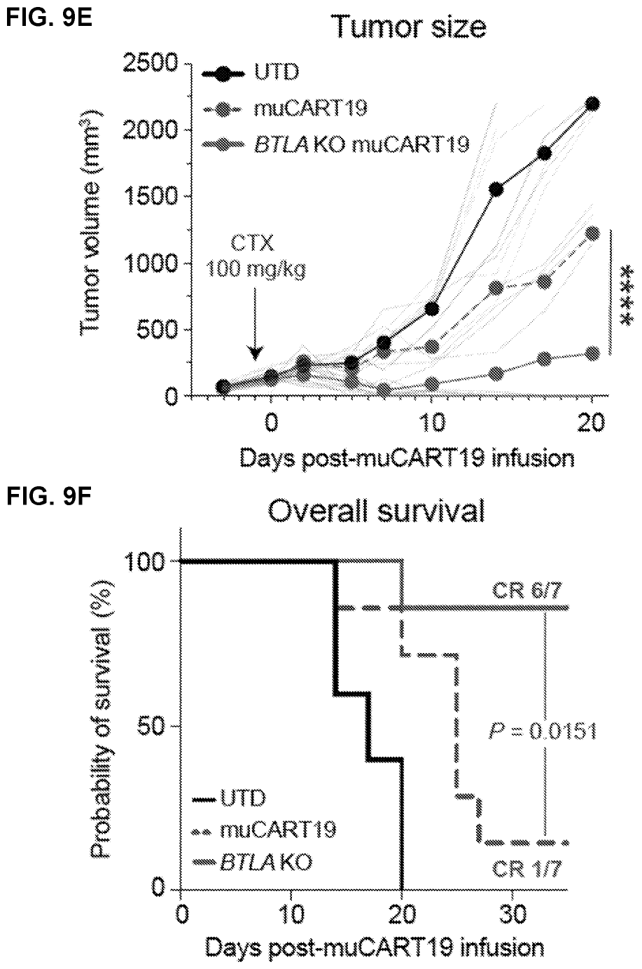

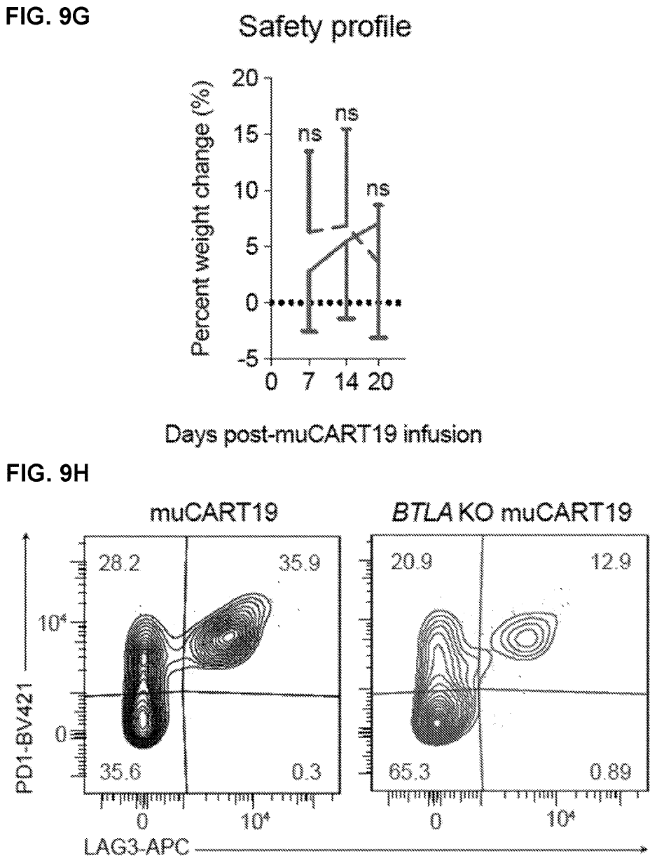

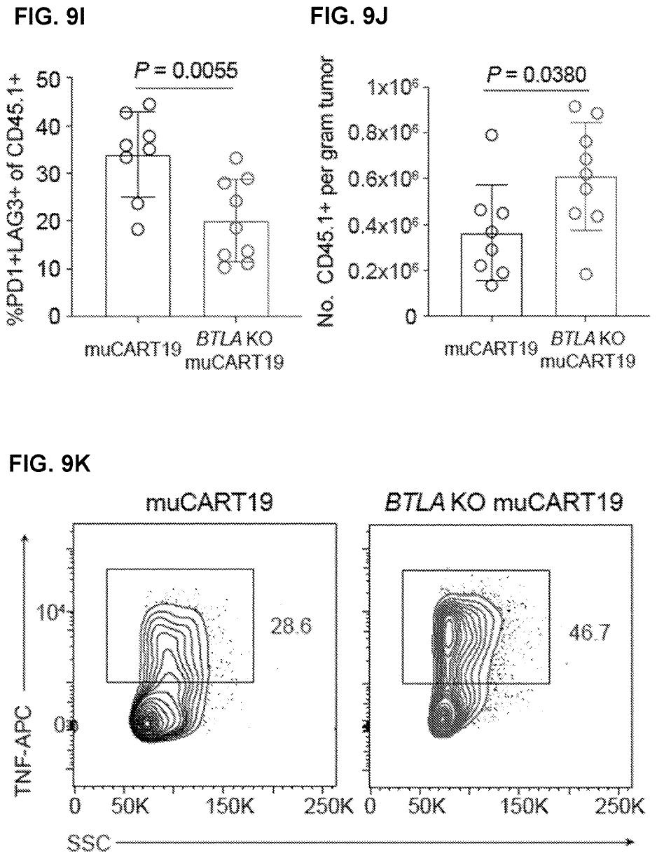

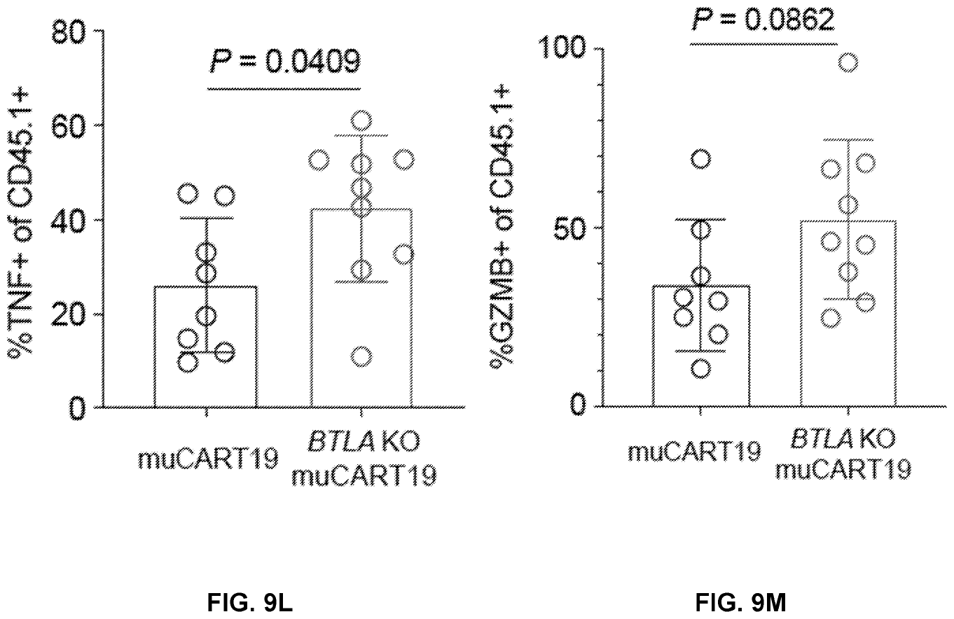

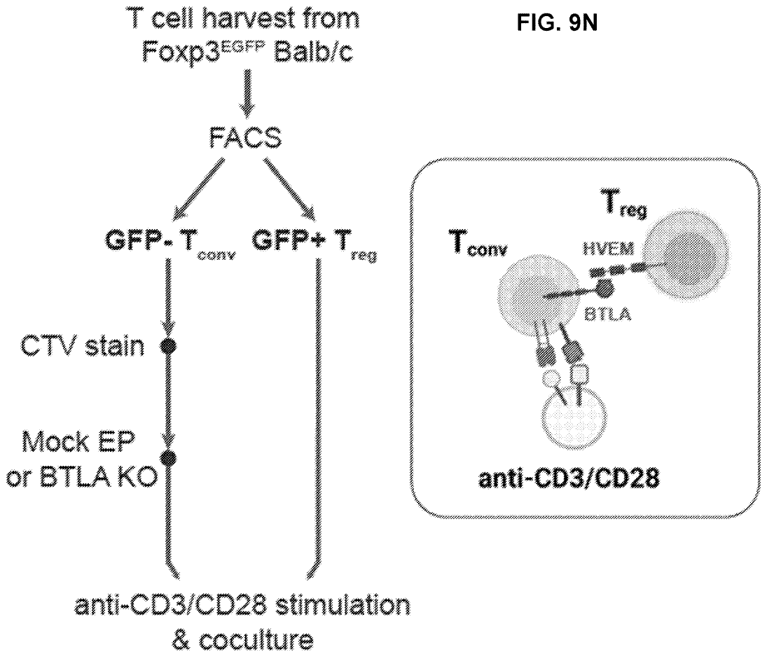

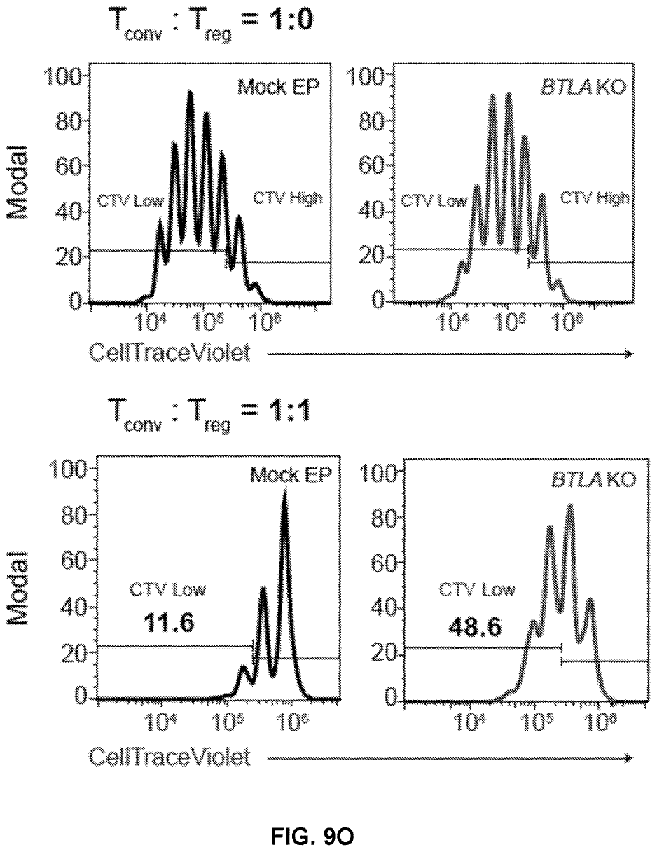

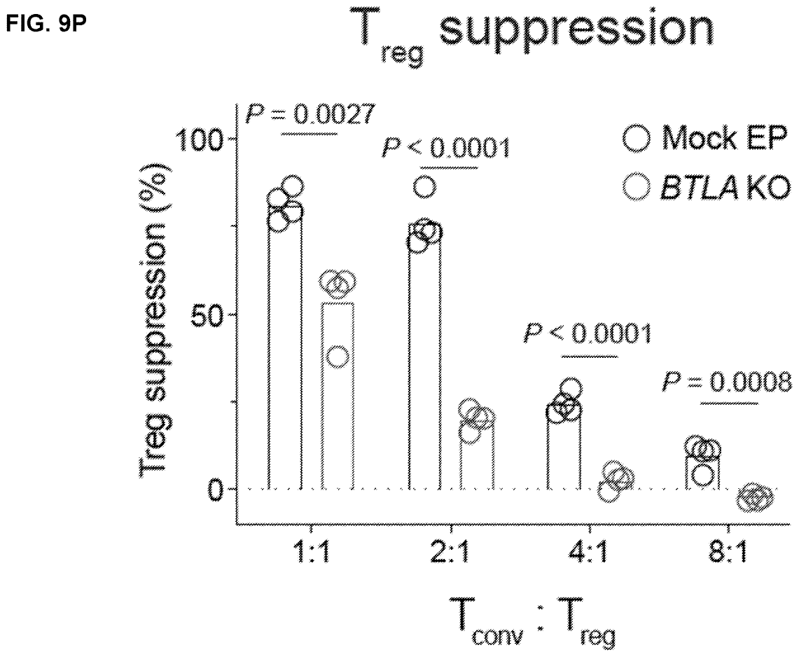

First, to confirm that BTLA is a negative regulator of anti-tumor function in an immunosuppressive TME, an agonistic BTLA antibody was utilized (namely, clone 6A6)—previously used in literature to suppress effector T cells and promote a dysfunctional Treg phenotype (Albring, et al., J Exp Med 207, 2551-2559 (2010); Stienne, et al., Cell Rep 38, 110553 (2022)). Clone 6A6 antibody was administered into A20 lymphoma bearing immunocompetent Balb/c mice. It was observed that the agonistic BTLA antibody accelerated tumor growth relative to an isotype control (P<0.0001 at day 14 post-tumor inoculation), suggesting that signaling through BTLA suppresses immune-mediated tumor control (FIG. 9A).

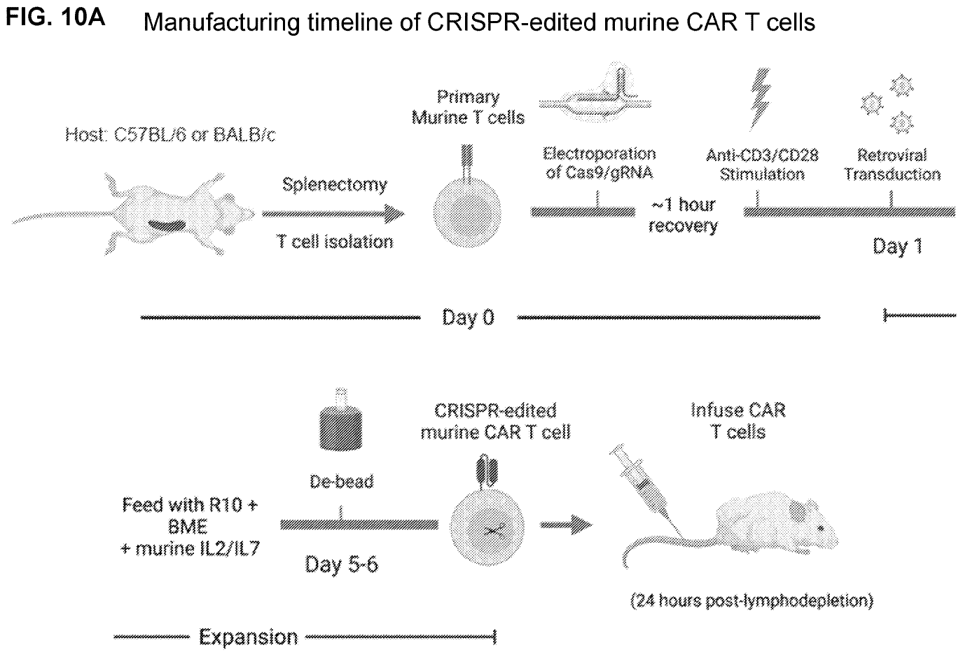

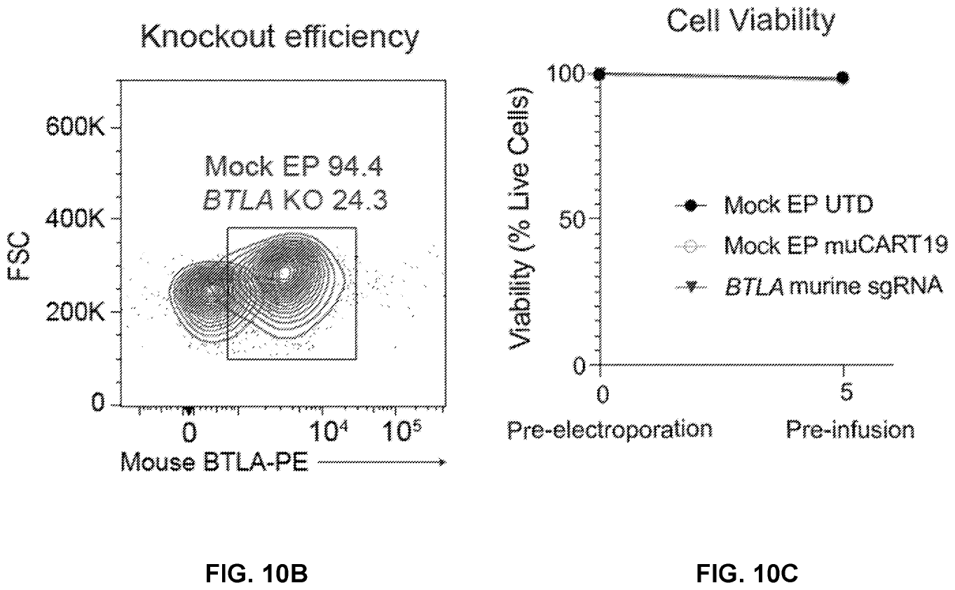

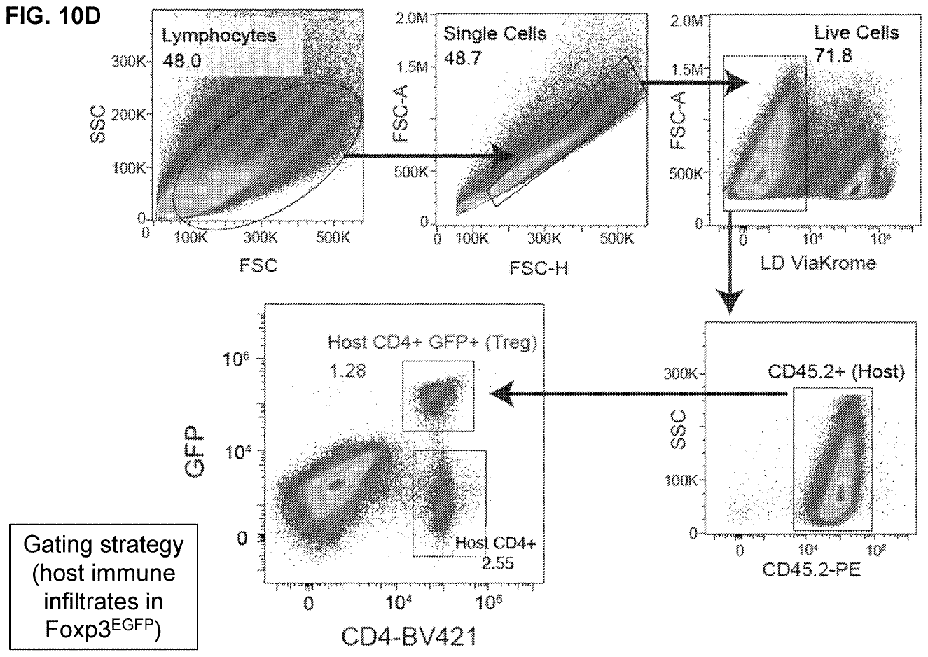

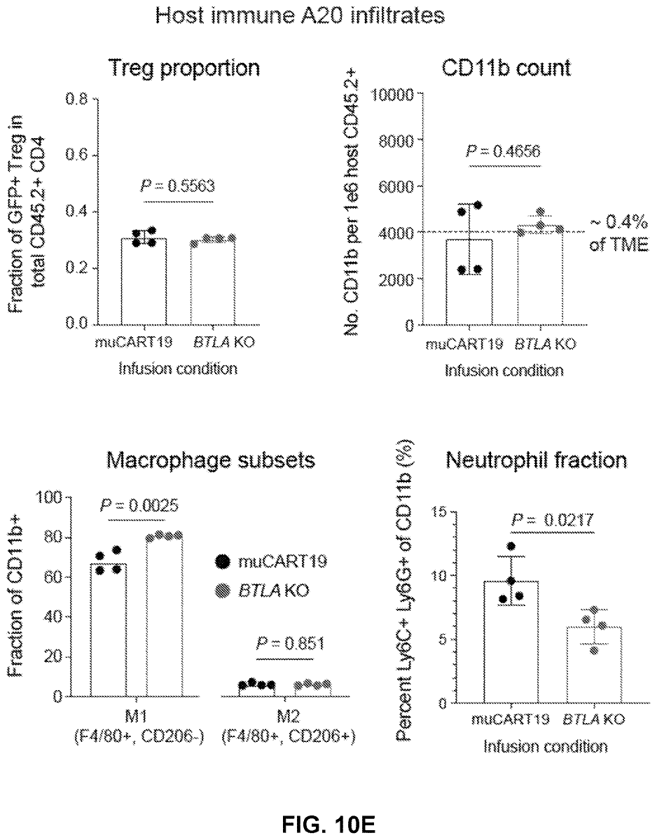

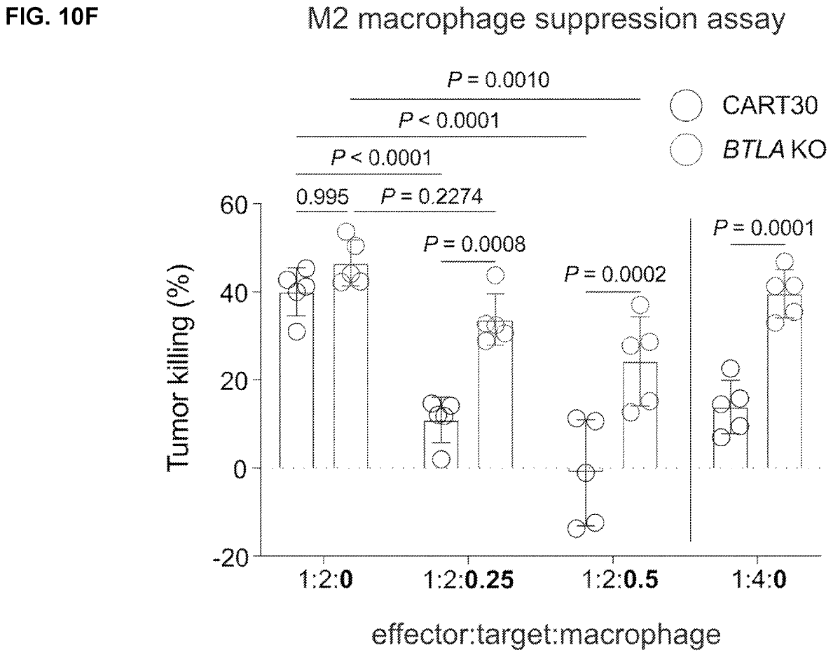

In order to characterize the function of BTLA-deficient CAR T cells in the immunosuppressive TME, a murine CART19 in vivo model that features CRISPR-Cas9 gene editing was implemented against A20 lymphoma. First, a sgRNA against murine BTLA was designed; the BTLA gene in mice (chromosome 16) resides in a genomic location distinct from that of human BTLA (chromosome 3) (FIG. 9B). Next, a gammaretroviral construct bearing a mouse anti-murine CD19 CAR construct (Uribe-Herranz, et al., Mol Ther 31, 686-700 (2023)) was transduced into murine Balb/c-derived T cells (FIG. 9C, FIGS. 10A-10C). During A20 engraftment and growth, manufacturing of murine BTLA KO CART19 cells began. One day prior to CAR T cell infusion, cyclophosphamide (100 mg/kg) was administered as a lymphodepletion. On the day of CART19 infusion, it was observed that electroporation with the murine BTLA sgRNA had preserved a higher proportion of CD62L+CD44+ central memory cells than mock electroporated murine CAR T cells (FIG. 9D). This observation in Balb/c T cells parallels the findings in human T cells at the end of BTLA knockout CART expansion, as shown herein in FIG. 1B. Mice were then randomized to receive either UTD, WT CART19, or BTLA KO CART19. Notably, in this immunocompetent model, BTLA KO murine CAR19 T cells showed superior tumor control as compared to controls (FIG. 9E). Accordingly, improved response rates were observed over their WT muCART19 counterpart mice (CR=6/7 in BTLA KO muCART19; CR=1/7 in WT muCART19, CR=0/5 in UTD; P=0.0151 between muCART19 and BTLA KO) (FIG. 9F). Furthermore, no significant weight fluctuation in BTLA KO muCART19-treated mice was observed relative to WT muCART19, implying that BTLA-deficient CAR T cells may not impose serious toxicities relative to WT CART19-treated mice (FIG. 9G).

In order to assess the phenotype of BTLA KO CAR T cells in the TME, CD45.1+ murine CART19 were infused into CD45.2+A20 bearing mice; seven days post-infusion, tumors were resected and the adoptively transferred CD45.1+ cells were characterized via flow cytometry. The results show that BTLA knockout murine CART19 cells had higher infiltration into A20 tumors, and had less co-expression of classic exhaustion markers (i.e., PD-1, LAG-3) (FIGS. 9H-9J). Furthermore, when stimulated ex vivo, BTLA knockout CD45.1+ infiltrates produced higher levels inflammatory cytokines (i.e., TNF, GZMB) (FIGS. 9K-9M). These data indicate that BTLA-deleted CAR T cells have heightened activity within an immunosuppressive TME. Indeed, HVEM expression on A20 cells is known to restricts T cell infiltration, and that HVEM deletion in A20 promotes the infiltration of potent, PD-1-negative T cells (Del Rio, et al., Front Immunol 14, 1113858 (2023)).

Given these results, experiments were performed to gauge how immunosuppressive cells in the TME, namely Treg and macrophages, interact with BTLA KO CAR T cells. First, it was observed that Treg are rich in the A20 TME, while macrophages are fewer (FIG. 10D, FIG. 10E). Thus, in order to study Balb/c Treg-T cell interactions ex vivo, GFP+ Treg cells were sorted both from C.Cg-Foxp3^{tm2Tch}/J (Foxp3^{EGFP}) transgenic Balb/c mice, and correspondingly the GFP− “conventional” T (T_{conv}) cells (FIG. 9N). Mock-electroporation was performed (WT) or BTLA knockout in T_{conv}, and these cells were stimulated with anti-CD3/CD28 beads in the presence or absence of Treg cells. In this in vitro Treg suppression assay (Zappasodi, et al., Nature 591, 652-658 (2021)), Treg had a greater suppressive effect on WT T cell proliferation than on BTLA KO T cell proliferation at 72 hr, and at all tested ratios of T_{conv} to Treg (FIG. 9O, FIG. 9P). Importantly, major differences were not observed in the composition of Treg or monocytes within the TME of WT muCART19-treated versus BTLA KO muCART19-treated A20 tumors (FIG. 10E). Second, to study the role of inhibitory M2 macrophages, HL was revisited. HL is a disease in which CD68+ macrophages substantially infiltrate the TME and associate with poor prognosis (Steidl, et al., New England Journal of Medicine 362, 875-885 (2010)). Given that the HDLM-2 HL model used in the previous experiments lacks a functional TME, and given that no established model of HL in immunocompetent mice exists, whether BTLA-deficient CAR T cells could improve anti-tumor function against HDLM-2 in the presence of immunosuppressive macrophages was next tested. WT or BTLA KO CAR T cells were therefore triple-cocultured against both in vitro-differentiated M2 macrophages (of the same donor) and HDLM-2 HL cells (FIG. 10F) (Ruella, et al., Cancer Discovery 7, 1154-1167 (2017)). The results show that BTLA KO CAR T cells were more resistant to suppression from M2 macrophages than their WT counterparts.

Taken together, these data highlight the potential for BTLA-deficient CAR T cells to better navigate established barriers of the immunosuppressive TME like Treg and macrophages.

### BTLA Knockout Drives CAR T Cell Effector Programming In Vivo

Because the benefit of BTLA-deficient CAR T cells was observed in several tumor models, key regulatory genes and pathways associated with their improved performance over WT CAR T cells were next identified by characterized both in vivo circulating CAR T cells (i.e., Proliferation model) or tumor-infiltrating CAR T cells (i.e., Exhaustion model) via scRNA-seq.

First, a model was used in which WT or BTLA KO CART19 cells were infused into NSG mice (n=8 WT, n=6 BTLA KO) 10 days after OCI-Ly18 implantation. In this model, curative doses of CAR T cells were used to induce substantial proliferation of CAR T cells in vivo without driving these CAR T cells into a state of exhaustion. On day 16 post-infusion, circulating CART19 cells were isolated from the peripheral blood of these mice and scRNA-seq was performed (FIG. 11A, FIG. 11B, FIG. 12A). Five major T cell subsets were identified, such as CD4+ and CD8+ T effector (T_{eff}) and naive/memory T cells (FIG. 11C). It was observed that BTLA-deficient CAR T cells represented a higher proportion in T_{eff} subsets (FIG. 11D) and were characterized by expression of PRF1, GZMB, and T-bet (FIG. 11E, FIG. 11F). Furthermore, in CD4 T_{eff} cells, BTLA KO CART19 showed an upregulation of cytotoxic markers (e.g., GZMB, GZMA, GZMK) and downregulation of immunosuppressive markers (e.g., TGFB1, CCR4) (FIG. 11G). Notably, BTLA KO CD4 T_{eff} exhibited downregulation of PTPN6 (SHP-1), further underlining a link between BTLA and its utilization of SHP-1. Similarly, like CD4 T_{eff}, BTLA KO CD8 T_{eff} were enriched in GZMB, IFNG, and PRF1, while downregulated TGFB1 and IL10RA (FIG. 11H). BTLA KO CD8 T_{eff} also showed upregulation of LAG-3 and CTLA4—likely reflective of their sustained activation status in this Proliferation model. To confirm this, pathway analysis was performed which demonstrated that BTLA knockout enriched pathways associated with effector T cell status (e.g., cytolytic granule, cell killing) and not exhaustion (FIG. 11I). To functionally corroborate the enhanced effector function of BTLA knockout CAR T cells, it was observed that BTLA KO cells from this experiment persisted at higher levels than WT CART19 over an extended time interval (days 31 and 45 post-infusion) (FIG. 11J). In summary, these findings indicate that BTLA deletion induces effector programming in CAR T cells in vivo and that this heightened effector function is sustainable.

Next, tumor-infiltrating CAR T cells were studied, now using suboptimal doses of CAR T cells that led to tumor persistence and CAR T exhaustion. Here, CAR T cells were infused 14 days after tumor implantation, when these tumors became palpable, in order to stress-test the CAR T cells. TILs were then sorted on day 11 post-infusion for scRNA-seq (FIG. 12B, FIG. 12C). Clusters were denoted by expression of key enriched genes (i.e., IFNG, IL13, GZM, TCF7) (FIGS. 12D-12F). Generally, TILs showed low expression of TCF7 and high expression of TOX, indicative of a transition to exhaustion in both conditions. However, in an IFNG-rich cluster, BTLA knockout TILs demonstrated upregulation of key effector cytokines (e.g., IL2, IL23A) (FIG. 12G). This was reflected in an enrichment of pro-stimulatory T cell signaling pathways (e.g., tyrosine phosphorylation of STAT, JAK/STAT) in BTLA KO TILs. Moreover, in a separate cluster marked by high IL13 expression, BTLA knockout TILs showed high expression of critical T cell activation genes (BATF3, PGK1) while downregulating exhaustion-associated markers (TIGIT) (FIG. 12H). Taken together, these results support that BTLA knockout can perpetuate CART intra-tumoral effector responses.

To further characterize the BTLA KO CAR T cells, a phosphorylated-kinase proteomic array of the cells was performed following 15 minutes of anti-CD3/CD28 stimulation in either WT CAR T cells or BTLA KO CAR T cells. This phospho-proteomic analysis revealed that BTLA KO enhances activation of PLC-gamma, STAT3, and STAT6 in CAR T cells (FIG. 12H), indicative of heightened activation and effector programming (Fraietta, J. A. et al., Nat Med 24, 563-571 (2018)). Notably, unlike PD1 and CTLA-4, BTLA is also capable of recruiting the pro-stimulatory Grb2 (Ritthipichai, K. et al., Clin Cancer Res 23, 6151-6164 (2017); Murphy, K. M., Nelson, C. A. & Šedý, J. R., Nature Reviews Immunology 6, 671-681 (2006)).

### BTLA Inhibits CAR Signaling Via ITIMs Y257 and Y282 in the Presence of HVEM

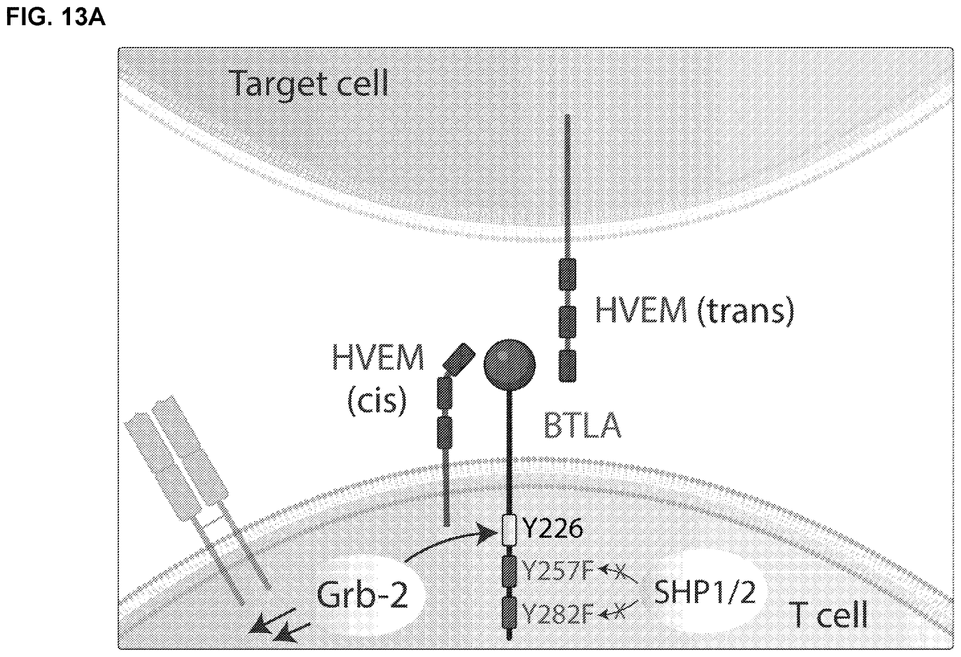

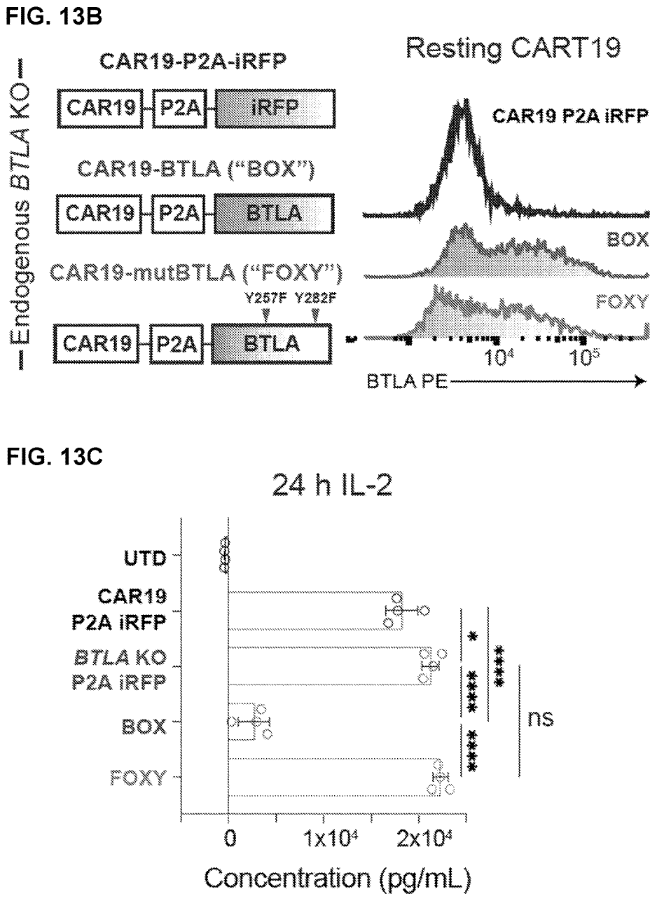

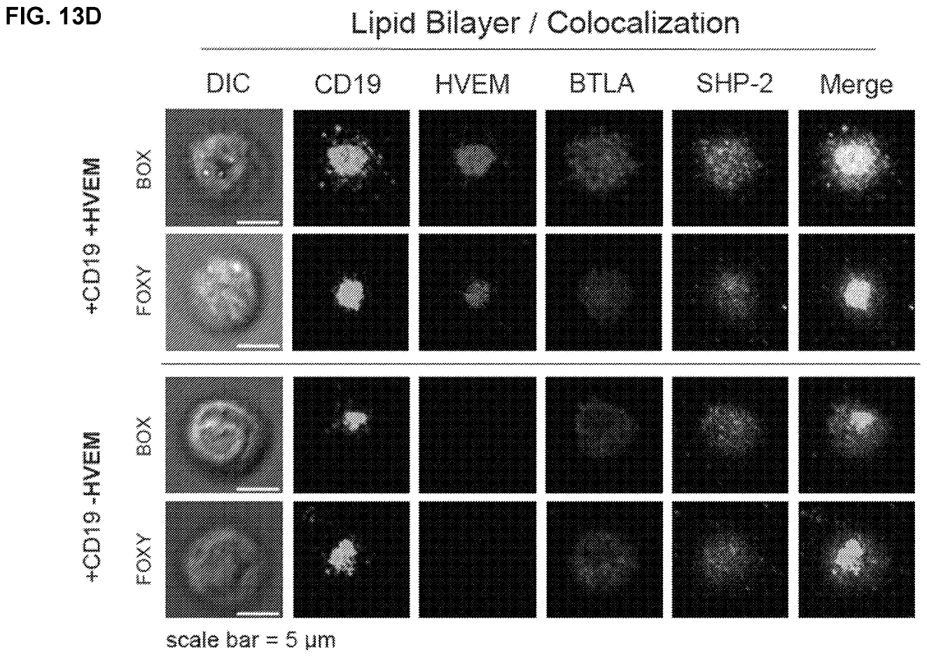

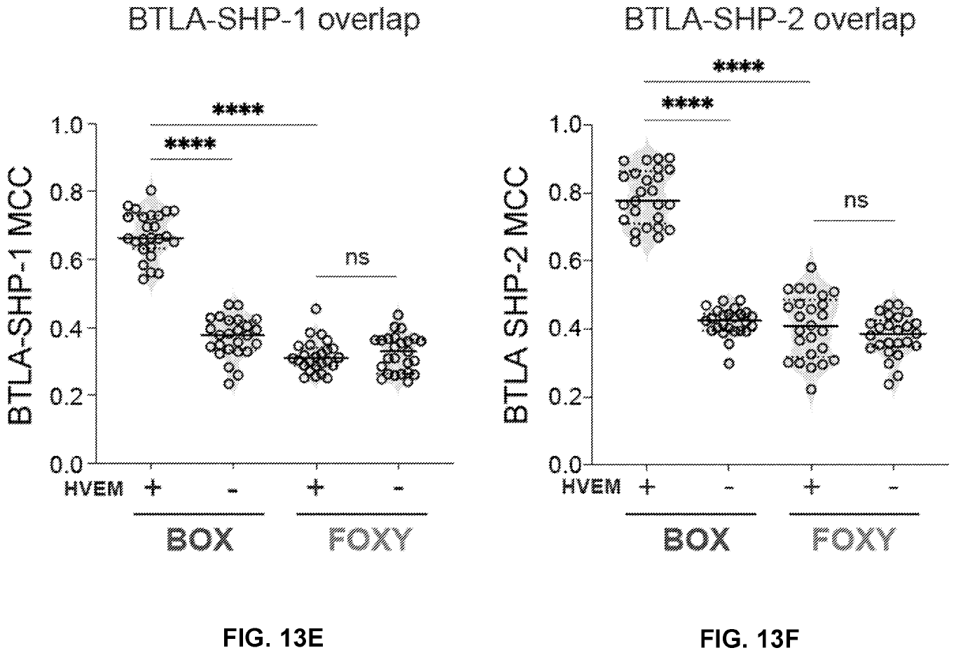

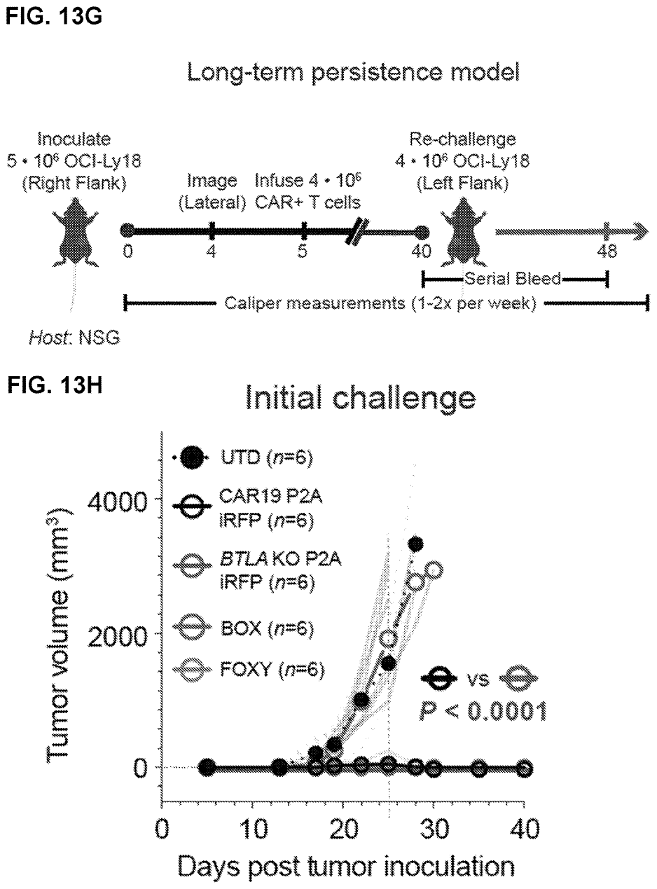

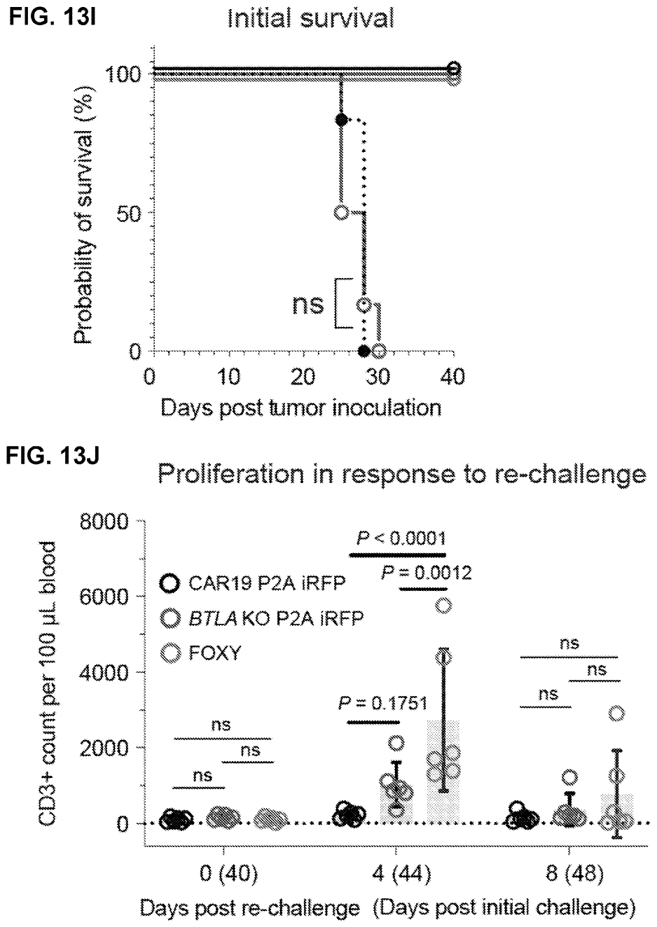

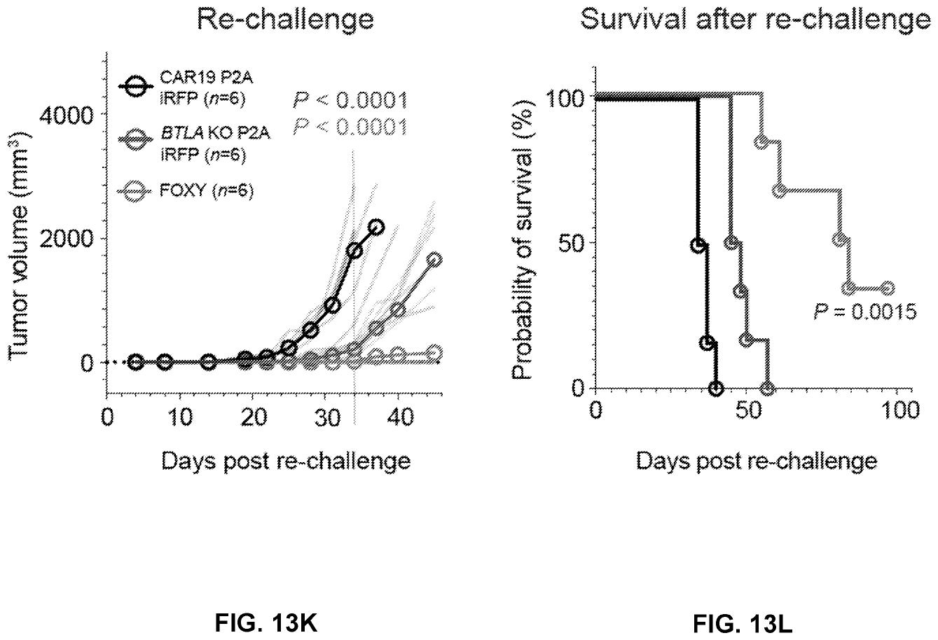

Next, the mechanisms preceding the functionality, phenotype and expression profile changes of BTLA KO CAR T cells were explored and defined. BTLA, PD-1, and CTLA-4 belong to the CD28 superfamily and recruit the phosphatases SHP-1 and SHP-2 via immunoreceptor tyrosine-based inhibitory motifs (ITIMs) to inhibit early T-cell signaling (Murphy, et al., Nature Reviews Immunology 6, 671-681 (2006)). Unlike PD-1 and CTLA-4, however, BTLA is also known to recruit Grb2, a pro-stimulatory molecule that induces activation of the NFkB/NFAT pathway (Schneider, et al., Biochem Biophys Res Commun 369, 616-621 (2008)). Studies have implied that exhausted T cells may even utilize BTLA for its ability to recruit Grb2 and induce the NFkB pathway, thus jolting longer-term persistence (Haymaker, et al., Oncoimmunology 4, e1014246 (2015); Ritthipichai, et al., Clin Cancer Res 23, 6151-6164 (2017)). To study the role of the BTLA intracellular domains on CAR T cell function, lentiviral constructs were designed which co-express CAR19 and either BTLA (BTLA overexpressing—“BOX”) or BTLA bearing two point-mutations at its ITIMs (Y257F, Y282F) to abolish recruitment of SHP-1 and SHP-2 but maintain Y226 for binding to Grb2 (tyrosine(Y)-to-phenylalanine(F) mutated BTLA overexpressing—“FOXY”) (FIG. 13A) (Xu, et al., eLife 10, e74276 (2021); Ritthipichai, et al., Clin Cancer Res 23, 6151-6164 (2017); Gavrieli, et al., Biochem Biophys Res Commun 312, 1236-1243 (2003); Chemnitz, et al., J Immunol 176, 6603-6614 (2006)). Thus, BOX CART19 should accelerate recruitment of SHP-1 and SHP-2 and shut off CAR T cell function, while FOXY CART19 should preserve stimulatory signaling transmission via Grb2 upon engagement with HVEM. First, T cells were transduced with either the BOX or FOXY constructs and activation-independent expression of BTLA at the surface of both models was confirmed (FIG. 13B). Initially, to test these constructs, a 24 h coculture with OCI-Ly18 was conducted in which it was observed that both BTLA KO and FOXY CART19 enhanced IL-2 production relative to WT CART19 (FIG. 13C). BOX CART19, on the other hand, demonstrated stifled IL-2 production, suggesting that overexpressing WT BTLA, but not its mutant, restrains CAR signaling.

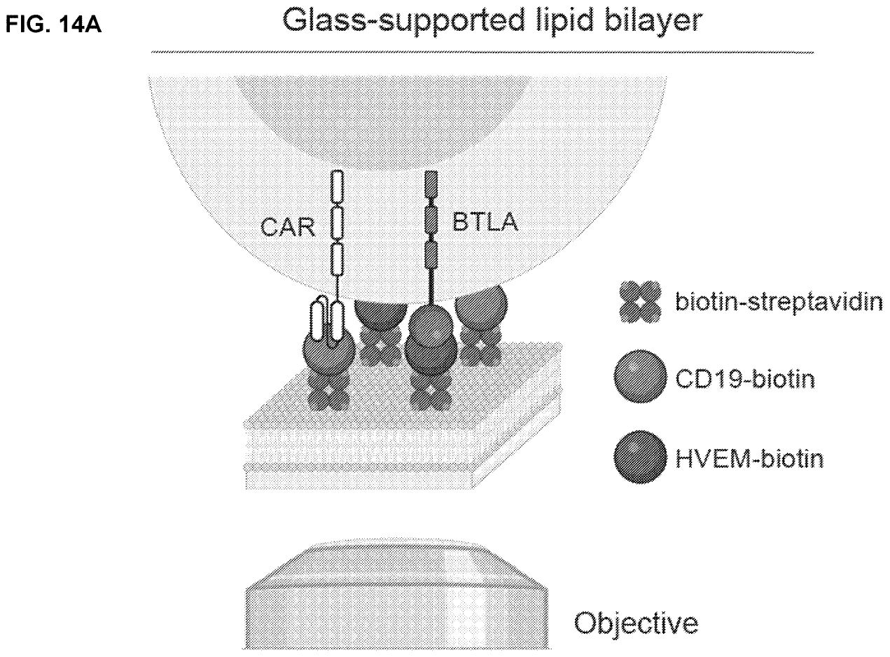

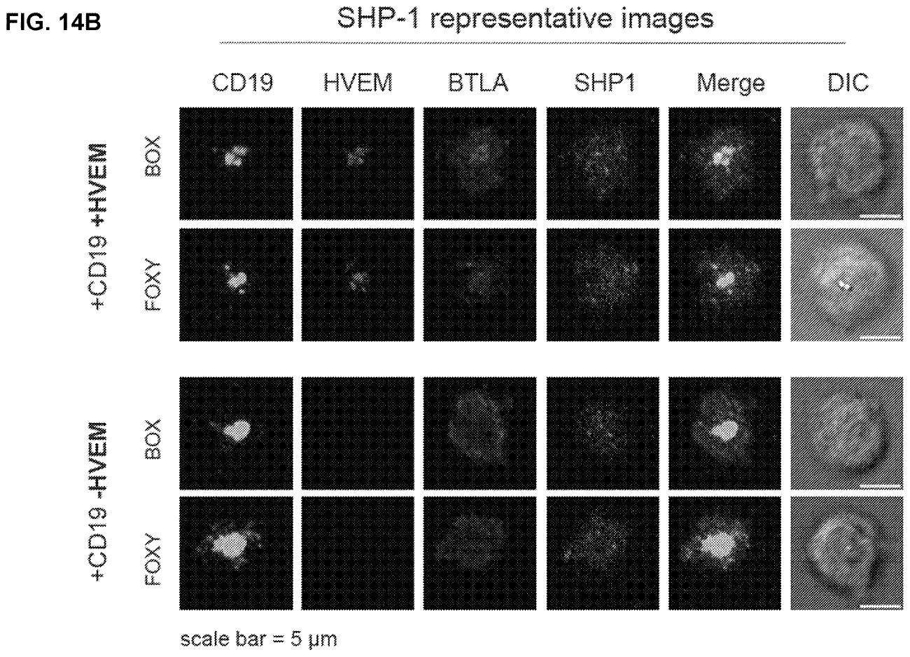

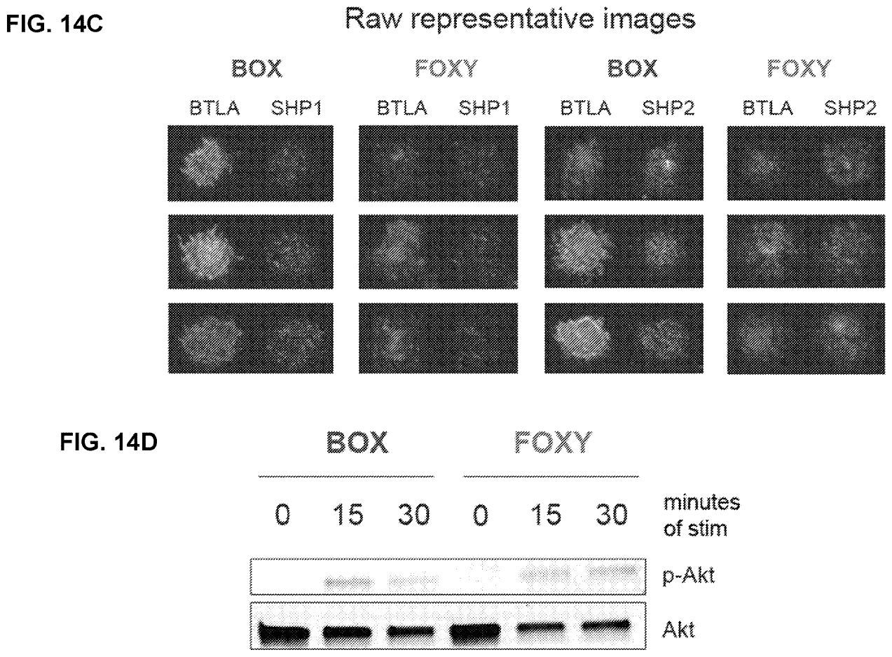

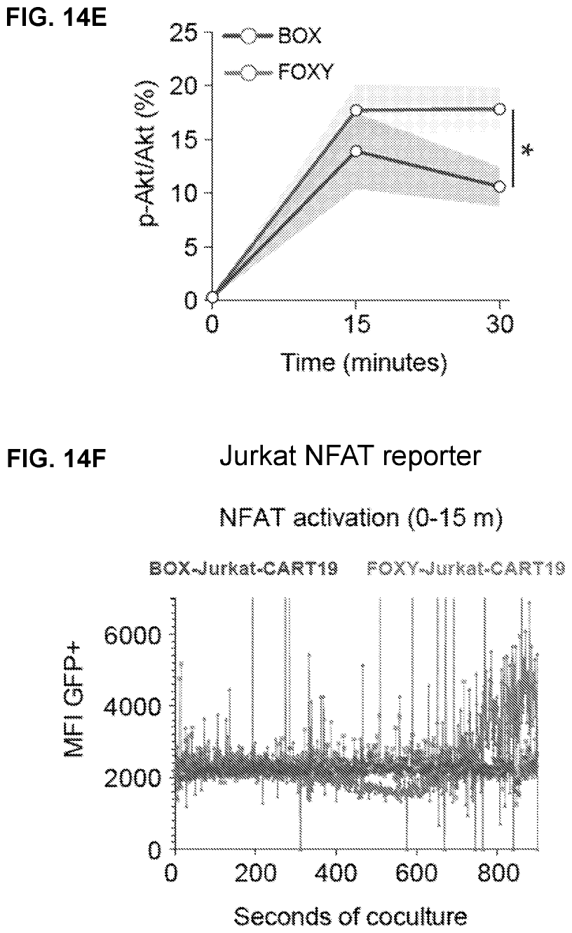

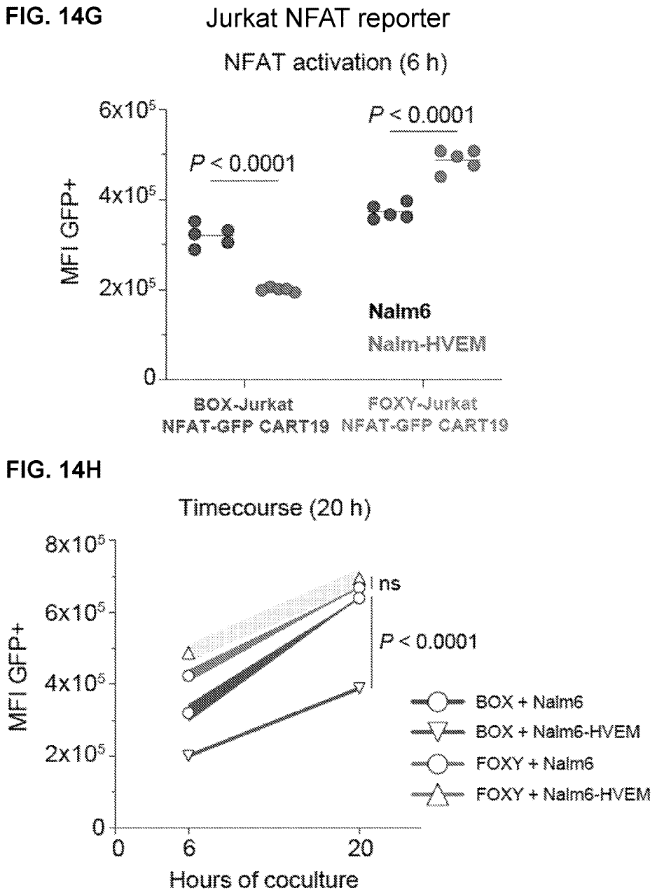

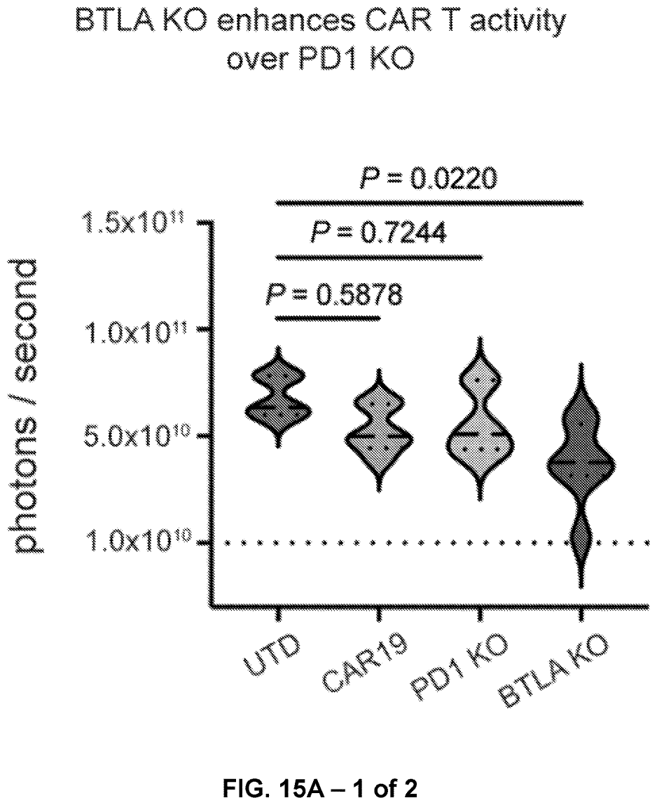

To validate that BOX promotes docking of SHP-1 and SHP-2 upon CAR engagement, and that FOXY disallows this, a lipid-bilayer model of the immunological synapse was utilized (FIG. 14A) (Bertolet, et al., Methods Mol Biol 1441, 151-165 (2016); Dustin, et al., Science 367, eaba7365 (2020)). Clustering of BTLA to the synapse in both BOX and FOXY CART19 was dependent on the presence of HVEM (FIG. 13D, FIG. 14B, FIG. 14C). In BOX CART19, BTLA colocalized with both SHP-1 and SHP-2, while in FOXY CART19, BTLA did not colocalize with either protein (FIG. 13E, FIG. 13F). These results confirm that Y257 and Y282 are the ITIMs in BTLA responsible of recruiting SHP-1 and SHP-2 to the inner membrane leaflet of CAR T cells (Mintz, et al., Immunity 51, 310-323.e317 (2019)). To measure if FOXY CART19 increase Grb2 activation over BOX CART19, both of which have the Y226 domain intact, activation of Akt, a major downstream target of Grb2, was measured in BOX or FOXY CART19 following CD19 and HVEM engagement (Sordo-Bahamonde, et al., Cancer Immunology, Immunotherapy 72, 2529-2539 (2023)). It was observed that FOXY CART19 maintained higher phosphorylation of Akt over time, indicating that FOXY CART19 are better capable of sustaining Grb2 activation (FIG. 14D, FIG. 14E). Finally, that the enhanced signaling in the FOXY construct leads to activation of key transcription factor NFAT relative to the BOX construct was demonstrated, which is also indicative of intact stimulatory signaling (FIGS. 14F-14H). These data confirm the relevance of Y257 and Y282 in the BTLA-mediated inhibition of CAR T cells.

To validate these in vitro signaling results, an in vivo experiment was performed to assess the long-term function of the BTLA-ITIM-modified FOXY CART19 cells (FIG. 13G). In the initial tumor challenge, OCI-Ly18 was engrafted subcutaneously into the right flank of NSG mice. On day 5, mice were infused with either UTD, WT CART19, BTLA KO CART19, BOX CART19, or FOXY CART19 cells. Shortly after CAR T cell infusion, as expected, UTD-treated and BOX CART19-treated mice lost tumor control, supporting the hypothesis that WT BTLA expression drives T cell dysfunction through SHP-1 and SHP-2 recruitment (FIG. 13H, FIG. 13I). At day 40, after tumors were cleared in the WT, BTLA KO, and FOXY CART19 groups, the surviving mice were subject to re-challenge with OCI-Ly18 now on the left flank. Serial bleeding of mice following re-challenge demonstrated heightened CAR T cell responsiveness to the tumor re-challenge in the FOXY CART19 group over WT and BTLA KO CART19 (FIG. 13J). Subsequently, WT CART19-treated mice lost tumor control and relapsed first, followed by relapse in mice treated with BTLA KO CART19 (FIG. 13K, FIG. 13L). Overall, these studies highlight the role of the SHP-recruitment domains in the inhibitory role of BTLA. Moreover, the results described herein demonstrate that inducing mutations in BTLA, rather than fully ablating BTLA, can preserve other protein functions and promote the long-term persistence of CAR T cells.

### Dual Inhibition of BTLA and PD-1 Promote Long-Lasting Response to CAR T

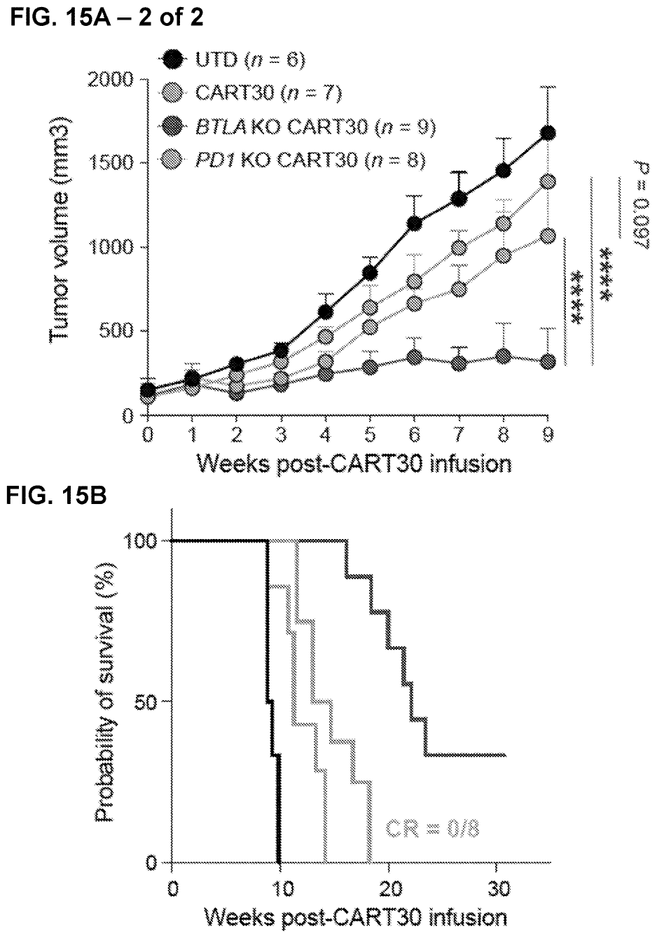

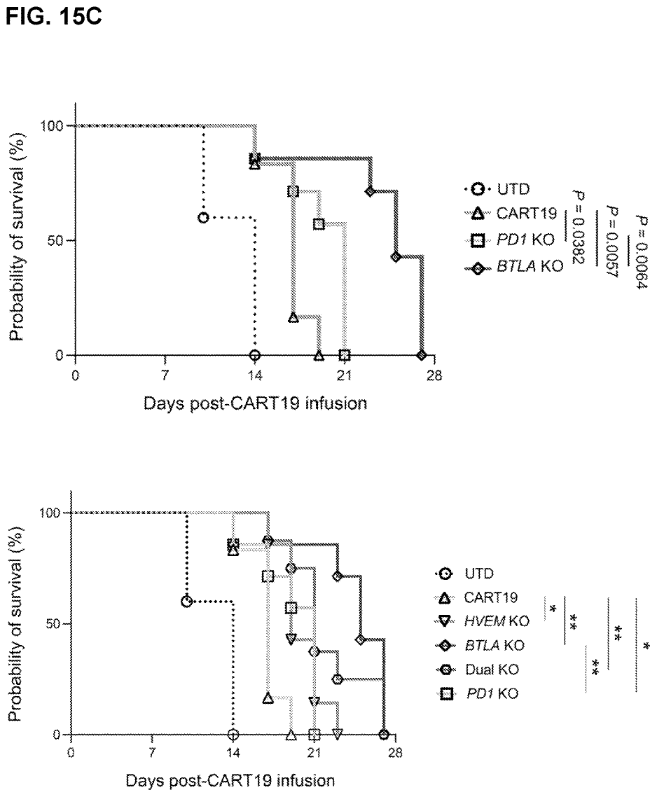

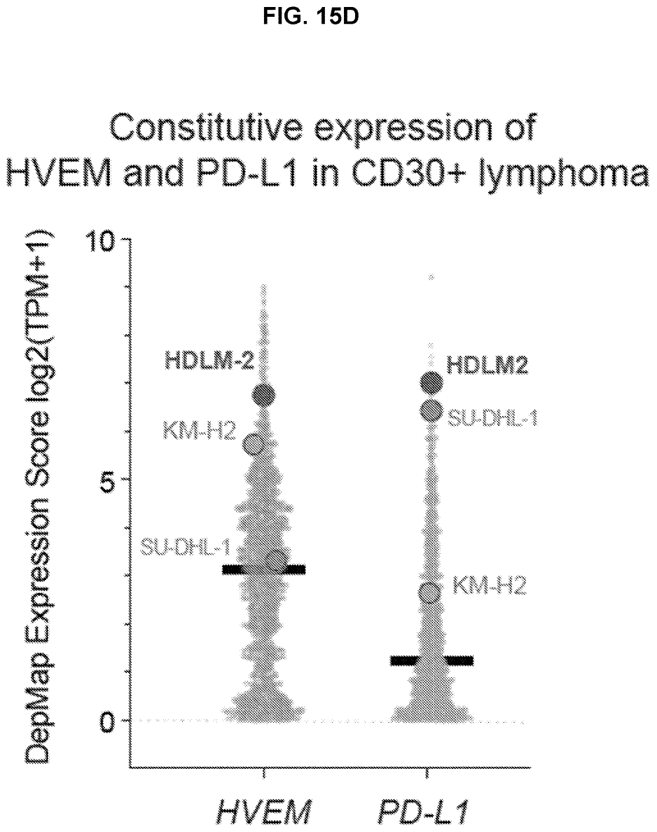

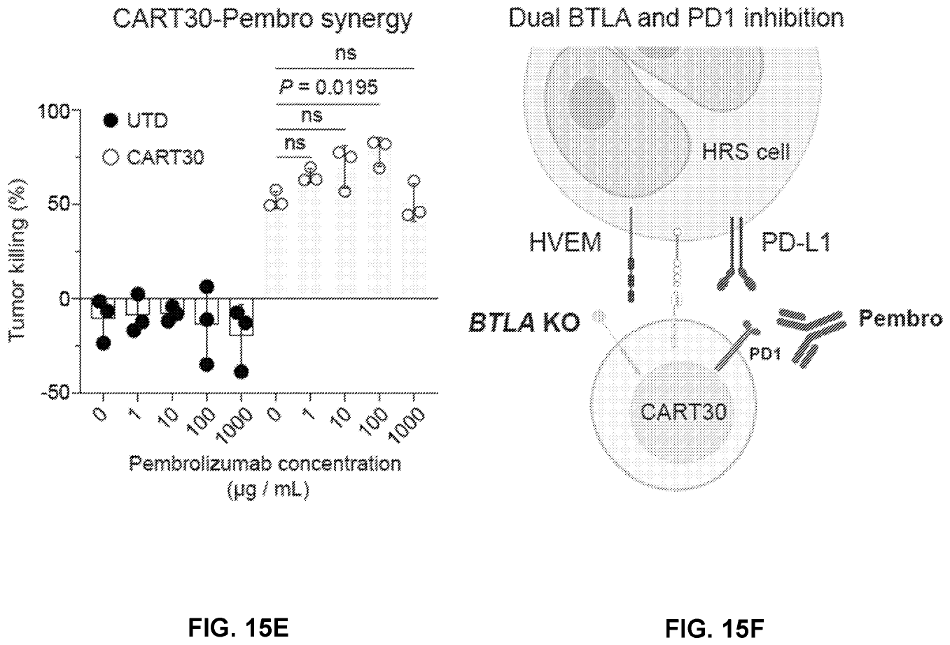

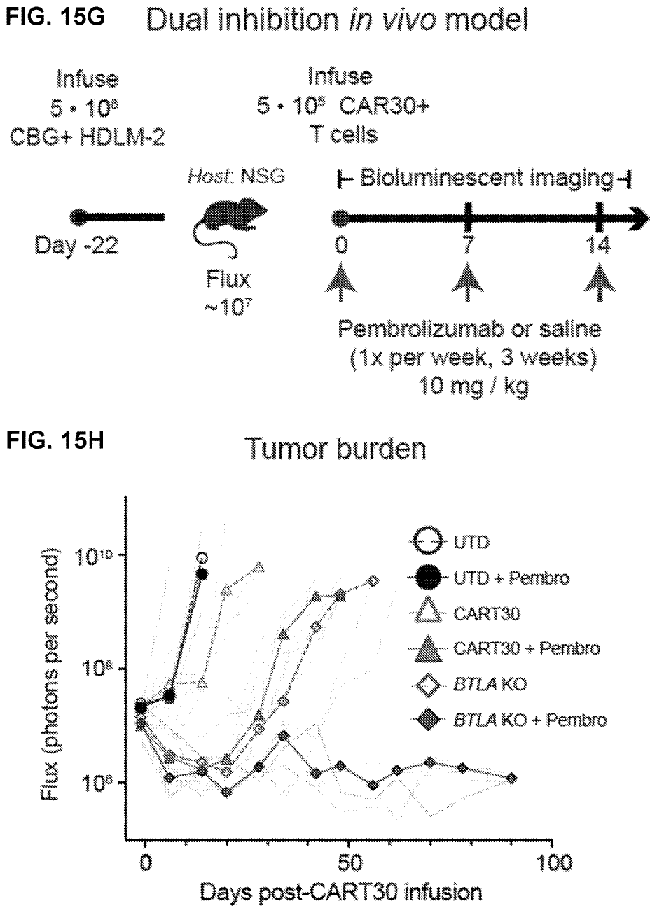

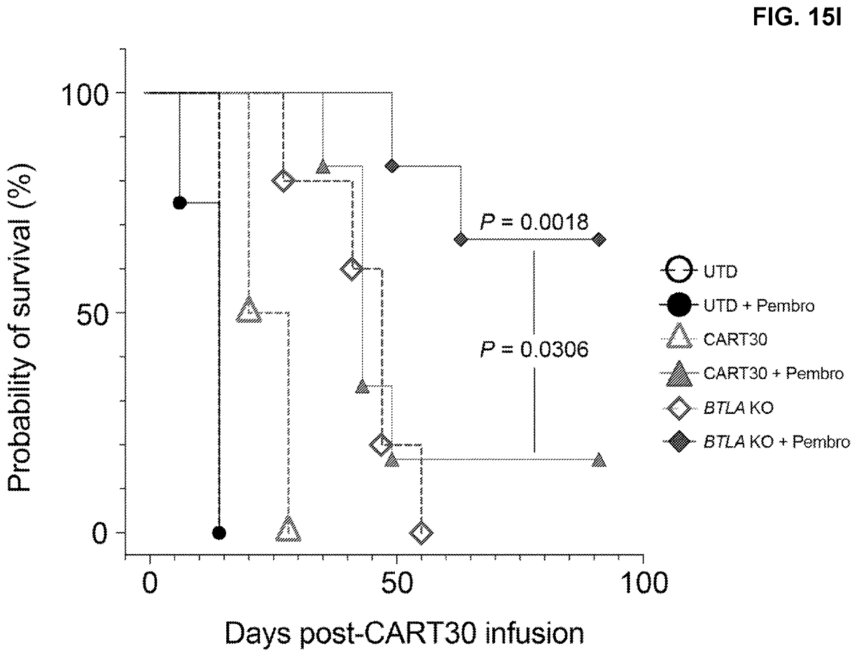

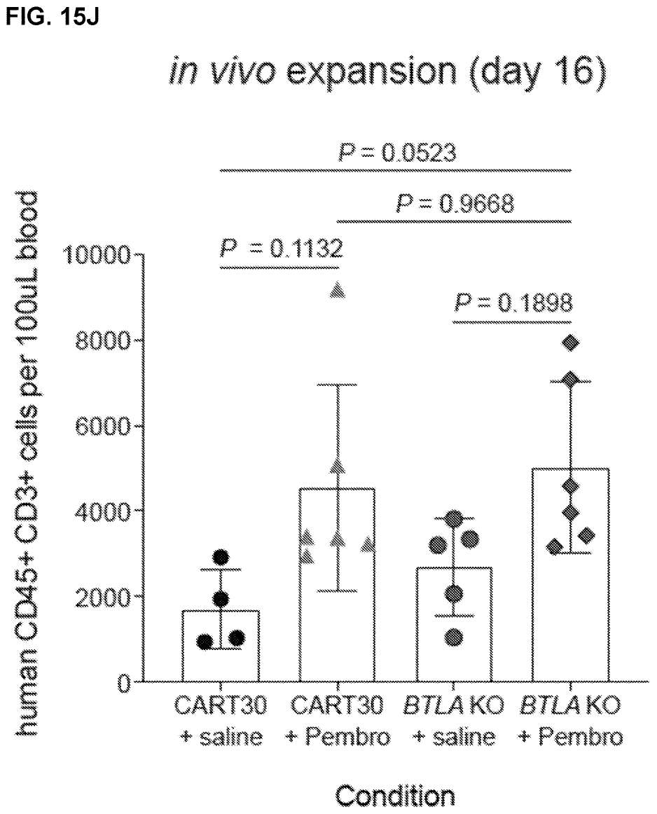

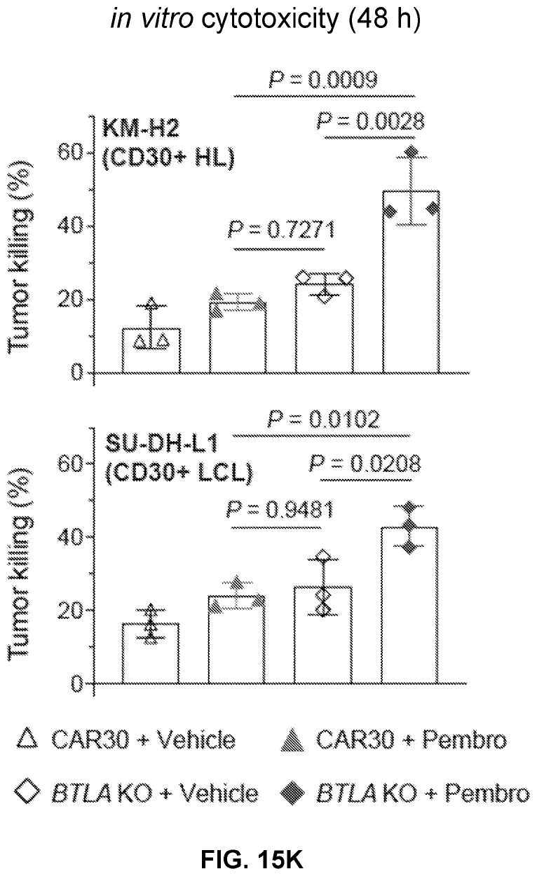

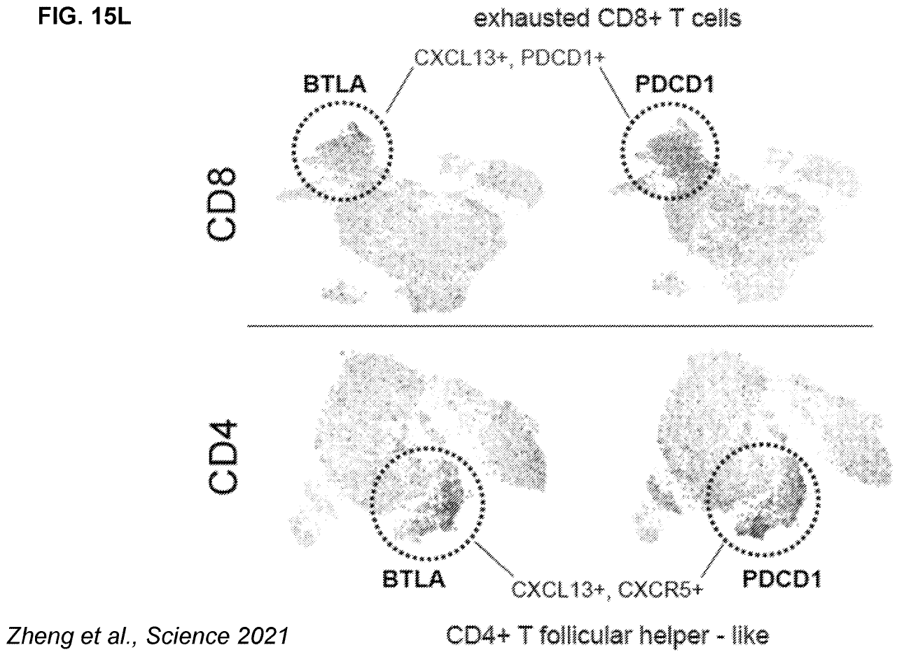

The canonical PD-1-PD-L1 axis has been the prototypic target of T cell cancer immunotherapies, including immune checkpoint blockade and even PD-1-deficient CAR T cells (Stadtmauer, et al., Science 367, eaba7365 (2020)). In particular, both the anti-PD-1 antibodies nivolumab and pembrolizumab (Pembro) have been FDA-approved for use in HL as numerous studies point to PD-L1 hyperexpression, both by tumor cells and in the myeloid compartment of the HL TME (Chen, et al., Blood 134, 1144-1153 (2019); Moskowitz, et al., Journal of Clinical Oncology 39, 3109-3117 (2021); Armand, et al., J Clin Oncol 36, 1428-1439 (2018)). Given the similarity of the PD-1-PD-L1 axis to the BTLA-HVEM axis in recruiting SHP-1 and SHP-2, and reports of clinical efficacy of PD-1 knockout in CAR T cells, whether BTLA-deficient CAR T cells were comparable in function to PD-1-deficient CAR T cells was investigated. In fact, a PD-1 knockout control was included in the studies evaluating CART30 in HL and CART19 in NHL (FIGS. 15A-15C). Unexpectedly, decreased tumor burden was observed in mice treated with BTLA KO CAR T cells over those treated with PD1 KO CAR T cells or controls (FIG. 15A). Thus, in these models, BTLA KO still conferred a functional advantage over PD-1 KO. Additionally, PD-1 KO CAR T improved tumor control over WT CART30 in the HDLM-2 model, although this difference was not statistically significant (P=0.097 between CART30 and PD-1 KO at week 9).

HL cell lines like HDLM-2 and KM-H2 show constitutive expression of both HVEM and PD-L1 (Wein, et al., Cancer Immunology Research 5, 1122-1132 (2017)), suggesting that strategic blockade of both PD-1 and BTLA may be synergistic in these models (FIG. 15D). Thus, whether Pembro could augment the function of CART30 in vitro was tested. A short-term coculture of CART30 and HDLM-2 with increasing doses of Pembro was performed, and a significant increase in tumor killing was observed at a clinically relevant dose (100 μg/mL) (FIG. 15E). Thus, a strategy to combine BTLA KO CAR30 T cells with Pembro was devised (FIG. 15F). To test this hypothesis of dual BTLA/PD-1 checkpoint inhibition in HL, systemic tumors of HDLM-2 were engrafted in NSG mice via tail vein injection into NSG mice (FIG. 15G) (Ruella, et al., Cancer Discovery 7, 1154-1167 (2017)). On day 22, mice were infused with UTD, WT CART30, or BTLA KO CART30 cells. Mice received intraperitoneal injections of either saline or Pembro once a week for three weeks. Tumor burden was controlled to the largest extent by the BTLA KO CAR30 T cells and Pembro combination treatment (FIG. 15H). The majority of mice treated with the BTLA KO+Pembro combination had elevated levels of circulating CAR T cells and entered remission with no measurable tumor burden (CR=4/6); this corresponded to long-lasting survival of these mice (FIG. 15I, FIG. 15J). Furthermore, synergy of BTLA knockout in CART30 and Pembro was observed in short-term coculture experiments with other CD30+ malignancies (i.e., KM-H2, SU-DHL-1) that also bear constitutive expression of HVEM and PD-L1 (FIG. 15K).

To better understand the targeted cellular compartments of the dual inhibition approach, the distribution of BTLA and PD-1 expression among exhausted T cells was explored in a pan-cancer TIL single-cell RNA sequencing atlas (FIG. 15L) (Zheng, et al., Science 374, abe6474 (2021)). The results indicate that both BTLA and PDCD1 are co-enriched in exhausted CD8+ TILs and T follicular helper (Tfh)-like exhausted CD4+ TILs. Interestingly, previous studies have associated BTLA expression as characteristic of Tfh, and independently, that Tfh promote immunosuppression against cancer (Mintz, et al., Immunity 51, 310-323.e317 (2019); Gu-Trantien, et al., JCI Insight 2 (2017); Dai, et al., Journal for Immuno Therapy of Cancer 9, e001823 (2021)). T cells co-expressing BTLA and PD-1 may thus mark specific, targetable subsets that are the subject of the synergistic approach described herein using BTLA knockout CAR T cells and PD-1 blockade. These results support the use of dual BTLA and PD-1 inhibition as a promising combination to treat tumors with expression of both HVEM and PD-L1 checkpoints (Choi, et al., Oncoimmunology 10, 1956142 (2021)).

### BTLA Modulation in CAR T Against Solid Tumors

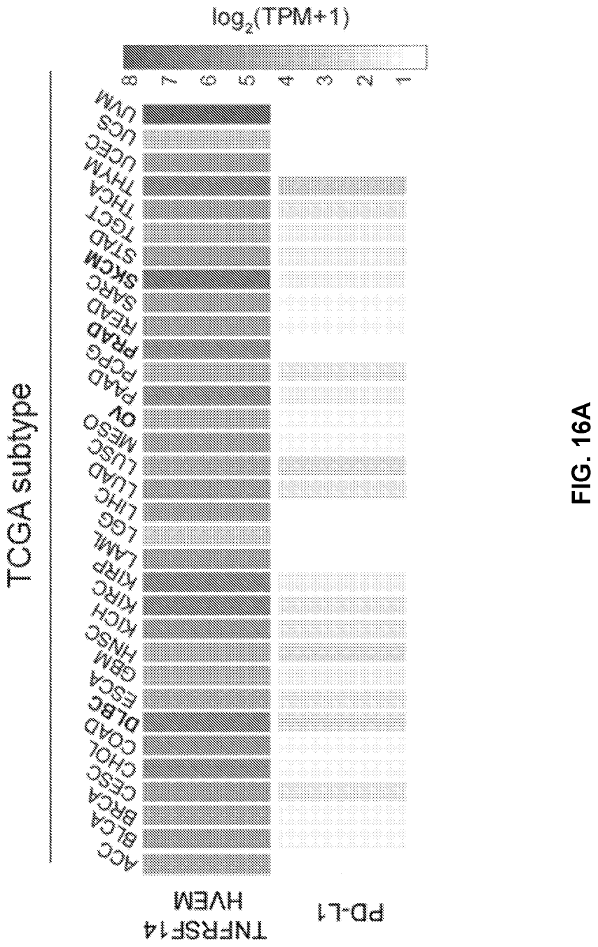

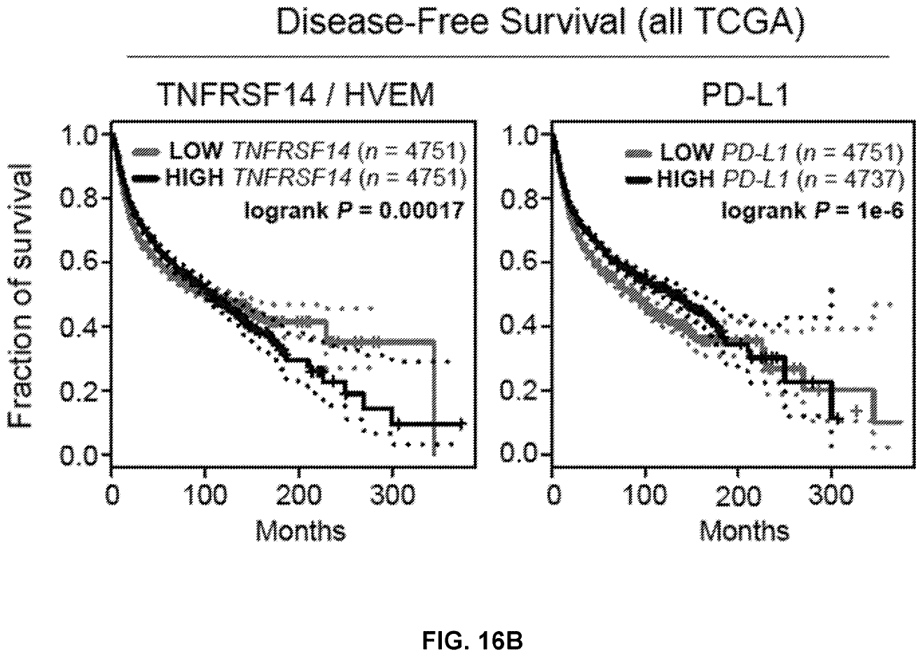

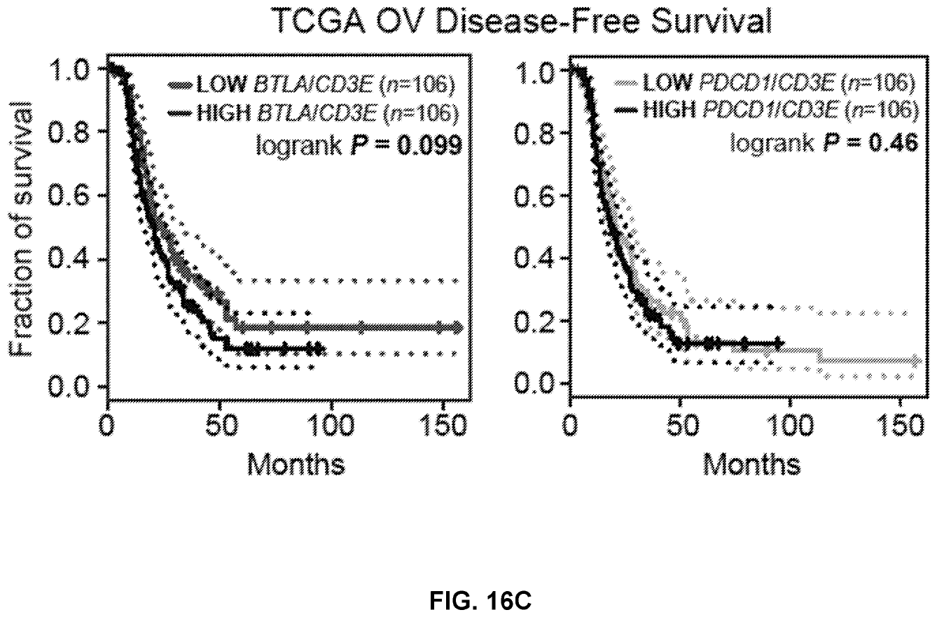

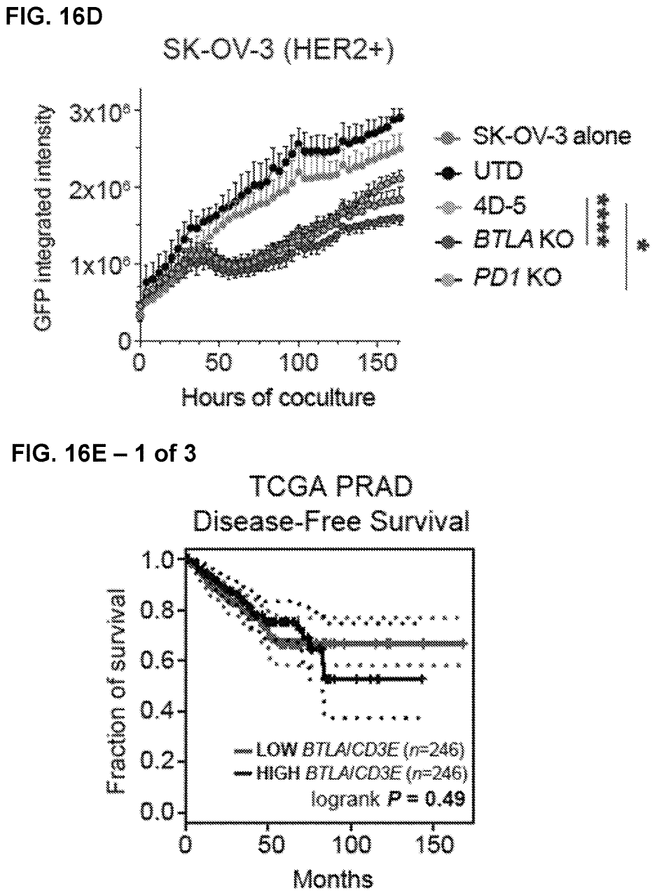

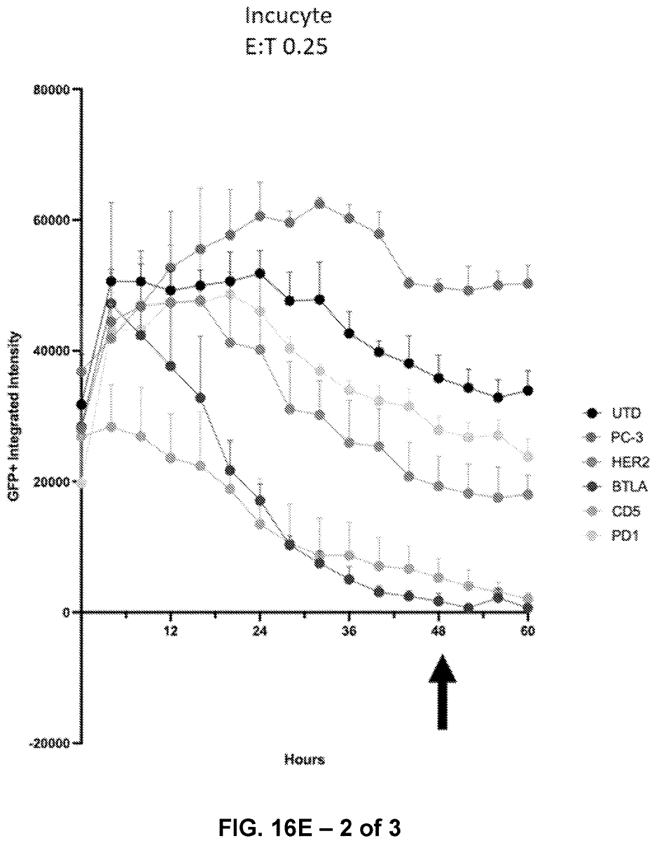

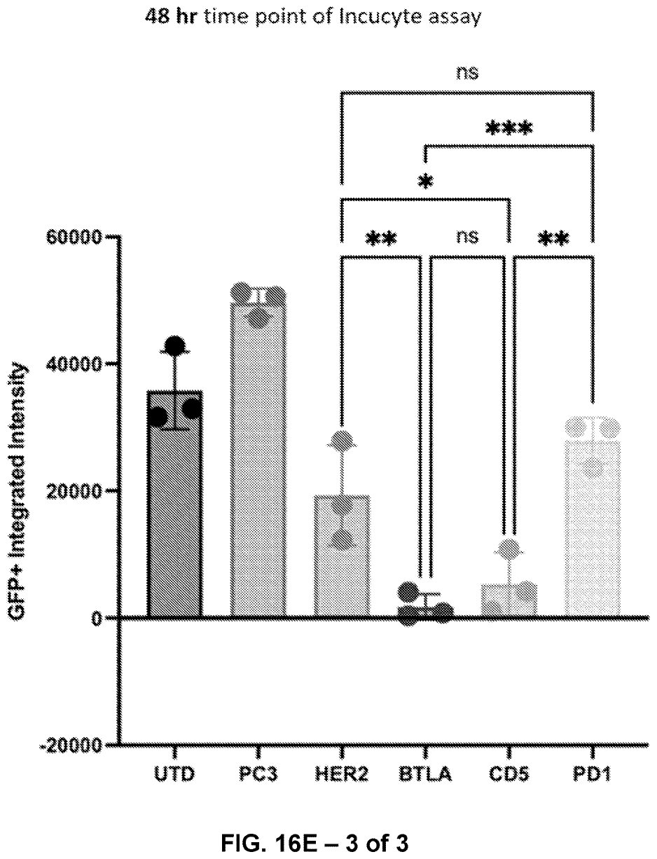

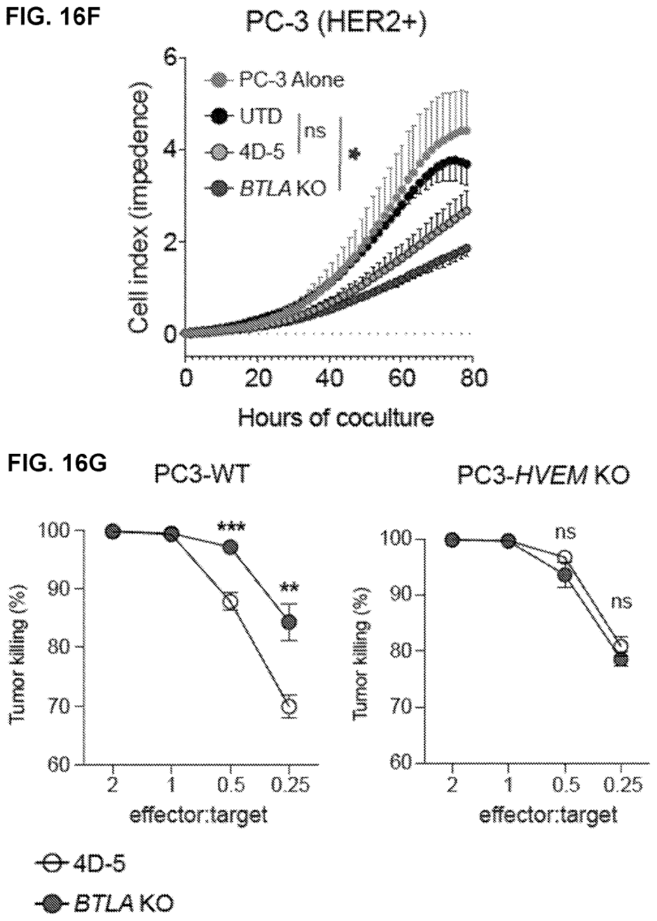

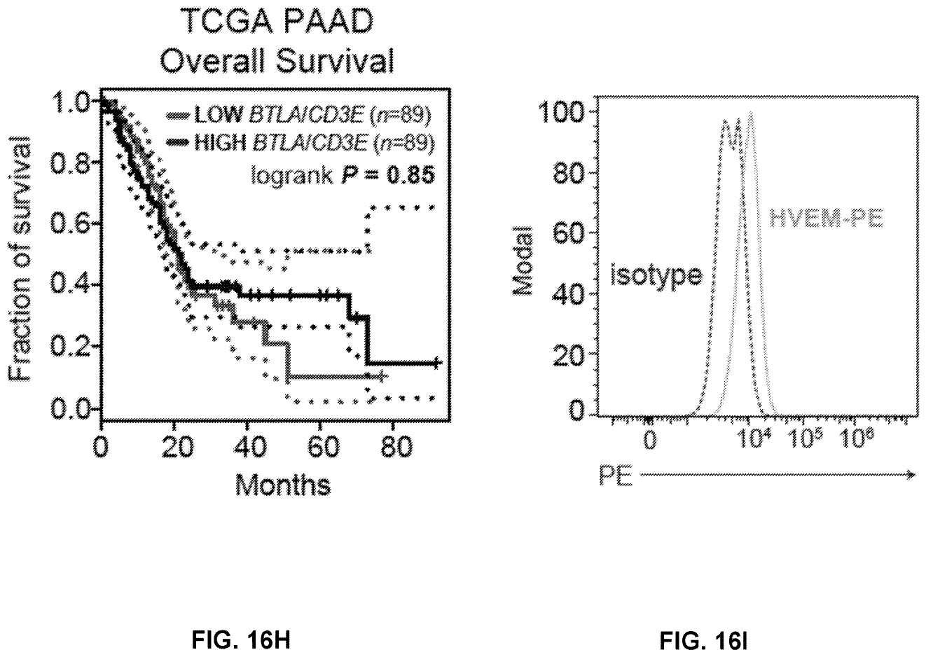

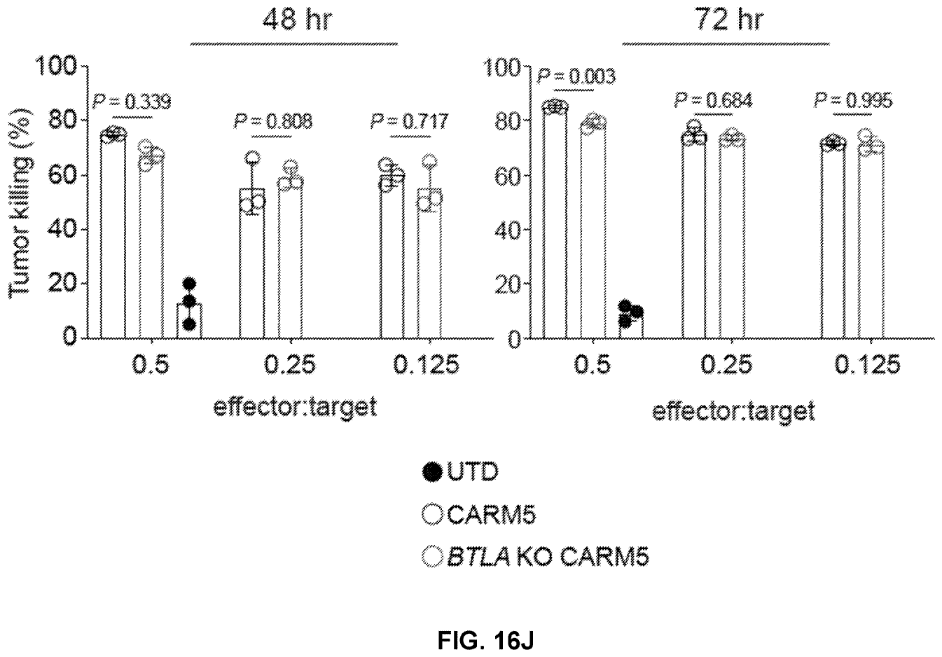

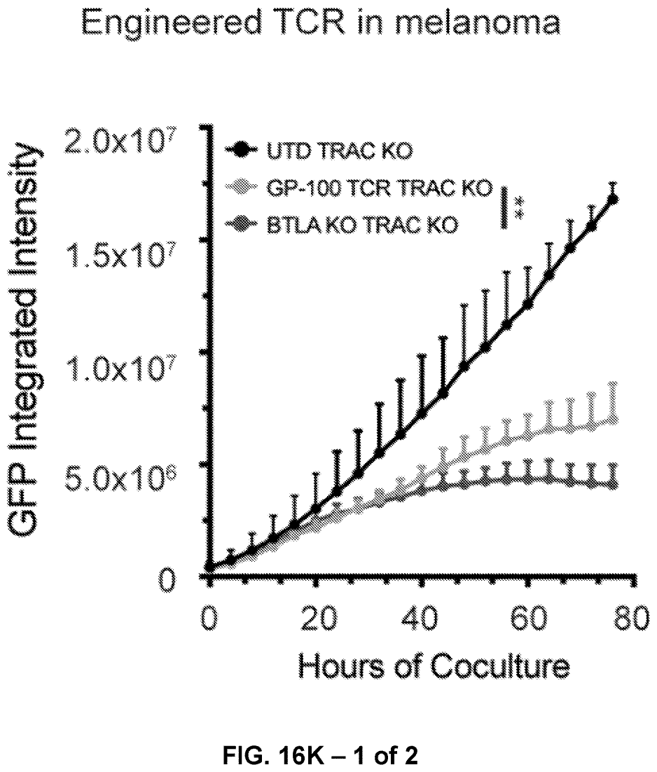

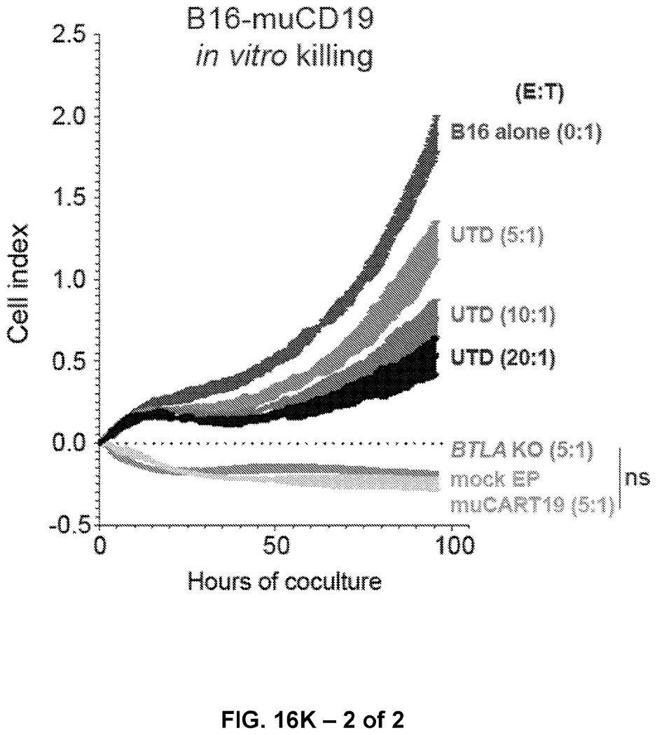

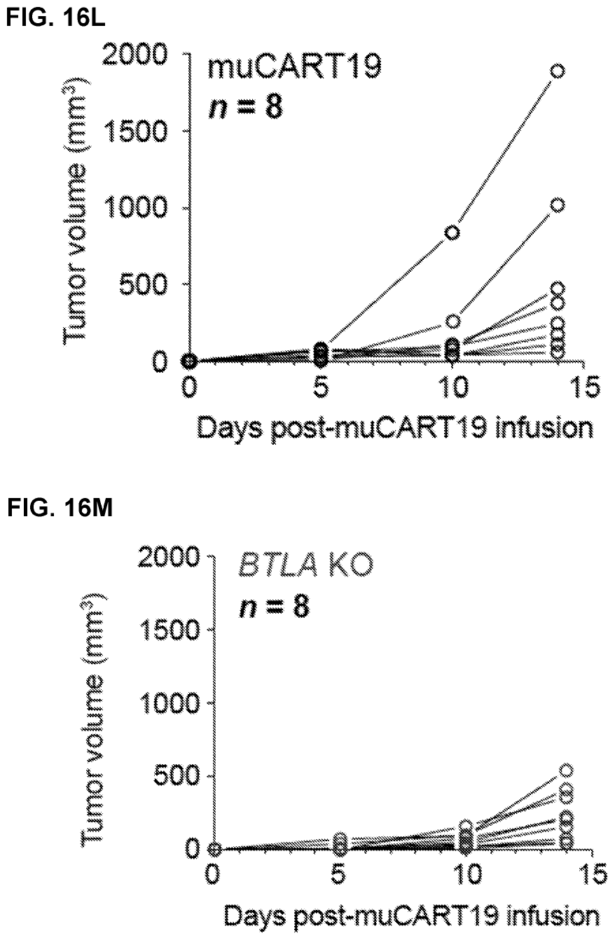

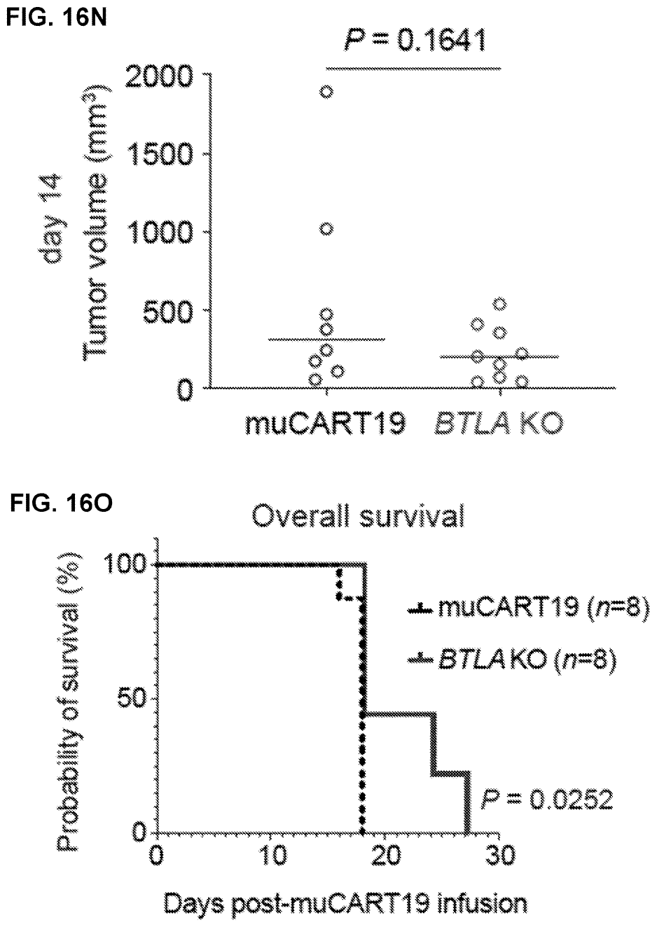

Given the advantages of BTLA KO in CAR T cells for multiple models of liquid cancers, and given the general evidence from TCGA described herein that low BTLA expression on T cell infiltrates in tumor biopsies associates with improved outcome, the role of BTLA modulation in CAR T against solid cancers was explored. Importantly, solid malignancies have been historically unresponsive to these treatments (Albelda, et al., Nature Reviews Clinical Oncology 21, 47-66 (2024); Newick, et al., Annu Rev Med 68, 139-152 (2017)). First, it was shown that across TCGA datasets and in particular cancers like ovarian (OV), prostate (PRAD), and melanoma (SKCM), HVEM is expressed more broadly than PD-L1 (Malissen, et al., Oncoimmunology 8, e1665976 (2019)), and demonstrates negative correlation with patient disease-free survival (FIG. 16A, FIG. 16B).

BTLA KO HER2-directed CAR T cells (clinically-employed scFv 4D5) (Zhao, et al., J Immunol 183, 5563-5574 (2009)) were generated, which demonstrated greater tumor control in models of ovarian cancer (SK-OV-3) and prostate cancer (PC-3) (FIG. 16D, FIG. 16E). Notably, PC-3 has been the subject of a study regarding improved tumor control upon administration of an anti-HVEM neutralizing antibody (Aubert, et al., Cancers (Basel) 13 (2021)). Thus, an HVEM-deficient PC-3 was also generated (FIG. 16F). No benefit was observed in killing from BTLA KO anti-HER2 CAR relative to WT against these target cells (FIG. 16G). Additionally, a mesothelin-positive pancreatic cancer model (AsPC-1) was evaluated. Notably, BTLA KO in anti-mesothelin CAR T cells (clinically-employed clone M5) did not confer any advantage against AsPC-1, also found to express low surface levels of HVEM (FIGS. 16H-16J).

Next, the effect of BTLA KO T cells was explored in a clinically-relevant melanoma model (DM-6). Anti-GP100 TCR cells were generated with simultaneous KO of the T cell receptor alpha locus (TRAC) and BTLA. To monitor tumor control, 4,000 GP100+ DM-6 cells were plated 24 hr prior to addition of 20,000 TCR+ T cells (E:T=5:1) and monitored via the Incucyte Live Cell Analysis System. Surprisingly, the BTLA KO anti-GP100 TCR T cells showed improved tumor control over control T cells in this melanoma model (FIG. 16K).

Finally, a syngeneic model of melanoma (B16-muCD19+) was utilized. Improved overall survival of mice treated with BTLA KO murine CAR T over mock-electroporated CAR T-treated mice was observed (P=0.0252) (FIGS. 16K-16O). Taken together, these results further suggest that BTLA-deficient CAR T should be tailored to tumor models that express HVEM. Overall, these studies demonstrate the utility of BTLA KO in CAR T cells across cancers with high HVEM expression extending beyond lymphoma.

### Clinical Role of BTLA in DLBCL Patients Receiving CART19

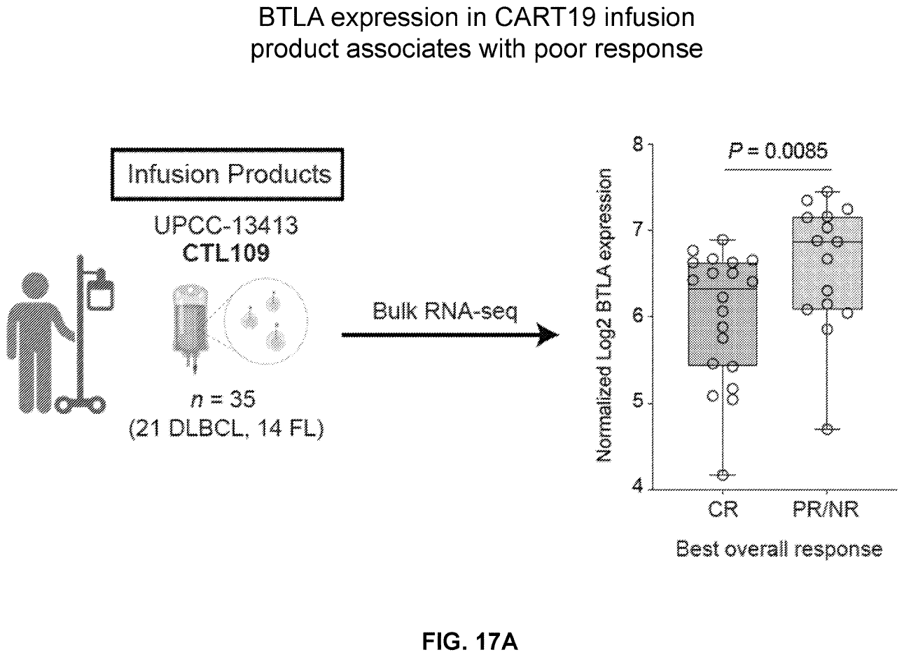

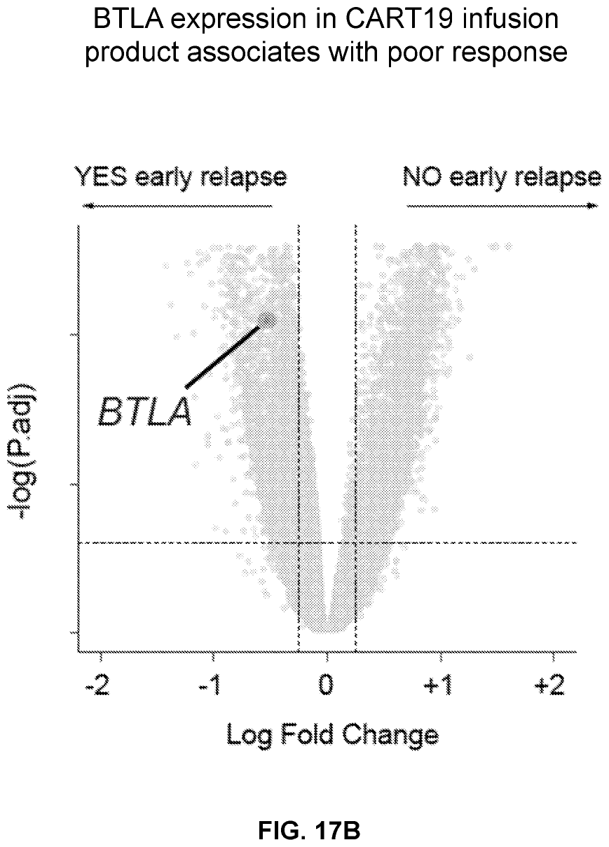

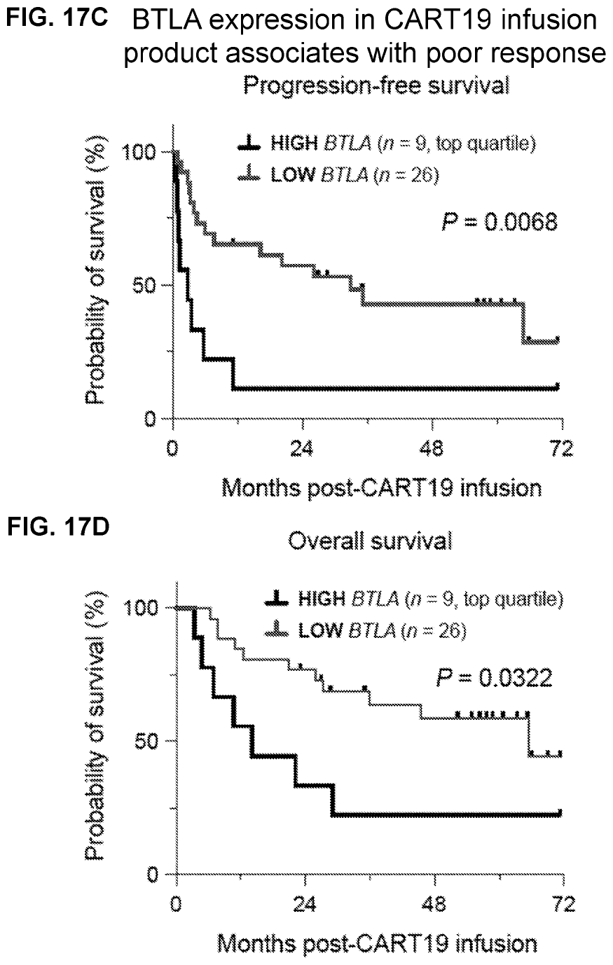

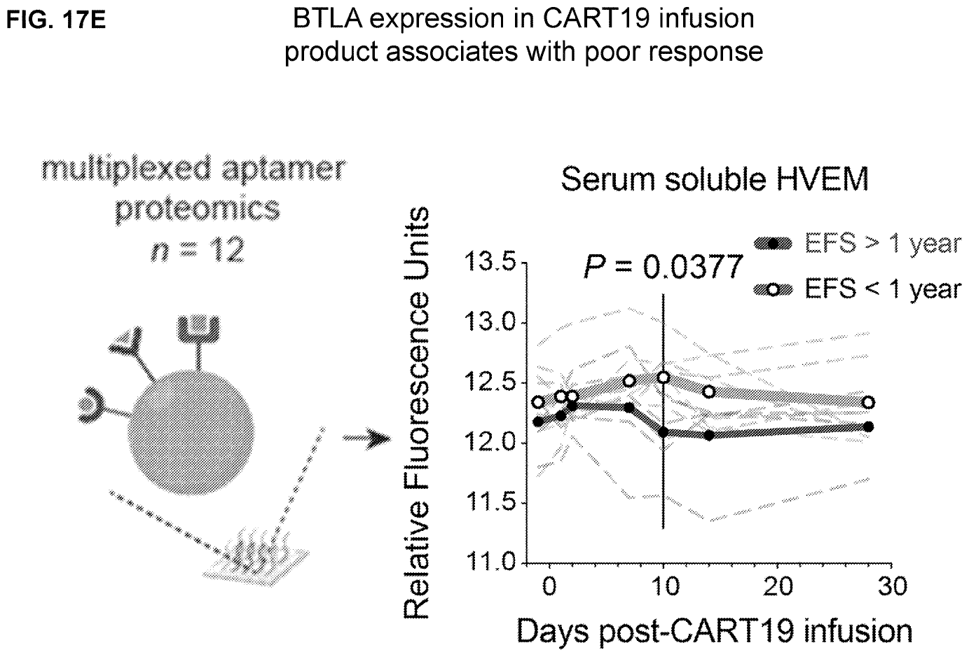

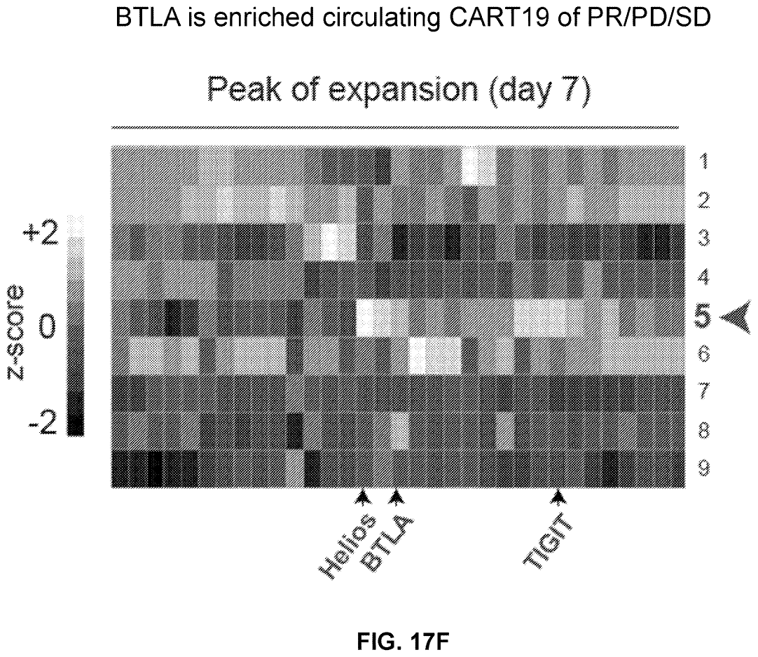

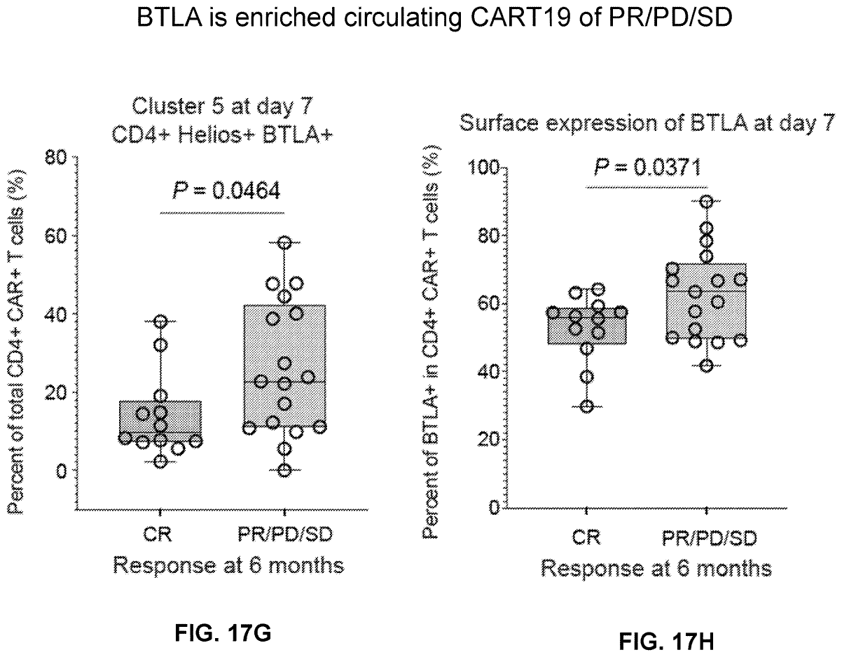

To examine the clinical relevance of BTLA expression in CAR T immunotherapy, bulk RNA sequencing was performed on the infusion products (IP) of n=35 patients receiving the 4-1BBζ CART19 product CTL019 (now known as tisagenlecleucel; NCT02030834 clinical trial (Chong, et al., N Engl J Med 384, 673-674 (2021); Schuster, S. J. et al., N Engl J Med 377, 2545-2554 (2017)) (FIG. 17A). The results indicate that BTLA expression was significantly lower in patients who achieved complete response to therapy and that heightened BTLA transcriptional expression in CAR T cells in the IP was associated with earlier relapse following CAR T cell infusion (FIG. 17A, FIG. 17B). High normalized BTLA expression in the IP correlated with poor patient progression-free and overall survival, supporting the role of BTLA as a mediator of CAR T failure (FIG. 17C, FIG. 17D). Of note, in these data, statistical correlation of BTLA expression to that of other characteristic IP markers of T cell dysfunction was found (e.g., LAG-3, HAVCR2) (Deng, et al., Nat Med 26, 1878-1887 (2020)). Furthermore, it is postulated herein that HVEM levels in the tumor would correlate with lower responses. Since there was no availability of HVEM expression in the tumor biopsies and since HVEM has a known soluble form (Heo, et al., Experimental & Molecular Medicine 44, 149-158 (2012)), serological levels of HVEM in the blood were studied. Notably, the results showed that elevated levels of soluble HVEM in the sera of patients during CART19 treatment correlated with shorter event-free survival (FIG. 17E). These data support the role of BTLA and HVEM as determinants of response to CART19 in patients with B cell lymphoma).

To further validate these findings at the proteomic-level, time-of-flight mass cytometry data were analyzed on the FACS-enriched CD28ζ CAR19+ T cells of n=32 LBCL patients (Good, Z. et al., Nature Medicine 28, 1860-1871 (2022)). The results indicate that BTLA was enriched in the pathologic Helios+CD57− CAR Treg subset identified by the authors that promote tumor progression and poor response (FIG. 17F, FIG. 17G). More broadly, it was found that heightened BTLA expression on the surface of circulating CD4+ CD28ζ CAR19 T cells at the peak of expansion (day 7) significantly correlates with poor outcome (FIG. 17H). These results support the role of BTLA in disabling CART19 and justify the novel approach described herein to ablate BTLA in CAR T cells.

Overall, the studies described herein highlight the significance of BTLA in T cells directed against various cancers and indicates that the BTLA-HVEM axis is a crucial immune checkpoint in these cancers.

### Example 2: T Cells Expressing an HVEM Variant

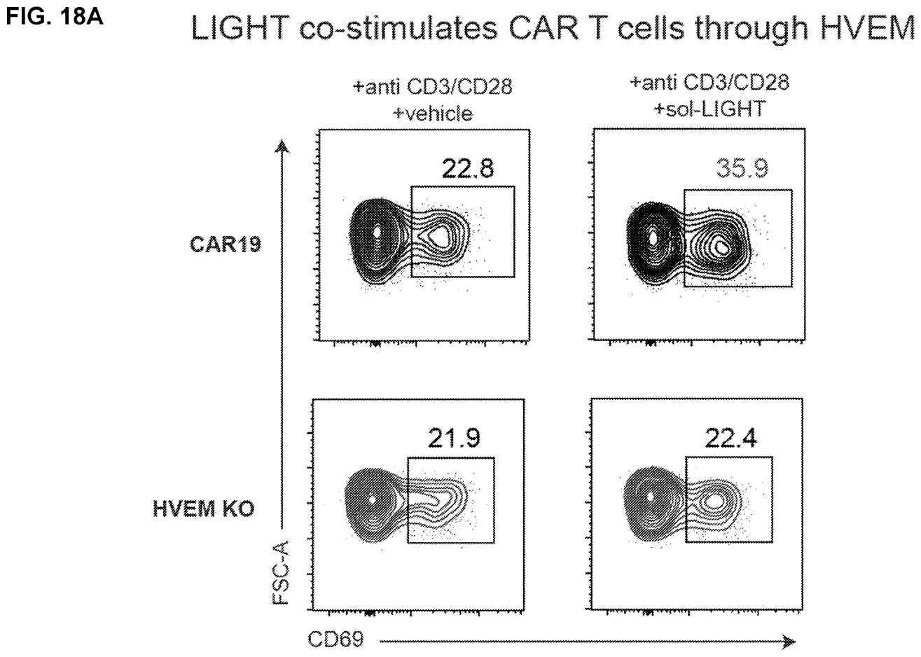

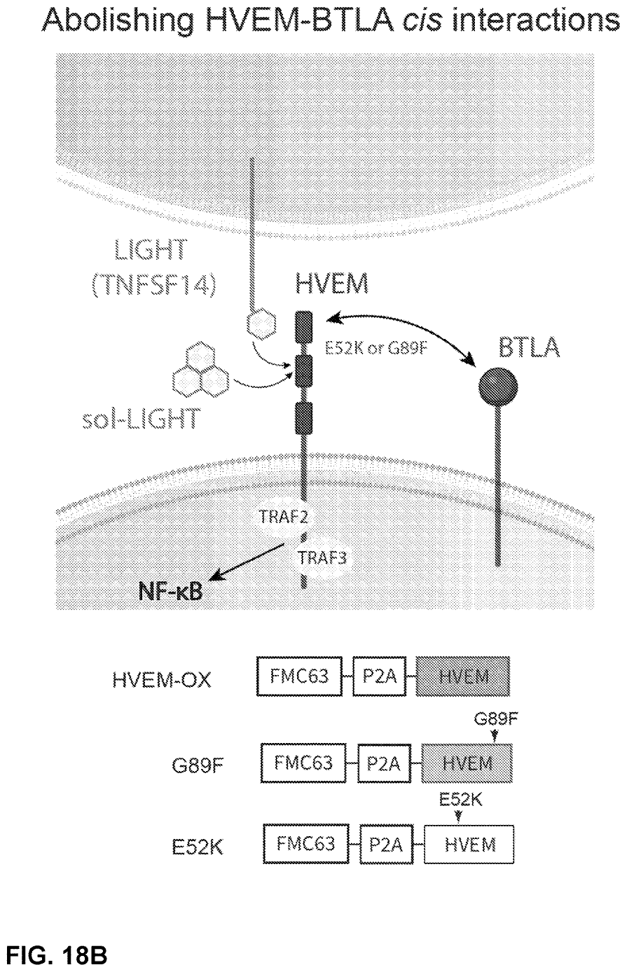

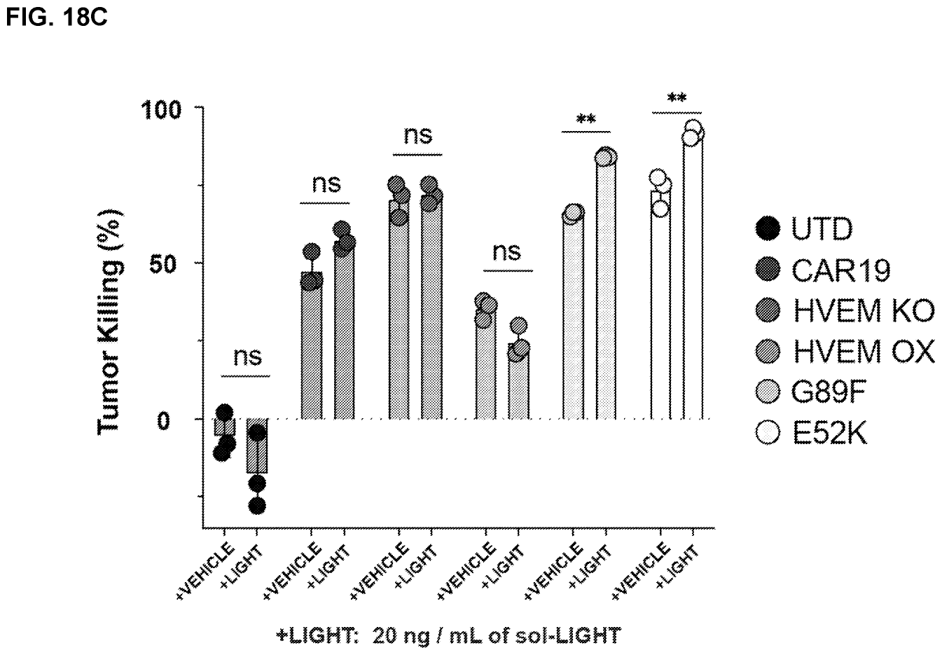

First, CAR19 T cells and HVEM KO CAR19T cells were used to demonstrate that LIGHT, a marker expressed on activated T cells, co-stimulates CAR T cells through HVEM (FIG. 18A).

Next, constructs were designed for expressing an anti-CD19 CAR and WT HVEM (“HVEM-OX”) or an HVEM switch receptor which abolishes HVEM-BTLA cis interactions but maintains HVEM binding to LIGHT (FIG. 18B). Two HVEM switch receptors, i.e., HVEM variants having amino acid point mutations E52K and G89F, respectively, in human HVEM were investigated. These HVEM variants reduce or eliminate HVEM binding to BTLA while maintaining or increasing HVEM binding to LIGHT (Shrestha, et al., Structure, 28(11):1197-1205.e2 (2020)). The HVEM-LIGHT interaction is thought to stimulate T-cells and to promote T cell persistence. The ability of the T cells expressing CAR19 and an HVEM switch receptor to kill DLBCL OCI-Ly18 tumor cells in vitro was investigated in the presence or absence of 20 ng/mL soluble LIGHT (FIG. 18C). Both HVEM switch receptors provided a significant enhancement in the cytotoxicity of the CAR19 T cells toward tumor cells in the presence of soluble LIGHT (FIG. 18C).

### ENUMERATED EMBODIMENTS

The following enumerated embodiments are provided, the numbering of which is not to be construed as designating levels of importance.

Embodiment 1: An immune cell genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM).

Embodiment 2: The immune cell of embodiment 1, further comprising a partial knockdown or knockout of expression of an endogenous BTLA gene.

Embodiment 3: The immune cell of any one of the preceding embodiments, further comprising a partial knockdown or knockout of expression of an endogenous HVEM gene.

Embodiment 4: The immune cell of any one of the preceding embodiments, wherein the knockout is mediated by a gene editing system.

Embodiment 5: The immune cell of any one of the preceding embodiments, wherein the knockout is mediated by a CRISPR system.

Embodiment 6: The immune cell of any one of the preceding embodiments, wherein the BTLA comprises reduced ability to bind SHP-1 and SHP-2, and wherein the HVEM comprises reduced ability to bind to BTLA.

Embodiment 7: The immune cell of any one of the preceding embodiments, wherein the immune cell comprises an expression vector comprising a first nucleotide sequence encoding the CAR or the TCR, a second nucleotide sequence encoding the BTLA or the HVEM, and a third nucleotide sequence comprising a ribosomal skipping sequence, wherein the third nucleotide sequence is located between the first and second nucleotide sequences.

Embodiment 8: The immune cell of any one of the preceding embodiments, wherein the immune cell comprises an expression vector comprising a first nucleotide sequence encoding the CAR or the TCR, and the immune cell further comprises a second nucleotide sequence encoding the BTLA or the HVEM, wherein the second nucleotide sequence is integrated into genomic DNA of the immune cell.

Embodiment 9: The immune cell of any one of the preceding embodiments, wherein the expression vector is a lentiviral vector.

Embodiment 10: The immune cell of any one of the preceding embodiments, wherein the second nucleotide sequence encoding the BTLA or the HVEM is integrated into genomic DNA of the immune cell via adenovirus-mediated gene transfer or CRISPR-mediated gene knock-in.

Embodiment 11: The immune cell of any one of the preceding embodiments, wherein the BTLA is a human BTLA variant comprising Y257F and Y282F amino acid substitutions.

Embodiment 12: The immune cell of any one of the preceding embodiments, wherein the HVEM is a human HVEM variant comprising E52K and/or G89F amino acid substitution(s).

Embodiment 13: The immune cell of any one of the preceding embodiments, wherein the immune cell is a T cell, an autologous cell, a human cell, or any combination thereof.

Embodiment 14: The immune cell of any one of the preceding embodiments, wherein the CAR comprises an extracellular antigen binding domain, a transmembrane domain, and an intracellular domain.

Embodiment 15: The immune cell of any one of the preceding embodiments, wherein the extracellular antigen binding domain comprises a single-chain variable fragment (scFv).

Embodiment 16: The immune cell of any one of the preceding embodiments, wherein the CAR or the TCR targets a tumor antigen.

Embodiment 17: The immune cell of any one of the preceding embodiments, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), gp100, HER2, HLA-A2, HPV E6, ICAM1, IL3Ra, IL13Ra2, LAGE-1, Lewis Y, LMP1 (EBV), MAGE-A1, MAGE-A3, MAGE-A4, MAGE-A6, MAGE-A10, MART-1, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PRAME, PSCA, PSMA, ROR1, ROR2, TIM-3, TM4SF1, TnMuc1, VEGFR2, and any combination thereof.

Embodiment 18: The immune cell of any one of the preceding embodiments, wherein the intracellular domain of the CAR comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 19: The immune cell of any one of the preceding embodiments, wherein the intracellular domain of the CAR comprises an intracellular signaling domain of a protein selected from the group consisting of a CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 20: The immune cell of any one of the preceding embodiments, wherein the intracellular domain of the CAR comprises a costimulatory domain of CD28, a costimulatory domain of 4-1BB, an intracellular signaling domain of CD3 zeta, or any combination thereof.

Embodiment 21: The immune cell of any one of the preceding embodiments, wherein the CAR targets CD19, CD30, or HER-2.

Embodiment 22: The immune cell of any one of the preceding embodiments, wherein the TCR targets gp100.

Embodiment 23: A pharmaceutical composition comprising a population of the immune cell of any one of the preceding embodiments and a pharmaceutically acceptable carrier.

Embodiment 24: The pharmaceutical composition of any one of the preceding embodiments, for use in a method of treating cancer in a subject.

Embodiment 25: A method of treating cancer in a subject in need thereof, the method comprising administering to the subject a therapeutically effective amount of immune cells genetically modified to express a chimeric antigen receptor (CAR) or a T cell receptor (TCR), wherein the immune cells comprise altered signaling of B and T lymphocyte attenuator (BTLA) and/or herpes virus entry mediator (HVEM).

Embodiment 26: The method of embodiment 24, wherein the immune cells further comprise a partial knockdown or knockout of expression of an endogenous BTLA gene.

Embodiment 27: The method of any one of the preceding embodiments, wherein the immune cells further comprise a partial knockdown or knockout of expression of an endogenous HVEM gene.

Embodiment 28: The method of any one of the preceding embodiments, wherein the knockout is mediated by a gene editing system.

Embodiment 29: The method of any one of the preceding embodiments, wherein the knockout is mediated by a CRISPR system.

Embodiment 30: The method of any one of the preceding embodiments, wherein the BTLA comprises reduced ability to bind SHP-1 and SHP-2, and wherein the HVEM comprises reduced ability to bind to BTLA.

Embodiment 31: The method of any one of the preceding embodiments, wherein the immune cells comprise an expression vector comprising a first nucleotide sequence encoding the CAR or the TCR, a second nucleotide sequence encoding the BTLA or the HVEM, and a third nucleotide sequence comprising a ribosomal skipping sequence, wherein the third nucleotide sequence is located between the first and second nucleotide sequences.

Embodiment 32: The method of any one of the preceding embodiments, wherein the immune cells comprise an expression vector comprising a first nucleotide sequence encoding the CAR or the TCR, and the immune cells further comprise a second nucleotide sequence encoding the BTLA or the HVEM, wherein the second nucleotide sequence is integrated into genomic DNA of the immune cell.

Embodiment 33: The method of any one of the preceding embodiments, wherein the expression vector is a lentiviral vector.

Embodiment 34: The method of any one of the preceding embodiments, wherein the second nucleotide sequence encoding the BTLA or the HVEM is integrated into genomic DNA of the immune cells via adenovirus-mediated gene transfer or CRISPR-mediated gene knock-in.

Embodiment 35: The method of any one of the preceding embodiments, wherein the BTLA is a human BTLA variant comprising Y257F and Y282F amino acid substitutions.

Embodiment 36: The method of any one of the preceding embodiments, wherein the HVEM is a human HVEM variant comprising E52K and/or G89F amino acid substitution(s).

Embodiment 37: The method of any one of the preceding embodiments, wherein the immune cells are T cells, autologous cells, human cells, or any combination thereof.

Embodiment 38: The method of any one of the preceding embodiments, wherein the CAR comprises an extracellular antigen binding domain, a transmembrane domain, and an intracellular domain.

Embodiment 39: The method of any one of the preceding embodiments, wherein the extracellular antigen binding domain comprises a single-chain variable fragment (scFv).

Embodiment 40: The method of any one of the preceding embodiments, wherein the CAR or the TCR targets a tumor antigen.

Embodiment 41: The method of any one of the preceding embodiments, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD 117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), gp100, HER2, HLA-A2, HPV E6, ICAM1, IL3Ra, IL13Ra2, LAGE-1, Lewis Y, LMP1 (EBV), MAGE-A1, MAGE-A3, MAGE-A4, MAGE-A6, MAGE-A10, MART-1, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PRAME, PSCA, PSMA, ROR1, ROR2, TIM-3, TM4SF1, TnMuc1, VEGFR2, and any combination thereof.

Embodiment 42: The method of any one of the preceding embodiments, wherein the intracellular domain of the CAR comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 43: The method of any one of the preceding embodiments, wherein the intracellular domain of the CAR comprises an intracellular signaling domain of a protein selected from the group consisting of a CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 44: The method of any one of the preceding embodiments, wherein the intracellular domain of the CAR comprises a costimulatory domain of CD28, a costimulatory domain of 4-1BB, an intracellular signaling domain of CD3 zeta, or any combination thereof.

Embodiment 45: The method of any one of the preceding embodiments, wherein the CAR targets CD19, CD30, or HER-2.

Embodiment 46: The method of any one of the preceding embodiments, wherein the TCR targets gp100.

Embodiment 47: The method of any one of the preceding embodiments, wherein the cancer is an HVEM-positive cancer.

Embodiment 48: The method of any one of the preceding embodiments, wherein the subject is a human.

### OTHER EMBODIMENTS

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An immune cell genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM).

2. The immune cell of claim 1, further comprising a partial knockdown or knockout of expression of an endogenous BTLA gene.

3. The immune cell of claim 2, further comprising a partial knockdown or knockout of expression of an endogenous HVEM gene.

4-5. (canceled)

6. The immune cell of claim 1, wherein the BTLA comprises reduced ability to bind SUP-1 and SHP-2, and wherein the HVEM comprises reduced ability to bind to BTLA.

7. The immune cell of claim 1, wherein the immune cell comprises an expression vector comprising a first nucleotide sequence encoding the CAR or the TCR, a second nucleotide sequence encoding the BTLA or the HVEM, and a third nucleotide sequence comprising a ribosomal skipping sequence, wherein the third nucleotide sequence is located between the first and second nucleotide sequences.

8. The immune cell of claim 1, wherein the immune cell comprises an expression vector comprising a first nucleotide sequence encoding the CAR or the TCR, and the immune cell further comprises a second nucleotide sequence encoding the BTLA or the HVEM, wherein the second nucleotide sequence is integrated into genomic DNA of the immune cell.

9-10. (canceled)

11. The immune cell of claim 1, wherein the BTLA is a human BTLA variant comprising Y257F and Y282F amino acid substitutions.

12. The immune cell of claim 1, wherein the HVEM is a human HVEM variant comprising E52K and/or G89F amino acid substitution(s).

13-22. (canceled)

23. A pharmaceutical composition comprising a population of the immune cell of claim 1 and a pharmaceutically acceptable carrier.

24. (canceled)

25. A method of treating cancer in a subject in need thereof, the method comprising administering to the subject a therapeutically effective amount of an immune cell genetically modified to express a chimeric antigen receptor (CAR) or a T cell receptor (TCR), wherein the immune cell comprises altered signaling of B and T lymphocyte attenuator (BTLA) and/or herpes virus entry mediator (HVEM).

26. The method of claim 25, wherein the immune cell further comprises a partial knockdown or knockout of expression of an endogenous BTLA gene.

27. The method of claim 26, wherein the immune cell further comprises a partial knockdown or knockout of expression of an endogenous HVEM gene.

28-29. (canceled)

30. The method of claim 25, wherein the BTLA comprises reduced ability to bind SUP-1 and SHP-2, and wherein the HVEM comprises reduced ability to bind to BTLA.

31. The method of claim 25, wherein the immune cell comprises an expression vector comprising a first nucleotide sequence encoding the CAR or the TCR, a second nucleotide sequence encoding the BTLA or the HVEM, and a third nucleotide sequence comprising a ribosomal skipping sequence, wherein the third nucleotide sequence is located between the first and second nucleotide sequences.

32. The method of claim 25, wherein the immune cell comprises an expression vector comprising a first nucleotide sequence encoding the CAR or the TCR, and the immune cell further comprises a second nucleotide sequence encoding the BTLA or the HVEM, wherein the second nucleotide sequence is integrated into genomic DNA of the immune cell.

33-34. (canceled)

35. The method of claim 25, wherein the BTLA is a human BTLA variant comprising Y257F and Y282F amino acid substitutions.

36. The method of claim 25, wherein the HVEM is a human HVEM variant comprising E52K and/or G89F amino acid substitution(s).

37-46. (canceled)

47. The method of claim 25, wherein the cancer is an HVEM-positive cancer.

48. The method of claim 25, wherein the subject is a human.


SEQUENCE LISTING (34 sequences)

SEQ ID NO: 1 - RNA, 20 nt, synthetic construct
  source 1..20: mol_type = other RNA
tgttagcacagtatttcaca

SEQ ID NO: 2 - RNA, 20 nt, synthetic construct
  source 1..20: mol_type = other RNA
caaggaggacgagtacccag

SEQ ID NO: 3 - protein, 289 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 4 - DNA, 867 bp, synthetic construct
  source 1..867: mol_type = other DNA
atgaaaacactcccggctatgttaggaaccggtaagctgttctgggtcttctttctcattccgtacttggatatttggaatattcacggcaaggagtcttgcgacgtgcaattgtacattaaacggcagagcgagcatagcatactggccggcgaccctttcgagctggagtgtcccgtcaagtattgcgccaatagaccacacgttacctggtgtaaattaaacggcactacttgcgttaagctggaagacaggcagaccagctggaaagaggaaaagaatatcagcttctttatcctgcacttcgagcctgtcttgccaaacgataacggtagttatagatgctcagccaacttccaatccaacctgatagagtcacattctaccaccctgtacgtaacggacgtcaagtcagctagtgagcgccctagtaaagatgagatggcctctcgcccttggcttctctaccgattgctcccactcggcgggttaccactgttaataaccacttgcttttgtttattttgttgtcttcgcagacatcaggggaaacagaacgagctgagcgataccgctggcagagagatcaatttggtcgatgcccatttgaaaagcgaacagaccgaggcctcaacacggcagaactcacaggtcttgctgtctgagaccggcatatacgacaacgatccggatctctgctttcggatgcaagagggatccgaggtatactccaacccctgtcttgaggagaataagcctggtatagtgtacgccagtcttaatcacagtgtgatcggtcctaattcccgcctcgcgcggaacgtcaaagaggcccctaccgagtacgctagtatttgcgtcagatct

SEQ ID NO: 5 - protein, 289 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVFASLNHSVIGPNSRLARNVKEAPTEYASICVRS

SEQ ID NO: 6 - protein, 289 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEFASICVRS

SEQ ID NO: 7 - protein, 289 aa, synthetic construct
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVFASLNHSVIGPNSRLARNVKEAPTEFASICVRS

SEQ ID NO: 8 - DNA, 867 bp, synthetic construct
  source 1..867: mol_type = other DNA
atgaaaacgcttcccgccatgctggggacaggaaagttattttgggtcttctttctcataccttacttggatatttggaatattcacggcaaggagagctgcgacgtccaattgtacattaaacggcagagtgagcatagtatactggctggcgaccctttcgagttggagtgtccagtaaagtattgcgccaatagaccacacgttacctggtgtaaattaaacggcactacctgcgtcaagctcgaggaccggcagaccagctggaaagaggaaaagaatatcagtttctttatcctgcacttcgagcccgtcttgccgaacgataacggcagctatagatgctccgctaacttccaatccaacctgatcgagtctcatagcactacactgtacgttaccgacgtgaagtccgctagtgagcggccttctaaagatgagatggcctctcgcccatggttgttgtacaggctgctccccctcggcgggctgcccttactgataacgacatgcttttgtttattttgttgtcttcgaagacatcaggggaaacagaacgagctgagcgataccgctgggcgagagatcaatctcgtggacgcccatctcaaaagcgaacagactgaggcttctactcggcagaactctcaggttctcctgtccgagaccggtatatacgacaacgatcccgatctctgctttcgcatgcaagagggatccgaggtgtacagtaacccttgtctcgaggagaataagcctgggatcgtctttgcctcattaaatcacagcgtgatcgggccaaatagccggctcgcgcgaaacgtcaaggaagctcctacggagtttgctagcatctgcgtacgatcc

SEQ ID NO: 9 - protein, 283 aa, synthetic construct
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 10 - DNA, 849 bp, synthetic construct
  source 1..849: mol_type = other DNA
atggaaccaccgggagattggggcccaccaccatggcgcagcacaccaaagacagatgttctccgcctcgttctttacctgacatttctcggtgctccttgttatgcacccgcattacctagttgtaaagaagatgaatatcccgtagggagtgaatgttgtcctaaatgttctcccggataccgggtcaaagaagcgtgtggtgaactcactggtaccgtatgcgagccttgtccacctggaacttatatcgctcatttgaacgggctgagtaaatgccttcaatgtcagatgtgcgatccggctatgggattgagggcctctaggaattgttcacggacggaaaatgcagtctgcgggtgttctcctgggcatttctgtattgtgcaagatggcgatcattgtgctgcatgtagggcatatgctacgagcagtcctgggcaaagagttcaaaagggcggaactgaaagccaagatacgctttgccaaaattgtcccccaggaacgtttagtccgaacggcacacttgaagagtgccaacatcaaacaaaatgttcctggttggtcactaaagcaggggcgggaacttcttcttctcattgggtttggtggttccttagcggcagtctggtgattgttatagtgtgttctaccgtcgggctgattatttgcgtaaagcggcggaaacctcggggagacgtggtgaaagttattgttagcgtacaacgcaagcgtcaagaagctgagggagaagctaccgtgatcgaagcattgcaagctccccctgatgtaacaaccgttgcagtagaagaaactattccgagctttactggccgatcacccaatcat

SEQ ID NO: 11 - protein, 283 aa, synthetic construct
MEPPGDWGPPPWRSTPKTDVLRLVLYLTFLGAPCYAPALPSCKEDEYPVGSKCCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 12 - DNA, 849 bp, synthetic construct
  source 1..849: mol_type = other DNA
atggaaccacctggggattggggcccgccaccatggagatcaacgccaaagacagatgtgctgagattggtactctacctgacttttctcggggcaccatgttatgcacctgcgctcccttcttgtaaagaagatgaatatcctgtcgggagtaaatgttgtccgaaatgttcacccgggtaccgggttaaagaagcatgtggcgaacttaccgggaccgtctgcgagccatgtccgccgggtacatatatcgcacatctgaacgggctctccaaatgccttcaatgtcagatgtgcgatcccgctatgggtcttagagcctctcgcaattgttcacgtaccgaaaatgctgtctgcggatgttcacccggacacttttgtatagtgcaagatggtgatcattgtgctgcttgtcgggcctatgctacaagcagtcccggtcaacgcgttcagaaaggcggaacagaatctcaagatacgctctgccaaaattgtccgcccggcacttttagccctaacggcacgttagaagagtgccaacatcaaacaaaatgttcctggctcgtcactaaagctggggccggcacttcaagttctcattgggtgtggtggttcttgagcggcagtctggtaattgtgatcgtgtgttcaactgtcgggctgataatttgcgttaagcggcgaaaaccccgcggggacgtcgtgaaagtcatagttagtgtgcaaagaaagaggcaagaagccgagggagaagctactgtaatagaagctcttcaagctcccccagatgttactactgttgctgtagaagaaaccatcccttcttttaccggccgttctcctaatcat

SEQ ID NO: 13 - protein, 283 aa, synthetic construct
MEPPGDWGPPPWRSTPKTDVLRLVLYLTFLGAPCYAPALPSCKEDEYPVGSECCPKCSPGYRVKEACGELTGTVCEPCPPGTYIAHLNFLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 14 - DNA, 849 bp, synthetic construct
  source 1..849: mol_type = other DNA
atggaaccacctggtgattggggccctcctccttggcgtagcactcccaagacggatgtactgagattagttctctacttgacctttctgggtgcgccatgttatgctccggcactcccctcttgtaaagaagatgaatatcccgttggaagcgaatgttgtccaaaatgttctcctgggtaccgagtcaaagaagcttgtggagaactcaccgggactgtttgcgagccttgtccacccggaacttatatcgctcatctgaactttctgagtaaatgccttcaatgtcagatgtgcgatcccgcaatgggacttagggcctcaagaaattgttctcggaccgaaaatgctgtctgcgggtgtagtcctgggcacttttgtattgttcaagatggtgatcattgtgcggcctgtagagcctatgcaacaagcagtccagggcaacgggtccagaaaggcggtactgaatcccaagatacattgtgccaaaattgtcccccaggaacattttcccctaacggcactctcgaagagtgccaacatcaaacaaaatgtagttggcttgtaactaaagcaggcgccggcacttcatcaagccattgggtctggtggttcctgagtggatctctggtgatagtgatcgtgtgtagcaccgtcggactcattatctgcgtcaagcgcaggaaacctcgaggcgacgtggtgaaagtcattgtgagtgtgcaacgaaagcggcaagaagccgagggagaagcaaccgtgatcgaagcgttgcaagctccacctgatgttactacagtcgcagtagaagaaacgattccaagttttacaggccgaagtcctaatcat

SEQ ID NO: 15 - protein, 283 aa, synthetic construct
MEPPGDWGPPPWRSTPKTDVLRLVLYLTFLGAPCYAPALPSCKEDEYPVGSKCCPKCSPGYRVKEACGELTGTVCEPCPPGTYIAHLNFLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 16 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - protein, 248 aa, synthetic construct
EVQLLESGGGLVQPGGSLRLSCAASGFTFSSYGMSWVRQAPGKGLEWVSSIYYYSGGTYYADSVKGRFTISRDNSKNTLYLQMNSLRAEDTAVYYCARNDYRNNSDFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYYPYYYVFGGGTKLTVLGGQAG

SEQ ID NO: 19 - protein, 247 aa, synthetic construct
EVQLLESGGGLVQPGGSLRLSCAASGFTFSYYYMSWVRQAPGKGLEWVSYIGGGGSGTYYADSVKGRFTISRDNSKNTLYLQMNSLRAEDTAVYYCARGPYYGYFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYPSYDSYVFGGGTKLTVLGGQAG

SEQ ID NO: 20 - protein, 249 aa, synthetic construct
EVKLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGGGSGGGGSGGGGSDIELTQSPKFMSTSVGDRVNVTCKASQNVGTNVAWFQQKPGQSPKVLIYSASYRYSGVPDRFTGSGSGTDFTLTISNVQSEDLAEYFCQQYHTYPLTFGGGTKLEIKGQAG

SEQ ID NO: 21 - DNA, 745 bp, synthetic construct
  source 1..745: mol_type = other DNA
gaggtgcagctgttggagtctgggggaggcttggtacagcctggggggtccctgagactctcctgtgcagcctctggattcacctttagctcttatggtatgagctgggtccgccaggctccagggaaggggctggagtgggtctcatctatctactactactctggtggtacgtattacgctgattctgtaaaaggtcggttcaccatctccagagacaattccaagaacacgctgtatctgcaaatgaacagcctgagagccgaggacacggccgtgtattactgtgcgcgtaacgactaccgtaacaactctgacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactacccgtactactatgtcttcggcggaggcaccaagctgacggtcctaggtggccaggccggcc

SEQ ID NO: 22 - DNA, 742 bp, synthetic construct
  source 1..742: mol_type = other DNA
gaggtgcagctgttggagtctgggggaggcttggtacagcctggggggtccctgagactctcctgtgcagcctctggattcacctttagctactattacatgagctgggtccgccaggctccagggaaggggctggagtgggtctcatacatcggtggtggtggttctggtacgtattacgctgattctgtaaaaggtcggttcaccatctccagagacaattccaagaacacgctgtatctgcaaatgaacagcctgagagccgaggacacggccgtgtattactgtgcgcgtggtccgtactacggttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacccgtcttacgactcttatgtcttcggcggaggcaccaagctgacggtcctaggtggccaggccggcc

SEQ ID NO: 23 - DNA, 748 bp, synthetic construct
  source 1..748: mol_type = other DNA
gaggtgaagcttcaacagtccggagcagaactcgcccgcccgggcgctagcgtgaaaatgagctgcaaggcctctggatataccttcaccacctataccattcactgggtgagacagagacctggccatgacctggagtggatcggctatataaacccaagcagtggctactccgactacaatcagaatttcaaaggcaaaacaacactgaccgcggacaagtcttcaaatactgcctacatgcagctgaactctctgacaagcgaggattcagcagtgtattactgcgctaggcgggctgactacggcaactacgagtatacctggtttgcatactggggccagggaacaacggttacagtgtcctcaggcggcggagggagcgggggtggtgggagtggaggcggggggagtgatatcgaactgacgcagtcacctaagtttatgagcacatccgtgggggatcgagtcaatgtcacgtgtaaggccagccagaatgtaggcactaacgtggcctggtttcaacagaagcccggtcaaagccccaaagttctgatctacagcgcttcctataggtacagtggcgtgccagatagattcaccggatccggaagcggaaccgatttcactctcactatttcaaacgttcaatcggaagatttggccgagtatttttgtcagcagtatcacacataccccttaactttcggtggcggtactaagctggaaattaaaggccaggccggcc

SEQ ID NO: 24 - protein, 493 aa, synthetic construct
MALPVTALLLPLALLLHAARPEVQLLESGGGLVQPGGSLRLSCAASGFTFSSYGMSWVRQAPGKGLEWVSSIYYYSGGTYYADSVKGRFTISRDNSKNTLYLQMNSLRAEDTAVYYCARNDYRNNSDFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYYPYYYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 492 aa, synthetic construct
MALPVTALLLPLALLLHAARPEVQLLESGGGLVQPGGSLRLSCAASGFTFSYYYMSWVRQAPGKGLEWVSYIGGGGSGTYYADSVKGRFTISRDNSKNTLYLQMNSLRAEDTAVYYCARGPYYGYFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYPSYDSYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 494 aa, synthetic construct
MALPVTALLLPLALLLHAARPEVKLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGGGSGGGGSGGGGSDIELTQSPKFMSTSVGDRVNVTCKASQNVGTNVAWFQQKPGQSPKVLIYSASYRYSGVPDRFTGSGSGTDFTLTISNVQSEDLAEYFCQQYHTYPLTFGGGTKLEIKGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1479 bp, synthetic construct
  source 1..1479: mol_type = other DNA
atggccttaccagtgaccgccttgctcctgccgctggccttgctgctccacgccgccaggccggaggtgcagctgttggagtctgggggaggcttggtacagcctggggggtccctgagactctcctgtgcagcctctggattcacctttagctcttatggtatgagctgggtccgccaggctccagggaaggggctggagtgggtctcatctatctactactactctggtggtacgtattacgctgattctgtaaaaggtcggttcaccatctccagagacaattccaagaacacgctgtatctgcaaatgaacagcctgagagccgaggacacggccgtgtattactgtgcgcgtaacgactaccgtaacaactctgacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactacccgtactactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 28 - DNA, 1476 bp, synthetic construct
  source 1..1476: mol_type = other DNA
atggccttaccagtgaccgccttgctcctgccgctggccttgctgctccacgccgccaggccggaggtgcagctgttggagtctgggggaggcttggtacagcctggggggtccctgagactctcctgtgcagcctctggattcacctttagctactattacatgagctgggtccgccaggctccagggaaggggctggagtgggtctcatacatcggtggtggtggttctggtacgtattacgctgattctgtaaaaggtcggttcaccatctccagagacaattccaagaacacgctgtatctgcaaatgaacagcctgagagccgaggacacggccgtgtattactgtgcgcgtggtccgtactacggttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacccgtcttacgactcttatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 29 - DNA, 1482 bp, synthetic construct
  source 1..1482: mol_type = other DNA
atggccttaccagtgaccgccttgctcctgccgctggccttgctgctccacgccgccaggccggaggtgaagcttcaacagtccggagcagaactcgcccgcccgggcgctagcgtgaaaatgagctgcaaggcctctggatataccttcaccacctataccattcactgggtgagacagagacctggccatgacctggagtggatcggctatataaacccaagcagtggctactccgactacaatcagaatttcaaaggcaaaacaacactgaccgcggacaagtcttcaaatactgcctacatgcagctgaactctctgacaagcgaggattcagcagtgtattactgcgctaggcgggctgactacggcaactacgagtatacctggtttgcatactggggccagggaacaacggttacagtgtcctcaggcggcggagggagcgggggtggtgggagtggaggcggggggagtgatatcgaactgacgcagtcacctaagtttatgagcacatccgtgggggatcgagtcaatgtcacgtgtaaggccagccagaatgtaggcactaacgtggcctggtttcaacagaagcccggtcaaagccccaaagttctgatctacagcgcttcctataggtacagtggcgtgccagatagattcaccggatccggaagcggaaccgatttcactctcactatttcaaacgttcaatcggaagatttggccgagtatttttgtcagcagtatcacacataccccttaactttcggtggcggtactaagctggaaattaaaggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 30 - DNA, 8787 bp, synthetic construct
  source 1..8787: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattccacgtcgacttatgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccactagtgccaccatggccttaccagtgaccgccttgctcctgccgctggccttgctgctccacgccgccaggccggaggtgcagctgttggagtctgggggaggcttggtacagcctggggggtccctgagactctcctgtgcagcctctggattcacctttagctcttatggtatgagctgggtccgccaggctccagggaaggggctggagtgggtctcatctatctactactactctggtggtacgtattacgctgattctgtaaaaggtcggttcaccatctccagagacaattccaagaacacgctgtatctgcaaatgaacagcctgagagccgaggacacggccgtgtattactgtgcgcgtaacgactaccgtaacaactctgacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactacccgtactactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgttgattaattaactcgagctcgagtctagagggcccgcggttcgaaggtaagcctatccctaaccctctcctcggtctcgattctacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 31 - DNA, 8784 bp, synthetic construct
  source 1..8784: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattccacgtcgacttatgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccactagtgccaccatggccttaccagtgaccgccttgctcctgccgctggccttgctgctccacgccgccaggccggaggtgcagctgttggagtctgggggaggcttggtacagcctggggggtccctgagactctcctgtgcagcctctggattcacctttagctactattacatgagctgggtccgccaggctccagggaaggggctggagtgggtctcatacatcggtggtggtggttctggtacgtattacgctgattctgtaaaaggtcggttcaccatctccagagacaattccaagaacacgctgtatctgcaaatgaacagcctgagagccgaggacacggccgtgtattactgtgcgcgtggtccgtactacggttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacccgtcttacgactcttatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgttgattaattaactcgagctcgagtctagagggcccgcggttcgaaggtaagcctatccctaaccctctcctcggtctcgattctacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 32 - DNA, 8790 bp, synthetic construct
  source 1..8790: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattccacgtcgacttatgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccactagtgccaccatggccttaccagtgaccgccttgctcctgccgctggccttgctgctccacgccgccaggccggaggtgaagcttcaacagtccggagcagaactcgcccgcccgggcgctagcgtgaaaatgagctgcaaggcctctggatataccttcaccacctataccattcactgggtgagacagagacctggccatgacctggagtggatcggctatataaacccaagcagtggctactccgactacaatcagaatttcaaaggcaaaacaacactgaccgcggacaagtcttcaaatactgcctacatgcagctgaactctctgacaagcgaggattcagcagtgtattactgcgctaggcgggctgactacggcaactacgagtatacctggtttgcatactggggccagggaacaacggttacagtgtcctcaggcggcggagggagcgggggtggtgggagtggaggcggggggagtgatatcgaactgacgcagtcacctaagtttatgagcacatccgtgggggatcgagtcaatgtcacgtgtaaggccagccagaatgtaggcactaacgtggcctggtttcaacagaagcccggtcaaagccccaaagttctgatctacagcgcttcctataggtacagtggcgtgccagatagattcaccggatccggaagcggaaccgatttcactctcactatttcaaacgttcaatcggaagatttggccgagtatttttgtcagcagtatcacacataccccttaactttcggtggcggtactaagctggaaattaaaggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgttgattaattaactcgagctcgagtctagagggcccgcggttcgaaggtaagcctatccctaaccctctcctcggtctcgattctacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 33 - RNA, 23 nt, synthetic construct
  source 1..23: mol_type = other RNA
ggcgccctggccagtcgtctggg

SEQ ID NO: 34 - RNA, 20 nt, synthetic construct
  source 1..20: mol_type = other RNA
agctgagggctaacctcaag